(19) United States
(12) Patent Application Publication
(10) Pub. No.: US 2016/0263087 A1
(43) Pub. Date: Sep. 15, 2016

(54) NOVEL 4-(INDOL-3-YL)-PYRAZOLE DERIVATIVES, PHARMACEUTICAL COMPOSITIONS AND METHODS FOR USE

(71) Applicant: ITEOS THERAPEUTICS, Charleroi (BE)

(72) Inventors: Stefano Crosignani, Nivelles (BE); Samdra Cauwenberghs, Halle (BE); Frederik Deroose, Destelbergen (BE); Gregory Driessens, Watermael-Boitsfort (BE)

(21) Appl. No.: 15/034,380

(22) PCT Filed: Nov. 7, 2014
(86) PCT No.: PCT/EP2014/074099

§ 371 Date: May 4, 2016

(30) Foreign Application Priority Data
Nov. 8, 2013 (EP) 13192224.7
Nov. 3, 2014 (BE) 2014/0845

(51) Int. Cl. A61K 31/4155; A61K 31/501; A61K 31/5377; A61K 31/454; A61K 31/4178; A61K 31/496

(52) U.S. Cl. CPC A61K 31/4155; A61K 31/4178; A61K 31/496; A61K 31/5377; A61K 31/454; A61K 31/501

(57) ABSTRACT

4-(Indol-3-yl)-pyrazole derivative compounds of Formula I or pharmaceutically acceptable enantiomers, salts or solvates thereof are provided. Further provided is the use of the compounds of Formula I as TDO2 inhibitors. Also provided herein is use of the compounds of Formula I for the treatment and/or prevention of cancer, neurodegenerative disorders such as Parkinson's disease, Alzheimer's disease and Huntington's disease, chronic viral infections such as HCV and HIV, depression, and obesity.

### FIELD OF INVENTION

The present invention relates to novel 4-(indol-3-yl)-pyrazole derivatives, including pharmaceutically acceptable enantiomers, salts and solvates thereof. Compounds of the invention are inhibitors of TDO2 (tryptophan 2,3-dioxygenase) and are useful as therapeutic compounds, particularly in the treatment and/or prevention of cancers.

### BACKGROUND OF INVENTION

Two decades after the importance of tryptophan catabolism for maintaining the immune privilege of the placenta was discovered (Munn, D. H. et al., Science, 1998, 281, 1191-1193), increasing evidence is extending its biological relevance beyond immune tolerance to non-self. According to the generally accepted concept, tryptophan, an essential amino acid, is catabolized in the local microenvironment of tumors, immune-privileged sites, or sites of inflammation (Mellor A L and Munn D H., Nat Rev Immunol, 2008, 8, 74-80). In these tissues, cancer cells, immune cells, or specialized epithelial cells (e.g., syncytiotrophoblasts in the placenta) create an immunosuppressive environment in tumors that shut down antitumor immune responses in tumors and in tumor-draining lymph nodes by inducing T-cell anergy and apoptosis through depletion of tryptophan and accumulation of immunosuppressive tryptophan catabolites (Munn D H et al., J Exp Med., 1999, 189, 1363-1372; Fallarino F et al., Cell Death Differ., 2002, 9, 1069-1077).

It has recently been discovered that a key enzyme in tryptophan catabolism, tryptophan 2,3-dioxygenase (TDO2), which is considered responsible for regulating systemic tryptophan levels in the liver, is constitutively expressed in a wide variety of cancers, such as for example in bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma, pancreatic carcinoma (Pilotte L et al., Proc Natl Acad Sci USA, 2012, 109(7), 2497-502).

TDO2 expression in tumor cells prevents tumor surveillance by the immune system and thus prevents tumor rejection by locally degrading tryptophan (Opitz C A et al., Nature, 2011, 478(7368), 197-203). The first evidence for this was provided through inhibition of TDO2 by a small molecule which inhibited tumor growth in a P815 mastocytoma tumor model with a prophylactic vaccination approach (Pilotte L et al., Proc Natl Acad Sci USA, 2012, 109(7), 2497-502). Here, P815mTDO2 expressing tumors were rejected less in comparison to P815 tumors transfected with an empty vector, clearly demonstrating a growth benefit for TDO2 expressing tumors. Inhibition with a TDO2 inhibitor strongly decreased tumor growth in P815mTDO2 implanted tumors. Antitumor activity with the TDO2 inhibitor was equally observed in the P815 control implanted tumors negative for TDO2, thus providing evidence for an effect of TDO2 expressed in the immune system of the animal. These experiments for the first time provided clear evidence for a role of TDO2 in regulating tumor growth through expression in the cancer cell as well as immune compartment.

In line with its expression profile in liver, TDO2 was found predominantly in hepatocellular carcinoma (HCC) (Pilotte L et al., Proc Natl Acad Sci USA, 2012, 109(7), 2497-502). Inhibition of tryptophan catabolism and thus restoration of tryptophan concentration and decreased production of downstream metabolites could prove beneficial in the context of liver disease progressing to the stage of liver carcinoma: (i) several reports have shown evidence that increased availability of tryptophan through supplementation is beneficial for eg cirrhotic livers allowing the direct use of tryptophan for protein synthesis (Ohta et al., Amino Acids, 1996, 10(4), 369-78); (ii) there is a correlation between increased downstream serum tryptophan metabolites, such as quinolinic acid, and hepatic dysfunction in patients with liver cirrhosis (Landou et al., Hum Immunol, 2013, 74(1), 60-6) (iii) increased secretion of another tryptophan metabolite, indole-3-lactic acid, has been associated with alcohol-induced liver disease in mice (Manna et al., J Proteome Res, 2011, 10(9), 4120-33). In the context of liver carcinoma itself, the very high RNA expression is a good indication for therapeutic evaluation of TDO2 inhibitors (Pilotte L et al., Proc Natl Acad Sci USA, 2012, 109(7), 2497-502). The above thus provides a clear rationale for TDO2 activity modulation in the control of liver tumor development.

In addition to expression in liver, TDO2 is expressed in neurons, microglia and astrocytes and the potential benefit of TDO2 inhibition in the context of glioma was shown in another animal model. Platten and collaborators demonstrated that the tryptophan catabolite kynurenine produced by TDO expressed in the tumor cells suppresses antitumour immune responses and promotes tumor-cell survival and motility through the AHR in an autocrine/paracrine fashion (Opitz C A et al., Nature, 2011, 478(7368), 197-203). The TDO-AHR pathway is active in human brain tumours and is associated with malignant progression and poor survival. Further evidence came from the accumulation of a downstream metabolite, quinolinic acid which accumulates in human gliomas and was associated with a malignant phenotype (Sahm et al., Cancer Res, 2013, 73(11), 3225-34). Here tryptophan catabolism was shown to occur in microglia cells as well. The above data thus provides evidence for TDO2 targeting in glioma with brain-penetrant small molecules.

Other tumor types in which TDO2 mRNA was found are breast carcinoma, bladder, renal cell, pancreatic, colorectal, head & neck carcinoma and lung carcinoma as well as melanoma thus broadening the scope of TDO2 targeting beyond HCC and glioma (Pilotte L et al., Proc Natl Acad Sci USA, 2012, 109(7), 2497-502).

The enhanced Tryptophan degradation observed in patients with gynaecological cancers (ovarian carcinoma, cervical cancer, endometrial cancer) provides additional rationale for TDO2 targeting in those cancers (Sperner-Unterweger B et al, Immunology, 2011, 216 (3); 296-301).

The tryptophan catabolism in some cancers might be also increased by the expression of indoleamine 2,3-dioxygenasel (IDO1) by tumor cells (Uyttenhove, C. et al., Nat. Med., 2003, 9, 1269-1274). Because tryptophan catabolism is induced by inflammatory mediators, notably IFN-gamma, it is thought to represent an endogenous mechanism that restricts excessive immune responses, thereby preventing immunopathology. However in the context of cancer, there is strong evidence that suppression of antitumor immune responses in precancerous lesions and established cancers by tryptophan catabolism promotes tumor growth, which would make such catabolism an attractive target for therapeutic intervention (Dolugie E and Frederick R., Expert Opin Ther Pat., 2013, 23(10), 1367-81). Hence, a considerable effort is being made to identify selective and efficient inhibitors of tryptophan catabolism to enhance the efficacy of conventional chemotherapy, immune checkpoints (Holmgaard R B et al., J Exp Med., 2013, 210(7), 1389-402) or therapeutic vaccines.

Some TDO2 inhibitors were proposed in WO2010/008427 and by Dolusic, E. et al. (Dolusic et al., J. Med. Chem., 2011, 54, 5320-5334), however either their affinity for the target is limited, or their pharmacokinetic properties are not suitable for development as a drug for human use.

Therefore, there is a need for new TDO2 inhibitors with improved efficacy for cancer treatment and/or prevention.

The present invention provides new TDO2 inhibitors which may be administered to any patient diagnosed with cancer, or any subject being at risk of developing a cancer.

### DETAILED DESCRIPTION

Compounds

This invention relates to compounds of Formula I:

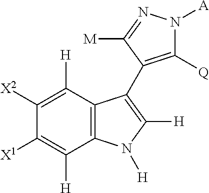

and pharmaceutically acceptable enantiomers, salts and solvates thereof, wherein:
X¹ and X² represent each independently H, halogen or haloalkyl, preferably H, F or CF₃, more preferably H or F;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl; preferably M and Q represent each independently H, methyl or CF₃, more preferably H or methyl;
A represents:
   a hydrogen atom;
   aryl, optionally substituted with halogen, hydroxyl, nitro, amido, carboxy, amino, cyano, haloalkoxy, haloalkyl, alkyl;
   heteroaryl, optionally substituted with halogen, hydroxyl, nitro, amido, carboxy, amino, cyano, haloalkoxy, haloalkyl, alkyl; preferably substituted or unsubstituted pyridyl or pyridazine, more preferably substituted or unsubstituted pyridyl;
   C1-C10 alkyl, linear or branched, optionally substituted with up to three substituents selected from the group comprising halogen, hydroxyl, COOR¹, CONR¹R², NR¹COR², NR¹R², SO₂R¹, SO₂NR¹R², NR¹SO₂R², SOR¹, wherein R¹ and R² represent each independently a hydrogen atom or a group, optionally substituted, selected from C1-C6 alkyl, aryl, heteroaryl, amino;
   heterocyclyl, preferably selected from azetidine, piperidine, morpholine, piperazine, tetrahydrofurane, tetrahydropyrane, tetrahydro-thiopyran-dioxide, dioxane, imidazolidinone, pyrrolidine, pyrrolidinone; optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me;
   C1-C3 alkyl-heterocyclyl, preferably selected from azetidine, piperidine, morpholine, piperazine, tetrahydrofurane, tetrahydropyrane, tetrahydro-thiopyran-dioxide, dioxane, imidazolidinone, pyrrolidine, pyrrolidinone; wherein both the C1-C3 alkyl and the heterocyclyl are optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group, optionally substituted, selected from C1-C6 alkyl, cycloalkyl, alkene, aryl, heteroaryl and amino, optionally substituted by one or more groups selected from halogen, hydroxyl, alkoxy, COOH, amino, SO₂Me;
   cycloalkyl, preferably cyclobutane or cyclohexyl, optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me;
   C1-C3 alkyl-cycloalkyl, optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a group selected from C1-C6 alkyl, cycloalkyl, alkene, aryl, heteroaryl and amino, optionally substituted by one or more groups selected from halogen, hydroxyl, alkoxy, COOH, amino, SO₂Me.

According to one embodiment, compounds of the invention are of Formula I as defined above, under the condition that the compound of Formula I is not:
2-(4-(1H-indol-3-yl)-3,5-dimethyl-1H-pyrazol-1-yl)ethanamine
3-(1-(tert-butyl)-1H-pyrazol-4-yl)-1H-indole
3-(1-ethyl-1H-pyrazol-4-yl)-1H-indole
3-(1-methyl-1H-pyrazol-4-yl)-1H-indole
3-(1-(4-fluorophenyl)-1H-pyrazol-4-yl)-1H-indole
3-(1-(4-chlorophenyl)-1H-pyrazol-4-yl)-1H-indole
3-(1-(4-bromophenyl)-1H-pyrazol-4-yl)-1H-indole
3-(1-(4-methoxyphenyl)-1H-pyrazol-4-yl)-1H-indole
3-(1-(p-tolyl)-1H-pyrazol-4-yl)-1H-indole
3-(1-phenyl-1H-pyrazol-4-yl)-1H-indole
3-(1H-pyrazol-4-yl)-1H-indole
4-(1H-indol-3-yl)-1H-pyrazol-3-ol.

According to a specific embodiment, in Formula I, group A is not H. According to a another specific embodiment, when X¹, X² and Q are hydrogen atoms in formula I, then, group A is not H.

According to a specific embodiment, in Formula I, group A is not an optionally substituted aryl group, more specifically, group A is not an optionally substituted phenyl group. According to a preferred specific embodiment, when X¹, X², M and Q are hydrogen atoms in formula I, then, group A is not an optionally substituted aryl group, more specifically, group A is not an optionally substituted phenyl group.

According to a specific embodiment, in Formula I, group A is not a C1-C10 alkyl, preferably, group A is not a C1-C4 alkyl, more preferably, group A is not methyl, ethyl or butyl. According to a preferred specific embodiment, when X¹, X², M and Q are hydrogen atoms in formula I, then, group A is not is not a C1-C10 alkyl, preferably, group A is not a C1-C4 alkyl.

According to a specific embodiment, in Formula I, group A is not a substituted C1-C10 alkyl, preferably, group A is not a substituted C1-C4 alkyl, more preferably, group A is not a substituted ethyl. According to a preferred specific embodiment, when X¹ and X² are hydrogen atoms and when M and Q are methyl groups in formula I, then, group A is not a substituted C1-C10 alkyl, preferably, group A is not a substituted C1-C4 alkyl, more preferably, group A is not a substituted ethyl.

In one embodiment, preferred compounds of Formula I are those of Formula Ia:

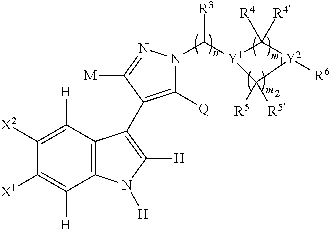

and pharmaceutically acceptable enantiomers, salts and solvates thereof, wherein:
X¹ and X² represent each independently H, halogen or haloalkyl, preferably H, F or CF₃, more preferably H or F;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl; preferably M and Q represent each independently H, methyl or CF₃, more preferably H or methyl;
n represents an integer equal to 0, 1, 2 or 3;
m₁ and m₂ represent each independently an integer equal to 1 or 2;
Y¹ and Y² represent each independently CR⁷, N, O, SO₂, wherein R⁷ represents H or hydroxyl;
R³ represents H, alkyl;
R⁴, R^{4′}, R⁵ and R^{5′} represent each independently H, hydroxyl, alkyl, alkoxy, haloalkyl or R⁴ and R^{4′} form together an oxo moiety or R⁵ and R^{5′} form together an oxo moiety;
R⁶ is absent or represents H, alkyl, the alkyl group being optionally substituted by one or more groups selected from halogen, hydroxyl or COOH; cycloalkyl, halogen, hydroxyl, oxo, COR¹, COOR¹, CONR¹R², NR¹COR², NR¹R², SO₂R¹, SO₂NR¹R², NRSO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me.

In one embodiment, preferred compounds of Formula I are those of Formula Ia-1:

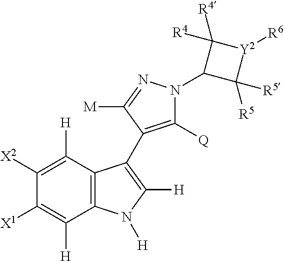

and pharmaceutically acceptable enantiomers, salts and solvates thereof, wherein:
X¹ and X² represent each independently H or F;
M and Q represent each independently H, C1-C6 alkyl optionally substituted by one or more halogen;
Y² represents N or CH;
R⁴, R^{4′}, R⁵ and R^{5′} represent each independently H, hydroxyl, alkyl, alkoxy, haloalkyl or R⁴ and R^{4′} form together an oxo moiety or R⁵ and R^{5′} form together an oxo moiety, preferably R⁴, R^{4′}, R⁵ and R^{5′} represent H or oxo;
R⁶ represents H, alkyl, the alkyl group being optionally substituted by one or more groups selected from halogen, hydroxyl or COOH; cycloalkyl, halogen, hydroxyl, oxo, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me; preferably R⁶ represents H, COOH, COMe, CONH₂, CONHMe.

In one embodiment, preferred compounds of Formula I are those of Formula Ia-2:

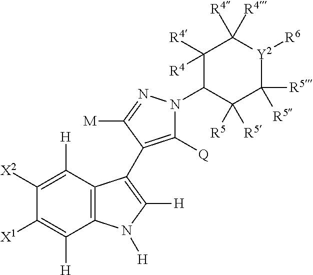

and pharmaceutically acceptable enantiomers, salts and solvates thereof, wherein:
X¹ and X² represent each independently H or F;
M and Q represent each independently H, C1-C6 alkyl optionally substituted one or more alogen;
Y² represents N or CH; preferably Y is N;
R⁴, R^{4′}, R^{4″}, R^{4′″}, R⁵, R^{5′}, R^{5″} and R^{5′″} represent each independently H, hydroxyl, alkyl, alkoxy, haloalkyl or R⁴ and R^{4′} form together an oxo moiety or R^{4″} and R^{4″} form together an oxo moiety or R⁵ and R^{5′} form together an oxo moiety or R^{5″} and R^{5′″} form together an oxo moiety, preferably R⁴, R^{4′}, R^{4″}, R^{4′″}, R⁵, R^{5′}, R⁵″ and R^{5′″} represent H or oxo;
R⁶ represents
   —H;
   alkyl, the alkyl group being optionally substituted by one or more groups selected from halogen, hydroxyl or COOH; preferably methyl or —CH₂—CH₂—OH;
   cycloalkyl;
   halogen;
   hydroxyl;
   oxo;
   COR¹, SO₂R¹, wherein R¹ represents a group selected from C1-C6 alkyl, preferably Me, Et, iPr, tBu; cycloalkyl, preferably cyclopropane; alkene, preferably ethylene; amino, preferably NMe₂; wherein R¹ groups are optionally substituted by one or more groups selected from halogen, preferably F; hydroxyl; alkoxy, preferably OMe; COOH; amino, preferably NMe₂ or NH₂; SO₂Me;
   COOR¹, wherein R¹ represents a group selected from C1-C6 alkyl, preferably Me, Et, iPr, tBu; cycloalkyl; alkene; amino; wherein R¹ groups are optionally substituted by one or more groups selected from halogen; hydroxyl; alkoxy; COOH; amino; SO₂Me.

In one embodiment, preferred compounds of Formula I are those of Formula Ia-2:

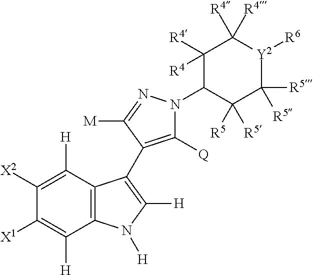

and pharmaceutically acceptable enantiomers, salts and solvates thereof, wherein:
X¹ and X² represent each independently H or F;
M and Q represent each independently H, C1-C6 alkyl optionally substituted one or more alogen;
Y² represents N or CH; preferably Y is N;
R⁴, R^{4′}, R^{4″}, R^{4′″}, R⁵, R^{5′}, R^{5″} and R^{5′″} represent each independently H, hydroxyl, alkyl, alkoxy, haloalkyl or R⁴ and R^{4′} form together an oxo moiety or R^{4″} and R^{4′″} form together an oxo moiety or R⁵ and R^{5′} form together an oxo moiety or R^{5″} and R^{5′″} form together an oxo moiety, preferably R⁴, R^{4′}, R^{4″}, R^{4′″}, R⁵, R^{5′}, R^{5″} and R^{5′″} represent H or oxo;
R⁶ represents
   H;
   alkyl, the alkyl group being optionally substituted by one or more groups selected from halogen, hydroxyl or COOH; preferably methyl or —CH₂—CH₂—OH;
   cycloalkyl;
   halogen;
   hydroxyl;
   oxo;
   COR¹, SO₂R¹, wherein R¹ represents a group selected from C1-C6 alkyl, preferably Me, Et, iPr, tBu; cycloalkyl, preferably cyclopropane; alkene, preferably ethylene; amino, preferably NMe₂; wherein R¹ groups are optionally substituted by one or more groups selected from halogen, preferably F; hydroxyl; alkoxy, preferably OMe; COOH; amino, preferably NMe₂ or NH₂; SO₂Me.

In one embodiment, preferred compounds of Formula Ia-2 are those of Formula Ia-2′:

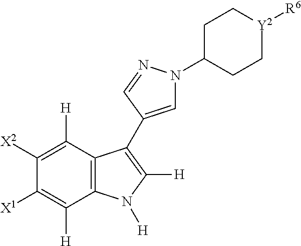

and pharmaceutically acceptable enantiomers, salts and solvates thereof, wherein:
X¹ and X² represent each independently H or F; preferably X¹ represents F and X² represents H;
Y² represents N or CH; preferably Y is N;
R⁶ represents
   H;
   alkyl, the alkyl group being optionally substituted by one or more groups selected from halogen, hydroxyl or COOH; preferably methyl or —CH₂—CH₂—OH;
   cycloalkyl;
   halogen;
   hydroxyl;
   oxo;
   COR¹, SO₂R¹, wherein R¹ represents a group selected from C1-C6 alkyl, preferably Me, Et, nPr, iPr, iBu, tBu; cycloalkyl, preferably cyclopropane; alkene, preferably ethylene; amino, preferably NH₂, NHMe or NMe₂; wherein R¹ groups are optionally substituted by one or more groups selected from halogen, preferably F; hydroxyl; alkoxy, preferably OMe; COOH; amino, preferably NMe₂ or NH₂; SO₂Me.

In a specific embodiment, in Formula Ia-2′, R⁶ represents COR¹ or SO₂R¹, wherein R¹ represents a group selected from C1-C6 alkyl, preferably Me, Et, nPr, iPr, iBu, tBu; cycloalkyl, preferably cyclopropane; alkene, preferably ethylene; amino, preferably NH₂, NHMe or NMe₂; wherein R¹ groups are optionally substituted by one or more groups selected from halogen, preferably F; hydroxyl; alkoxy, preferably OMe; COOH; amino, preferably NMe₂ or NH₂; SO₂Me.

In a specific embodiment, in Formula Ia-2′, R⁶ represents COR¹, wherein R¹ represents a group selected from C1-C6 alkyl, preferably Me, Et or nPr; wherein R¹ groups are optionally substituted by SO₂Me.

In a specific embodiment, in Formula Ia-2′, R⁶ represents SO₂R¹, wherein R¹ represents a group selected from C1-C6 alkyl, preferably Me, Et, nPr, iPr, tBu; wherein R¹ groups are optionally substituted by one or more groups selected from halogen, preferably F; hydroxyl; alkoxy, preferably OMe; COOH; amino, preferably NMe₂ or NH₂; SO₂Me.

In one embodiment, preferred compounds of Formula I are those of Formula Ia-3:

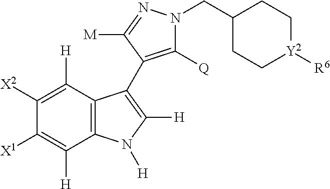

and pharmaceutically acceptable enantiomers, salts and solvates thereof, wherein:
X¹ and X² represent each independently H or F;
M and Q represent each independently H, C1-C6 alkyl optionally substituted one or more halogen;
Y² represents N or CH; preferably Y is N;
R⁶ represents
   H;
   alkyl, the alkyl group being optionally substituted by one or more groups selected from halogen, hydroxyl or COOH;
   cycloalkyl, preferably cyclopropane;
   COR¹, SO₂R¹, wherein R¹ represents a group selected from C1-C6 alkyl, preferably methyl or ethyl; cycloalkyl; alkene; amino; wherein R¹ groups are optionally substituted by one or more groups selected from halogen; hydroxyl; alkoxy; COOH; amino; SO₂Me.

In one embodiment, preferred compounds of Formula I are those of Formula Ib:

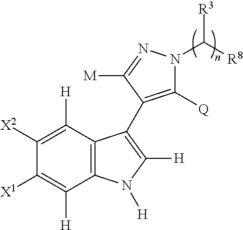

and pharmaceutically acceptable enantiomers, salts and solvates thereof, wherein:
X¹ and X² represent each independently H, halogen or haloalkyl, preferably H, F or CF₃, more preferably H or F;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl; preferably M and Q represent each independently H, methyl or CF₃, more preferably H or methyl;
n represents an integer equal to 1, 2 or 3, preferably 1 or 2;
R³ represents H, alkyl;
R⁵ represents H, alkyl, the alkyl group being optionally substituted by one or more groups selected from halogen, hydroxyl, COOH, CONH₂; cycloalkyl, halogen, hydroxyl, oxo, COR¹, COOR¹, CONR¹R², NR¹COR², NR¹R², SO₂R¹, SO₂NR¹R², NR¹SO₂R², SOR, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me.

In one embodiment, preferred compounds of Formula I are those of Formula Ic:

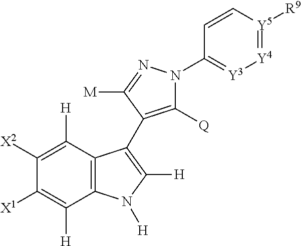

and pharmaceutically acceptable enantiomers, salts and solvates thereof, wherein:
X¹ and X² represent each independently H, halogen or haloalkyl, preferably H, F or CF₃, more preferably H or F;
M and Q represent each independently H, halogen, hydroxyl, C1-C6 alkyl optionally substituted one or more substituents selected from the group comprising halogen, hydroxyl, CONR¹R¹, NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl; preferably M and Q represent each independently H, methyl or CF₃, more preferably H or methyl;
Y³, Y⁴, Y⁵ represent each independently N or CH;
R⁹ is absent or represents H; halogen, preferably Cl; amino, preferably NH₂.

In a preferred embodiment, in compounds of Formula Ic of the invention at least one of Y³, Y⁴, Y⁵ represent N.

Particularly preferred compounds of Formula I of the invention are those listed in Table 1 hereafter.

TABLE 1

| Cpd n ° | Structure | Chemical name | MW |
|---|---|---|---|
| 1 | 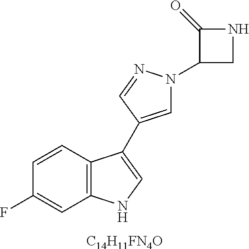 | 3-(4-(6-fluoro-1H-indol- 3-yl)-1H-pyrazol-1- yl)azetidin-2-one | 270.26 |
| 2 | 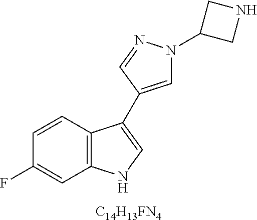 | 3-(1-(azetidin-3-yl)-1H- pyrazol-4-yl)-6-fluoro-1H- indole | 256.28 |
| 3 | 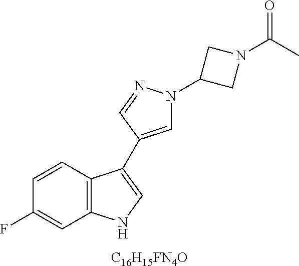 | 1-(3-(4-(6-fluoro-1H-indol- 3-yl)-1H-pyrazol-1- yl)azetidin-1-yl)ethanone | 298.31 |
| 4 | 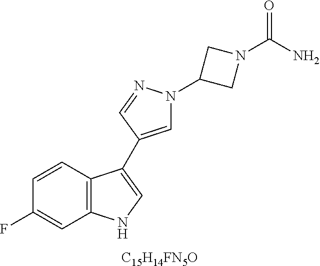 | 3-(4-(6-fluoro-1H-indol-3- yl)-1H-pyrazol-1- yl)azetidine-1-carboxamide | 299.30 |
| 5 | 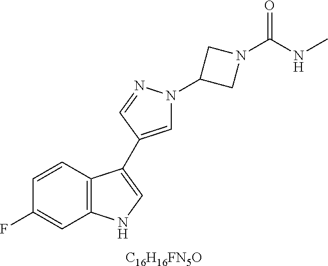 | 3-(4-(6-fluoro-1H-indol-3- yl)-1H-pyrazol-1-yl)-N- methylazetidine-1- carboxamide | 313.33 |
| 6 | 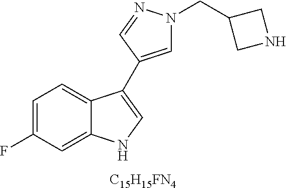 | 3-(1-(azetidin-3-ylmethyl)- 1H-pyrazol-4-yl)-6-fluoro- 1H-indole | 270.30 |
| 7 | 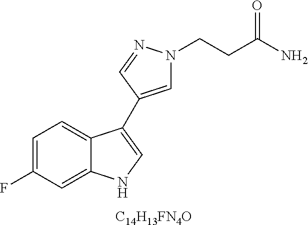 | 3-(4-(6-fluoro-1H-indol-3- yl)-1H-pyrazol-1- yl)propanamide | 272.28 |
| 8 | 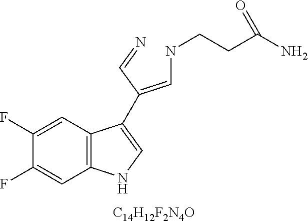 | 3-(4-(5,6-difluoro-1H-indol- 3-yl)-1H-pyrazol-1- yl)propanamide | 290.27 |
| 9 | 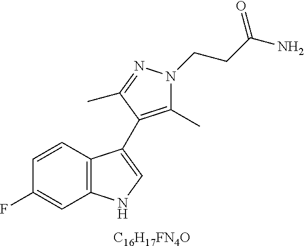 | 3-(4-(6-fluoro-1H-indol-3- yl)-3,5-dimethyl-1H- pyrazol-1-yl)propanamide | 300.33 |
| 10 | 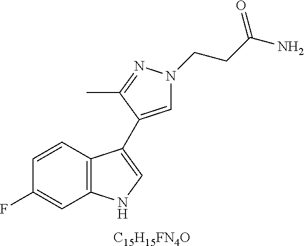 | 3-(4-(6-fluoro-1H-indol-3- yl)-3-methyl-1H-pyrazol-1- yl)propanamide | 286.30 |
| 11 | 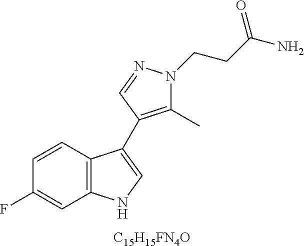 | 3-(4-(6-fluoro-1H-indol-3- yl)-5-methyl-1H-pyrazol- 1-yl)propanamide | 286.30 |
| 12 | 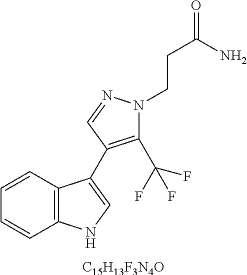 | 3-(4-(1H-indol-3-yl)-5- (trifluoromethyl)-1H- pyrazol-1-yl)propanamide | 322.29 |
| 13 | 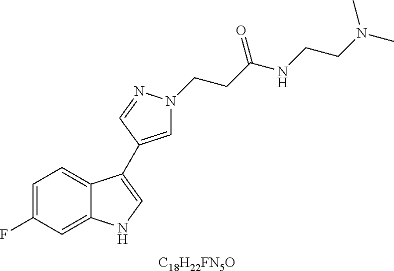 [SMILES: CN(C)CCNC(=O)CCn1cc(-c2c[nH]c3cc(F)ccc23)cn1 ; "C18H22FN5O"] | N-(2- (dimethylamino)ethyl)-3- (4-(6-fluoro-1H-indol-3- yl)-1H-pyrazol-1- yl)propanamide | 343.40 |
| 14 | 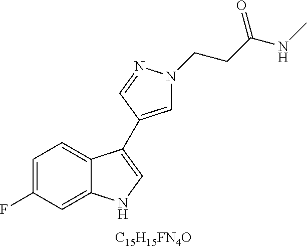 | 3-(4-(6-fluoro-1H-indol-3- yl)-1H-pyrazol-1-yl)-N- methylpropanamide | 286.30 |
| 15 | 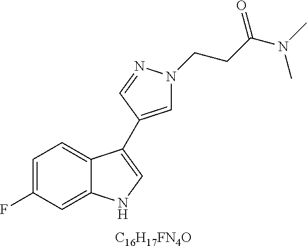 | 3-(4-(6-fluoro-1H-indol-3- yl)-1H-pyrazol-1-yl)-N,N- dimethylpropanamide | 300.33 |
| 16 | 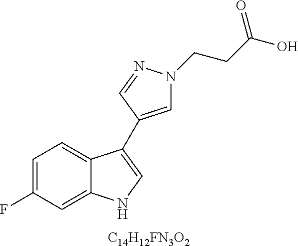 | 3-(4-(6-fluoro-1H-indol-3- yl)-1H-pyrazol-1- yl)propanoic acid | 273.26 |
| 17 | 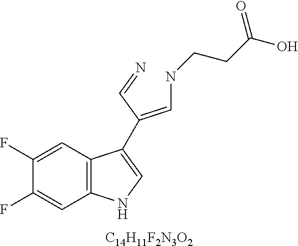 | 3-(4-(5,6-difluoro-1H- indol-3-yl)-1H-pyrazol-1- yl)propanoic acid | 291.25 |
| 18 | 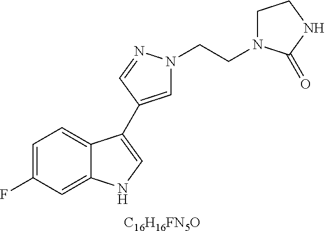 | 1-(2-(4-(6-fluoro-1H-indol- 3-yl)-1H-pyrazol-1- yl)ethyl)imidazolidin-2-one | 313.33 |
| 19 | 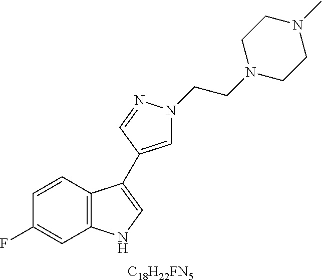 | 6-fluoro-3-(1-(2-(4- methylpiperazin-1-yl)ethyl)- 1H-pyrazol-4-yl)-1H-indole | 327.40 |
| 20 | 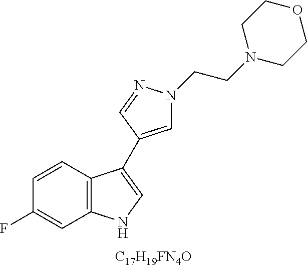 | 4-(2-(4-(6-fluoro-1H-indol- 3-yl)-1H-pyrazol-1- yl)ethyl)morpholine | 314.36 |
| 21 | 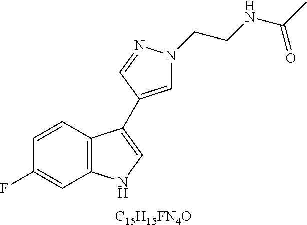 | N-(2-(4-(6-fluoro-1H-indol- 3-yl)-1H-pyrazol-1- yl)ethyl)acetamide | 286.30 |
| 22 | 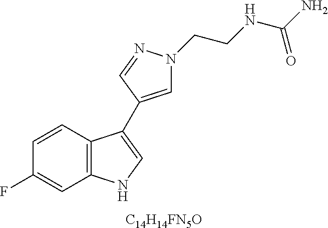 | 1-(2-(4-(6-fluoro-1H-indol- 3-yl)-1H-pyrazol-1- yl)ethyl)urea | 287.29 |
| 23 | 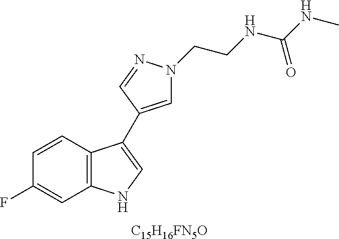 | 1-(2-(4-(6-fluoro-1H-indol- 3-yl)-1H-pyrazol-1- yl)ethyl)-3-methylurea | 301.32 |
| 24 | 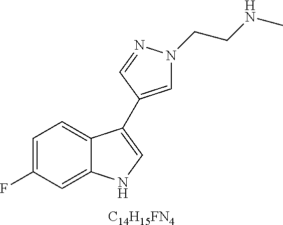 | 2-(4-(6-fluoro-1H-indol-3- yl)-1H-pyrazol-1-yl)-N- methylethanamine | 258.29 |
| 25 | 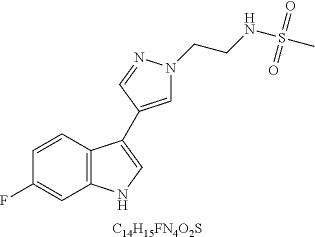 | N-(2-(4-(6-fluoro-1H- indol-3-yl)-1H-pyrazol-1- yl)ethyl)methanesulfon- amide | 322.36 |
| 26 | 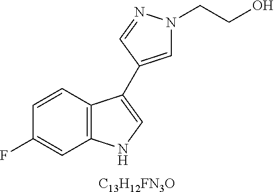 | 2-(4-(6-fluoro-1H-indol- 3-yl)-1H-pyrazol-1- yl)ethanol | 245.25 |
| 27 | 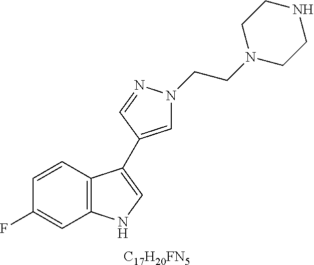 | 6-fluoro-3-(1-(2-(piperazin- 1-yl)ethyl)-1H-pyrazol-4- yl)-1H-indole | 313.37 |
| 28 | 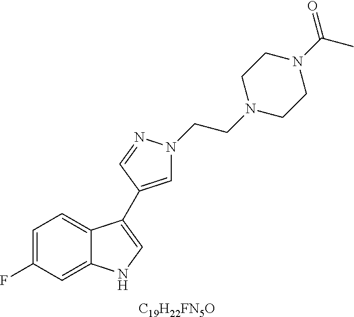 | 1-(4-(2-(4-(6-fluoro-1H- indol-3-yl)-1H-pyrazol-1- yl)ethyl)piperazin-1- yl)ethanone | 355.41 |
| 29 | 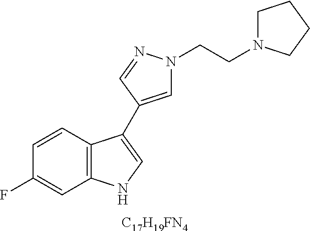 | 6-fluoro-3-(1-(2- (pyrrolidin-1-yl)ethyl)-1H- pyrazol-4-yl)-1H-indole | 298.36 |
| 30 | 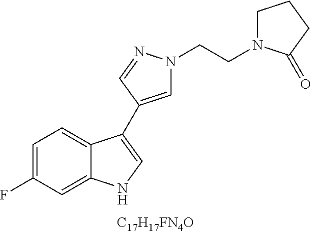 | 1-(2-(4-(6-fluoro-1H-indol- 3-yl)-1H-pyrazol-1- yl)ethyl)pyrrolidin-2-one | 312.34 |
| 31 | 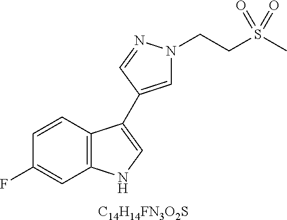 | 6-fluoro-3-(1-(2- (methylsulfonyl)ethyl)-1H- pyrazol-4-yl)-1H-indole | 307.34 |
| 32 | 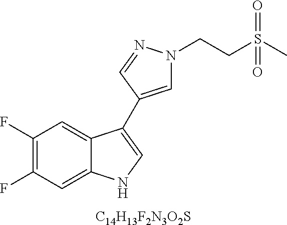 | 5,6-difluoro-3-(1-(2- (methylsulfonyl)ethyl)-1H- pyrazol-4-yl)-1H-indole | 325.33 |
| 33 | 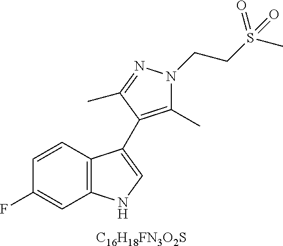 | 3-(3.5-dimethyl-1-(2- (methylsulfonyl)ethyl)-1H- pyrazol-4-yl)-6-fluoro-1H- indole | 335.40 |
| 34 | 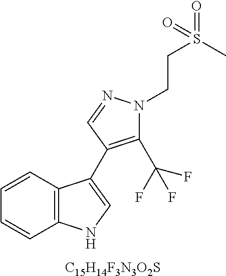 | 3-(1-(2- (methylsulfonyl)ethyl)-5- (trifluoromethyl)-1H- pyrazol-4-yl)-1H-indole | 357.35 |
| 35 | 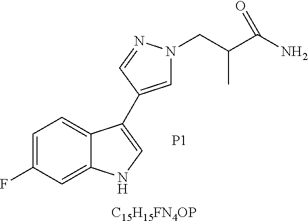 | 3-(4-(6-fluoro-1H-indol-3- yl)-1H-pyrazol-1-yl)-2- methylpropanamide (first eluted enantiomer) | 320.30 |
| 36 | 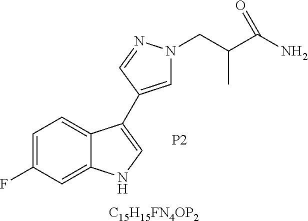 | 3-(4-(6-fluoro-1H-indol-3- yl)-1H-pyrazol-1-yl)-2- methylpropanamide (second eluted enantiomer) | 352.28 |
| 37 | 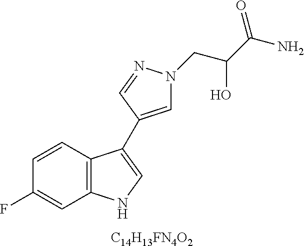 | 3-(4-(6-fluoro-1H-indol-3- yl)-1H-pyrazol-1-yl)-2- hydroxypropanamide | 288.28 |
| 38 | 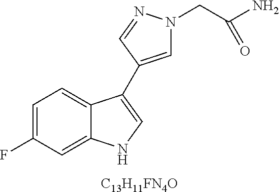 | 2-(4-(6-fluoro-1H-indol-3- yl)-1H-pyrazol-1- yl)acetamide | 258.25 |
| 39 | 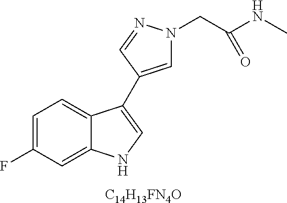 | 2-(4-(6-fluoro-1H-indol-3- yl)-1H-pyrazol-1-yl)-N- methylacetamide | 272.28 |
| 40 | 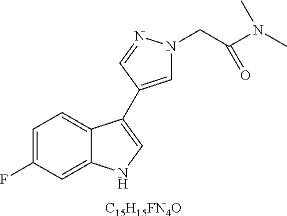 | 2-(4-(6-fluoro-1H-indol-3- yl)-1H-pyrazol-1-yl)-N,N- dimethylacetamide | 286.30 |
| 41 | 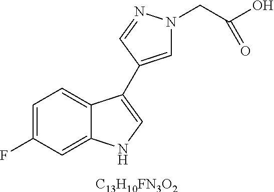 | 2-(4-(6-fluoro-1H-indol-3- yl)-1H-pyrazol-1-yl)acetic acid | 259.24 |
| 42 | 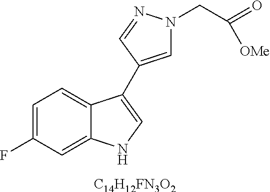 | methyl 2-(4-(6-fluoro-1H- indol-3-yl)-1H-pyrazol-1- yl)acetate | 273.26 |
| 43 | 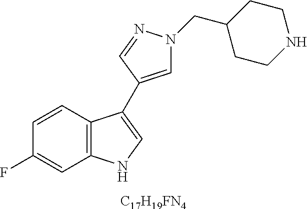 | 6-fluoro-3-(1-(piperidin-4- ylmethyl)-1H-pyrazol-4- yl)-1H-indole | 298.36 |
| 44 | 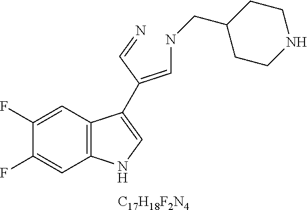 | 5,6-difluoro-3-(1-(piperidin- 4-ylmethyl)-1H-pyrazol-4- yl)-1H-indole | 316.35 |
| 45 | 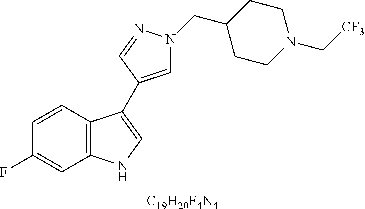 | 6-fluoro-3-(1-((1-(2,2,2- trifluoroethyl)piperidin-4- yl)methyl)-1H-pyrazol-4- yl)-1H-indole | 380.38 |
| 46 | 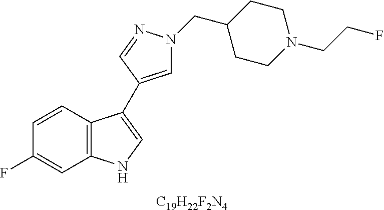 [SMILES: FCCN1CCC(Cn2cc(-c3c[nH]c4cc(F)ccc34)cn2)CC1 ; "C19H22F2N4"] | 6-fluoro-3-(1-((1-(2- fluoroethyl)piperidin-4- yl)methyl)-1H-pyrazol-4- yl)-1H-indole | 344.40 |
| 47 | 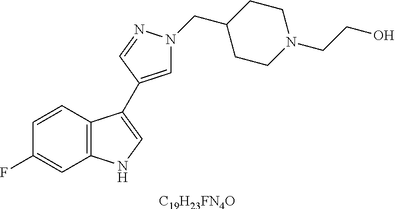 [SMILES: OCCN1CCC(Cn2cc(-c3c[nH]c4cc(F)ccc34)cn2)CC1 ; "C19H23FN4O"] | 2-(4-((4-(6-fluoro-1H-indol- 3-yl)-1H-pyrazol-1- yl)methyl)piperidin-1- yl)ethanol | 342.41 |
| 48 | 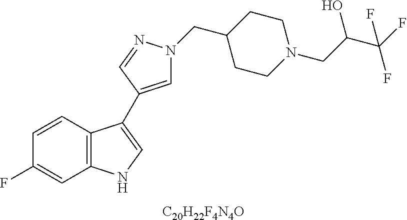 [SMILES: OC(CN1CCC(Cn2cc(-c3c[nH]c4cc(F)ccc34)cn2)CC1)C(F)(F)F ; "C20H22F4N4O"] | 1,1,1-trifluoro-3-(4-((4-(6- fluoro-1H-indol-3-yl)-1H- pyrazol-1- yl)methyl)piperidin-1- yl)propan-2-ol | 410.41 |
| 49 | 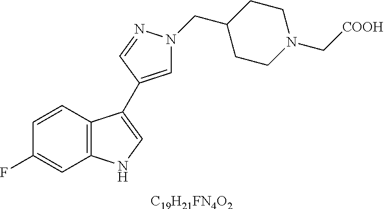 [SMILES: O=C(O)CN1CCC(Cn2cc(-c3c[nH]c4cc(F)ccc34)cn2)CC1 ; "C19H21FN4O2"] | 2-(4-((4-(6-fluoro-1H-indol- 3-yl)-1H-pyrazol-1- yl)methyl)piperidin-1- yl)acetic acid | 356.39 |
| 50 | 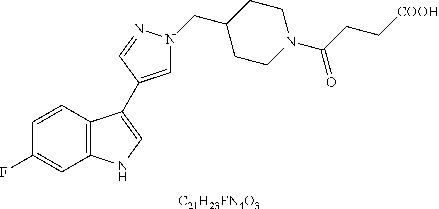 [SMILES: O=C(O)CCC(=O)N1CCC(Cn2cc(-c3c[nH]c4cc(F)ccc34)cn2)CC1 ; "C21H23FN4O3"] | 4-(4-((4-(6-fluoro-1H-indol- 3-yl)-1H-pyrazol-1- yl)methyl)piperidin-1-yl)-4- oxobutanoic acid | 398.43 |
| 51 | 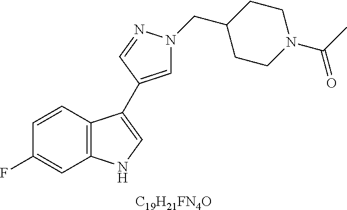 | 1-(4-((4-(6-fluoro-1H-indol- 3-yl)-1H-pyrazol-1- yl)methyl)piperidin-1- yl)ethanone | 340.39 |
| 52 | 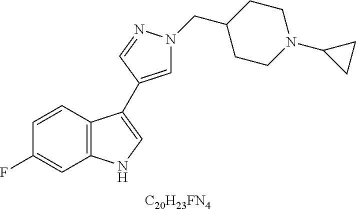 | 3-(1-((1- cyclopropylpiperidin-4- yl)methyl)-1H-pyrazol-4- yl)-6-fluoro-1H-indole | 338.42 |
| 53 | 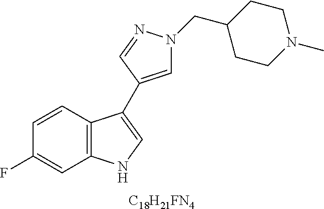 | 6-fluoro-3-(1-((1- methylpiperidin-4-yl)methyl)- 1H-pyrazol-4-yl)-1H-indole | 312.38 |
| 54 | 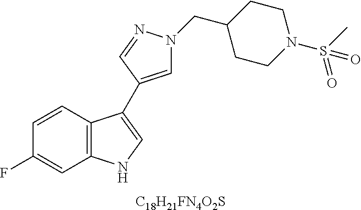 | 6-fluoro-3-(1-((1- (methylsulfonyl)piperidin-4- yl)methyl)-1H-pyrazol-4-yl)- 1H-indole | 376.45 |
| 55 | 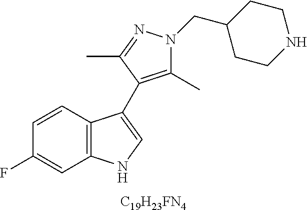 | 3-(3,5-dimethyl-1- (piperidin-4-ylmethyl)-1H- pyrazol-4-yl)-6-fluoro-1H- indole | 326.41 |
| 56 | 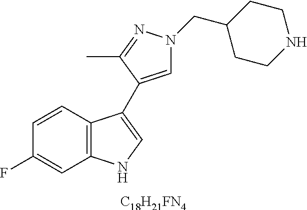 | 6-fluoro-3-(3-methyl-1- (piperidin-4-ylmethyl)-1H- pyrazol-4-yl)-1H-indole | 312.38 |
| 57 | 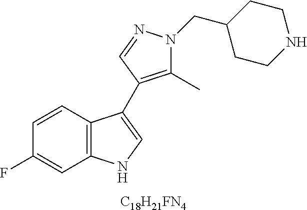 | 6-fluoro-3-(5-methyl-1- (piperidin-4-ylmethyl)-1H- pyrazol-4-yl)-1H-indole | 312.38 |
| 58 | 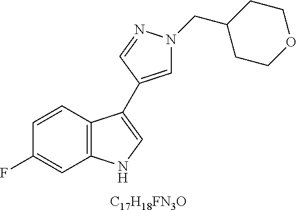 | 6-fluoro-3-(1-((tetrahydro- 2H-pyran-4-yl)methyl)-1H- pyrazol-4-yl)-1H-indole | 299.34 |
| 59 | 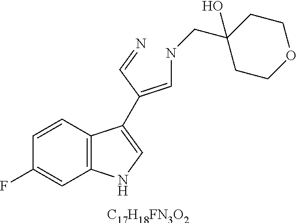 | 4-((4-(6-fluoro-1H-indol-3- yl)-1H-pyrazol-1- yl)methyl)tetrahydro-2H- pyran-4-ol | 315.34 |
| 60 | 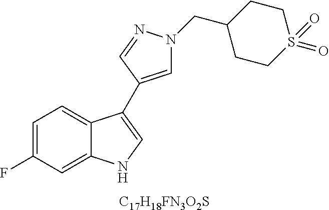 | 4-((4-(6-fluoro-1H-indol-3- yl)-1H-pyrazol-1- yl)methyl)tetrahydro-2H- thiopyran 1,1-dioxide | 347.41 |
| 61 | 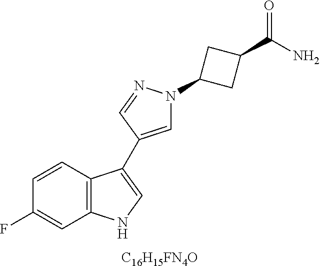 | (1S,3R)-3-(4-(6-fluoro-1H- indol-3-yl)-1H-pyrazol-1- yl)cyclobutanecarboxamide | 298.31 |
| 62 | 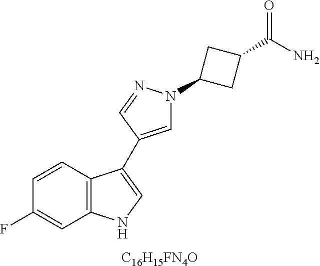 | (1R,3R)-3-(4-(6-fluoro-1H- indol-3-yl)-1H-pyrazol-1- yl)cyclobutanecarboxamide | 298.31 |
| 63 | 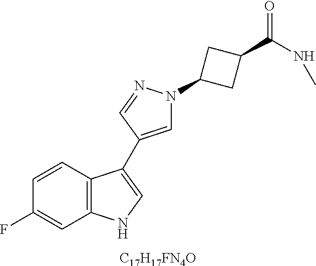 | (1S,3S)-3-(4-(6-fluoro-1H- indol-3-yl)-1H-pyrazol-1- yl)-N- methylcyclobutanecarbox- amide | 312.34 |
| 64 | 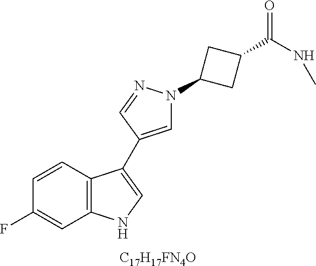 | (1R,3R)-3-(4-(6-fluoro-1H- indol-3-yl)-1H-pyrazol-1- yl)-N-methylcyclobutane- carboxamide | 312.34 |
| 65 | 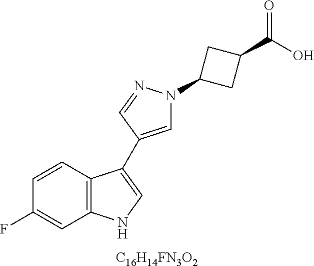 | (1S,3S)-3-(4-(6-fluoro-1H- indol-3-yl)-1H-pyrazol-1- yl)cyclobutanecarboxylic acid | 299.30 |
| 66 | 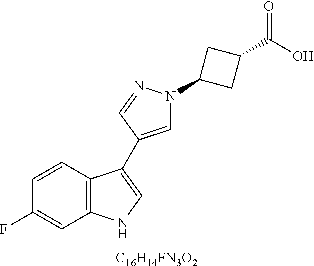 | (1R,3R)-3-(4-(6-fluoro-1H- indol-3-yl)-1H-pyrazol-1- yl)cyclobutanecarboxylic acid | 299.30 |
| 67 | 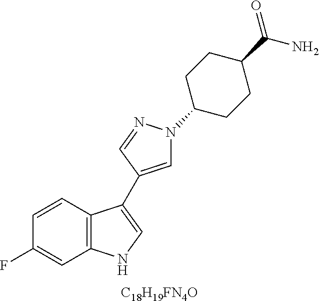 | (1R,4R)-4-(4-(6-fluoro-1H- indol-3-yl)-1H-pyrazol-1- yl)cyclohexanecarboxamide | 326.37 |
| 68 | 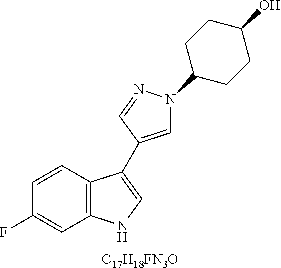 | (1R,4R)-4-(4-(6-fluoro-1H- indol-3-yl)-1H-pyrazol-1- yl)cyclohexanol | 299.34 |
| 69 | 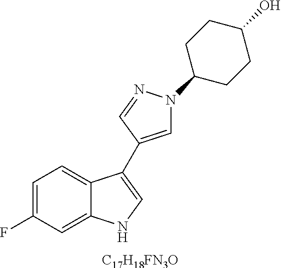 | (1R,4R)-4-(4-(6-fluoro-1H- indol-3-yl)-1H-pyrazol-1- yl)cyclohexanol | 299.34 |
| 70 | 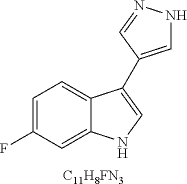 [SMILES: Fc1ccc2c(-c3cn[nH]c3)c[nH]c2c1 ; "C11H8FN3"] | 6-fluoro-3-(1H-pyrazol-4- yl)-1H-indole | 201.20 |
| 71 | 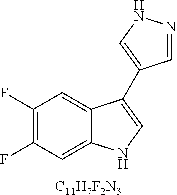 [SMILES: Fc1cc2[nH]cc(-c3cn[nH]c3)c2cc1F ; "C11H7F2N3"] | 5,6-difluoro-3-(1H-pyrazol- 4-yl)-1H-indole | 219.19 |
| 72 | 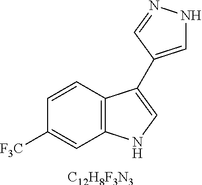 [SMILES: FC(F)(F)c1ccc2c(-c3cn[nH]c3)c[nH]c2c1 ; "C12H8F3N3"] | 3-(1H-pyrazol-4-yl)-6- (trifluoromethyl)-1H-indole | 251.21 |
| 73 | 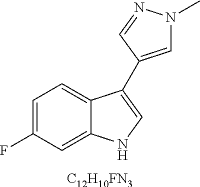 [SMILES: Cn1cc(-c2c[nH]c3cc(F)ccc23)cn1 ; "C12H10FN3"] | 6-fluoro-3-(1-methyl-1H- pyrazol-4-yl)-1H-indole | 215.23 |
| 74 | 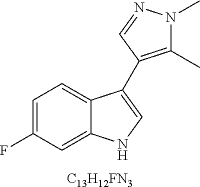 [SMILES: Cc1c(-c2c[nH]c3cc(F)ccc23)cnn1C ; "C13H12FN3"] | 3-(1,5-dimethyl-1H- pyrazol-4-yl)-6-fluoro- 1H-indole | 229.25 |
| 75 | 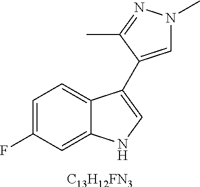 [SMILES: Cc1nn(C)cc1-c1c[nH]c2cc(F)ccc12 ; "C13H12FN3"] | 3-(1,3-dimethyl-1H- pyrazol-4-yl)-6-fluoro- 1H-indole | 229.25 |
| 76 | 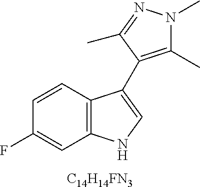 [SMILES: Cc1nn(C)c(C)c1-c1c[nH]c2cc(F)ccc12 ; "C14H14FN3"] | 6-fluoro-3-(1,3,5-trimethyl- 1H-pyrazol-4-yl)-1H-indole | 243.28 |
| 77 | 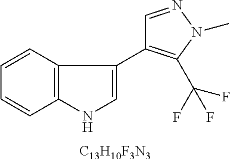 | 3-(1-methyl-5- (trifluoromethyl)-1H- pyrazol-4-yl)-1H-indole | 265.23 |
| 78 | 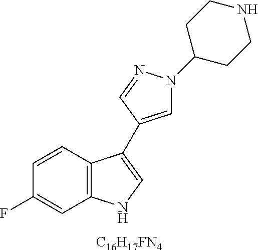 | 6-fluoro-3-(1-(piperidin-4- yl)-1H-pyrazol-4-yl)-1H- indole | 284.33 |
| 79 | 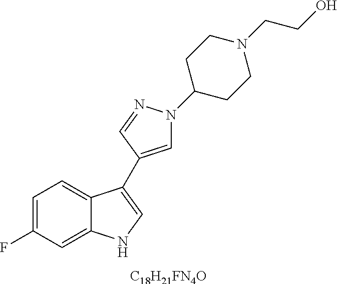 | 2-(4-(4-(6-fluoro-1H-indol- 3-yl)-1H-pyrazol-1- yl)piperidin-1-yl)ethanol | 328.38 |
| 80 | 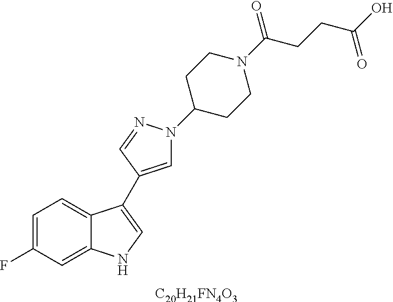 [SMILES: O=C(O)CCC(=O)N1CCC(n2cc(-c3c[nH]c4cc(F)ccc34)cn2)CC1 ; "C20H21FN4O3"] | 4-(4-(4-(6-fluoro-1H-indol- 3-yl)-1H-pyrazol-1- yl)piperidin-1-yl)-4- oxobutanoic acid | 384.40 |
| 81 | 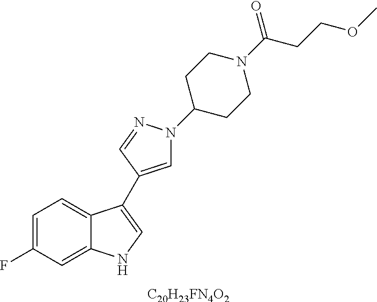 | 1-(4-(4-(6-fluoro-1H-indol- 3-yl)-1H-pyrazol-1- yl)piperidin-1-yl)-3- methoxypropan-1-one | 370.42 |
| 82 | 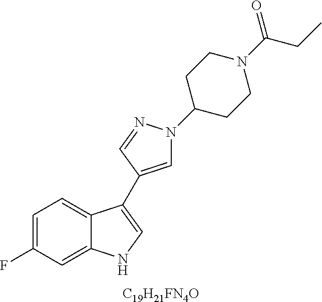 | 1-(4-(4-(6-fluoro-1H-indol- 3-yl)-1H-pyrazol-1- yl)piperidin-1-yl)propan-1- one | 340.39 |
| 83 | 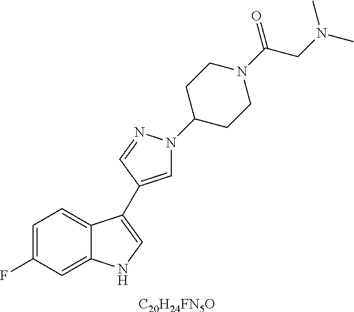 | 2-(dimethylamino)-1-(4-(4- (6-fluoro-1H-indol-3-yl)- 1H-pyrazol-1-yl)piperidin- 1-yl)ethanone | 369.44 |
| 84 | 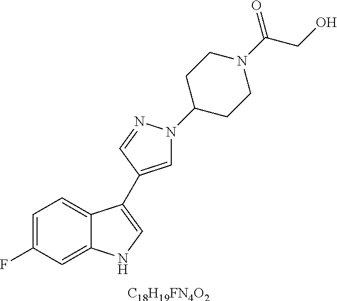 | 1-(4-(4-(6-fluoro-1H-indol- 3-yl)-1H-pyrazol-1- yl)piperidin-1-yl)-2- hydroxyethanone | 342.37 |
| 85 | 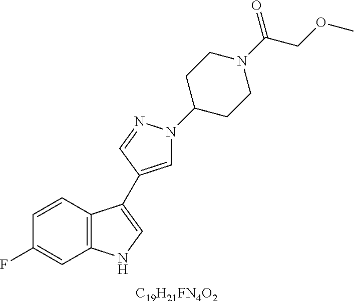 | 1-(4-(4-(6-fluoro-1H-indol- 3-yl)-1H-pyrazol-1- yl)piperidin-1-yl)-2- methoxyethanone | 356.39 |
| 86 | 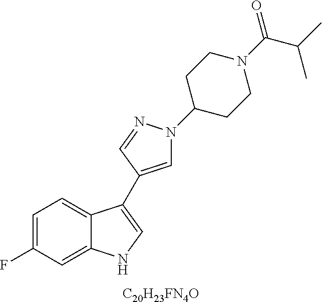 | 1-(4-(4-(6-fluoro-1H-indol- 3-yl)-1H-pyrazol-1- yl)piperidin-1-yl)-2- methylpropan-1-one | 354.42 |
| 87 | 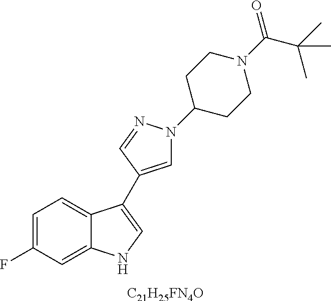 | 1-(4-(4-(6-fluoro-1H-indol- 3-yl)-1H-pyrazol-1- yl)piperidin-1-yl)-2,2- dimethylpropan-1-one | 368.45 |
| 88 | 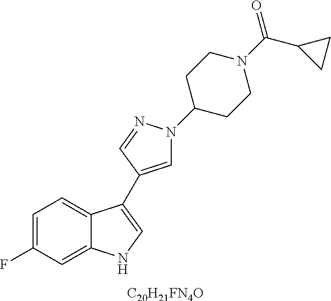 | cyclopropyl(4-(4-(6-fluoro- 1H-indol-3-yl)-1H-pyrazol- 1-yl)piperidin-1- yl)methanone | 352.41 |
| 89 | 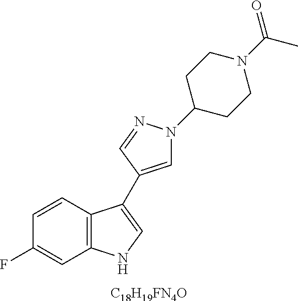 | 1-(4-(4-(6-fluoro-1H-indol- 3-yl)-1H-pyrazol-1- yl)piperidin-1-yl)ethanone | 326.37 |
| 90 | 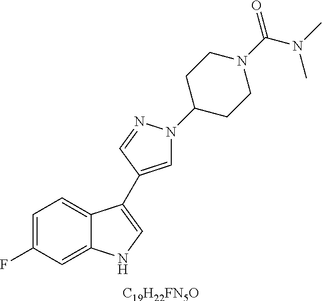 | 4-(4-(6-fluoro-1H-indol-3- yl)-1H-pyrazol-1-yl)-N,N- dimethylpiperidine-1- carboxamide | 355.41 |
| 91 | 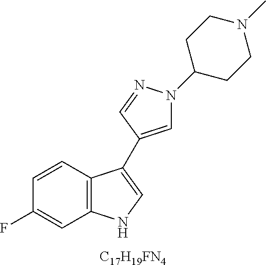 | 6-fluoro-3-(1-(1- methylpiperidin-4-yl)-1H- pyrazol-4-yl)-1H-indole | 298.36 |
| 92 | 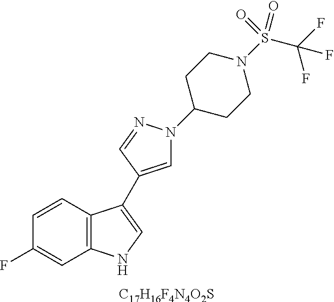 | 6-fluoro-3-(1-(1-((trifluoro- methyl)sulfonyl)piperidin- 4-yl)-1H-pyrazol-4-yl)-1H- indole | 416.39 |
| 93 | 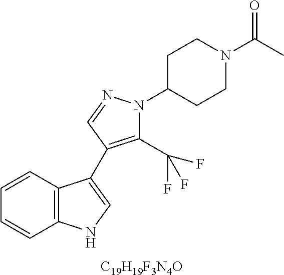 | 1-(4-(4-(1H-indol-3-yl)-5- (trifluoromethyl)-1H- pyrazol-1-yl)piperidin-1- yl)ethanone | 376.38 |
| 94 | 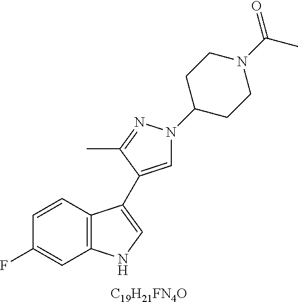 | 1-(4-(4-(6-fluoro-1H-indol- 3-yl)-3-methyl-1H-pyrazol- 1-yl)piperidin-1-yl)ethanone | 340.39 |
| 95 | 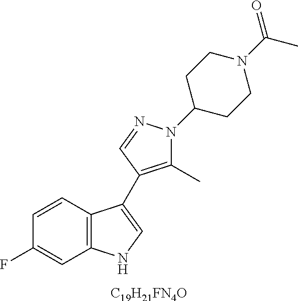 | 1-(4-(4-(6-fluoro-1H-indol- 3-yl)-5-methyl-1H-pyrazol- 1-yl)piperidin-1-yl)ethanone | 340.39 |
| 96 | 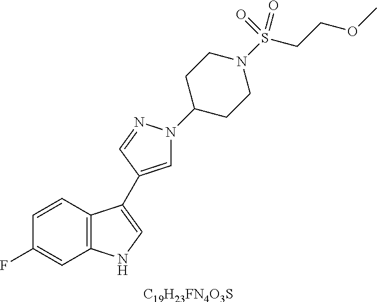 | 6-fluoro-3-(1-(1-((2- methoxyethyl)sulfonyl)piper- idin-4-yl)-1H-pyrazol-4-yl)- 1H-indole | 406.47 |
| 97 | 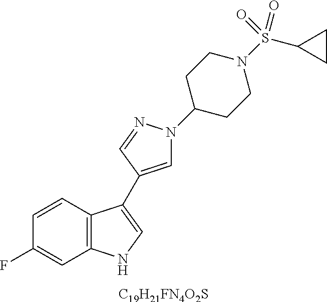 | 3-(1-(1- (cyclopropylsulfonyl)piper- idin-4-yl)-1H-pyrazol-4-yl)- 6-fluoro-1H-indole | 388.46 |
| 98 | 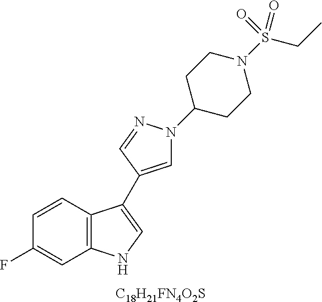 | 3-(1-1- (ethylsulfonyl)piperidin-4- yl)-1H-pyrazol-4-yl)-6- fluoro-1H-indole | 376.45 |
| 99 | 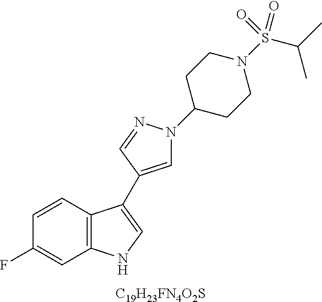 | 6-fluoro-3-(1-(1- (isopropylsulfonyl)piper- idin-4-yl)-1H-pyrazol-4- yl)-1H-indole | 390.47 |
| 100 | 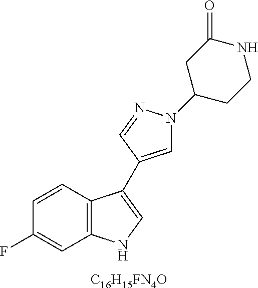 | 4-(4-(6-fluoro-1H-indol-3- yl)-1H-pyrazol-1- yl)piperidin-2-one | 298.31 |
| 101 | 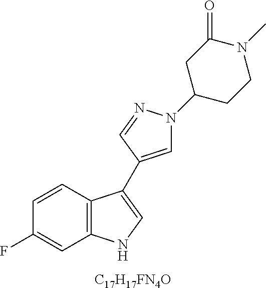 | 4-(4-(6-fluoro-1H-indol-3- yl)-1H-pyrazol-1-yl)-1- methylpiperidin-2-one | 312.34 |
| 102 | 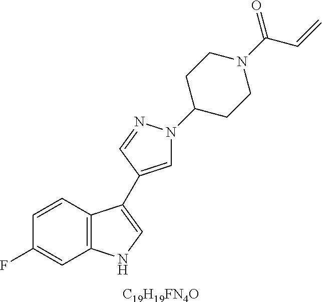 | 1-(4-(4-(6-fluoro-1H-indol- 3-yl)-1H-pyrazol-1- yl)piperidin-1-yl)prop-2-en- 1-one | 338.38 |
| 103 | 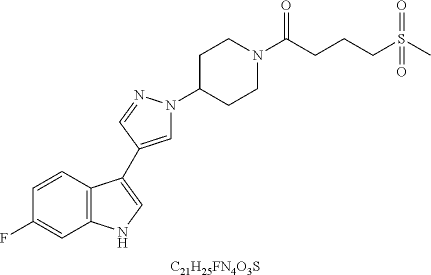 [SMILES: CS(=O)(=O)CCCC(=O)N1CCC(n2cc(-c3c[nH]c4cc(F)ccc34)cn2)CC1 ; "C21H25FN4O3S"] | 1-(4-(4-(6-fluoro-1H-indol- 3-yl)-1H-pyrazol-1- yl)piperidin-1-yl)-4- (methylsulfonyl)butan-1-one | 432.51 |
| 104 | 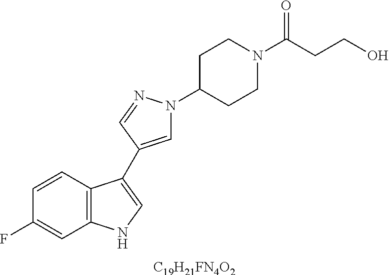 [SMILES: O=C(CCO)N1CCC(n2cc(-c3c[nH]c4cc(F)ccc34)cn2)CC1 ; "C19H21FN4O2"] | 1-(4-(4-(6-fluoro-1H-indol- 3-yl)-1H-pyrazol-1- yl)piperidin-1-yl)-3- hydroxypropan-1-one | 356.39 |
| 105 | 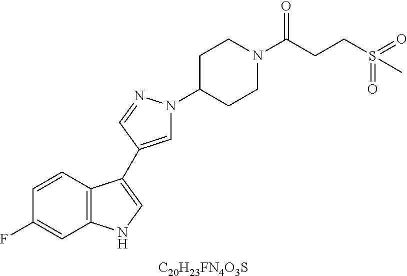 [SMILES: CS(=O)(=O)CCC(=O)N1CCC(n2cc(-c3c[nH]c4cc(F)ccc34)cn2)CC1 ; "C20H23FN4O3S"] | 1-(4-(4-(6-fluoro-1H-indol- 3-yl)-1H-pyrazol-1- yl)piperidin-1-yl)-3- (methylsulfonyl)propan-1- one | 418.49 |
| 106 | 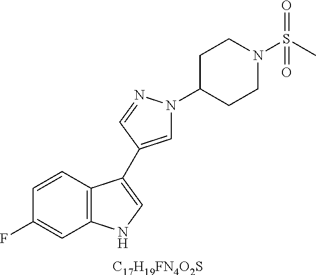 | 6-fluoro-3-(1-(1- (methylsulfonyl)piperidin- 4-yl)-1H-pyrazol-4-yl)-1H- indole | 362.42 |
| 107 | 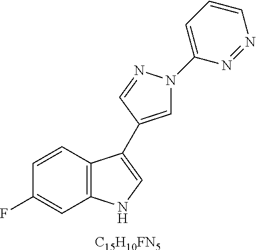 | 6-fluoro-3-(1-(pyridazin-3- yl)-1H-pyrazol-4-yl)-1H- indole | 279.27 |
| 108 | 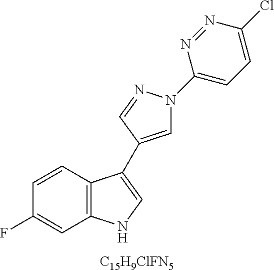 | 3-(1-(6-chloropyridazin-3- yl)-1H-pyrazol-4-yl)-6- fluoro-1H-indole | 313.72 |
| 109 | 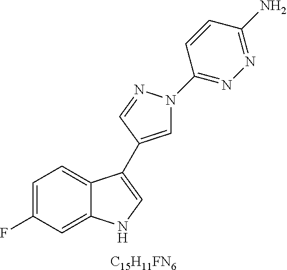 | 6-(4-(6-fluoro-1H-indol-3- yl)-1H-pyrazol-1- yl)pyridazin-3-amine | 294.29 |
| 110 | 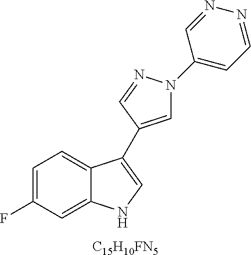 | 6-fluoro-3-(1-(pyridazin-4- yl)-1H-pyrazol-4-yl)-1H- indole | 279.27 |
| 111 | 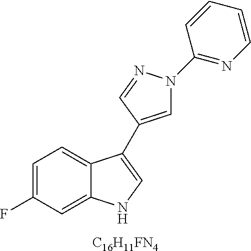 | 6-fluoro-3-(1-(pyridin-2-yl)- 1H-pyrazol-4-yl)-1H-indole | 278.28 |
| 112 | 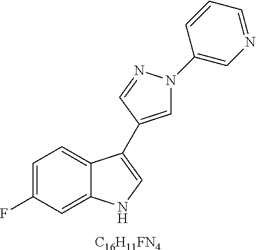 | 6-fluoro-3-(1-(pyridin-3-yl)- 1H-pyrazol-4-yl)-1H-indole | 278.28 |
| 113 | 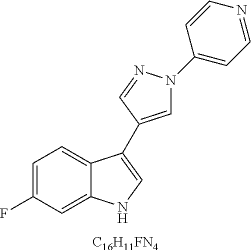 | 6-fluoro-3-(1-(pyridin-4-yl)- 1H-pyrazol-4-yl)-1H-indole | 278.28 |
| 114 | 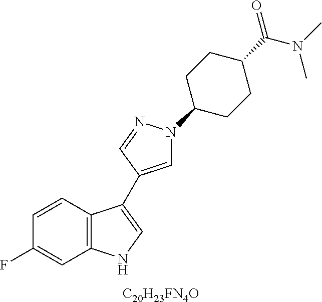 | (1R,4R)-4-(4-(6-fluoro-1H- indol-3-yl)-1H-pyrazol-1- yl)-N,N- dimethylcyclohexanecarbox- amide | 354.42 |
| 115 | 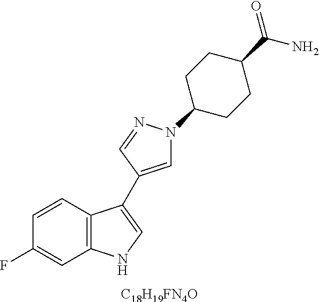 | (1S,4S)-4-(4-(6-fluoro-1H- indol-3-yl)-1H-pyrazol-1- yl)cyclohexanecarboxamide | 326.37 |
| 116 | 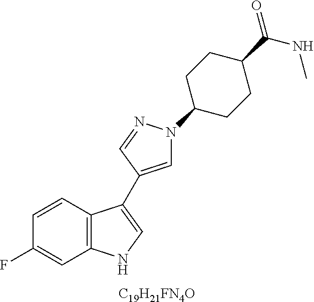 | (1S,4S)-4-(4-(6-fluoro-1H- indol-3-yl)-1H-pyrazol-1- yl)-N- methylcyclohexanecarbox- amide | 340.39 |
| 117 | 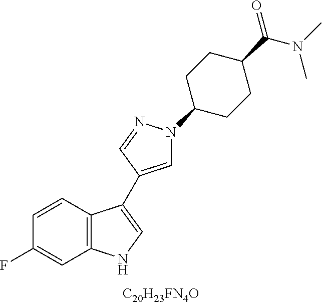 | (1S,4S)-4-(4-(6-fluoro-1H- indol-3-yl)-1H-pyrazol-1- yl)-N,N- dimethylcyclohexanecarbox- amide | 354.42 |
| 118 | 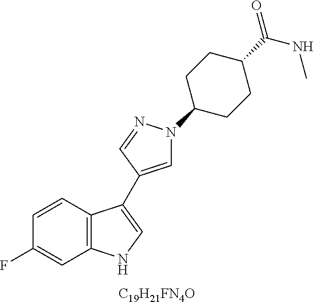 | (1R,4R)-4-(4-(6-fluoro-1H- indol-3-yl)-1H-pyrazol-1- yl)-N- methylcyclohexanecarbox- amide | 340.39 |
| 119 | 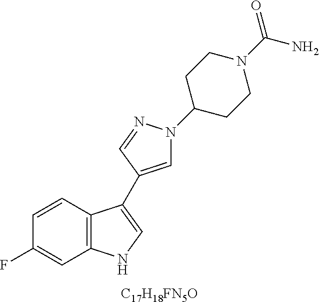 | 4-(4-(6-fluoro-1H-indol-3- yl)-1H-pyrazol-1- yl)piperidine-1-carboxamide | 327.36 |
| 120 | 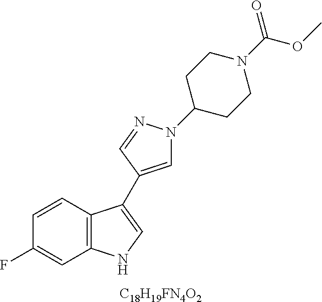 | methyl 4-(4-(6-fluoro-1H- indol-3-yl)-1H-pyrazol-1- yl)piperidine-1-carboxylate | 342.37 |
| 121 | 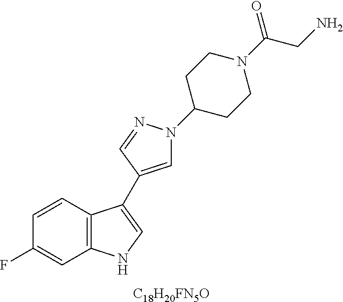 | 2-amino-1-(4-(4-(6-fluoro- 1H-indol-3-yl)-1H-pyrazol- 1-yl)piperidin-1-yl)ethanone | 341.38 |
| 122 | 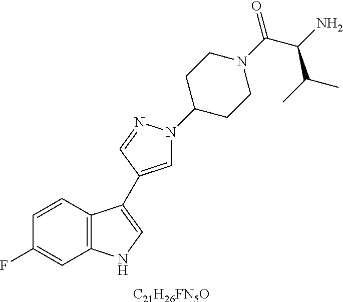 | (S)-2-amino-1-(4-(4-(6- fluoro-1H-indol-3-yl)-1H- pyrazol-1-yl)piperidin-1-yl)- 3-methylbutan-1-one | 383.46 |
| 123 | 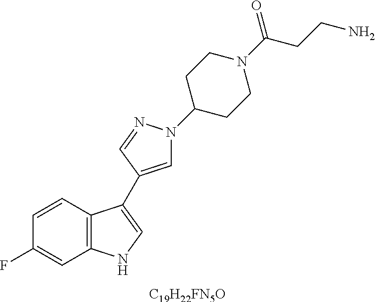 [SMILES: NCCC(=O)N1CCC(n2cc(-c3c[nH]c4cc(F)ccc34)cn2)CC1 ; "C19H22FN5O"] | 3-amino-1-(4-(4-(6-fluoro- 1H-indol-3-yl)-1H-pyrazol- 1-yl)piperidin-1-yl)propan- 1-one | 355.41 |
| 124 | 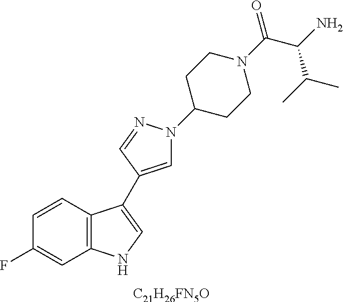 | (R)-2-amino-1-(4-(4-(6- fluoro-1H-indol-3-yl)-1H- pyrazol-1-yl)piperidin-1-yl)- 3-methylbutan-1-one | 383.46 |
| 125 | 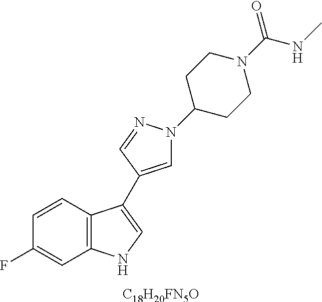 | 4-(4-(6-fluoro-1H-indol-3- yl)-1H-pyrazol-1-yl)-N- methylpiperidine-1- carboxamide | 341.38 |
| 126 | 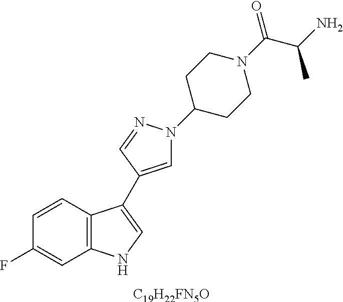 | (S)-2-amino-1-(4-(4-(6- fluoro-1H-indol-3-yl)-1H- pyrazol-1-yl)piperidin-1- yl)propan-1-one | 355.41 |

or pharmaceutically acceptable enantiomers, salts and solvates thereof.

In Table 1, the term “Cpd” means compound.

The compounds of Table 1 were named using ChemBioDraw® Ultra version 12.0 (PerkinElmer).

The compounds of Formula I and subformulae thereof may contain an asymmetric center and thus may exist as different stereoisomeric forms. Accordingly, the present invention includes all possible stereoisomers and includes not only racemic compounds but the individual enantiomers and their non-racemic mixtures as well. When a compound is desired as a single enantiomer, such may be obtained by stereospecific synthesis, by resolution of the final product or any convenient intermediate, or by chiral chromatographic methods as each are known in the art. Resolution of the final product, an intermediate, or a starting material may be performed by any suitable method known in the art.

The compounds of the invention may be in the form of pharmaceutically acceptable salts. Pharmaceutically acceptable salts of the compounds of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula I may be prepared by one or more of these methods:

(i) by reacting the compound of Formula I with the desired acid;

(ii) by reacting the compound of Formula I with the desired base;

(iii) by removing an acid- or base-labile protecting group from a suitable precursor of the compound of Formula I or by ring-opening a suitable cyclic precursor, for example, a lactone or lactam, using the desired acid; or

(iv) by converting one salt of the compound of Formula I to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

These salts may be prepared by standard procedures, e.g. by reacting a free acid with a suitable organic or inorganic base. Where a basic group is present, such as amino, an acidic salt, i.e. hydrochloride, hydrobromide, acetate, palmoate, and the like, can be used as the dosage form.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

The compounds of the present invention may be administered in the form of pharmaceutically acceptable solvate. The term “solvates” is taken to mean adductions of inert solvent molecules onto the compounds which form owing to their mutual attractive force. The term “pharmaceutically acceptable solvate” is intended to include all acceptable solvates such as hydrates or alcoholates and the like. Depending on the particular structures of the compound of the present invention, pharmaceutically acceptable solvates of the compounds of this invention include those formed from (but not limited to) water, ethanol, 1-propanol, 2-propanol, 2-methyl-2-propanol, acetic acid, ethyl acetate, 1-propyl acetate or 2-propyl acetate.

These solvates may be prepared by standard procedures, e.g. by crystallizing or precipitating a compound of the invention in presence of a solvent such as, but not limited to, water, ethanol, 1-propanol, 2-propanol, 2-methyl-2-propanol, acetic acid, ethyl acetate, 1-propyl acetate or 2-propyl acetate.

All references to compounds of formula I include references to enantiomers, salts, solvates, polymorphs, multi-component complexes and liquid crystals thereof.

The compounds of the invention include compounds of formula I as hereinbefore defined, including all polymorphs and crystal habits thereof, prodrugs and isomers thereof (including optical, geometric and tautomeric isomers) and isotopically-labeled compounds of formula I.

In addition, although generally, with respect to the salts of the compounds of the invention, pharmaceutically acceptable salts are preferred, it should be noted that the invention in its broadest sense also included non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I above.

The invention also generally covers all pharmaceutically acceptable predrugs and prodrugs of the compounds of Formula I.

Process for Manufacturing

The compounds of Formula I can be prepared by different ways with reactions known to a person skilled in the art.

(A) Process Involving a Protection of the Indole Amine

The invention further relates to a process (A) for manufacturing of compounds of Formula I,

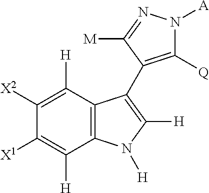

and pharmaceutically acceptable enantiomers, salts and solvates thereof, wherein X¹, X², M, Q and A are as defined above;

comprising deprotecting the indole amine of compound of Formula IV:

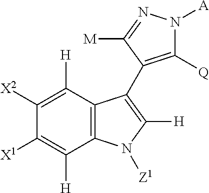

wherein
X¹, X², M, Q and A are as defined above; and
Z¹ represents an amino-protecting group such as for example an arylsulphonyl, a tert-butoxy carbonyl, a methoxymethyl, a para-methoxy benzyl, a benzyl or any other suitable protecting group known by those skilled in the art; to afford compound of Formula I.

According to one embodiment, deprotection step of the process (A) of the invention, depending on the nature of the group Z¹, may be performed by treatment with bases, such as but not limited to sodium hydroxide, potassium hydroxide, potassium carbonate, in the presence or absence of a suitable solvent such as but not limited to methanol, ethanol, isopropanol, tert-butanol, THF, DMF, dioxane, water or a mixture thereof, at a temperature between about 20° C. to about 100° C., preferably at about 85° C., for a few hours, e.g. one hour to 24 h. Alternatively, depending on the nature of the group Z¹, step b) may be performed in the presence of strong acids, such as but not limited to HCl, TFA, HF, HBr, in the presence or absence of a suitable solvent such as methanol, ethanol, isopropanol, tert-butanol, THF, DMF, Dioxane, water or a mixture thereof, at a temperature between about 20° C. to about 100° C., for a period comprised between 10 minutes and a few hours, e.g. 10 minutes to 24 h.

According to one embodiment, the process (A) of the invention further comprises a preliminary step, selected for step (a1) or step (a2), according to the following general scheme:

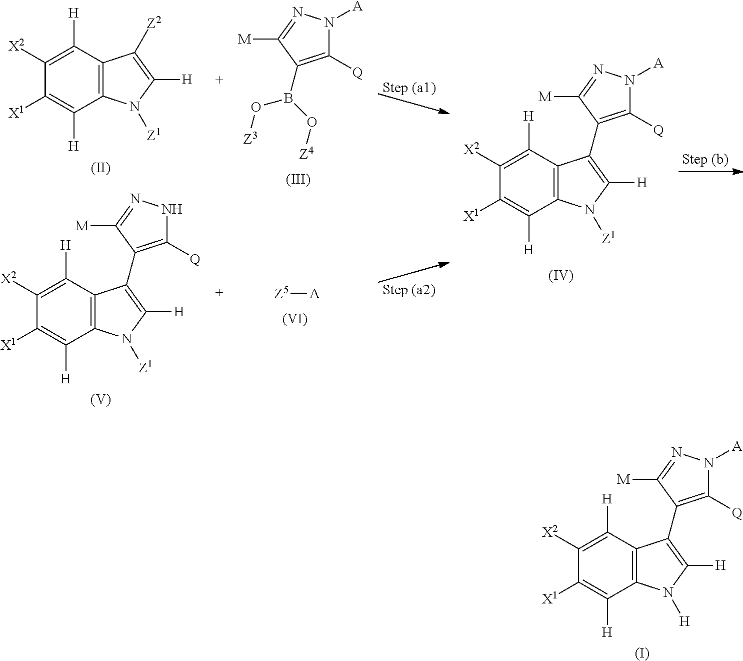

The invention relates to a first process (A1) of manufacturing of compounds of Formula I, according to the following general scheme:

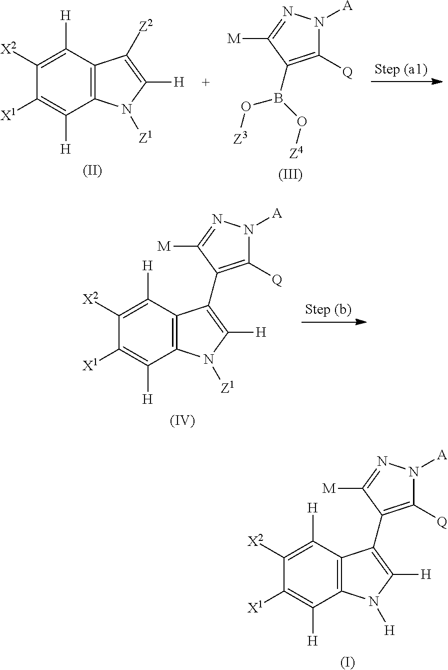

According to an embodiment, the first process (A1) of manufacturing of compounds of Formula I:

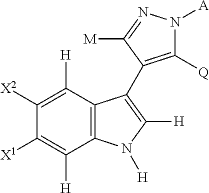

and pharmaceutically acceptable enantiomers, salts and solvates thereof, wherein X¹, X², M, Q and A are as defined above;

is characterized in that it comprises the following steps:

(a1) reacting a compound of Formula II,

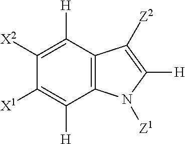

wherein
X¹ and X² are defined as above;
Z¹ represents an amino-protecting group such as for example an arylsulphonyl, a tert-butoxy carbonyl, a methoxymethyl, a para-methoxy benzyl, a benzyl or any other suitable protecting group known by those skilled in the art;
Z² represents an halogen (preferably iodine, bromine or chlorine), an alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy);

with a compound of Formula III

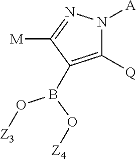

wherein
M, Q and A are defined as above;
Z³ and Z⁴ represent alkyl groups, with the possibility for Z³ and Z⁴ to form together a ring;

so as to obtain a compound of Formula IV,

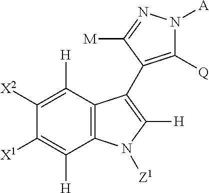

wherein X¹, X², M, Q, A and Z¹ are defined as above;

(b) deprotecting the indole amine of compound of Formula IV, to afford compound of Formula I.

According to one embodiment, step (a1) of the process (A1) of the invention may be performed with or without a catalyst such as but not limited to Pd₂(dba)₃, Pd(PPh₃)₄, dichlorobis(triphenylphosphine)palladium(II) or 1,1′-bis(diphenylphosphino)ferrocenedichloro palladium(II), Pd(OAc)₂, or Pd/C in the presence or absence of an additional ligand, such as but not limited to X-Phos, S-Phos, P(oTol)₃, PPh₃, BINAP, P(tBu)₃ or any other suitable phosphine ligand known to those skilled in the art.

According to one embodiment, step (a1) of the process (A1) of the invention is preferably performed in the presence of bases such as but not limited to K₃PO₄, K₂CO₃, Na₂CO₃.

According to one embodiment, step (a1) of the process (A1) of the invention is preferably performed in the presence of a suitable solvent such as but not limited to dioxane, THF, DMF, water or mixtures thereof, preferably in a mixture of dioxane or THF and water.

According to one embodiment, step (a1) of the process (A1) of the invention may be carried out at a temperature ranging from about 20° C. to about 180° C., with or without microwave irradiation, for a period ranging from 10 minutes to a few hours, preferably from 10 minutes to 24 h.

The invention further relates to a second process (A2) of manufacturing of compounds of Formula I, according to the following general scheme:

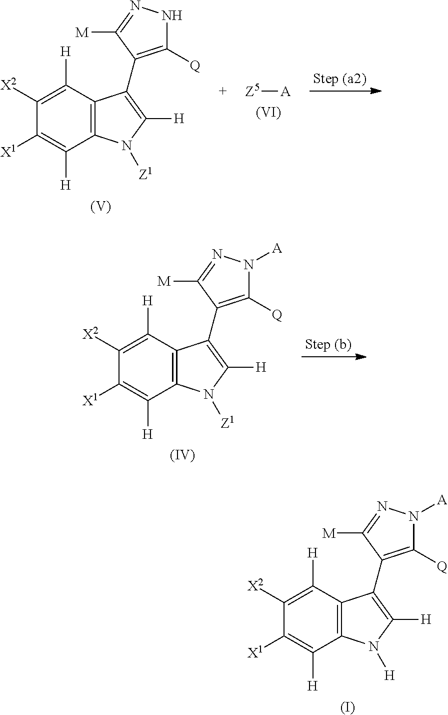

According to an embodiment, the second process (A2) of manufacturing of compounds of Formula I:

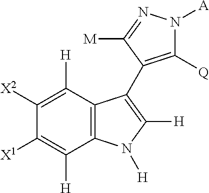

and pharmaceutically acceptable enantiomers, salts and solvates thereof, wherein X¹, X², M, Q and A are as defined above;

is characterized in that it comprises the following steps:

(a2) reacting a compound of Formula V,

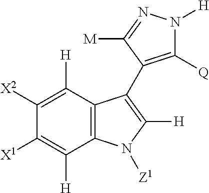

wherein
X¹, X², M and Q are defined as above; and
Z¹ represents an amino-protecting group such as for example an arylsulphonyl, a tert-butoxy carbonyl, a methoxymethyl, a para-methoxy benzyl, a benzyl or any other suitable protecting group known by those skilled in the art;

with a compound of Formula VI

Z⁵-A
wherein
A is defined as above; and
Z⁵ represents an halogen (preferably iodine, bromine or chlorine), alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy), or any other leaving group known to those skilled in the art;

so as to obtain a compound of Formula IV,

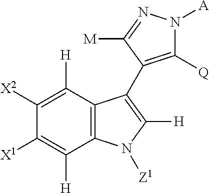

wherein X¹, X², M, Q, A and Z¹ are defined as above;

(b) deprotecting the indole amine of compound of Formula IV, to afford compound of Formula I.

According to one embodiment, step (a2) of the process (A2) of the invention may be performed in the presence of bases such as but not limited to cesium carbonate, sodium carbonate, potassium carbonate, sodium hydride, sodium hydroxide, potassium hydroxide.

According to one embodiment, step (a2) of the process (A2) of the invention may be performed in the presence of a suitable solvent such as but not limited to DMF, methanol, ethanol, isopropanol, tert-butanol, THF, dioxane, dichloromethane, water.

According to one embodiment, step (a2) of the process (A2) of the invention may be performed in the presence or absence of catalytic amounts of appropriate iodide salts, such as but not limited to tetrabutylammonium iodide.

According to one embodiment, step (a2) of the process (A2) of the invention may be carried out at a temperature between about 20° C. to about 180° C., with or without microwave irradiation, for a period comprised between 10 minutes and a few hours, e.g. 10 minutes to 24 h.

(B) General Process

The invention further relates to a general process (B1) for manufacturing of compounds of Formula I,

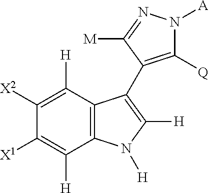

and pharmaceutically acceptable enantiomers, salts and solvates thereof, wherein X¹, X², M, Q and A are as defined above;

characterized in that it comprises the following steps:

(a3) reacting a compound of Formula II,

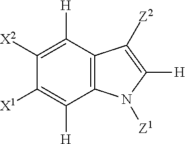

wherein
X¹ and X² are defined as above;
Z¹ represents H or an amino-protecting group such as for example an arylsulphonyl, a tert-butoxy carbonyl, a methoxymethyl, a para-methoxy benzyl, a benzyl or any other suitable protecting group known by those skilled in the art;
Z² represents an halogen (preferably iodine, bromine or chlorine), an alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy);

with a compound of Formula III

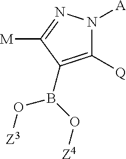

wherein
M, Q and A are defined as above;
Z³ and Z⁴ represent alkyl groups, with the possibility for Z³ and Z⁴ to form together a ring;

so as to obtain a compound of Formula IV,

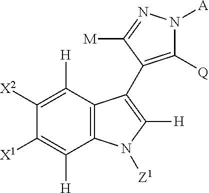

wherein X¹, X², M, Q, A and Z¹ are defined as above; and

(b) in the case wherein Z¹ is not H, deprotecting the indole amine of compound of Formula IV, to afford compound of Formula I.

According to one embodiment, step (a3) of the process (B1) of the invention may be performed as described above for step (a1) of the process (A1).

According to one embodiment, step (b) of the process (B1) of the invention may be performed as described above for step (b) of the process (A).

The invention further relates to a general process (B2) for manufacturing of compounds of Formula I,

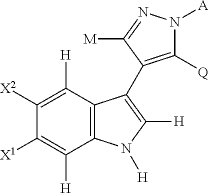

and pharmaceutically acceptable enantiomers, salts and solvates thereof, wherein X¹, X², M, Q and A are as defined above;

characterized in that it comprises the following steps:

(a4) reacting a compound of Formula V,

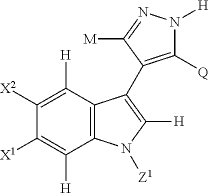

wherein
X¹, X², M and Q are defined as above; and
Z¹ represents H or an amino-protecting group such as for example an arylsulphonyl, a tert-butoxy carbonyl, a methoxymethyl, a para-methoxy benzyl, a benzyl or any other suitable protecting group known by those skilled in the art;

with a compound of Formula VI

Z⁵-A
wherein
A is defined as above; and
Z⁵ represents an halogen (preferably iodine, bromine or chlorine), alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy), or any other leaving group known to those skilled in the art;

so as to obtain a compound of Formula IV,

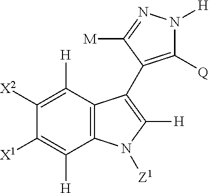

wherein X¹, X², M, Q, A and Z¹ are defined as above; and

(b) in the case wherein Z¹ is not H, deprotecting the indole amine of compound of Formula IV, to afford compound of Formula I.

According to one embodiment, step (a4) of the process (B2) of the invention may be performed as described above for step (a2) of the process (A2).

According to one embodiment, step (b) of the process (B2) of the invention may be performed as described above for step (b) of the process (A).

In general, the synthesis pathways for any individual compound of Formula (I) will depend on the specific substituents of each molecule and upon the ready availability of intermediates necessary; again such factors being appreciated by those of ordinary skill in the art.

According to a further general process, compounds of Formula I can be converted to alternative compounds of Formula I, employing suitable interconversion techniques well known by a person skilled in the art.

Compounds of the formula (I) and related formulae can furthermore be obtained by liberating compounds of the formula (I) from one of their functional derivatives by treatment with a solvolysing or hydrogenolysing agent.

Preferred starting materials for the solvolysis or hydrogenolysis are those which conform to the formula I and related formulae, but contain corresponding protected amino and/or hydroxyl groups instead of one or more free amino and/or hydroxyl groups, preferably those which carry an amino-protecting group instead of an H atom bonded to an N atom, in particular those which carry an R*—N group, in which R* denotes an amino-protecting group, instead of an HN group, and/or those which carry a hydroxyl-protecting group instead of the H atom of a hydroxyl group, for example those which conform to the formula I, but carry a —COOR** group, in which R** denotes a hydroxyl-protecting group, instead of a —COOH group.

It is also possible for a plurality of—identical or different—protected amino and/or hydroxyl groups to be present in the molecule of the starting material. If the protecting groups present are different from one another, they can in many cases be cleaved off selectively.

The term “amino-protecting group” is known in general terms and relates to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. Since the amino-protecting groups are removed after the desired reaction (or reaction sequence), their type and size are furthermore not crucial; however, preference is given to those having 1-20, in particular 1-8, carbon atoms. The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and tolyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl, such as Mtr. Preferred amino-protecting groups are BOC and Mtr, furthermore CBZ, Fmoc, benzyl and acetyl.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical of such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, 4-methoxybenzyl, p-nitrobenzoyl, p-toluenesulfonyl, tert-butyl and acetyl, where benzyl and tert-butyl are particularly preferred.

The compounds of the formula I and related formulae are liberated from their functional derivatives—depending on the protecting group used—by using for example strong inorganic acids, such as hydrochloric acid, perchloric acid or sulfuric acid, strong organic carboxylic acids, such as trichloroacetic acid, TFA or sulfonic acids, such as benzene- or p-toluenesulfonic acid. The presence of an additional inert solvent is possible, but is not always necessary. Suitable inert solvents are preferably organic, for example carboxylic acids, such as acetic acid, ethers, such as tetrahydrofuran or dioxane, amides, such as DMF, halogenated hydrocarbons, such as dichloromethane, furthermore also alcohols, such as methanol, ethanol or isopropanol, and water. Mixtures of the above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0 and about 50° C., preferably between 15 and 30° C. (room temperature).

The BOC, OtBu and Mtr groups can, for example, preferably be cleaved off using TFA in dichloromethane or using approximately 3 to 5N HCl in dioxane at 15-30° C., and the FMOC group can be cleaved off using an approximately 5 to 50% solution of dimethylamine, diethylamine or piperidine in DMF at 15-30° C.

Protecting groups which can be removed hydrogenolytically (for example CBZ, benzyl or the liberation of the amidino group from the oxadiazole derivative thereof) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between about 0 and 100° C. and pressures between about 1 and 200 bar, preferably at 20-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 20-30° C.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, trifluoromethylbenzene, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide, N-methylpyrrolidone (NMP) or dimethyl [symbol] formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Esters can be hydrolysed, for example, using HCl, H₂SO₄, or using LiOH, NaOH or KOH in water, water/THF, water/THF/ethanol or water/dioxane, at temperatures between 0 and 100° C.

Free amino groups can furthermore be acylated in a conventional manner using an acyl chloride or anhydride or alkylated using an unsubstituted or substituted alkyl halide, advantageously in an inert solvent, such as dichloromethane or THF and/or in the presence of a base, such as triethylamine or pyridine, at temperatures between −60° C. and +30° C.

For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999.

Reaction schemes as described in the example section are illustrative only and should not be construed as limiting the invention in any way.

Use

The invention is further directed to the use of the compounds of the invention or pharmaceutically acceptable enantiomers, salts and solvates thereof as TDO2 inhibitors.

Accordingly, in a particularly preferred embodiment, the invention relates to the use of compounds of Formula I and subformulae in particular those of Table 1 above, or pharmaceutically acceptable enantiomers, salts and solvates thereof, as TDO2 inhibitors.

Accordingly, in another aspect, the invention relates to the use of these compounds or enantiomers, salts and solvates thereof for the synthesis of pharmaceutical active ingredients, such as TDO2 inhibitors.

In one embodiment, the invention relates to the use of compounds of Formula I and subformulae in particular those of Table 1 above, or pharmaceutically acceptable enantiomers, salts and solvates thereof, for increasing immune recognition and destruction of the cancer cells.

The compounds of the invention are therefore useful as medicaments, in particular in the prevention and/or treatment of cancer.

In one embodiment, compounds of the invention or pharmaceutically acceptable enantiomers, salts or solvates thereof are for use in the treatment and/or prevention of cancer, neurodegenerative disorders such as Parkinson's disease, Alzheimer's disease and Huntington's disease, chronic viral infections such as HCV and HIV, depression, and obesity.

The invention further relates to a method for treatment or prevention of cancer, neurodegenerative disorders such as Parkinson's disease, Alzheimer's disease and Huntington's disease, chronic viral infections such as HCV and HIV, depression, and obesity, which comprises administering to a mammalian species in need thereof a therapeutically effective amount of the compound according to the invention or a pharmaceutically acceptable enantiomers, salts or solvates thereof.

In the context of neurological brain disorders, TDO2 expression has been demonstrated in neurons, brain vasculature and additionally in the case of schizophrenia in astroglial cells (Miller C et al., 2004, Neurobiology Dis, 15(3):618-29). The kynurenine pathway is now considered as a therapeutic target in cognitive diseases like bipolar disorder or Tourette syndrome and neurodegenerative disorders like Alzheimer, motor neuron disease like Amyotrophic lateral sclerosis, Multiple sclerosis, Huntington or Parkinson's disease (Stone T W, 2013, Br J of Pharmacol, 169(6): 1211-27; Wu et al, 2013, Plos One, 8(4):e59749; Fivesi et al, 2012, J Neural Transm, 119(2):225-34; Widner et al, 2002, J Neural Transm, 109(2):181-9; Comings et al, 1996, Pharmacogenetics, 6(4):307-18; Forrest 2010, J Neurochem, 112(1):112-22).

Cognitive changes related to Tryptophan catabolism have also been shown in patients infected with human immunodeficiency virus type-1 (HIV), called HIV-associated neurocognitive disorder (HAND) (Davies et al, 2010, Int J of Tryptophan Res, 3:121-40). In addition, T cell hyporesponsiveness has been recently associated with the Tryptophan catabolic pathway in HIV-infected patients with possibly extension to other chronic infectious diseases like e.g. Hepatitis C.

Various cancers are known in the art. The cancer may be metastatic or non-metastatic. The cancer may be may be familial or sporadic. In some embodiments, the cancer is selected from the group consisting of: leukemia and multiple myeloma. Additional cancers that can be treated using the methods of the invention include, for example, benign and malignant solid tumours and benign and malignant non-solid tumours.

Examples of solid tumours include, but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumour), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumours (seminomas, and non-seminomas such as teratomas and choriocarcinomas), stromal tumours, germ cell tumours, and thyroid cancer (including thyroid adenocarcinoma and medullary carcinoma).

Examples of non-solid tumours include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

In one embodiment, compounds of the invention or pharmaceutically acceptable enantiomers, salts or solvates thereof are for use in the treatment and/or prevention of a cancer selected from bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma, pancreatic carcinoma, gynaecological cancers (ovarian carcinoma, cervical cancer, endometrial cancer).

In one embodiment, compounds of the invention or pharmaceutically acceptable enantiomers, salts or solvates thereof are for use in the treatment and/or prevention of a cancer selected from bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma, pancreatic carcinoma.

In one embodiment, compounds of the invention or pharmaceutically acceptable enantiomers, salts or solvates thereof are for use in the treatment and/or prevention of a cancer selected gynaecological cancers such as for example ovarian carcinoma, cervical cancer, endometrial cancer.

In a specific embodiment, compounds of the invention or pharmaceutically acceptable enantiomers, salts or solvates thereof are for use in the treatment and/or prevention of a cancer selected from hepatocarcinoma and glioblastoma.

The invention further relates to a method for treatment or prevention of a cancer selected from bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma, pancreatic carcinoma, gynaecological cancers (ovarian carcinoma, cervical cancer, endometrial cancer), which comprises administering to a mammalian species in need thereof a therapeutically effective amount of the compound according to the invention or a pharmaceutically acceptable enantiomers, salts or solvates thereof.

The invention further relates to a method for treatment or prevention of a cancer selected from bladder carcinoma, hepatocarcinoma, melanoma, mesothelioma, neuroblastoma, sarcoma, breast carcinoma, leukemia, renal cell carcinoma, colorectal carcinoma, head & neck carcinoma, lung carcinoma, brain tumor, glioblastoma, astrocytoma, myeloma, pancreatic carcinoma, which comprises administering to a mammalian species in need thereof a therapeutically effective amount of the compound according to the invention or a pharmaceutically acceptable enantiomers, salts or solvates thereof.

The invention further relates to a method for treatment or prevention of a cancer selected from gynaecological cancers such as for example ovarian carcinoma, cervical cancer, endometrial cancer; which comprises administering to a mammalian species in need thereof a therapeutically effective amount of the compound according to the invention or a pharmaceutically acceptable enantiomers, salts or solvates thereof.

In a specific embodiment, the method for treatment or prevention is a method for treatment or prevention of a cancer selected from hepatocarcinoma and glioblastoma, which comprises administering to a mammalian species in need thereof a therapeutically effective amount of the compound according to the invention or a pharmaceutically acceptable enantiomers, salts or solvates thereof.

The invention also provides for a method for delaying in patient the onset of cancer comprising the administration of a pharmaceutically effective amount of a compound of Formula I or pharmaceutically acceptable enantiomer, salt and solvate thereof to a patient in need thereof.

Preferably, the patient is a warm-blooded animal, more preferably a human.

The compounds of the invention are especially useful in the treatment and/or prevention of cancer.

In a specific embodiment, the compounds of the invention are especially useful in the treatment and/or prevention of cancer.

The invention further provides the use of a compound of Formula I or a pharmaceutically acceptable enantiomer, salt and solvate thereof for the manufacture of a medicament for treating and/or preventing cancer.

According to a further feature of the present invention there is provided a method for modulating TDO2 activity, in a patient, preferably a warm blooded animal, and even more preferably a human, in need of such treatment, which comprises administering to said patient an effective amount of compound of the present invention, or a pharmaceutically acceptable enantiomer, salt and solvate thereof.

Formulations

The invention also provides pharmaceutical compositions comprising a compound of Formula I or a pharmaceutically acceptable enantiomer, salt and solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable enantiomer, salt and solvate thereof as active ingredient, additional therapeutic agents and/or active ingredients.

Another object of this invention is a medicament comprising at least one compound of the invention, or a pharmaceutically acceptable enantiomer, salt and solvate thereof, as active ingredient.

According to a further feature of the present invention there is provided the use of a compound of Formula I or a pharmaceutically acceptable enantiomer, salt and solvate thereof for the manufacture of a medicament for modulating TDO2 activity in a patient, in need of such treatment, which comprises administering to said patient an effective amount of compound of the present invention, or a pharmaceutically acceptable enantiomer, salt and solvate thereof.

Generally, for pharmaceutical use, the compounds of the invention may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

Depending on the condition to be prevented or treated and the route of administration, the active compound of the invention may be administered as a single daily dose, divided over one or more daily doses, or essentially continuously, e.g. using a drip infusion.

### DEFINITIONS

In the present invention, the following terms have the following meanings:

Where groups may be substituted, such groups may be substituted with one or more substituents, and preferably with one, two or three substituents. Substituents may be selected from but not limited to, for example, the group comprising halogen, hydroxyl, oxo, nitro, amido, carboxy, amino, cyano haloalkoxy, and haloalkyl.

The term “halogen” means fluoro, chloro, bromo, or iodo. Preferred halo groups are fluoro and chloro.

The term “alkyl” by itself or as part of another substituent refers to a hydrocarbyl radical of Formula CₙH₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms. Alkyl groups may be linear or branched and may be substituted as indicated herein. Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

The term “haloalkyl” alone or in combination, refers to an alkyl radical having the meaning as defined above wherein one or more hydrogens are replaced with a halogen as defined above. Non-limiting examples of such haloalkyl radicals include fluoromethyl, difluoromethyl, trifluoro methyl and the like.

The term “cycloalkyl” as used herein is a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl, with cyclopropyl being particularly preferred.

Where at least one carbon atom in a cycloalkyl group is replaced with a heteroatom, the resultant ring is referred to herein as “heterocyclyl”.

The terms “heterocyclyl” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. Any of the carbon atoms of the heterocyclic group may be substituted by oxo (for example piperidone, pyrrolidinone). The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, azetidinyl, tetrahydropyranyl, piperazinyl, imidazolinyl, morpholinyl, oxetanyl, pyrazolidinyl imidazolidinyl, isoxazolinyl, oxazolidinyl, isoxazolidinyl, thiazolidinyl, isothiazolidinyl, indolyl, indolinyl, isoindolinyl, tetrahydrofuranyl, tetrahydroquinolinyl, thiomorpholinyl, thiomorpholinylsulfoxide, thiomorpholinylsulfone, pyrrolizinyl.

The term “alkene” as used herein refers to an unsaturated hydrocarbyl group, which may be linear or branched, comprising one or more carbon-carbon double bonds. Suitable alkenyl groups comprise between 2 and 6 carbon atoms, preferably between 2 and 4 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “aryl” as used herein refers to a polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenylnaphthalenyl, indenyl.

The term “heteroaryl” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyridazinyl, pyridinyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, oxazinyl, dioxinyl, thiazinyl, triazinyl, indolyl, indolizinyl, isoindolyl, benzofuranyl, isobenzofuranyl, benzothiophenyl, isobenzothiophenyl, indazolyl, benzimidazolyl, quinolinyl, isoquinolinyl, cinnolinyl, quinazolinyl, quinoxalinyl.

The term “arylalkyl” refers to any group -alkyl-aryl. The term “alkylaryl” refers to any group -aryl-alkyl.

The term “heteroarylalkyl” refers to any group -alkyl-heteroaryl. The term “alkylheteroaryl” refers to any group -heteroaryl-alkyl.

The term “alkoxy” refers to any group O-alkyl. The term“haloalkoxy” refers to any group O-haloalkyl.

The term “oxo” refers to a ═O moiety.

The term “amino” refers to a —NH₂ group or any group derived thereof by substitution of one nor two hydrogen atom by an organic aliphatic or aromatic group. Preferably, groups derived from —NH₂ are alkylamino groups, i.e. N-alkyl groups, comprising monoalkylamino and dialkylamino. According to a specific embodiment, the term “amino” refers to NH₂, NHMe or NMe₂.

The term “amino-protecting group” refers to a protecting group for an amine function. According to a preferred embodiment, the amino-protecting group is selected in the groups comprising: arylsulphonyl, tert-butoxy carbonyl, methoxymethyl, para-methoxy benzyl or benzyl.

The term “leaving group” refers to a molecular fragment that departs with a pair of electrons in heterolytic bond cleavage. According to a preferred embodiment, the leaving group is selected in the groups comprising: halogen, preferably iodine, bromine or chlorine; alkylsulfonyloxy having 1-6 carbon atoms, preferably methylsulfonyloxy or trifluoromethylsulfonyloxy; or arylsulfonyloxy having 6-10 carbon atoms, preferably phenyl- or p-tolylsulfonyloxy.

The term “solvate” is used herein to describe a compound in this invention that contains stoichiometric or sub-stoichiometric amounts of one or more pharmaceutically acceptable solvent molecule such as ethanol.

The term “hydrate” refers to when the said solvent is water.

The compounds of the invention include compounds of Formula I as hereinbefore defined, including all polymorphs and crystal habits thereof, predrugs and prodrugs thereof and isotopically-labeled compounds of Formula I.

The invention also generally covers all pharmaceutically acceptable predrugs and prodrugs of the compounds of Formula I.

The term “prodrug” as used herein means the pharmacologically acceptable derivatives of compounds of Formula I, such as for example esters, whose in vivo biotransformation product generates the biologically active drug. Prodrugs are generally characterized by increased bio-availability and are readily metabolized into biologically active compounds in vivo.

The term “predrug”, as used herein, means any compound that will be modified to form a drug species, wherein the modification may take place either inside or outside of the body, and either before or after the predrug reaches the area of the body where administration of the drug is indicated.

The term “patient” refers to a warm-blooded animal, more preferably a human, who/which is awaiting the receipt of, or is receiving medical care or is/will be the object of a medical procedure.

The term “human” refers to a subject of both genders and at any stage of development (i.e. neonate, infant, juvenile, adolescent, adult).

The terms “treat”, “treating” and “treatment”, as used herein, are meant to include alleviating, attenuating or abrogating a condition or disease and/or its attendant symptoms.

The terms “prevent”, “preventing” and “prevention”, as used herein, refer to a method of delaying or precluding the onset of a condition or disease and/or its attendant symptoms, barring a patient from acquiring a condition or disease, or reducing a patient's risk of acquiring a condition or disease.

The term “therapeutically effective amount” (or more simply an “effective amount”) as used herein means the amount of active agent or active ingredient that is sufficient to achieve the desired therapeutic or prophylactic effect in the patient to which/whom it is administered.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the patient thereof.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, and liposomes.

### BRIEF DESCRIPTION OF THE DRAWINGS

FIG. 1 is a histogram the tryptophan concentration in plasma of mice after administration of a vehicle or of compound 89 at various doses (*=p<0.05; **=p<0.001).

### EXAMPLES

The present invention will be better understood with reference to the following examples. These examples are intended to representative of specific embodiments of the invention, and are not intended as limiting the scope of the invention.

### I. Chemistry Examples

The MS data provided in the examples described below were obtained as followed: Mass spectrum: LC/MS Agilent 6110 (ESI) or a Waters Acquity SQD (ESI).

The NMR data provided in the examples described below were obtained as followed: Bruker Ultrashield™ 400 PLUS and Bruker Fourier 300 MHz and TMS was used as an internal standard.

The microwave chemistry was performed on a single mode microwave reactor Initiator Microwave System EU from Biotage.

Preparative HPLC purifications were performed with a mass directed autopurification Fractionlynx from Waters equipped with a Xbridge™ Prep C18 OBD column 19×150 mm 5 μm, unless otherwise reported. All HPLC purifications were performed with a gradient of CH₃CN/H₂O/NH₄HCO₃ (5 mM), CH₃CN/H₂O/TFA (0.1%), or CH₃CN/H₂O/NH₃H₂O (0.1%).

### I.1. Synthesis of Intermediate Compounds

### Intermediate 1: 6-fluoro-1-(phenylsulfonyl)-1H-indole

The title compound was prepared using the same procedure as reported (Bioorg. Med. Chem. 2011, 19, 4782-4795).
Intermediate 2: 3-bromo-6-fluoro-1-(phenylsulfonyl)-1H-indole

To a solution of 6-fluoro-1-(phenylsulfonyl)-1H-indole (Intermediate 1; 1.0 g; 3.6 mmol) in DCM (20 mL) at 0° C. was added a solution of bromine (0.64 g; 4.0 mmol) in DCM (20 mL) dropwise. The mixture was stirred at 0° C. for 0.5 h, then added saturated aqueous Na₂S₂O₃ (10 mL), and stirred at r.t. for 10 minute. The organic layer was separated and the aqueous layer was extracted with DCM (10 mL×2). The combined organic layers were washed with saturated aqueous NaHCO₃ (20 mL×2), water (20 mL×2), brine (20 mL), dried over anhydrous Na₂SO₄, filtered, and concentrated to afford 1.26 g (99%) of the title compound as a pink solid.

¹H NMR (400 MHz, DMSO-d₆) δ [ppm]: 8.21 (s, 1H), 8.13-8.11 (m, 2H), 7.83-7.73 (m, 2H), 7.66-7.62 (m, 2H), 7.52-7.49 (m, 1H), 7.30-7.26 (m, 1H).

### Intermediate 3: 6-fluoro-3-iodo-1-(phenylsulfonyl)-1H-indole

The title compound was prepared using the same procedure as reported (WO2010/136491A1).

### Intermediate 4: tert-butyl 4-(6-fluoro-1-(phenylsulfonyl)-1H-indol-3-yl)-1H-pyrazole-1-carboxylate

To a solution of 6-fluoro-3-iodo-1-(phenylsulfonyl)-1H-indole (Intermediate 3; 535 mg; 1.33 mmol), tert-butyl 4-(4,4,5,5-tetramethyl-1,3,2-dioxaborolan-2-yl)-1H-pyrazole-1-carboxylate (588 mg; 2.0 mmol), K₃PO₄ (848 mg; 4.0 mmol) in dioxane (20 mL) and water (2 mL) was added Pd(dppf)Cl₂ (110 mg; 0.13 mmol) under nitrogen. The mixture was stirred at 90° C. overnight. The mixture was filtered through Celite, diluted with EtOAc (100 mL) and water (100 mL). The aqueous layer was extracted with EtOAc (50 mL×2). The combined organic layers were washed with brine (100 mL), dried over anhydrous Na₂SO₄, concentrated, and purified by a silica gel chromatography (petroleum ether/EtOAc=10/1-2/1) to afford 357 mg (61%) of the title compound as a yellow solid.

LC-MS: m/z 442.1 [M+H]⁺.

### Intermediate 5: 6-fluoro-1-(phenylsulfonyl)-3-(1H-pyrazol-4-yl)-1H-indole

To a solution of tert-butyl 4-(6-fluoro-1-(phenylsulfonyl)-1H-indol-3-yl)-1H-pyrazole-1-carboxylate (Intermediate 4; 310 mg; 0.70 mmol) in methanol (2 mL) was added saturated HCl in Et₂O (10 mL). The resulting mixture was stirred for 30 minutes. The reaction was concentrated to dryness under reduced pressure, diluted with water (10 mL), neutralized with saturated aqueous NaHCO₃, and extracted with EtOAc (10 mL×3). The combined organic layers were washed with brine (5 mL), dried over anhydrous Na₂SO₄, filtered, and concentrated to afford 234 mg (89%) of the title compound as a yellow solid.

¹H NMR (300 MHz, DMSO-d₆) δ [ppm]: 13.06 (s, 1H), 8.31 (s, 1H), 8.09 (s, 1H), 8.08-8.00 (m, 3H), 7.91 (dd, J=8.7, 5.4 Hz, 1H), 7.77 (dd, J=9.6, 2.3 Hz, 1H), 7.74-7.67 (m, 1H), 7.64-7.57 (m, 2H), 7.21 (dt, J=8.7, 2.4 Hz, 1H).

### Intermediate 6: 6-fluoro-3-(1-methyl-1H-pyrazol-4-yl)-1-(phenylsulfonyl)-1H-indole

A mixture of 3-bromo-6-fluoro-1-(phenylsulfonyl)-1H-indole (Intermediate 2; 1.0 g; 2.8 mmol), 1-methyl-4-(4,4,5,5-tetramethyl-1,3,2-dioxaborolan-2-yl)-1H-pyrazole (1.7 g; 8.4 mmol), KOAc (1.4 g; 14.0 mmol) and Pd(dppf)Cl₂.CH₂Cl₂ (0.15 g; 0.18 mmol) in DMF (25 mL) flushed with nitrogen was heated to 90° C. overnight. The mixture was filtered through Celite, diluted with EtOAc (10 mL) and water (10 mL). The aqueous layer was extracted with EtOAc (20 mL×3). The combined organic layers were washed with brine, dried over anhydrous Na₂SO₄, filtered, concentrated, and purified by a silica gel chromatography (petroleum ether/EtOAc=1/1) to afford 0.20 g (23%) of the title compound as a yellow solid.

¹H NMR (400 MHz, DMSO-d₆) δ [ppm]: 8.22 (s, 1H), 8.09-8.05 (m, 3H), 7.96 (s, 1H), 7.90-7.87 (m, 1H), 7.79-7.76 (m, 1H), 7.72-7.69 (m, 1H), 7.63-7.59 (m, 2H), 7.25-7.20 (m, 1H), 3.89 (s, 3H).

### Intermediate 7: 4-(1H-pyrazol-1-yl)pyridine

The title compound was prepared using the same procedure as reported (Tetrahedron Lett. 2012, 53, 948-951).

### Intermediate 8: 4-(4-bromo-1H-pyrazol-1-yl)pyridine

To a solution of 4-(1H-pyrazol-1-yl)pyridine (Intermediate 7; 1.1 g; 7.5 mmol) in acetic acid (10 mL) was added a solution of bromine (6 mL) in acetic acid (10 mL) dropwise. The reaction mixture was stirred for 4 hours, diluted with saturated aqueous Na₂S₂O₃ (20 mL), and extracted with DCM (10 mL×3). The combined organic layers were washed with saturated aqueous NaHCO₃ (20 mL), water (20 mL), brine (20 mL), dried over anhydrous Na₂SO₄, filtered, and concentrated to afford 936 mg (55%) of the title compound as a pink solid.

¹H NMR (400 MHz, DMSO-d₆) δ [ppm]: 9.03 (s, 1H), 8.68 (d, J=6.0 Hz, 2H), 8.02 (s, 1H), 7.86 (d, J=6.1 Hz, 2H).

### Intermediate 9: 4-(4-(4,4,5,5-tetramethyl-1,3,2-dioxaborolan-2-yl)-1H-pyrazol-1-yl)pyridine

A mixture of 4-(4-bromo-1H-pyrazol-1-yl)pyridine (Intermediate 8; 400 mg; 1.8 mmol), 4,4,4′,4′,5,5,5′,5′-octamethyl-2,2′-bi(1,3,2-dioxaborolane (911 mg; 3.6 mmol), Pd(dppf)Cl₂ (147 mg; 0.2 mmol), KOAc (878 mg; 8.9 mmol) in 1,4-dioxane (40 mL) was stirred at 80° C. overnight under argon. The combined organic solution was washed with brine (20 mL), dried over anhydrous Na₂SO₄, filtered, and concentrated to afford the title compound as a yellow oil, which was used directly without further purification.

LC-MS: m/z 272.1 [M+H]⁺.

### Intermediate 10: 6-fluoro-1-(phenylsulfonyl)-3-(1-(pyridin-4-yl)-1H-pyrazol-4-yl)-1H-indole

A mixture of 3-bromo-6-fluoro-1-(phenylsulfonyl)-1H-indole (Intermediate 2; 317 mg; 0.9 mmol) and 4-(4-(4,4,5,5-tetramethyl-1,3,2-dioxaborolan-2-yl)-1H-pyrazol-1-yl)pyridine (Intermediate 9; 485 mg; 1.80 mmol), Pd₂dba₃ (48 mg; 0.05 mmol) and X-phos (96 mg; 0.20 mmol), and K₃PO₄ (954 mg; 4.5 mmol) in 1,4-dioxane (8 mL) and water (1 mL) was flushed with argon and reacted at 125° C. for 25 minutes in a microwave reactor. The reaction mixture was filtered through Celite, washed with EtOAc (50 mL), concentrated, and purified by a silica gel chromatography (petroleum ether/EtOAc=1/1) to afford 363 mg (96%) of the title compound as a yellow solid.

¹H NMR (300 MHz, DMSO-d₆) δ [ppm]: 9.24 (s, 1H), 8.70 (s, 2H), 8.50 (s, 1H), 8.37 (s, 1H), 8.16 (dd, J=8.5, 5.3 Hz, 1H), 8.10 (d, J=7.6 Hz, 2H), 8.00 (d, J=5.6 Hz, 2H), 7.82 (d, J=9.7 Hz, 1H), 7.73 (d, J=7.3 Hz, 1H), 7.64 (t, J=7.6 Hz, 2H), 7.30 (t, J=9.0 Hz, 1H).

### Intermediate 11: 3-(4-(4,4,5,5-tetramethyl-1,3,2-dioxaborolan-2-yl)-1H-pyrazol-1-yl)pyridine

The title compound was prepared using the same procedure as reported (US2011/166143 A1).

### Intermediate 12: 6-fluoro-1-(phenylsulfonyl)-3-(1-(pyridin-3-yl)-1H-pyrazol-4-yl)-1H-indole

Following the general method as outlined in Intermediate 10, starting from 3-(4-(4,4,5,5-tetramethyl-1,3,2-dioxaborolan-2-yl)-1H-pyrazol-1-yl)pyridine (Intermediate 11; 485 mg; 1.80 mmol), 362 mg (96%) of the title compound was obtained as a brown solid after purification by a silica gel chromatography (petroleum ether/EtOAc=1/1).

LC-MS: m/z 419.1 [M+H]⁺.

### Intermediate 13: 2-(4-bromo-1H-pyrazol-1-yl)pyridine

The title compound was prepared using the same procedure as reported (J. Med. Chem. 2004, 47, 4645-4648).

### Intermediate 14: 2-(4-(4,4,5,5-tetramethyl-1,3,2-dioxaborolan-2-yl)-1H-pyrazol-1-yl)pyridine

A mixture of 2-(4-bromo-1H-pyrazol-1-yl)pyridine (Intermediate 13; 400 mg; 1.80 mmol), 4,4,4′,4′,5,5,5′,5′-octamethyl-2,2′-bi(1,3,2-dioxaborolane (911 mg; 3.6 mmol), Pd(dppf)Cl₂ (147 mg; 0.2 mmol), KOAc (878 mg; 8.9 mmol) in 1,4-dioxane (40 mL) was stirred at 80° C. overnight under argon. The mixture was filtered through Celite and washed with EtOAc (50 mL). The combined organic solution was washed with brine (20 mL), dried over anhydrous Na₂SO₄, filtered, and concentrated to afford the title compound as a yellow oil, which was used directly without further purification.

LC-MS: m/z 272.1 [M+H]⁺.

### Intermediate 15: 6-fluoro-1-(phenylsulfonyl)-3-(1-(pyridin-2-yl)-1H-pyrazol-4-yl)-1H-indole

Following the general method as outlined in Intermediate 10, starting from 2-(4-(4,4,5,5-tetramethyl-1,3,2-dioxaborolan-2-yl)-1H-pyrazol-1-yl)pyridine (Intermediate 14; 485 mg; 1.80 mmol), 234 mg (62%) of the title compound was obtained as a yellow solid after purification by a silica gel chromatography (petroleum ether/EtOAc=1/1).

LC-MS: m/z 419.1 [M+H]⁺.

### Intermediate 16: tert-butyl 4-(4-(6-fluoro-1-(phenylsulfonyl)-1H-indol-3-yl)-1H-pyrazol-1-yl)piperidine-1-carboxylate and tert-butyl 4-(4-(6-fluoro-1H-indol-3-yl)-1H-pyrazol-1-yl)piperidine-1-carboxylate

To a solution of 6-fluoro-1-(phenylsulfonyl)-3-(1H-pyrazol-4-yl)-1H-indole (Intermediate 5; 939 mg; 2.75 mmol) and Cs₂CO₃ (2.23 g; 6.84 mmol) in DMF (20 mL) was added tert-butyl 4-(methylsulfonyloxy)piperidine-1-carboxylate (1.00 g; 3.58 mmol) under nitrogen. The reaction mixture was stirred at 85° C. overnight, concentrated, and purified by a silica gel chromatography (petroleum ether/EtOAc=2/1) to afford 880 mg (61%) and 230 mg (22%) of the 16A and 16B as a yellow solid.

For 16A: LC-MS: m/z 469.1 [M+H−tBu]⁺.

¹H NMR (400 MHz, DMSO-d₆) δ [ppm]: 8.39 (s, 1H), 8.09 (s, 1H), 8.04 (d, J=7.5 Hz, 2H), 7.99 (s, 1H), 7.94 (dd, J=8.8, 5.4 Hz, 1H), 7.76 (dd, J=9.8, 2.2 Hz, 1H), 7.70 (t, J=7.5 Hz, 1H), 7.60 (t, J=7.8 Hz, 2H), 7.21 (td, J=9.1, 2.3 Hz, 1H), 4.38 (dd, J=9.6, 5.7 Hz, 1H), 4.12-3.98 (m, 3H), 3.07-2.81 (m, 3H), 2.08-2.00 (m, 2H), 1.86 (td, J=12.1, 4.1 Hz, 2H), 1.43 (s, 9H).

For 16B: LC-MS: m/z 385.2 [M+H]⁺.

¹H NMR (400 MHz, DMSO-d₆) δ [ppm]: 11.16 (s, 1H), 8.16 (s, 1H), 7.88-7.72 (m, 2H), 7.53 (d, J=2.3 Hz, 1H), 7.16 (dd, J=10.1, 2.3 Hz, 1H), 6.90 (td, J=9.7, 2.3 Hz, 1H), 4.45-4.29 (m, 1H), 4.13-3.96 (m, 2H), 3.07-2.87 (m, 3H), 2.08-1.98 (m, 2H), 1.91-1.75 (m, 2H), 1.40 (s, 9H).

### Intermediate 17: 6-fluoro-1-(phenylsulfonyl)-3-(1-(piperidin-4-yl)-1H-pyrazol-4-yl)-1H-indole hydrochloride

To a solution of tert-butyl 4-(4-(6-fluoro-1-(phenylsulfonyl)-1H-indol-3-yl)-1H-pyrazol-1-yl)piperidine-1-carboxylate (Intermediate 16A; 580 mg; 1.11 mmol) in dioxane (3 mL) was added conc. aqueous HCl (3 mL; 36%). The reaction mixture was stirred for 0.5 hour and concentrated afford 580 mg (>100%) of the title compound as a white solid.

LC-MS: m/z 425.1 [M+H]⁺.

¹H NMR (400 MHz, DMSO-d₆) δ [ppm]: 8.37 (s, 1H), 8.13 (s, 1H), 8.05 (t, J=3.7 Hz, 3H), 7.92 (dd, J=8.8, 5.3 Hz, 1H), 7.77 (dd, J=9.8, 2.2 Hz, 1H), 7.71 (t, J=7.4 Hz, 1H), 7.61 (dd, J=9.7, 5.8 Hz, 2H), 7.23 (td, J=9.1, 2.2 Hz, 1H), 4.55-4.51 (m, 1H), 3.38-3.41 (m, 2H), 3.14-3.02 (m, 2H), 2.31-2.12 (m, 4H).

### Intermediate 18: tert-butyl 2-(4-(6-fluoro-1-(phenylsulfonyl)-1H-indol-3-yl)-1H-pyrazol-1-yl)ethylcarbamate

To a solution of 6-fluoro-1-(phenylsulfonyl)-3-(1H-pyrazol-4-yl)-1H-indole (Intermediate 5; 500 mg; 1.46 mmol) in DMF (30 mL) was added tert-butyl 2-bromoethylcarbamate (658 mg; 2.94 mmol) and Cs₂CO₃ (1.43 g; 4.39 mmol) and catalytic amount of tetrabutylammonium iodide (107 mg; 0.29 mmol) under nitrogen. The reaction mixture was stirred at 85° C. for 24 hours. Most of DMF was removed and the residue was diluted with EtOAc (100 mL), washed with water (50 ml×3), brine (50 mL), dried over anhydrous Na₂SO₄, filtered, concentrated, and purified by reversed phase flash chromatography to afford 500 mg (70%) of the title compound as a yellow solid.

LC-MS: m/z 485.2 [M+H]⁺.

¹H NMR (400 MHz, DMSO-d₆) δ [ppm]: 8.26 (s, 1H), 8.11-8.01 (m, 3H), 7.99 (s, 1H), 7.93-7.85 (m, 1H), 7.77 (dd, J=10.1, 1.7 Hz, 1H), 7.69 (d, J=6.6 Hz, 1H), 7.60 (t, J=7.5 Hz, 2H), 7.22 (td, J=9.2, 2.5 Hz, 1H), 6.95 (s, 1H), 4.44 (t, J=4.9 Hz, 2H), 4.17 (t, J=6.5 Hz, 2H), 1.32 (s, 9H).

### Intermediate 19: 2-(4-(6-fluoro-1-(phenylsulfonyl)-1H-indol-3-yl)-1H-pyrazol-1-yl)ethanamine hydrochloride

To a solution of tert-butyl 2-(4-(6-fluoro-1-(phenylsulfonyl)-1H-indol-3-yl)-1H-pyrazol-1-yl)ethylcarbamate (260 mg; 0.54 mmol) in dioxane (5 mL) was added conc. aqueous HCl (5 mL; 36%). The reaction mixture was stirred for 1 hours and concentrated to afford 260 mg (68%) of the title compound as a yellow solid, which was used directly without further purification.

LC-MS: m/z 385.2 [M+H]⁺.

### Intermediate 20: tert-butyl 4-((4-(6-fluoro-1-(phenylsulfonyl)-1H-indol-3-yl)-1H-pyrazol-1-yl)methyl)piperidine-1-carboxylate

To a solution of 6-fluoro-1-(phenylsulfonyl)-3-(1H-pyrazol-4-yl)-1H-indole (Intermediate 5; 1.20 g; 3.52 mmol) and Cs₂CO₃ (2.26 g; 6.94 mmol) in DMF (20 mL) was added tert-butyl 4-((methylsulfonyloxy)methyl)piperidine-1-carboxylate (1.32 g; 4.50 mmol) under nitrogen. The reaction mixture was stirred at 90° C. for 2 hours, cooled to r.t., added to water (200 mL) with vigorous stirring. The resulting solid was collected by vacuum filtration to afford 1.60 g (84%) of the title compound as a yellow solid.

¹H NMR (300 MHz, DMSO-d₆) δ [ppm]: 8.31 (s, 1H), 8.10 (s, 1H), 8.05 (d, J=7.4 Hz, 2H), 7.99 (s, 1H), 7.88 (dd, J=8.8, 5.3 Hz, 1H), 7.73 (dd, J=22.0, 7.7 Hz, 2H), 7.61 (t, J=7.5 Hz, 2H), 7.23 (s, 1H), 4.03 (dd, J=7.0, 3.6 Hz, 2H), 3.94 (s, 2H), 2.67 (s, 2H), 2.07 (s, 1H), 1.49 (d, J=12.7 Hz, 2H), 1.08 (d, J=12.0 Hz, 2H).

### Intermediate 21: 6-fluoro-1-(phenylsulfonyl)-3-(1-(piperidin-4-ylmethyl)-1H-pyrazol-4-yl)-1H-indole

To a solution of tert-butyl 4-((4-(6-fluoro-1-(phenylsulfonyl)-1H-indol-3-yl)-1H-pyrazol-1-yl)methyl)piperidine-1-carboxylate (Intermediate 20; 1.60 g; 2.97 mmol) in THF (10 mL) was added conc. aqueous HCl (5 mL; 36%). The reaction mixture was stirred for 1 hour and concentrated, neutralized with saturated aqueous Na₂CO₃ (500 mL), extracted with EtOAc (200 ml×2). The combined organic layers were dried over anhydrous Na₂SO₄, filtered, concentrated, and purified by reversed phase flash chromatography to afford 1.08 g (83%) of the title compound as a red oil.

¹H NMR (300 MHz, DMSO-d₆) δ [ppm]: 8.31 (s, 1H), 8.09 (s, 1H), 8.05 (d, J=7.6 Hz, 2H), 7.97 (s, 1H), 7.88 (s, 1H), 7.77 (d, J=10.0 Hz, 1H), 7.71 (s, 1H), 7.62 (d, J=7.5 Hz, 2H), 7.23 (s, 1H), 3.99 (d, J=7.1 Hz, 2H), 2.88 (s, 2H), 2.39 (s, 2H), 1.93 (s, 2H), 1.42 (d, J=14.5 Hz, 2H), 1.07 (d, J=7.0 Hz, 2H).

### Intermediate 22: 3-(1-(2-bromoethyl)-1H-pyrazol-4-yl)-6-fluoro-1-(phenylsulfonyl)-1H-indole

To a solution of 6-fluoro-1-(phenylsulfonyl)-3-(1H-pyrazol-4-yl)-1H-indole (Intermediate 5; 1.0 g; 2.93 mmol) in DMF (50 mL) was added NaH (176 mg; 4.4 mmol; 60%) at 0° C. under nitrogen. The reaction mixture was stirred at 0° C. for 1 hour, added 1,2-dibromoethane (0.83 g; 4.4 mmol), warmed to r.t. and stirred overnight. The mixture was poured into ice-water (50 mL) and extracted by EtOAc (50 mL×3). The combined organic layers was washed with brine (100 mL), dried over anhydrous Na₂SO₄, concentrated, and purified by a silica gel chromatography (petroleum ether/EtOAc=2/1) to afford 0.48 g (37%) of the title compound as a yellow solid.

¹H NMR (300 MHz, DMSO-d₆) δ [ppm]: 8.41 (s, 1H), 8.12 (s, 1H), 8.07-8.05 (m, 3H), 7.95-7.86 (m, 1H), 7.79-7.75 (m, 1H), 7.73-7.68 (m, 1H). 7.63-7.58 (m, 2H), 7.27-7.20 (m, 1H), 4.57-4.37 (t, 2H), 3.93-3.89 (t, 2H).

### Intermediate 23: tert-butyl 3-(4-(6-fluoro-1-(phenylsulfonyl)-1H-indol-3-yl)-1H-pyrazol-1-yl)azetidine-1-carboxylate

Following the general method as outlined in Intermediate 16, starting from 6-fluoro-1-(phenylsulfonyl)-3-(1H-pyrazol-4-yl)-1H-indole (Intermediate 5; 812 mg; 2.38 mmol) and tert-butyl 3-iodoazetidine-1-carboxylate (674 mg; 2.38 mmol), 1.19 g (100%) of the title compound was obtained as a yellow solid, which was used directly without further purification.

LC-MS: m/z 397 [M+H−Boc]⁺.

### Intermediate 24: 3-(1-(azetidin-3-yl)-1H-pyrazol-4-yl)-6-fluoro-1-(phenylsulfonyl)-1H-indole hydrochloride

Following the general method as outlined in Intermediate 17, starting from tert-butyl 3-(4-(6-fluoro-1-(phenylsulfonyl)-1H-indol-3-yl)-1H-pyrazol-1-yl)azetidine-1-carboxylate (Intermediate 23; 1.32 g; 2.66 mmol), 1.30 g of the title compound was obtained as a brown solid, which was used directly without further purification.

LC-MS: m/z 397 [M+H]⁺.

### Intermediate 25: 1,5-dimethyl-4-(4,4,5,5-tetramethyl-1,3,2-dioxaborolan-2-yl)-1H-pyrazole and 1,3-dimethyl-4-(4,4,5,5-tetramethyl-1,3,2-dioxaborolan-2-yl)-1H-pyrazole

A solution of 3-methyl-4-(4,4,5,5-tetramethyl-1,3,2-dioxaborolan-2-yl)-1H-pyrazole (0.60 g; 2.9 mmol) and K₂CO₃ (2.0 g; 14 mmol) in MeCN (20 mL) was stirred overnight under nitrogen and added iodomethane (1.0 mL; 16 mmol). The reaction mixture was stirred overnight, diluted with EtOAc (20 mL), filtered, and concentrated to afford 727 mg of an inseparable mixture of the title compounds as a light yellow solid.

LC-MS: m/z 223.1 [M+H]⁺.

### Intermediate 26: 3-(1,5-dimethyl-1H-pyrazol-4-yl)-6-fluoro-1-(phenylsulfonyl)-1H-indole and 3-(1,3-dimethyl-1H-pyrazol-4-yl)-6-fluoro-1-(phenylsulfonyl)-1H-indole

Following the general method as outlined in Intermediate 4, starting from 1,5-dimethyl-4-(4,4,5,5-tetramethyl-1,3,2-dioxaborolan-2-yl)-1H-pyrazole and 1,3-dimethyl-4-(4,4,5,5-tetramethyl-1,3,2-dioxaborolan-2-yl)-1H-pyrazole (Intermediate 25; 670 mg; 3.02 mmol) and 6-fluoro-3-iodo-1-(phenylsulfonyl)-1H-indole (Intermediate 3; 807 mg; 2.01 mmol), 382 mg (51%) of an inseparable mixture of the title compounds as a light yellow solid.

LC-MS: m/z 370.1 [M+H]⁺.

### Intermediate 27: 5,6-difluoro-3-iodo-1H-indole

To a mixture of 5,6-difluoro-1H-indole (500 mg; 3.27 mmol) and KOH (458 mg; 8.18 mmol) in DMF (6.2 mL) was added a solution of iodine (837.5 mg; 3.3 mmol) in DMF (6.3 mL). The mixture was stirred at r.t. for 12 hours. It was poured into an ice-water mixture (60 mL) and extracted with EtOAc (20 mL×3). The combined organic layers was washed with brine (100 mL) and dried over anhydrous Na₂SO₄, filtered, concentrated to afford 930 mg (100%) of the title compound as a red solid. It was used to next step without further purification.

¹H NMR (400 MHz, DMSO-d₆) δ [ppm]: 11.68 (s, 1H), 7.61 (d, J=2.5 Hz, 1H), 7.47 (dd, J=11.0, 6.9 Hz, 1H), 7.21 (dd, J=10.8, 7.9 Hz, 1H).

### Intermediate 28: 5,6-difluoro-3-iodo-1-(phenylsulfonyl)-1H-indole

To a solution of 5,6-difluoro-3-iodo-1H-indole (Intermediate 27; 930 mg; 3.33 mmol) in THF (20 mL) at 0° C. was added NaH (266.4 mg; 60%; 6.66 mmol) under nitrogen. The reaction mixture was stirred at r.t. for 15 minutes before a solution of benzenesulfonyl chloride (763.4 mg; 4.32 mmol) in THF (2 mL) was added dropwise. The reaction was stirred at r.t. for 12 hours, quenched with an ice-water mixture (60 mL) and extracted with EtOAc (30 mL×3). The combined organic layers were washed with brine (100 mL), dried over anhydrous Na₂SO₄, filtered, concentrated, and purified by a silica gel chromatography (petroleum ether/EtOAc=10/1-3/1) to afford 1.01 g (72%) of the title compound as a yellow solid.

¹H NMR (400 MHz, DMSO-d₆) δ [ppm]: 8.19 (s, 1H), 8.12 (d, J=7.9 Hz, 2H), 8.07-8.00 (m, 1H), 7.78-7.72 (m, 1H), 7.64 (t, J=8.0 Hz, 2H), 7.41 (dd, J=10.2, 7.8 Hz, 1H).

### Intermediate 29: tert-butyl 4-(5,6-difluoro-1-(phenylsulfonyl)-1H-indol-3-yl)-1H-pyrazole-1-carboxylate

To a solution of 5,6-difluoro-3-iodo-1-(phenylsulfonyl)-1H-indole (Intermediate 28; 300 mg; 0.716 mmol), tert-butyl 4-(4,4,5,5-tetramethyl-1,3,2-dioxaborolan-2-yl)-1H-pyrazole-1-carboxylate (273.6 mg; 0.93 mmol), K₃PO₄ (455 mg; 2.15 mmol) in dioxane (10 mL) and water (1 mL) was added Pd(dppf)Cl₂ (59 mg; 0.072 mmol) under argon. The reaction mixture was stirred at 90° C. for 12 hours. The mixture was filtered through Celite and washed with EtOAc (50 mL). The combined organic layers were washed with brine (100 mL), dried over anhydrous Na₂SO₄, concentrated, and purified by a silica gel chromatography (petroleum ether/EtOAc=1/1) to afford 203 mg (62%) of the title compound as a yellow solid.

¹H NMR (400 MHz, DMSO-d₆) δ [ppm]: 8.83 (s, 1H), 8.45 (s, 1H), 8.41 (d, J=0.6 Hz, 1H), 8.10 (dd, J=5.3, 3.4 Hz, 2H), 8.07-7.99 (m, 2H), 7.72 (d, J=7.4 Hz, 1H), 7.63 (t, J=7.8 Hz, 2H), 1.62 (s, 9H).

### Intermediate 30: 5,6-difluoro-1-(phenylsulfonyl)-3-(1H-pyrazol-4-yl)-1H-indole hydrochloride

To a solution of tert-butyl 4-(5,6-difluoro-1-(phenylsulfonyl)-1H-indol-3-yl)-1H-pyrazole-1-carboxylate (Intermediate 29; 203 mg; 0.44 mmol) in EtOAc (2 mL) was added HCl in EtOAc (4 M; 1 mL). The resulting mixture was stirred for 2 hours. The reaction mixture was concentrated to dryness under reduced pressure to afford 150 mg (88%) of the title compound as a red solid.

LC-MS: m/z 360.1 [M+H]⁺.

¹H NMR (400 MHz, DMSO-d₆) δ [ppm]: 13.08 (s, 1H), 8.36 (s, 1H), 8.18 (s, 1H), 8.10-8.05 (m, 2H), 7.99 (m, 3H), 7.71 (dd, J=8.4, 6.5 Hz, 1H), 7.61 (t, J=7.7 Hz, 2H).

### Intermediate 31: 2-(2-oxoimidazolidin-1-yl)ethyl methanesulfonate

To a solution of 1-(2-hydroxyethyl)imidazolidin-2-one (100 mg; 0.77 mmol) and Et₃N (81 mg; 0.80 mmol) in dry DCM (10 mL) was added MsCl (137 mg; 1.20 mmol) under nitrogen. The reaction mixture was stirred overnight, diluted with DCM (20 mL), washed with saturated aqueous Na₂CO₃, dried over anhydrous Na₂SO₄, concentrated to afford 72 mg (44%) of the title compound as a yellow oil, which was used directly without further purification.

### Intermediate 32: benzyl 3-hydroxycyclobutanecarboxylate

NaBH₄ (215 mg; 5.68 mmol) was added to the solution of benzyl 3-oxocyclobutanecarboxylate (2.3 g; 11.3 mmol) in THF (30 mL) and MeOH (1.5 mL). The reaction mixture was stirred for 0.5 hour at 0° C., diluted with water (20 mL), and extracted with DCM (50 mL×2). The combined organic layers were dried over anhydrous Na₂SO₄, filtered, concentrated, and purified by reverse phase flash chromatography to afford 1.24 g (53%) of the title compound as a yellow oil.

¹H NMR (400 MHz, CDCl₃) δ [ppm]: 7.49-7.22 (m, 5H), 5.12 (d, J=4.1 Hz, 2H), 4.28-4.08 (m, 1H), 2.72-2.50 (m, 3H), 2.41-2.09 (m, 3H).

### Intermediate 33: benzyl 3-((methylsulfonyl)oxy)cyclobutanecarboxylate

To a solution of benzyl 3-hydroxycyclobutanecarboxylate (Intermediate 32; 0.90 g; 4.4 mmol), Et₃N (1.20 mL; 8.6 mmol) in DCM (50 mL) was added MsCl (0.40 mL; 5.2 mmol) under nitrogen. The reaction mixture was stirred for 1 hour, quenched with water (20 mL), and extracted with DCM (50 mL×2). The combined organic layers were dried over anhydrous Na₂SO₄, filtered, and concentrated to afford 1.24 g (100%) of the title compound as a yellow oil, which was used directly without further purification.

¹H NMR (300 MHz, DMSO-d₆) δ [ppm]: 7.45-7.28 (m, 5H), 5.16-5.07 (m, 2H), 4.93 (t, J=7.4 Hz, 1H), 3.16 (s, 3H), 2.86 (dd, J=16.7, 8.5 Hz, 1H), 2.67 (dtd, J=10.2, 7.5, 2.8 Hz, 2H), 2.44-2.28 (m, 2H).

### Intermediate 34A and B: trans- and cis-benzyl 3-(4-(6-fluoro-1-(phenylsulfonyl)-1H-indol-3-yl)-1H-pyrazol-1-yl)cyclobutanecarboxylate

A mixture of benzyl 3-((methylsulfonyl)oxy)cyclobutanecarboxylate (Intermediate 33; 1.24 g; 4.36 mmol), 6-fluoro-1-(phenylsulfonyl)-3-(1H-pyrazol-4-yl)-1H-indole (Intermediate 5; 1.35 g; 3.95 mmol), and Cs₂CO₃ (2.59 g; 7.95 mmol) in DMF (20 mL) was stirred at 90° C. overnight under nitrogen, cooled to r.t., diluted with water (50 mL), and extracted with EtOAc (100 ml×2). The combined organic layers were dried over anhydrous Na₂SO₄, filtered, concentrated, and purified by a silica gel chromatography (petroleum ether/EtOAc=4/1) to afford 600 mg (29%) of Intermediate 34A and 500 mg (24%) of Intermediate 34B as colorless oils.

LC-MS: m/z 530 [M+H]⁺.

### Intermediate 35: trans-3-(4-(6-fluoro-1-(phenylsulfonyl)-1H-indol-3-yl)-1H-pyrazol-1-yl)cyclobutanecarboxylic acid

A mixture of trans-benzyl 3-(4-(6-fluoro-1-(phenylsulfonyl)-1H-indol-3-yl)-1H-pyrazol-1-yl)cyclobutanecarboxylate (Intermediate 34A; 500 mg; 0.944 mmol) and Pd/C (50 mg) in MeOH (50 mL) was stirred overnight under a hydrogen balloon. The reaction mixture was filtered and the filtrate was concentrated and purified by reverse phase flash chromatography to afford 113 mg (27%) of the title compound as a yellow oil.

LC-MS: m/z 440 [M+H]⁺.

### Intermediate 36: cis-3-(4-(6-fluoro-1-(phenylsulfonyl)-1H-indol-3-yl)-1H-pyrazol-1-yl)cyclobutanecarboxylic acid

Following the general method as outlined in Intermediate 35, starting from cis-benzyl 3-(4-(6-fluoro-1-(phenylsulfonyl)-1H-indol-3-yl)-1H-pyrazol-1-yl)cyclobutanecarboxylate (Intermediate 34B; 600 mg; 1.13 mmol), 440 mg (88%) of the title compound was obtained as a yellow oil, which was used directly without further purification.

LC-MS: m/z 440 [M+H]⁺.

### Intermediate 37: tert-butyl 3-((4-(6-fluoro-1-(phenylsulfonyl)-1H-indol-3-yl)-1H-pyrazol-1-yl)methyl)azetidine-1-carboxylate

Following the general method as outlined in Intermediate 20, starting from 6-fluoro-1-(phenylsulfonyl)-3-(1H-pyrazol-4-yl)-1H-indole (Intermediate 5; 1.22 g; 3.56 mmol) and tert-butyl 3-(((methylsulfonyl)oxy)methyl)azetidine-1-carboxylate (1.40 g; 5.28 mmol), 1.37 g (76%) of the title compound as a yellow solid.

LC-MS: m/z 455.0 [M+H⁺−tBu]⁺.

¹H NMR (400 MHz, DMSO-d₆) δ [ppm]: 8.39 (s, 1H), 8.00-8.10 (m, 4H), 7.94-7.84 (m, 1H), 7.77 (d, J=9.3 Hz, 1H), 7.70 (dd, J=12.9, 6.3 Hz, 1H), 7.61 (t, J=7.6 Hz, 3H), 7.23 (dd, J=13.7, 4.9 Hz, 1H), 4.36 (d, J=7.1 Hz, 2H), 3.98-3.83 (m, 2H), 3.69-3.71 (m, 3H), 2.99-3.01 (m, 2H), 1.33 (s, 9H).

### Intermediate 38: 3-(1-(azetidin-3-ylmethyl)-1H-pyrazol-4-yl)-6-fluoro-1-(phenylsulfonyl)-1H-indole

To a solution of tert-butyl 3-((4-(6-fluoro-1-(phenylsulfonyl)-1H-indol-3-yl)-1H-pyrazol-1-yl)methyl)azetidine-1-carboxylate (Intermediate 37; 330 mg; 0.65 mmol) in DCM (10 mL) was added TFA (5 mL) dropwise. The reaction mixture was stirred for 0.5 hour and concentrated, neutralized with saturated aqueous Na₂CO₃ (50 mL), extracted with EtOAc (50 ml×2). The combined organic layers were dried over anhydrous Na₂SO₄, filtered, concentrated and purified by preparative TLC to afford 265 mg (100%) of the title compound as a brown solid.

LC-MS: m/z 411.0 [M+H]⁺.

### Intermediate 39: 3-iodo-6-(trifluoromethyl)-1H-indole

Following the general method as outlined in Intermediate 3, starting from 6-(trifluoromethyl)-1H-indole (0.97 g; 5.24 mmol), 1.60 g (98%) of the title compound was obtained as a red solid, which was used directly without further purification.

¹H NMR (400 MHz, DMSO-d₆) δ [ppm]: 11.95 (s, 1H), 7.79 (d, J=16.1 Hz, 2H), 7.49 (d, J=8.3 Hz, 1H), 7.40 (d, J=8.4 Hz, 1H).

### Intermediate 40: 3-iodo-1-(phenylsulfonyl)-6-(trifluoromethyl)-1H-indole

Following the general method as outlined in Intermediate 28, starting from 3-iodo-6-(trifluoromethyl)-1H-indole (Intermediate 39; 1.60 g; 5.14 mmol), 1.06 g (45%) of the title compound was obtained as a yellow solid.

¹H NMR (400 MHz, DMSO-d₆) δ [ppm]: 8.39 (s, 1H), 8.20 (s, 1H), 8.09 (d, J=7.6 Hz, 2H), 7.78-7.68 (m, 2H), 7.66 (d, J=7.9 Hz, 2H), 7.64-7.58 (m, 1H).

### Intermediate 41: tert-butyl 4-(1-(phenylsulfonyl)-6-(trifluoromethyl)-1H-indol-3-yl)-1H-pyrazole-1-carboxylate

Following the general method as outlined in Intermediate 4, starting from 3-iodo-1-(phenylsulfonyl)-6-(trifluoromethyl)-1H-indole (Intermediate 40; 300 mg; 0.66 mmol) 0.40 g (94%) of the title compound was obtained as a black oil, which was used directly without further purification.

LC-MS: m/z 392.1 [M+H⁺−Boc]⁺.

### Intermediate 42: 1-(phenylsulfonyl)-3-(1H-pyrazol-4-yl)-6-(trifluoromethyl)-1H-indole

Following the general method as outlined in Intermediate 5, starting from tert-butyl 4-(1-(phenylsulfonyl)-6-(trifluoromethyl)-1H-indol-3-yl)-1H-pyrazole-1-carboxylate (Intermediate 41; 400 mg; 0.81 mmol), 300 mg (95%) of the title compound was obtained as a yellow oil, which was used directly without further purification.

LC-MS: m/z 392.1 [M+H⁺]⁺.

### Intermediate 43: 2-(2-oxopyrrolidin-1-yl)ethyl methanesulfonate

Following the general method as outlined in Intermediate 31, starting from 1-(2-hydroxyethyl)pyrrolidin-2-one (500 mg; 3.87 mmol), 400 mg (50%) of the title compound as a yellow oil, which was used directly without further purification.

### Intermediate 44: 3-(1-(6-chloropyridazin-3-yl)-1H-pyrazol-4-yl)-6-fluoro-1-(phenylsulfonyl)-1H-indole

A mixture of 6-fluoro-1-(phenylsulfonyl)-3-(1H-pyrazol-4-yl)-1H-indole (Intermediate 5; 512 mg; 1.50 mmol), 3,6-dichloropyridazine (450 mg; 3.02 mmol) and K₂CO₃ (828 mg; 5.99 mmol) in MeCN (15.0 mL) was stirred at 100° C. for 16 hours under nitrogen. The reaction mixture was diluted with EtOAc (30 mL) and water (10 mL). The organic layer was separated, washed with brine (10 mL), dried over anhydrous Na₂SO₄, filtered, concentrated, and purified by a silica gel column chromatography (petroleum ether/EtOAc=4/1 to 2/1) to afford 620 mg (91%) of the title compound as a white solid.

LC-MS: m/z 454 [M+H]⁺.

¹H NMR (400 MHz, DMSO-d₆) δ [ppm]: 9.33 (s, 1H), 8.59 (s, 1H), 8.43 (s, 1H), 8.33 (d, J=9.0 Hz, 1H), 8.14-8.03 (m, 4H), 7.80 (dd, J=9.6, 2.4 Hz, 1H), 7.74-7.69 (m, 1H), 7.67-7.59 (m, 2H), 7.26 (dt, J=9.0, 2.4 Hz, 1H).

### Intermediate 45: tert-butyl 4-((4-(5,6-difluoro-1-(phenylsulfonyl)-1H-indol-3-yl)-1H-pyrazol-1-yl)methyl)piperidine-1-carboxylate

Following the general method as outlined in Intermediate 20, starting from 5,6-difluoro-1-(phenylsulfonyl)-3-(1H-pyrazol-4-yl)-1H-indole hydrochloride (Intermediate 30; 300 mg; 0.76 mmol), 330 mg (78%) of the title compound was obtained as a yellow solid after purification by a silica gel chromatography (petroleum ether/EtOAc=5/1).

LC-MS: m/z 501.1 [M+H⁺−tBu]⁺.

### Intermediate 46: 5,6-difluoro-1-(phenylsulfonyl)-3-(1-(piperidin-4-ylmethyl)-1H-pyrazol-4-yl)-1H-indole

Following the general method as outlined in Intermediate 21, starting from tert-butyl 4-((4-(5,6-difluoro-1-(phenylsulfonyl)-1H-indol-3-yl)-1H-pyrazol-1-yl)methyl)piperidine-1-carboxylate (Intermediate 45; 330 mg; 0.59 mmol), 830 mg (>100%) of the title compound was obtained as a yellow solid, which was used directly without further purification.

LC-MS: m/z 457.1 [M+H⁺]⁺.

### Intermediate 47: 3-(1-(1,4-dioxaspiro[4.5]decan-8-yl)-1H-pyrazol-4-yl)-6-fluoro-1-(phenylsulfonyl)-1H-indole

Following the general method as outlined in Intermediate 16, starting from 6-fluoro-1-(phenylsulfonyl)-3-(1H-pyrazol-4-yl)-1H-indole (Intermediate 5; 600 mg; 1.76 mmol) and 1,4-dioxaspiro[4.5]decan-8-yl methanesulfonate (498 mg; 2.11 mmol), 500 mg (59%) of the title compound was obtained as a white solid after purification by reverse phase flash chromatography.

LC-MS: m/z 482 [M+H]⁺.

### Intermediate 48: 4-(4-(6-fluoro-1-(phenylsulfonyl)-1H-indol-3-yl)-1H-pyrazol-1-yl)cyclohexanone

To a solution of 3-(1-(1,4-dioxaspiro[4.5]decan-8-yl)-1H-pyrazol-4-yl)-6-fluoro-1-(phenylsulfonyl)-1H-indole (Intermediate 47; 500 mg; 1.04 mmol) in THF (20 mL) was added HCl (0.35 mL; 35%). The reaction mixture was stirred overnight, concentrated, and purified by reverse phase flash chromatography to afford 197 mg (43%) of the title compound as a white solid.

LC-MS: m/z 438 [M+H]⁺.

### Intermediate 49: trans-4-(4-(6-fluoro-1-(phenylsulfonyl)-1H-indol-3-yl)-1H-pyrazol-1-yl)cyclohexanol

To a solution of 4-(4-(6-fluoro-1-(phenylsulfonyl)-1H-indol-3-yl)-1H-pyrazol-1-yl)cyclohexanone (Intermediate 48; 197 mg; 0.45 mmol) in MeOH (15 mL) was added NaBH₄ (34 mg; 0.90 mmol). The reaction mixture was stirred overnight, concentrated, and purified by preparative TLC (DCM/MeOH=20/1) to afford 32 mg (16%) of the title compound as a white solid.

¹H NMR (400 MHz, MeOH-d₄) δ [ppm]: 8.10 (s, 1H), 7.99 (dd, J=8.5, 1.1 Hz, 2H), 7.86 (d, J=11.3 Hz, 2H), 7.82-7.74 (m, 2H), 7.69-7.62 (m, 1H), 7.56 (t, J=7.7 Hz, 2H), 7.17-7.09 (m, 1H), 4.25 (dd, J=9.9, 5.8 Hz, 1H), 3.79-3.62 (m, 1H), 2.25-2.09 (m, 4H), 2.04-1.90 (m, 2H), 1.53 (dd, J=16.9, 6.0 Hz, 2H).

### Intermediate 50: 3-(3,5-dimethyl-1H-pyrazol-4-yl)-6-fluoro-1-(phenylsulfonyl)-1H-indole

### Step 1

A mixture of 6-fluoro-3-iodo-1-(phenylsulfonyl)-1H-indole (Intermediate 3; 4.90 g; 12.2 mmol), tert-butyl 3,5-dimethyl-4-(4,4,5,5-tetramethyl-1,3,2-dioxaborolan-2-yl)-1H-pyrazole-1-carboxylate (3.30 g; 10.2 mmol), K₃PO₄ (6.50 g; 30.6 mmol), X-Phos (242 mg; 0.51 mmol) and Pd₂(dba)₃ (466 mg; 0.51 mmol) in dioxane (25 mL) and water (3 mL) was stirred at 120° C. under N₂ atmosphere for 80 minutes in a microwave reactor. The reaction mixture was cooled, diluted with EtOAc (150 mL), dried over anhydrous Na₂SO₄, filtered, and concentrated to afford 5.70 g of a brown oil, which was used directly without further purification.

LCMS indicated the crude product was a mixture of 3-(3,5-dimethyl-1H-pyrazol-4-yl)-6-fluoro-1-(phenylsulfonyl)-1H-indole and tert-butyl 4-(6-fluoro-1-(phenylsulfonyl)-1H-indol-3-yl)-3,5-dimethyl-1H-pyrazole-1-carboxylate.

### Step 2

A mixture of crude 3-(3,5-dimethyl-1H-pyrazol-4-yl)-6-fluoro-1-(phenylsulfonyl)-1H-indole and tert-butyl 4-(6-fluoro-1-(phenylsulfonyl)-1H-indol-3-yl)-3,5-dimethyl-1H-pyrazole-1-carboxylate (Step 1; 5.70 g) in HCl/dioxane (20 mL; 80 mmol; 4.0 M) was stirred at 30° C. for 2 hours. The mixture was concentrated, neutralized with saturated aqueous NaHCO₃ (30 mL), and extracted with EtOAc (80 mL×2). The combined organic layers were washed with brine (50 mL×2), dried over anhydrous Na₂SO₄, filtered, concentrated, and purified by a silica gel chromatography (DCM to petroleum ether/EtOAc=1/1) to afford 1.50 g (33%) of the title compound as a brown oil.

¹H NMR (300 MHz, CDCl₃) δ [ppm]: 7.95-7.88 (m, 2H), 7.78 (dd, J=9.7, 2.2 Hz, 1H), 7.63-7.55 (m, 1H), 7.53-7.45 (m, 2H), 7.41 (s, 1H), 7.22 (dd, J=8.7, 5.3 Hz, 1H), 7.00 (dd, J=9.7, 8.7, 2.2 Hz, 1H), 2.17 (s, 6H).

### Intermediate 51: tert-butyl 4-((4-(6-fluoro-1-(phenylsulfonyl)-1H-indol-3-yl)-3-methyl-1H-pyrazol-1-yl)methyl)piperidine-1-carboxylate and tert-butyl 4-((4-(6-fluoro-1-(phenylsulfonyl)-1H-indol-3-yl)-5-methyl-1H-pyrazol-1-yl)methyl) piperidine-1-carboxylate

Following the general method as outlined in Intermediate 20, starting from a mixture of 6-fluoro-3-(3-methyl-1H-pyrazol-4-yl)-1-(phenylsulfonyl)-1H-indole and 6-fluoro-3-(5-methyl-1H-pyrazol-4-yl)-1-(phenylsulfonyl)-1H-indole (Intermediate 16; 500 mg; 1.41 mmol), 1.00 g (>100%) of a mixture of the crude title compounds were obtained as a black oil, which were used directly without further purification.

LC-MS: m/z 552.8 [M+H⁺]⁺

### Intermediate 52: 4-(4-(6-fluoro-1H-indol-3-yl)-1H-pyrazol-1-yl)cyclohexane carboxylic acid (as a Mixture of Cis and Trans Isomers)

To a solution of 4-(4-(6-fluoro-1-(phenylsulfonyl)-1H-indol-3-yl)-1H-pyrazol-1-yl)cyclohexanecarboxylate (as a mixture of cis and trans isomers) (Intermediate 33; 400 mg; 0.81 mmol) in MeOH (10 mL) was added aqueous NaOH (2 M; 2.0 mL; 4.0 mmol). The mixture was refluxed for 30 minutes, cooled, concentrated, diluted with water (15 mL), and extracted with petroleum ether/EtOAc=5/1 (20 mL). The aqueous layer was acified with aqueous HCl (1 M) to pH=5 and extracted with EtOAc (30 mL×3). The combined organic layers were washed with brine (20 mL×2), dried over anhydrous Na₂SO₄, filtered, and concentrated to afford 216 mg (82%) of the title compound as a gray solid, which was used directly without further purification.

¹H NMR (300 MHz, DMSO-d₆) δ [ppm]: 12.18 (brs, 1H), 11.16 (brs, 1H), 8.11-8.07 (m, 1H), 7.80-7.74 (m, 2H), 7.55-7.51 (m, 1H), 7.19-7.12 (m, 1H), 6.94-6.85 (m, 1H), 4.28-4.10 (m, 1H), 2.36-2.25 (m, 1H), 2.20-1.75 (m, 6H), 1.74-1.45 (m, 2H).

### Intermediate 53: 3-(1-(1,4-dioxaspiro[4.5]decan-8-yl)-1H-pyrazol-4-yl)-6-fluoro-1H-indole

A mixture of 3-(1-(1,4-dioxaspiro[4.5]decan-8-yl)-1H-pyrazol-4-yl)-6-fluoro-1-(phenylsulfonyl)-1H-indole (Intermediate 35; 1.41 g; 2.93 mmol) and NaOH (468 mg; 11.7 mmol) in MeOH (20 mL) and H₂O (1 mL) was stirred for 0.5 hour at 85° C. The reaction mixture was diluted with water (100 mL) and extracted with EtOAc (100 mL×2). The combined organic layers were washed with brine (50 mL), dried over anhydrous Na₂SO₄, filtered, and concentrated to afford 1.00 g (100%) of the title compound as a yellow oil, which was used directly without further purification.

LC-MS: m/z 342 [M+H]⁺

### Intermediate 54: 1-(4-(4-(6-fluoro-1-(phenylsulfonyl)-1H-indol-3-yl)-3-methyl-1H-pyrazol-1-yl)piperidin-1-yl)ethanon and 1-(4-(4-(6-fluoro-1-(phenylsulfonyl)-1H-indol-3-yl)-5-methyl-1H-pyrazol-1-yl)piperidin-1-yl)ethanone

To a mixture of 6-fluoro-3-(3-methyl-1-(piperidin-4-yl)-1H-pyrazol-4-yl)-1-(phenylsulfonyl)-1H-indole and 6-fluoro-3-(5-methyl-1-(piperidin-4-yl)-1H-pyrazol-4-yl)-1-(phenylsulfonyl)-1H-indole (Intermediate 50; 400 mg crude; 0.54 mmol) and Et₃N (258 mg; 2.55 mmol) in THF (20 mL) at 0° C. was added acetyl chloride (74 mg; 0.94 mmol). The reaction mixture was stirred at room temperature for 30 minutes, diluted with water (30 mL), and extracted with EtOAc (50 mL×3). The combined organic layers were dried over anhydrous Na₂SO₄, filtered, and concentrated to afford 450 mg (>100%) of a mixture of title compounds as a yellow oil, which were used directly without further purification.

LC-MS: m/z 480.8 [M+H]⁺

### 1.2. Synthesis of Final Compounds

### Compound 2: 3-(1-(azetidin-3-yl)-1H-pyrazol-4-yl)-6-fluoro-1H-indole

### Step 1: tert-butyl 3-(4-(6-fluoro-1H-indol-3-yl)-1H-pyrazol-1-yl)azetidine-1-carboxylate

A mixture of 6-fluoro-1-(phenylsulfonyl)-3-(1H-pyrazol-4-yl)-1H-indole (Intermediate 5; 346 mg; 1.0 mmol), tert-butyl 3-iodoazetidine-1-carboxylate (283 mg; 1.0 mmol) and NaH (100 mg; 2.5 mmol; 60% w/w) in DMF (5 mL) was heated to 120° C. for 0.5 hour in a microwave reactor. The reaction mixture was added NaOH (100 mg; 2.50 mmol) in water (0.5 mL) and stirred for 0.5 hour at 85° C. The mixture was concentrated and purified by a silica gel chromatography (petroleum ether/EtOAc=1/1) to afford 220 mg (62%) of the title compound as a yellow solid.

LC-MS: m/z 357 [M+H]⁺.

### Step 2

To a solution of tert-butyl 3-(4-(6-fluoro-1H-indol-3-yl)-1H-pyrazol-1-yl)azetidine-1-carboxylate (Step 1; 220 mg; 0.62 mmol) in MeOH (10 mL) was added saturated HCl in Et₂O (5 mL). The mixture was stirred for 3 h, concentrated, and purified by preparative HPLC to afford 27.8 mg (18%) of the title compound as a white solid.

¹H NMR (400 MHz, DMSO-d₆) δ [ppm]: 11.20 (s, 1H), 8.23 (s, 1H), 7.86 (d, J=4.0 Hz, 1H), 7.78 (dd, J=8.7, 5.4 Hz, 1H), 7.56 (d, J=2.1 Hz, 1H), 7.17 (dd, J=10.1, 2.1 Hz, 1H), 7.01-6.80 (m, 1H), 5.31-5.11 (m, 1H), 3.97 (t, J=7.6 Hz, 2H), 3.73 (t, J=7.9 Hz, 2H), 2.96 (s, 1H). m.p. 205.6-206.7° C.

### Compound 3: 1-(3-(4-(6-fluoro-1H-indol-3-yl)-1H-pyrazol-1-yl)azetidin-1-yl)ethanone

### Step 1: 1-(3-(4-(6-fluoro-1-(phenylsulfonyl)-1H-indol-3-yl)-1H-pyrazol-1-yl)azetidin-1-yl)ethanone

Following the general method as outlined in the synthesis of compound 89, starting from 3-(1-(azetidin-3-yl)-1H-pyrazol-4-yl)-6-fluoro-1-(phenylsulfonyl)-1H-indole hydrochloride (Intermediate 24; 350 mg; 0.81 mmol), 355 mg (100%) of the title compound was obtained as a brown solid, which was used directly without further purification.

LC-MS: m/z 439 [M+H]⁺.

### Step 2

Following the general method as outlined in the synthesis of compound 70, starting from 1-(3-(4-(6-fluoro-1-(phenylsulfonyl)-1H-indol-3-yl)-1H-pyrazol-1-yl)azetidin-1-yl)ethanone (Step 1; 355 mg; 0.81 mmol), 146 mg (61%) of the title compound was obtained as a yellow solid after purification by preparative HPLC.

¹H NMR (400 MHz, DMSO-d₆) δ [ppm]: 11.22 (s, 1H), 8.31 (s, 1H), 7.93 (s, 1H), 7.80 (dd, J=8.7, 5.5 Hz, 1H), 7.59 (d, J=2.4 Hz, 1H), 7.17 (dd, J=10.0, 2.3 Hz, 1H), 6.99-6.86 (m, 1H), 5.27 (d, J=5.4 Hz, 1H), 4.59 (t, J=8.4 Hz, 1H), 4.46 (dd, J=8.8, 5.5 Hz, 1H), 4.32 (t, J=9.0 Hz, 1H), 4.17 (dd, J=9.8, 5.4 Hz, 1H), 1.84 (s, 3H). m.p. 82.3-83.4° C.

### Compound 4: 3-(4-(6-fluoro-1H-indol-3-yl)-1H-pyrazol-1-yl)azetidine-1-carboxamide

Step 1: 3-(4-(6-fluoro-1-(phenylsulfonyl)-1H-indol-3-yl)-1H-pyrazol-1-yl)azetidine-1-carboxamide

A mixture of 3-(1-(azetidin-3-yl)-1H-pyrazol-4-yl)-6-fluoro-1-(phenylsulfonyl)-1H-indole hydrochloride (Intermediate 24; 350 mg; 0.81 mmol), Et₃N (0.34 ml; 2.44 mmol) and TMSNCO (121 mg; 1.05 mmol) was stirred for 1.5 hours. The reaction mixture was concentrated to afford 355 mg (100%) of the title compound as a brown solid, which was used directly without further purification.

LC-MS: m/z 440 [M+H]⁺.

### Step 2

Following the general method as outlined in the synthesis of compound 70, starting from 3-(4-(6-fluoro-1-(phenylsulfonyl)-1H-indol-3-yl)-1H-pyrazol-1-yl)azetidine-1-carboxamide (Step 1; 355 mg; 0.81 mmol), 90 mg (37%) of the title compound was obtained as a yellow solid after purification by preparative HPLC.

¹H NMR (400 MHz, DMSO-d₆) δ [ppm]: 11.21 (s, 1H), 8.24 (s, 1H), 7.92 (s, 1H), 7.79 (dd, J=8.7, 5.4 Hz, 1H), 7.58 (d, J=2.3 Hz, 1H), 7.17 (dd, J=10.1, 2.3 Hz, 1H), 6.97-6.86 (m, 1H), 6.00 (s, 2H), 5.28-5.14 (m, 1H), 4.24 (t, J=8.3 Hz, 2H), 4.15 (dd, J=8.6, 5.7 Hz, 2H). m.p. 263.5-264.2° C.

### Compound 5: 3-(4-(6-fluoro-1H-indol-3-yl)-1H-pyrazol-1-yl)-N-methylazetidine-1-carboxamide

### Step 1: 3-(4-(6-fluoro-1-(phenylsulfonyl)-1H-indol-3-yl)-1H-pyrazol-1-yl)-N-methylazetidine-1-carboxamide

A mixture of CDI (62 mg; 0.38 mmol) and MeNH₂ (0.20 ml; 0.40 mmol; 2.0 M in THF) of THF (10 mL) was stirred for 1 hour and added a premixed mixture of 3-(1-(azetidin-3-yl)-1H-pyrazol-4-yl)-6-fluoro-1-(phenylsulfonyl)-1H-indole hydrochloride (Intermediate 24; 200 mg; 0.46 mmol) and Et₃N (0.10 ml; 0.72 mmol) in THF (2 mL) was stirred at rt for 10 min. The reaction mixture was stirred overnight and concentrated. The residue was diluted with EtOAc (50 mL), washed with water (20 mL), dried over anhydrous Na₂SO₄, filtered, and concentrated to afford 174 mg (100%) of the title compound as a yellow oil, which was used directly without further purification.

LC-MS: m/z 454 [M+H]⁺.

### Step 2

Following the general method as outlined in the synthesis of compound 70, starting from 3-(4-(6-fluoro-1-(phenylsulfonyl)-1H-indol-3-yl)-1H-pyrazol-1-yl)-N-methylazetidine-1-carboxamide (Step 1; 174 mg; 0.38 mmol), 61 mg (51%) of the title compound was obtained as a white solid after purification by preparative HPLC.

¹H NMR (400 MHz, DMSO-d₆) δ [ppm]: 11.22 (s, 1H), 8.25 (s, 1H), 7.91 (s, 1H), 7.79 (dd, J=9.0, 5.4 Hz, 1H), 7.58 (d, J=2.1 Hz, 1H), 7.17 (dd, J=10.0, 2.0 Hz, 1H), 6.91 (dd, J=13.2, 5.4 Hz, 1H), 6.39 (d, J=5.0 Hz, 1H), 5.22 (dd, J=9.3, 3.5 Hz, 1H), 4.23 (t, J=8.1 Hz, 2H), 4.19-4.12 (m, 2H), 2.58 (t, J=4.4 Hz, 3H). m.p. 69.3-70.1° C.

### Compound 6: 3-(1-(azetidin-3-ylmethyl)-1H-pyrazol-4-yl)-6-fluoro-1H-indole

Following the general method as outlined in the synthesis of compound 70, starting from 3-(1-(azetidin-3-ylmethyl)-1H-pyrazol-4-yl)-6-fluoro-1-(phenylsulfonyl)-1H-indole (Intermediate 38; 265 mg; 0.65 mmol), 80 mg (46%) of the title compound was obtained as a white solid after purification by preparative HPLC.

LC-MS: m/z 271.1 [M+H]⁺.

¹H NMR (400 MHz, MeOH-d₄) δ [ppm]: 8.03 (s, 1H), 7.86 (s, 1H), 7.72 (dd, J=8.7, 5.3 Hz, 1H), 7.43 (s, 1H), 7.12 (dd, J=9.9, 2.3 Hz, 1H), 6.90 (ddd, J=9.6, 8.8, 2.3 Hz, 1H), 4.48 (d, J=6.6 Hz, 2H), 4.13 (dt, J=19.1, 11.4 Hz, 3H), 3.51 (dd, J=14.1, 7.1 Hz, 1H).

### Compound 7: 3-(4-(6-fluoro-1H-indol-3-yl)-1H-pyrazol-1-yl)propanamide

### Step 1: 3-(4-(6-fluoro-1-(phenylsulfonyl)-1H-indol-3-yl)-1H-pyrazol-1-yl)propanamide

A mixture of 6-fluoro-1-(phenylsulfonyl)-3-(1H-pyrazol-4-yl)-1H-indole (Intermediate 5; 225 mg; 0.66 mmol), 3-bromopropanamide (302 mg; 1.99 mmol), KI (20 mg; 0.12 mmol) and K₂CO₃ (274 mg; 1.98 mmol) in CH₃CN (20 mL) and DMF (10 mL) was stirred at 100° C. overnight. The reaction mixture was cooled to r.t., filtered, and concentrated to afford 272 mg (100%) of the title compound as a white solid, which was used directly without further purification.

LC-MS: m/z 413.1 [M+H]⁺.

### Step 2

Following the general method as outlined in the synthesis of compound 70, starting from 3-(4-(6-fluoro-1-(phenylsulfonyl)-1H-indol-3-yl)-1H-pyrazol-1-yl)propanamide (Step 1; 272 mg; 0.66 mmol), 150 mg (83%) of the title compound was obtained as a white solid after purification by preparative HPLC.

LC-MS: m/z 273.1 [M+H]⁺.

¹H NMR (400 MHz, DMSO-d₆) δ [ppm]: 11.17 (s, 1H), 8.03 (s, 1H), 7.77 (s, 1H), 7.73 (dd, J=8.7, 5.4 Hz, 1H), 7.53 (d, J=2.2 Hz, 1H), 7.41 (s, 1H), 7.16 (dd, J=10.0, 2.3 Hz, 1H), 6.96-6.81 (m, 2H), 4.33 (t, J=7.0 Hz, 2H), 2.67 (t, J=7.0 Hz, 2H). m.p. 173.3-175.0° C.

### Compound 8: 3-(4-(5,6-difluoro-1H-indol-3-yl)-1H-pyrazol-1-yl)propanamide

### Step 1: 3-(4-(5,6-difluoro-1-(phenylsulfonyl)-1H-indol-3-yl)-1H-pyrazol-1-yl)propanamide

Following the general method as outlined in the synthesis of compound 7, starting from 5,6-difluoro-1-(phenylsulfonyl)-3-(1H-pyrazol-4-yl)-1H-indole hydrochloride (Intermediate 30; 180 mg; 0.45 mmol), 600 mg (>100%) of the title compound as a white solid, which was used directly without further purification.

LC-MS: m/z 431.1 [M+H]⁺.

### Step 2

Following the general method as outlined in the synthesis of compound 70, starting from 3-(4-(5,6-difluoro-1-(phenylsulfonyl)-1H-indol-3-yl)-1H-pyrazol-1-yl)propanamide (Step 1; 500 mg), 34.5 mg (26%) of the title compound was obtained as a white solid after purification by reverse phase flash chromatography.

¹H NMR (400 MHz, DMSO-d₆) δ [ppm]: 11.26 (s, 1H), 8.09 (s, 1H), 7.78 (s, 1H), 7.71 (dd, J=11.6, 8.0 Hz, 1H), 7.61 (s, 1H), 7.39 (dd, J=11.2, 7.0 Hz, 2H), 6.89 (s, 1H), 4.33 (t, J=7.0 Hz, 2H), 2.67 (t, J=7.0 Hz, 2H).

¹⁹F NMR (377 MHz, DMSO-d₆) δ [ppm]: −145.50 (d, J=22.2 Hz, 1H), −148.62 (d, J=22.1 Hz, 1H). m.p. 214.9-215.5° C.

### Compound 9: 3-(4-(6-fluoro-1H-indol-3-yl)-3,5-dimethyl-1H-pyrazol-1-yl)propanamide

### Step 1: 3-(4-(6-fluoro-1-(phenylsulfonyl)-1H-indol-3-yl)-3, 5-dimethyl-1H-pyrazol-1-yl)propanamide

A mixture of 3-(3,5-dimethyl-1H-pyrazol-4-yl)-6-fluoro-1-(phenylsulfonyl)-1H-indole (Intermediate 50; 100 mg; 0.27 mmol), 3-bromopropanamide (411 mg; 2.70 mmol), KI (45 mg; 0.27 mmol), and K₂CO₃ (186 mg; 1.35 mmol) in DMF (3.0 mL) was stirred at 70° C. for 16 hours. The mixture was cooled, diluted with EtOAc (80 mL), washed with water (50 mL), brine (30 mL×2), dried over anhydrous Na₂SO₄, filtered, and concentrated to afford 110 mg (92%) of the title compound as brown oil.

LC-MS: m/z 440.8 [M+H]⁺.

### Step 2

Following the general method as outlined in the synthesis of compound 3, starting from 3-(4-(6-fluoro-1-(phenylsulfonyl)-1H-indol-3-yl)-3,5-dimethyl-1H-pyrazol-1-yl)propanamide (Step 1; 110 mg; 0.25 mmol), 35 mg (47%) of the title compound was obtained as a white solid after purification by preparative HPLC.

LC-MS: m/z 301.1 [M+H]⁺.

¹H NMR (300 MHz, DMSO-d₆) δ [ppm]: 11.26 (brs, 1H), 7.43 (brs, 1H), 7.23 (d, J=2.4 Hz, 1H), 7.22 (dd, J=9.6, 5.4 Hz, 1H), 7.17 (dd, J=10.1, 2.3 Hz, 1H), 6.90 (brs, 1H), 6.84 (ddd, J=9.7, 8.7, 2.3 Hz, 1H), 4.17 (t, J=7.0 Hz, 2H), 2.62 (t, J=7.0 Hz, 2H), 2.15 (s, 3H), 2.04 (s, 3H).

### Compound 13: N-(2-(dimethylamino)ethyl)-3-(4-(6-fluoro-1H-indol-3-yl)-1H-pyrazol-1-yl)propanamide

Following the general method as outlined in the synthesis of compound 39, starting from 3-(4-(6-fluoro-1H-indol-3-yl)-1H-pyrazol-1-yl)propanoic acid (Compound 16; 110 mg; 0.40 mmol) and N,N-dimethylethylenediamine (0.08 mL; 0.8 mmol), 20 mg (15%) of the title product was obtained as a yellow solid purified by preparative HPLC.

¹H NMR (400 MHz, MeOH-d₄) δ [ppm]: 7.88 (s, 1H), 7.80 (s, 1H), 7.70 (d, J=14.0 Hz, 1H), 7.40 (s, 1H), 7.11 (d, J=7.6 Hz, 1H), 6.88 (dd, J=10.0, 8.4 Hz, 1H), 4.49 (t, J=6.5 Hz, 2H), 3.27 (t, J=6.9 Hz, 2H), 2.77 (t, J=6.5 Hz, 2H), 2.31 (t, J=6.9 Hz, 2H), 2.13 (s, 6H).

### Compound 14: 3-(4-(6-fluoro-1H-indol-3-yl)-1H-pyrazol-1-yl)-N-methylpropanamide

Following the general method as outlined in the synthesis of compound 39, starting from 3-(4-(6-fluoro-1H-indol-3-yl)-1H-pyrazol-1-yl)propanoic acid (Compound 16; 200 mg; 0.73 mmol), 43 mg (21%) of the title product was obtained as a yellow solid purified by preparative TLC (DCM/MeOH=10/1).

¹H NMR (400 MHz, DMSO-d₆) δ [ppm]: 11.17 (s, 1H), 8.02 (s, 1H), 7.88 (d, J=3.2 Hz, 1H), 7.79-7.70 (m, 2H), 7.53 (d, J=2.2 Hz, 1H), 7.16 (dd, J=10.1, 2.1 Hz, 1H), 6.95-6.87 (m, 1H), 4.35 (t, J=7.0 Hz, 2H), 2.67 (t, J=7.0 Hz, 2H), 2.57 (d, J=4.6 Hz, 3H). m.p. 185.3-186.0° C.

### Compound 15: 3-(4-(6-fluoro-1H-indol-3-yl)-1H-pyrazol-1-yl)-N,N-dimethylpropanamide

Following the general method as outlined in the synthesis of compound 40, starting from 3-(4-(6-fluoro-1H-indol-3-yl)-1H-pyrazol-1-yl)propanoic acid (Compound 16; 200 mg; 0.73 mmol), 40 mg (18%) of the title compound was obtained as a light-yellow solid after purification by preparative TLC (DCM/MeOH=30/1).

¹H NMR (300 MHz, DMSO-d₆) δ [ppm]: 11.16 (s, 1H), 8.08 (s, 1H), 7.81-7.69 (m, 2H), 7.53 (d, J=2.2 Hz, 1H), 7.16 (dd, J=10.1, 2.2 Hz, 1H), 6.91 (s, 1H), 4.35 (t, J=7.0 Hz, 2H), 2.97-2.87 (m, 5H), 2.82 (s, 3H). m.p. 157.2-158.1° C.

### Compound 16: 3-(4-(6-fluoro-1H-indol-3-yl)-1H-pyrazol-1-yl)propanoic acid

### Step 1: tert-butyl 3-(4-(6-fluoro-1-(phenylsulfonyl)-1H-indol-3-yl)-1H-pyrazol-1-yl)propanoate

A mixture of 6-fluoro-1-(phenylsulfonyl)-3-(1H-pyrazol-4-yl)-1H-indole (Intermediate 5; 100 mg; 0.29 mmol), tert-butyl acrylate (77 mg; 0.60 mmol) and Cs₂CO₃ (293 mg; 0.90 mmol) in MeCN (10 mL) was stirring at 80° C. overnight under nitrogen. The reaction mixture was filtered to remove solid and concentrated to afford 112 mg (82%) of the title compound as a yellow solid, which was used directly without further purification.

LC-MS: m/z 470.1 [M+H]⁺.

### Step 2

Following the general method as outlined in the synthesis of compound 70, starting from tert-butyl 3-(4-(6-fluoro-1-(phenylsulfonyl)-1H-indol-3-yl)-1H-pyrazol-1-yl)propanoate (Step 1; 112 mg; 0.24 mmol), 62 mg (96%) of the title compound was obtained as a yellow solid after aqueous acid base extraction and concentration without further purification.

¹H NMR (400 MHz, DMSO-d₆) δ [ppm]: 12.38 (s, 1H), 11.17 (s, 1H), 8.08 (s, 1H), 7.80-7.70 (m, 2H), 7.53 (d, J=2.4 Hz, 1H), 7.16 (dd, J=10.0, 2.4 Hz, 1H), 6.96-6.86 (m, 1H), 4.35 (t, J=6.8 Hz, 2H), 2.84 (t, J=6.8 Hz, 2H).

### Compound 17: 3-(4-(5,6-difluoro-1H-indol-3-yl)-1H-pyrazol-1-yl)propanoic acid

The title compound (28.6 mg; 22%) was obtained as a white solid after purification by reverse phase flash chromatography in step 2 of the synthesis of compound 55.

¹H NMR (400 MHz, DMSO-d₆) δ [ppm]: 11.28 (s, 1H), 8.12 (s, 1H), 7.77 (s, 1H), 7.71 (dd, J=11.7, 7.9 Hz, 1H), 7.60 (d, J=2.4 Hz, 1H), 7.38 (dd, J=11.2, 7.1 Hz, 1H), 4.31 (t, J=7.0 Hz, 2H), 2.73 (t, J=7.0 Hz, 3H).

¹⁹F NMR (377 MHz, DMSO-d₆) δ [ppm]: −145.55 (d, J=22.1 Hz, 1H), -148.66 (d, J=22.3 Hz, 1H).

### Compound 18: 1-(2-(4-(6-fluoro-1H-indol-3-yl)-1H-pyrazol-1-yl)ethyl)imidazolidin-2-one

### Step 1: 1-(2-(4-(6-fluoro-1-(phenylsulfonyl)-1H-indol-3-yl)-1H-pyrazol-1-yl)ethyl)imidazolidin-2-one

A mixture of 6-fluoro-1-(phenylsulfonyl)-3-(1H-pyrazol-4-yl)-1H-indole (Intermediate 5; 300 mg; 0.88 mmol), 2-(2-oxoimidazolidin-1-yl)ethyl methanesulfonate (Intermediate 31; 274 mg; 1.32 mmol), NaHCO₃ (403 mg; 4.80 mmol) in EtOH (10 mL) was stirred at 90° C. overnight. The reaction mixture was concentrated, diluted with EtOAc (30 mL), washed with water (30 mL), brine (30 mL), dried over anhydrous Na₂SO₄, filtered, and concentrated to afford 100 mg (25%) of the title compound as a yellow solid, which was used directly used without further purification.

LC-MS: m/z 454.1 [M+H]⁺.

### Step 2

Following the general method as outlined in the synthesis of compound 70, starting from 1-(2-(4-(6-fluoro-1-(phenylsulfonyl)-1H-indol-3-yl)-1H-pyrazol-1-yl)ethyl)imidazolidin-2-one (Step 1; 100 mg; 0.22 mmol), 24 mg (35%) of the title compound was obtained as a yellow solid after purification by preparative HPLC.

¹H NMR (400 MHz, DMSO-d₆) δ [ppm]: 11.18 (s, 1H), 8.14 (s, 1H), 7.84-7.71 (m, 2H), 7.55 (d, J=2.3 Hz, 1H), 7.16 (dd, J=10.1, 2.3 Hz, 1H), 6.91 (td, J=9.7, 2.4 Hz, 1H), 6.33 (s, 1H), 4.24 (t, J=6.1 Hz, 2H), 3.49 (t, J=6.1 Hz, 2H), 3.16 (s, 4H).

### Compound 19: 6-fluoro-3-(1-(2-(4-methylpiperazin-1-yl)ethyl)-1H-pyrazol-4-yl)-1H-indole

### Step 1: 6-fluoro-3-(1-(2-(4-methylpiperazin-1-yl)ethyl)-1H-pyrazol-4-yl)-1-(phenylsulfonyl)-1H-indole

To a solution of 3-(1-(2-bromoethyl)-1H-pyrazol-4-yl)-6-fluoro-1-(phenylsulfonyl)-1H-indole (Intermediate 22; 100 mg; 0.22 mmol) in MeCN (10 mL) was added 1-methylpiperazine (44 mg; 0.44 mmol), K₂CO₃ (62 mg; 0.45 mmol), and NaI (33 mg; 0.22 mmol). The mixture was stirred at 80° C. for 4 hours, filtered, concentrated, and purified by preparative TLC (DCM/MeOH=10/1) to afford 70 mg (68%) of the title compound as a yellow solid.

¹H NMR (300 MHz, DMSO-d₆) δ [ppm]: 8.32 (s, 1H), 8.05-8.03 (m, 3H), 7.95 (s, 1H), 7.89-7.85 (m, 1H), 7.79-7.68 (m, 2H). 7.63-7.57 (m, 2H), 7.26-7.19 (m, 1H), 4.26-4.22 (t, 2H), 2.76-2.71 (t, 2H), 2.50-2.27 (m, 8H), 2.15 (s, 3H).

### Step 2

Following the general method as outlined in the synthesis of compound 70, starting from 6-fluoro-3-(1-(2-(4-methylpiperazin-1-yl)ethyl)-1H-pyrazol-4-yl)-1-(phenylsulfonyl)-1H-indole (Step 1; 80 mg; 0.17 mmol), 31 mg (55%) of the title compound was obtained as a yellow solid after purification by preparative TLC (DCM/MeOH=10/1).

¹H NMR (300 MHz, DMSO-d₆) δ [ppm]: 11.25 (s, 1H), 8.13 (s, 1H), 7.75-7.72 (m, 2H), 7.54-7.53 (d, 1H), 7.19-7.15 (m, 1H), 6.94-6.87 (m, 1H), 4.28-4.24 (t, 2H), 2.92-2.52 (m, 8H), 2.52-2.50 (t, 2H), 2.49 (s, 3H).

### Compound 20: 4-(2-(4-(6-fluoro-1H-indol-3-yl)-1H-pyrazol-1-yl)ethyl)morpholine

### Step 1: 4-(2-(4-(6-fluoro-1-(phenylsulfonyl)-1H-indol-3-yl)-1H-pyrazol-1-yl)ethyl)morpholine

Following the general method as outlined in the synthesis of compound 41 Step 1, starting from 6-fluoro-1-(phenylsulfonyl)-3-(1H-pyrazol-4-yl)-1H-indole (Intermediate 5; 250 mg; 0.73 mmol) and 4-(2-chloroethyl)-morpholine hydrochloride (272 mg; 1.46 mmol), 233 mg (70%) of the title compound was obtained as a brown solid after purification by a silica gel chromatography (petroleum ether/EtOAc=1/1).

¹H NMR (300 MHz, DMSO-d₆) δ [ppm]: 8.34 (s, 1H), 8.11-8.02 (m, 3H), 7.97 (s, 1H), 7.88 (dd, J=8.8, 5.3 Hz, 1H), 7.82-7.67 (m, 2H), 7.61 (t, J=7.5 Hz, 2H), 7.29-7.19 (m, 1H), 4.26 (t, J=6.6 Hz, 2H), 3.57-3.51 (m, 4H), 2.74 (dd, J=8.8, 4.4 Hz, 2H), 2.42 (s, 4H).

### Step 2

Following the general method as outlined in the synthesis of compound 70, starting from 4-(2-(4-(6-fluoro-1-(phenylsulfonyl)-1H-indol-3-yl)-1H-pyrazol-1-yl)ethyl)morpholine (Step 1; 233 mg; 0.51 mmol), 91 mg (57%) of the title compound was obtained as a yellow solid after purification by preparative HPLC.

¹H NMR (300 MHz, DMSO-d₆) δ [ppm]: 11.17 (s, 1H), 8.12 (s, 1H), 7.82-7.69 (m, 2H), 7.54 (d, J=2.4 Hz, 1H), 7.17 (dd, J=10.1, 2.2 Hz, 1H), 6.98-6.84 (m, 1H), 4.26 (t, J=6.6 Hz, 2H), 3.62-3.51 (m, 4H), 2.75 (t, J=6.6 Hz, 2H), 2.47-2.38 (m, 4H). m.p. 144.2-1145.6° C.

### Compound 21: N-(2-(4-(6-fluoro-1H-indol-3-yl)-1H-pyrazol-1-yl)ethyl)acetamide

### Step 1: N-(2-(4-(6-fluoro-1-(phenylsulfonyl)-1H-indol-3-yl)-1H-pyrazol-1-yl)ethyl)acetamide

To the solution of 2-(4-(6-fluoro-1-(phenylsulfonyl)-1H-indol-3-yl)-1H-pyrazol-1-yl)ethanamine hydrochloride (Intermediate 19; 260 mg; 0.62 mmol) in pyridine (3 mL), was added Ac₂O (2.00 mL; 2.13 mmol) under nitrogen. The reaction mixture was stirred overnight. The reaction mixture was concentrated and the residue was trituated with a small amount of EtOAc to afford 150 mg (52%) of the title compound as a red solid, which was used directly without further purification.

¹H NMR (400 MHz, DMSO-d₆) δ [ppm]: 8.31 (s, 1H), 8.09 (s, 1H), 8.05 (d, J=7.9 Hz, 2H), 8.02-7.94 (m, 2H), 7.91 (dd, J=8.7, 5.3 Hz, 1H), 7.77 (dd, J=9.9, 1.9 Hz, 1H), 7.71 (t, J=7.4 Hz, 1H), 7.61 (t, J=7.7 Hz, 2H), 7.23 (td, J=8.9, 1.9 Hz, 1H), 4.19 (t, J=6.2 Hz, 2H), 3.47 (q, J=5.9 Hz, 2H), 1.79 (s, 3H).

### Step 2

Following the general method as outlined in the synthesis of compound 70, starting from N-(2-(4-(6-fluoro-1-(phenylsulfonyl)-1H-indol-3-yl)-1H-pyrazol-1-yl)ethyl)acetamide (Step 1; 150 mg; 0.35 mmol), 66 mg (66%) of the title compound was obtained as a white solid after purification by preparative HPLC.

LC-MS: m/z 287.2 [M+H]⁺.

¹H NMR (400 MHz, DMSO-d₆) δ [ppm]: 11.17 (s, 1H), 8.08 (s, 1H), 8.01 (t, J=5.4 Hz, 1H), 7.80 (s, 1H), 7.77 (dd, J=8.7, 5.4 Hz, 1H), 7.54 (d, J=2.2 Hz, 1H), 7.16 (dd, J=10.1, 2.2 Hz, 1H), 6.98-6.82 (m, 1H), 4.18 (t, J=6.3 Hz, 2H), 3.47 (q, J=6.1 Hz, 2H), 1.80 (s, 3H). m.p. 179.5-180.8° C.

### Compound 22: 1-(2-(4-(6-fluoro-1H-indol-3-yl)-1H-pyrazol-1-yl)ethyl)urea

### Step 1: 1-(2-(4-(6-fluoro-1-(phenylsulfonyl)-1H-indol-3-yl)-1H-pyrazol-1-yl)ethyl)urea

Following the general method as outlined in the synthesis of compound 4, starting from 2-(4-(6-fluoro-1-(phenylsulfonyl)-1H-indol-3-yl)-1H-pyrazol-1-yl)ethanamine hydrochloride (Intermediate 19; 100 mg; 0.24 mmol), 161 mg (>100%) of the title compound was obtained as a yellow solid, which used directly without further purification.

LC-MS: m/z 428.1 [M+H]⁺.

### Step 2

Following the general method as outlined in the synthesis of compound 70, starting from 1-(2-(4-(6-fluoro-1-(phenylsulfonyl)-1H-indol-3-yl)-1H-pyrazol-1-yl)ethyl)urea (Step 1; 161 mg), 41 mg (60%) of the title compound was obtained as a yellow solid after purification by preparative TLC (EtOAc).

¹H NMR (400 MHz, DMSO-d₆) δ [ppm]: 1.21 (s, 1H), 8.07 (s, 1H), 7.79 (s, 1H), 7.77 (dd, J=8.7, 5.5 Hz, 1H), 7.53 (d, J=2.3 Hz, 1H), 7.16 (dd, J=10.1, 2.3 Hz, 1H), 6.93-6.86 (m, 1H), 6.08 (t, J=6.0 Hz, 1H), 5.54 (s, 2H), 4.15 (t, J=6.1 Hz, 2H), 3.42 (dd, J=12.3, 6.1 Hz, 2H).

### Compound 23: 1-(2-(4-(6-fluoro-1H-indol-3-yl)-1H-pyrazol-1-yl)ethyl)-3-methylurea

### Step 1: 2-(4-(6-fluoro-1H-indol-3-yl)-1H-pyrazol-1-yl)ethanamine

Following the general method as outlined in the synthesis of compound 70, starting from 2-(4-(6-fluoro-1-(phenylsulfonyl)-1H-indol-3-yl)-1H-pyrazol-1-yl)ethanamine hydrochloride (Intermediate 19; 427 mg; 1.01 mmol), 169 mg (68%) of the title compound was obtained as a yellow oil after purification by preparative TLC (DCM/MeOH=20/1).

LC-MS: m/z 245.1 [M+H]⁺.

### Step 2

Following the general method as outlined in the synthesis of compound 5, starting from 2-(4-(6-fluoro-1H-indol-3-yl)-1H-pyrazol-1-yl)ethanamine (Step 1; 113 mg; 0.46 mmol), 21.4 mg (15%) of the title compound was obtained as a yellow solid after purification by preparative HPLC.

¹H NMR (400 MHz, DMSO-d₆) δ [ppm]: 11.15 (s, 1H), 8.06 (s, 1H), 7.79 (s, 1H), 7.76 (dd, J=8.7, 5.4 Hz, 1H), 7.75 (s, 1H), 7.53 (d, J=2.3 Hz, 1H), 7.16 (dd, J=10.0, 2.3 Hz, 1H), 6.90 (td, J=9.6, 2.3 Hz, 1H), 5.96 (t, J=5.7 Hz, 1H), 5.84 (d, J=4.5 Hz, 1H), 4.15 (t, J=6.2 Hz, 2H), 3.45 (q, J=6.1 Hz, 2H), 2.54 (d, J=4.7 Hz, 3H). m.p. 197.6-198.7° C.

### Compound 24: 2-(4-(6-fluoro-1H-indol-3-yl)-1H-pyrazol-1-yl)-N-methylethanamine

To a slurry of LiAlH₄ (100 mg; 2.64 mmol) in THF (4 mL) was added a solution of tert-butyl 2-(4-(6-fluoro-1-(phenylsulfonyl)-1H-indol-3-yl)-1H-pyrazol-1-yl)ethylcarbamate (Intermediate 18; 300 mg; 0.62 mmol) in THF (6 mL) dropwise under argon. The reaction mixture was stirred at r.t. overnight and then at reflux for 4 hours. The reaction was added sequentially water (0.1 mL), 10% aqueous NaOH (0.2 mL), water (5 mL), filtered and washed with EtOAc. The filtrate was extracted with EtOAc (30 mL×2). The combined organic layers were dried over anhydrous Na₂SO₄, filtered, concentrated, and purified by a silica gel chromatography (DCM/MeOH=20/1) to afford the 94 mg (62%) of title compound as a yellow solid.

LC-MS: m/z 259 [M+H]⁺.

¹H NMR (400 MHz, DMSO-d₆) δ [ppm]: 11.15 (s, 1H), 8.08 (s, 1H), 7.77 (s, 1H), 7.76-7.73 (m, 1H), 7.53 (d, J=2.4 Hz, 1H), 7.16 (dd, J=10.1, 2.4 Hz, 1H), 6.93-6.87 (m, 1H), 4.19 (t, J=6.4 Hz, 2H), 2.90 (t, J=6.4 Hz, 2H), 2.30 (s, 3H). m.p. 94.2-95.2° C.

### Compound 25: N-(2-(4-(6-fluoro-1H-indol-3-yl)-1H-pyrazol-1-yl)ethyl)methanesulfonamide

### Step 1: N-(2-(4-(6-fluoro-1-(phenylsulfonyl)-1H-indol-3-yl)-1H-pyrazol-1-yl)ethyl)methanesulfonamide

To the solution of 2-(4-(6-fluoro-1-(phenylsulfonyl)-1H-indol-3-yl)-1H-pyrazol-1-yl)ethanamine hydrochloride (Intermediate 19; 228 mg; 0.54 mmol) and DMAP (3 mg; 0.02 mmol) in pyridine (4 mL), was added MsCl (0.07 mL; 0.90 mmol) under nitrogen at 0° C. The reaction was warmed to r.t. and stirred overnight. The reaction mixture was concentrated and the residue was diluted with EtOAc (20 mL), washed with aqueous HCl (10 mL; 1 M), water (10 mL), brine (10 mL). The organic layer was dried over anhydrous Na₂SO₄, filtered, and concentrated to afford 300 mg of the title compound as a yellow solid, which was used directly without further purification.

LC-MS: m/z 460.8 [M−H]⁻.

### Step 2

Following the general method as outlined in the synthesis of compound 70, starting from N-(2-(4-(6-fluoro-1-(phenylsulfonyl)-1H-indol-3-yl)-1H-pyrazol-1-yl)ethyl)methanesulfonamide (Step 1; 300 mg; 0.65 mmol), 54 mg (26%) of the title compound was obtained as a yellow solid after purification by preparative TLC (DCM/MeOH=10/1).

LC-MS: m/z 323.1 [M+H]⁺.

¹H NMR (400 MHz, DMSO-d₆) δ [ppm]: 8.00 (s, 1H), 7.83 (s, 1H), 7.74-7.68 (m, 1H), 7.41 (s, 1H), 7.09 (dd, J=8.0, 1.9 Hz, 1H), 6.88 (m, 1H), 4.34 (t, J=6.0 Hz, 2H), 3.57 (q, J=6.0 Hz, 2H), 2.85 (s, 3H).

### Compound 26: 2-(4-(6-fluoro-1H-indol-3-yl)-1H-pyrazol-1-yl)ethanol

### Step 1: 2-(4-(6-fluoro-1-(phenylsulfonyl)-1H-indol-3-yl)-1H-pyrazol-1-yl)ethanol

A solution of 6-fluoro-1-(phenylsulfonyl)-3-(1H-pyrazol-4-yl)-1H-indole (Intermediate 5; 1.20 mg; 0.35 mmol), 2-bromoethanol (157 mg; 1.26 mmol) and K₂CO₃ (145 mg; 1.05 mmol) in MeCN (4 mL) was heated to 165° C. for 1 h in a microwave reactor. The reaction mixture was filtered and washed with MeCN. The combined filtrate was concentrated and purified by preparative TLC (petroleum ether/EtOAc=1/1) to afford 41 mg (30%) of the title compound as a yellow semi-solid.

¹H NMR (400 MHz, CDCl₃) δ [ppm]: 7.90 (d, J=7.6 Hz, 2H), 7.75 (m, 3H), 7.56 (m, 3H), 7.46 (t, J=7.7 Hz, 2H), 7.03 (td, J=8.9, 2.3 Hz, 1H), 4.31 (m, 2H), 4.05 (m, 2H).

### Step 2

Following the general method as outlined in the synthesis of compound 70, starting from 2-(4-(6-fluoro-1-(phenylsulfonyl)-1H-indol-3-yl)-1H-pyrazol-1-yl)ethanol (Step 1; 61 mg; 0.16 mmol), 17 mg (44%) of the title compound was obtained as a yellow solid after purification by preparative TLC (petroleum ether/EtOAc=1/1).

¹H NMR (400 MHz, MeOH-d₄) δ [ppm]: 7.85 (s, 1H), 7.69 (s, 1H), 7.59 (dd, J=8.7, 5.3 Hz, 1H), 7.28 (s, 1H), 6.98 (dd, J=9.9, 2.3 Hz, 1H), 6.76 (td, J=9.5, 2.3 Hz, 1H), 4.18 (t, J=5.4 Hz, 2H), 3.83 (t, J=5.4 Hz, 2H). m.p. 140.2-140.8° C.

### Compound 27: 6-fluoro-3-(1-(2-(piperazin-1-yl)ethyl)-1H-pyrazol-4-yl)-1H-indole

### Step 1: 6-fluoro-1-(phenylsulfonyl)-3-(1-(2-(piperazin-1-yl)ethyl)-1H-pyrazol-4-yl)-1H-indole

Following the general method as outlined in the synthesis of compound 28, starting from 3-(1-(2-bromoethyl)-1H-pyrazol-4-yl)-6-fluoro-1-(phenylsulfonyl)-1H-indole (Intermediate 22; 100 mg; 0.22 mmol) and piperazine (38 mg; 0.44 mmol), 68 mg (68%) of the title compound was obtained as a yellow solid after purification by preparative TLC (DCM/MeOH=10/1).

¹H NMR (400 MHz, DMSO-d₆) δ [ppm]: 8.32 (s, 1H), 8.08-8.04 (m, 3H), 7.96 (s, 1H), 7.89-7.85 (m, 1H), 7.78-7.75 (m, 1H). 7.72-7.68 (m, 1H), 7.62-7.58 (m, 2H), 7.25-7.20 (m, 1H), 4.26-4.23 (t, 2H), 2.77-2.72 (m, 6H), 2.43 (s, 4H).

### Step 2

Following the general method as outlined in the synthesis of compound 70, starting from 6-fluoro-1-(phenylsulfonyl)-3-(1-(2-(piperazin-1-yl)ethyl)-1H-pyrazol-4-yl)-1H-indole (Step 1; 80 mg; 0.18 mmol), 33 mg (60%) of the title compound was obtained as a yellow solid after purification by preparative TLC (DCM/MeOH=10/1).

¹H NMR (400 MHz, DMSO-d₆) δ [ppm]: 11.20 (s, 1H), 8.117-8.116 (d, 1H), 7.76-7.73 (m, 2H), 7.539-7.533 (d, 1H), 7.18-7.15 (dd, 1H). 6.93-6.88 (m, 1H), 4.25-4.22 (t, 2H), 2.77-2.72 (m, 6H), 2.44-2.43 (d, 4H).

### Compound 28: 1-(4-(2-(4-(6-fluoro-1H-indol-3-yl)-1H-pyrazol-1-yl)ethyl)piperazin-1-yl)ethanone

### Step 1: 1-(4-(2-(4-(6-fluoro-1-(phenylsulfonyl)-1H-indol-3-yl)-1H-pyrazol-1-yl)ethyl)piperazin-1-yl)ethanone

Following the general method as outlined in the synthesis of compound 89, starting from 6-fluoro-1-(phenylsulfonyl)-3-(1-(2-(piperazin-1-yl)ethyl)-1H-pyrazol-4-yl)-1H-indole (Compound obtained in Step 1 of the synthesis of Compound 27; 138 mg; 0.30 mmol), 140 mg (94%) of the title compound was obtained as a yellow oil, which was used directly without further purification.

LC-MS: m/z 496.2 [M+H]⁺.

### Step 2

Following the general method as outlined in the synthesis of compound 70, starting from 1-(4-(2-(4-(6-fluoro-1-(phenylsulfonyl)-1H-indol-3-yl)-1H-pyrazol-1-yl)ethyl)piperazin-1-yl)ethanone (Step 1; 140 mg; 0.28 mmol), 80 mg (81%) of the title compound was obtained as a yellow solid after purification by preparative TLC (DCM/MeOH=20/1).

LC-MS: m/z 356.2 [M+H]⁺.

¹H NMR (400 MHz, MeOH-d₄) δ [ppm]: 7.87 (s, 1H), 7.67 (s, 1H), 7.58 (dd, J=8.7, 5.3 Hz, 1H), 7.28 (s, 1H), 6.98 (dd, J=9.9, 2.3 Hz, 1H), 6.76 (td, J=9.3, 2.3 Hz, 1H), 4.23 (t, J=6.4 Hz, 2H), 3.52-3.31 (m, 4H), 2.77 (t, J=6.4 Hz, 2H), 2.48-2.31 (m, 4H), 1.96 (s, 3H).

¹⁹F NMR (377 MHz, MeOH-d₄) δ [ppm]: −124.26 (s, 1H).

### Compound 29: 3-(1-(azetidin-3-yl)-1H-pyrazol-4-yl)-6-fluoro-1H-indole

### Step 1: 6-fluoro-1-(phenylsulfonyl)-3-(1-(2-(pyrrolidin-1-yl)ethyl)-1H-pyrazol-4-yl)-1H-indole

To a solution of 6-fluoro-1-(phenylsulfonyl)-3-(1H-pyrazol-4-yl)-1H-indole (Intermediate 5; 240 mg; 0.70 mmol) and Cs₂CO₃ (688 mg; 2.11 mmol) in DMF (20 mL) was added 1-(2-chloroethyl)-pyrrolidine (186 mg; 1.39 mmol). The reaction mixture was stirred at 65° C. for 2 hours, cooled to r.t., diluted with 10% aqueous NH₄Cl (20 mL), and extracted with EtOAc (20 mL×3). The combined organic layers were washed with water (20 mL), brine (20 mL), dried over Na₂SO₄ anhydrous, and concentrated to afford 300 mg (98%) of the title compound as a yellow solid, which was used directly without further purification.

LC-MS: m/z 439.2 [M+H]⁺.

### Step 2

Following the general method as outlined in the synthesis of compound 70, starting from 6-fluoro-3-(1-(2-(methylsulfonyl)ethyl)-1H-pyrazol-4-yl)-1-(phenylsulfonyl)-1H-indole (Step 1; 300 mg; 0.68 mmol), 170 mg (84%) of the title compound was obtained as a brown semi-solid after recrystallization from MeOH and petroleum ether.

¹H NMR (400 MHz, MeOH-d₄) δ [ppm]: 8.09 (s, 1H), 7.90 (s, 1H), 7.74 (dd, J=8.7, 5.3 Hz, 1H), 7.45 (s, 1H), 7.12 (dd, J=9.8, 2.3 Hz, 1H), 6.90 (td, J=9.5, 2.3 Hz, 1H), 4.59 (t, J=6.0 Hz, 2H), 3.62 (t, J=6.0 Hz, 2H), 3.25 (s, 4H), 2.05 (m, 4H).

### Compound 30: 1-(2-(4-(6-fluoro-1H-indol-3-yl)-1H-pyrazol-1-yl)ethyl)pyrrolidin-2-one

### Step 1: 1-(2-(4-(6-fluoro-1-(phenylsulfonyl)-1H-indol-3-yl)-1H-pyrazol-1-yl)ethyl)pyrrolidin-2-one

A mixture of 6-fluoro-1-(phenylsulfonyl)-3-(1H-pyrazol-4-yl)-1H-indole (Intermediate 5; 443 mg; 1.30 mmol), 2-(2-oxopyrrolidin-1-yl)ethyl methanesulfonate (Intermediate 43; 400 mg; 1.93 mmol), Cs₂CO₃ (1.30 g; 3.99 mmol) in DMF (10 mL) was stirred at 90° C. overnight. The reaction mixture was diluted with water (40 mL) and extracted with EtOAc (40 mL×3). The combined organic layers were washed with brine (30 mL), dried over anhydrous Na₂SO₄, filtered, and concentrated to afford 285 mg (48%) of the title compound as a brown solid, which was used directly used without further purification.

LC-MS: m/z 453.1 [M+H]⁺.

### Step 2

Following the general method as outlined in the synthesis of compound 70, starting from 1-(2-(4-(6-fluoro-1-(phenylsulfonyl)-1H-indol-3-yl)-1H-pyrazol-1-yl)ethyl)pyrrolidin-2-one (Step 1; 285 mg; 0.63 mmol), 23 mg (12%) of the title compound was obtained as a brown solid after purification by preparative HPLC.

¹H NMR (400 MHz, DMSO-d₆) δ [ppm]: 11.23 (s, 1H), 8.14 (s, 1H), 7.87-7.72 (m, 2H), 7.55 (s, 1H), 7.17 (d, J=9.2 Hz, 1H), 6.91 (t, J=8.8 Hz, 1H), 4.27 (t, J=9.5 Hz, 2H), 3.61 (d, J=4.5 Hz, 2H), 3.13 (t, J=6.7 Hz, 2H), 2.23-2.09 (m, 2H), 1.84 (dd, J=14.0, 7.3 Hz, 2H).

### Compound 31: 6-fluoro-3-(1-(2-(methylsulfonyl)ethyl)-1H-pyrazol-4-yl)-1H-indole

### Step 1: 6-fluoro-3-(1-(2-(methylsulfonyl)ethyl)-1H-pyrazol-4-yl)-1-(phenylsulfonyl)-1H-indole

A mixture of 6-fluoro-1-(phenylsulfonyl)-3-(1H-pyrazol-4-yl)-1H-indole (Intermediate 5; 210 mg; 0.62 mmol), methyl vinyl sulfone (654 mg; 6.16 mmol) and Et₃N (0.26 mL; 1.85 mmol) in methanol (15 mL) was heated at 130° C. for 0.5 h in a microwave reactor. The reaction mixture was cooled to r.t., concentrated to give a residue which was triturated with MeOH (2 mL) to afford 160 mg (58%) of the title product as a white solid.

LC-MS: m/z 448.0 [M+H]⁺.

¹H NMR (400 MHz, DMSO-d₆) δ [ppm]: 8.44 (s, 1H), 8.13 (s, 1H), 8.06 (dd, J=6.0, 2.7 Hz, 3H), 7.89 (dd, J=8.8, 5.3 Hz, 1H), 7.78 (dd, J=9.8, 2.2 Hz, 1H), 7.71 (t, J=7.5 Hz, 1H), 7.61 (t, J=7.7 Hz, 2H), 7.25 (td, J=9.1, 2.3 Hz, 1H), 4.59 (t, J=6.9 Hz, 2H), 3.75 (t, J=6.9 Hz, 2H), 2.91 (s, 3H).

### Step 2

Following the general method as outlined in the synthesis of compound 70, starting from 6-fluoro-3-(1-(2-(methylsulfonyl)ethyl)-1H-pyrazol-4-yl)-1-(phenylsulfonyl)-1H-indole (Step 1; 160 mg; 0.36 mmol), 70 mg (63%) of the title compound was obtained as a yellow solid after purification by preparative TLC (EtOAc).

LC-MS: m/z 308.1 [M+H]⁺.

¹H NMR (400 MHz, MeOH-d₄) δ [ppm]: 7.94 (s, 1H), 7.75 (s, 1H), 7.59 (dd, J=8.7, 5.3 Hz, 1H), 7.30 (s, 1H), 6.98 (dd, J=9.8, 2.1 Hz, 1H), 6.77 (td, J=9.7, 2.3 Hz, 1H), 4.59 (t, J=6.5 Hz, 2H), 3.64 (t, J=6.5 Hz, 2H), 2.67 (s, 3H). m.p. 153.3-155.1° C.

### Compound 32: 5,6-difluoro-3-(1-(2-(methylsulfonyl)ethyl)-1H-pyrazol-4-yl)-1H-indole

### Step 1: 5,6-difluoro-3-(1-(2-(methylsulfonyl)ethyl)-1H-pyrazol-4-yl)-1-(phenylsulfonyl)-1H-indole

Following the general method as outlined in the synthesis of compound 31, starting from 5,6-difluoro-1-(phenylsulfonyl)-3-(1H-pyrazol-4-yl)-1H-indole hydrochloride (Intermediate 30; 320 mg; 0.81 mmol), 186 mg (49%) of the title product was obtained as a white solid.

LC-MS: m/z 466.1 [M+H]⁺.

¹H NMR (400 MHz, DMSO-d₆) δ [ppm]: 8.47 (s, 1H), 8.21 (s, 1H), 8.05-8.07 (m, 3H), 8.04-7.98 (m, 1H), 7.94 (dd, J=10.8, 7.8 Hz, 1H), 7.72 (t, J=7.4 Hz, 1H), 7.61 (t, J=7.8 Hz, 2H), 4.58 (t, J=6.9 Hz, 2H), 3.75 (t, J=6.9 Hz, 2H), 2.91 (s, 3H).

### Step 2

Following the general method as outlined in the synthesis of compound 70, starting from 6-fluoro-3-(1-(2-(methylsulfonyl)ethyl)-1H-pyrazol-4-yl)-1-(phenylsulfonyl)-1H-indole (Step 1; 185 mg; 0.40 mmol), 77 mg (60%) of the title compound was obtained as a yellow solid after purification by preparative HPLC.

LC-MS: m/z 326.1 [M+H]⁺.

¹H NMR (400 MHz, MeOH-d₄) δ [ppm]: 11.30 (s, 1H), 8.27 (s, 1H), 7.86 (s, 1H), 7.75 (dd, J=11.6, 8.0 Hz, 1H), 7.65 (d, J=2.4 Hz, 1H), 7.40 (dd, J=11.2, 7.1 Hz, 1H), 4.58 (t, J=6.9 Hz, 2H), 3.76 (t, J=6.9 Hz, 2H), 2.89 (s, 3H). m.p. 170.6-171.9° C.

### Compound 33: 3-(3,5-dimethyl-1-(2-(methylsulfonyl)ethyl)-1H-pyrazol-4-yl)-6-fluoro-1H-indole

### Step 1: 3-(3,5-dimethyl-1-(2-(methylsulfonyl)ethyl)-1H-pyrazol-4-yl)-6-fluoro-1-(phenylsulfonyl)-1H-indole

Following the general method as outlined in the synthesis of compound 31, starting from 3-(3,5-dimethyl-1H-pyrazol-4-yl)-6-fluoro-1-(phenylsulfonyl)-1H-indole (Intermediate 14; 37 mg; 0.10 mmol), 47 mg (100%) of the title product was obtained as a yellow oil after concentration, which was used directly without further purification.

LC-MS: m/z 475.8 [M+H]⁺.

### Step 2

Following the general method as outlined in the synthesis of compound 3, starting from 3-(3,5-dimethyl-1-(2-(methylsulfonyl)ethyl)-1H-pyrazol-4-yl)-6-fluoro-1-(phenylsulfonyl)-1H-indole (Step 1; 47 mg; 0.10 mmol), 18 mg (54%) of the title compound was obtained as a white solid after purification by preparative HPLC.

LC-MS: m/z 336.0 [M+H]⁺.

¹H NMR (300 MHz, CDCl₃) δ [ppm]: 8.27 (brs, 1H), 7.23 (dd, J=8.8, 5.2 Hz, 1H), 7.12 (dd, J=9.5, 2.1 Hz, 1H), 7.06 (d, J=2.2 Hz, 1H), 6.88 (ddd, J=9.7, 8.8, 2.1 Hz, 1H), 4.53 (t, J=6.0 Hz, 2H), 3.70 (t, J=6.0 Hz, 2H), 2.59 (s, 3H), 2.27 (s, 3H), 2.17 (s, 3H).

### Compound 35: (−)-3-(4-(6-fluoro-1H-indol-3-yl)-1H-pyrazol-1-yl)-2-methylpropanamide

### Step 1: methyl 3-(4-(6-fluoro-1-(phenylsulfonyl)-1H-indol-3-yl)-1H-pyrazol-1-yl)-2-methylpropanoate

Following the general method as outlined in the synthesis of compound 6 Step 1, starting from 6-fluoro-1-(phenylsulfonyl)-3-(1H-pyrazol-4-yl)-1H-indole (Intermediate 5; 683 mg; 2.00 mmol) and methyl 2-methyl-3-((methylsulfonyl)oxy)propanoate (588 mg; 3.00 mmol), 341 mg (39%) of the title compound was obtained as a colorless oil after purification by a silica gel chromatography (petroleum ether/EtOAc=3/1 to 2/1).

LC-MS: m/z 442.1 [M+H]⁺.

### Step 2: 3-(4-(6-fluoro-1-(phenylsulfonyl)-1H-indol-3-yl)-1H-pyrazol-1-yl)-2-methylpropanoic acid

Following the general method as outlined in the synthesis of compound 6 Step 2, starting from methyl 3-(4-(6-fluoro-1-(phenylsulfonyl)-1H-indol-3-yl)-1H-pyrazol-1-yl)-2-methylpropanoate (Step 1; 341 mg; 0.77 mmol), 329 mg (100%) of the title compound was obtained as a yellow oil, which was used directly without further purification.

LC-MS: m/z 428.1 [M+H]⁺.

### Step 3: 3-(4-(6-fluoro-1-(phenylsulfonyl)-1H-indol-3-yl)-1H-pyrazol-1-yl)-2-methylpropanamide

Following the general method as outlined in the synthesis of compound 40 Step 1, starting from 3-(4-(6-fluoro-1-(phenylsulfonyl)-1H-indol-3-yl)-1H-pyrazol-1-yl)-2-methylpropanoic acid (Step 2; 329 mg; 0.77 mmol), 328 mg (100%) of the title compound was obtained as a yellow oil, which was used directly without further purification.

LC-MS: m/z 427.1 [M+H]⁺.

### Step 4

Following the general method as outlined in the synthesis of compound 70, starting from 3-(4-(6-fluoro-1-(phenylsulfonyl)-1H-indol-3-yl)-1H-pyrazol-1-yl)-2-methylpropanamide (Step 3; 328 mg; 0.77 mmol), 84 mg (38%) of the racemic title compound was obtained as a white solid after purification by preparative HPLC.

Compound 35 (26.4 mg) was obtained as the first eluting enantiomer after chiral preparative HPLC (Chiralpak IA column, 250 mm×20 mm 5 μm; mobile phase: hexane/isopropyl alcohol/Et₂NH 80/20/0.3; flow: 12 mL/min).

Chiral purity (HPLC; Chiralpak IA 250 mm×4.6 mm 5 μm; Mobile phase: Hexane/IPA 70/30; flow: 1.0 mL/min; UV detection at 230 nm; Retention time 6.10 min): 98.6% e.e.

Optical rotation: [α]²⁵⁴_{D}=−7.0 (c=1.0 g/100 ml, MeOH/MeCN=1/1).

LC-MS: m/z 287.1 [M+H]⁺.

¹H NMR (400 MHz, MeOH-d₄) δ [ppm]: 8.11 (s, 1H), 8.01 (d, J=4.7 Hz, 1H), 7.90 (dd, J=8.7, 5.4 Hz, 1H), 7.60 (s, 1H), 7.31 (dd, J=9.9, 2.1 Hz, 1H), 7.14-7.04 (m, 1H), 4.67 (dd, J=13.8, 8.4 Hz, 2H), 4.40 (dd, J=13.7, 6.2 Hz, 2H), 3.44-3.19 (m, 3H), 1.41 (dd, J=13.8, 6.5 Hz, 3H). m.p. 184.5-185.5° C.

### Compound 36: (+)-3-(4-(6-fluoro-1H-indol-3-yl)-1H-pyrazol-1-yl)-2-methylpropanamide

The title compound (second eluting enantiomer, 20.6 mg) was isolated from the chiral preparative HPLC as described in the synthesis of compound 35.

Chiral purity (HPLC; Chiralpak IA 250 mm×4.6 mm 5 μm; Mobile phase: Hexane/IPA 70/30; flow: 1.0 mL/min; UV detection at 230 nm; Retention time 7.39 min): 93.9% e.e.

Optical rotation [α]²⁵⁴_{D}=+0.3 (c=, MeOH/MeCN=1/1).

LC-MS: m/z 287.1 [M+H]⁺.

¹H NMR (400 MHz, MeOH-d₄) δ [ppm]: 11.16 (s, 1H), 8.01 (s, 1H), 7.78 (s, 1H), 7.72 (dd, J=8.7, 5.3 Hz, 1H), 7.53 (d, J=2.3 Hz, 1H), 7.40 (s, 1H), 7.16 (dd, J=10.0, 2.3 Hz, 1H), 6.94-6.87 (m, 1H), 6.86 (s, 1H), 4.32 (dd, J=13.5, 7.5 Hz, 1H), 4.03 (dd, J=13.4, 7.1 Hz, 1H), 2.92 (q, J=14.2, 7.1 Hz, 1H), 1.01 (d, J=7.0 Hz, 3H). m.p. 184.5-185.5° C.

### Compound 37: 3-(4-(6-fluoro-1H-indol-3-yl)-1H-pyrazol-1-yl)-2-hydroxy propanamide

### Step 1: 3-(1-(2, 2-diethoxyethyl)-1H-pyrazol-4-yl)-6-fluoro-1-(phenylsulfonyl)-1H-indole

A mixture of 6-fluoro-1-(phenylsulfonyl)-3-(1H-pyrazol-4-yl)-1H-indole (Intermediate 5; 500 mg; 1.46 mmol), 2-bromo-1,1-diethoxyethane (578 mg; 2.93 mmol) and Cs₂CO₃ (1.44 g; 4.42 mmol) in DMF (10.0 mL) was heated to 150° C. for 1 hour in a microwave reactor. The reaction mixture was poured into water and extracted with EtOAc (50 mL×3). The combined organic layers were washed with brine (50 mL), dried over anhydrous Na₂SO₄, filtered, concentrated, and purified by s by a silica gel chromatography (petroleum ether/EtOAc=20/1) to afford 250 mg (37%) of the title compound as a yellow solid.

LC-MS: m/z 458.1 [M+H]⁺

¹H NMR (300 MHz, DMSO-d₆) δ [ppm]: 8.31 (s, 1H), 8.11 (s, 1H), 8.07-8.04 (m, 2H), 8.00 (s, 1H), 7.87 (dd, J=8.7, 5.4 Hz, 1H), 7.77 (dd, J=9.9, 2.2 Hz, 1H), 7.72-7.67 (m, 1H), 7.64-7.57 (m, 2H), 7.24 (ddd, J=9.7, 8.7, 2.2 Hz, 1H), 4.86 (t, J=5.5 Hz, 1H), 4.21 (d, J=5.5 Hz, 2H), 3.70-3.58 (m, 2H), 3.48-3.36 (m, 2H), 1.06 (t, J=7.0 Hz, 6H).

### Step 2: 2-(4-(6-fluoro-1-(phenylsulfonyl)-1H-indol-3-yl)-1H-pyrazol-1-yl)acetaldehyde

A mixture of 3-(1-(2,2-diethoxyethyl)-1H-pyrazol-4-yl)-6-fluoro-1-(phenylsulfonyl)-1H-indole (Step 1; 250 mg; 0.55 mmol) in aqueous HCl (10 mL; 20 mmol; 2 M) and THF (5 mL) was stirred at 80° C. for 2 hours. The organic solvent was removed and the residue was adjusted to pH=9 with saturated aqueous NaHCO₃. The resulting mixture was extracted with EtOAc (50 mL×3). The combined organic layers were washed with brine (50 mL), dried over anhydrous Na₂SO₄, filtered, and concentrated to afford 200 mg (95%) of the title compound as a brown oil, which was used directly without further purification.

### Step 3: 3-(4-(6-fluoro-1-(phenylsulfonyl)-1H-indol-3-yl)-1H-pyrazol-1-yl)-2-hydroxy propanenitrile

A mixture of 2-(4-(6-fluoro-1-(phenylsulfonyl)-1H-indol-3-yl)-1H-pyrazol-1-yl)acetaldehyde (Step 2; 390 mg; 1.02 mmol), TMSCN (111 mg; 1.12 mmol), and DIPEA (263 mg; 2.03 mmol) in DCM (10.0 mL) was stirred at room temperature for 2 hours. The mixture was purified by a silica gel chromatography (petroleum ether/EtOAc=10/1) to afford 343 mg (82%) of the title compound as a yellow solid.

LC-MS: m/z 410.9 [M+H]⁺

¹H NMR (300 MHz, DMSO-d₆) δ [ppm]: 8.37 (s, 1H), 8.14 (s, 1H), 8.08-8.03 (m, 3H), 7.86 (dd, J=8.7, 5.4 Hz, 1H), 7.77 (dd, J=9.8, 2.2 Hz, 1H), 7.72-7.68 (m, 1H), 7.65-7.57 (m, 2H), 7.25 (ddd, J=9.7, 8.7, 2.2 Hz, 1H), 6.88 (d, J=6.3 Hz, 1H), 5.03-4.95 (m, 1H), 4.50-4.44 (m, 2H)

### Step 4: 3-(4-(6-fluoro-1-(phenylsulfonyl)-1H-indol-3-yl)-1H-pyrazol-1-yl)-2-hydroxypropanamide

A mixture of 3-(4-(6-fluoro-1-(phenylsulfonyl)-1H-indol-3-yl)-1H-pyrazol-1-yl)-2-hydroxypropanenitrile (Step 3; 183 mg; 0.45 mmol), PdCl₂ (8.0 mg; 0.045 mmol), and acetamide (119 mg; 2.00 mmol) in THF (5 mL) and water (1.5 mL) was stirred at room temperature overnight. The mixture was diluted with EtOAc (50 mL), washed with brine (50 mL), dried over anhydrous Na₂SO₄, filtered, concentrated, and purified by reverse phase flash chromatography to afford 20 mg (10%) of the title compound as a yellow solid.

LC-MS: m/z 429.0 [M+H]⁺

¹H NMR (400 MHz, DMSO-d₆) δ [ppm]: 8.24 (s, 1H), 8.10-8.03 (m, 3H), 7.99 (s, 1H), 7.89-7.85 (m, 1H), 7.78-7.75 (m, 1H), 7.71-7.68 (m, 1H), 7.62-7.58 (m, 2H), 7.38-7.32 (m, 2H), 7.25-7.21 (m, 1H), 5.86-5.84 (m, 1H), 4.44-4.41 (m, 1H), 4.28-4.15 (m, 2H).

### Step 5

Following the general method as outlined in the synthesis of compound 3, starting from 3-(4-(6-fluoro-1-(phenylsulfonyl)-1H-indol-3-yl)-1H-pyrazol-1-yl)-2-hydroxypropanamide (Step 4; 20 mg×3; 0.047 mmol×3), 8.6 mg (7%) of the title compound was obtained as a light yellow solid after purification by preparative HPLC.

LC-MS: m/z 289.0 [M+H]⁺

¹H NMR (300 MHz, DMSO-d₆) δ [ppm]: 11.17 (s, 1H), 8.02 (s, 1H), 7.79 (s, 1H), 7.73 (dd, J=8.8, 5.3 Hz, 1H), 7.53 (d, J=2.4 Hz, 1H), 7.38 (s, 1H), 7.32 (s, 1H), 7.16 (dd, J=9.8, 2.4 Hz, 1H), 6.91 (ddd, J=9.7, 8.8, 2.4 Hz, 1H), 5.85 (d, J=5.6 Hz, 1H), 4.45-4.39 (m, 1H), 4.28-4.14 (m, 2H).

### Compound 38: 2-(4-(6-fluoro-1H-indol-3-yl)-1H-pyrazol-1-yl)acetamide

### Step 1: 2-(4-(6-fluoro-1-(phenylsulfonyl)-1H-indol-3-yl)-1H-pyrazol-1-yl)acetamide

Following the general method as outlined in the synthesis of compound 41, starting from 6-fluoro-1-(phenylsulfonyl)-3-(1H-pyrazol-4-yl)-1H-indole (Intermediate 5; 250 mg; 0.73 mmol), K₂CO₃ (274 mg; 1.98 mmol), and 2-bromoacetamide (274 mg; 1.99 mmol) and MeCN (20 mL), 241 mg (83%) of the title compound was obtained as a white solid after filtration and concentration.

LC-MS: m/z 399.1 [M+H]⁺.

### Step 2

Following the general method as outlined in the synthesis of compound 70, starting from 2-(4-(6-fluoro-1-(phenylsulfonyl)-1H-indol-3-yl)-1H-pyrazol-1-yl)acetamide (Step 1; 241 mg; 0.60 mmol), 13 mg (8%) of the title compound was obtained as a light yellow solid after purification by preparative TLC (DCM/MeOH=5/1).

¹H NMR (400 MHz, MeOH-d₄) δ [ppm]: 68.02 (s, 1H), 7.87 (s, 1H), 7.74 (dd, J=8.7, 5.4 Hz, 1H), 7.44 (s, 1H), 7.11 (m, 1H), 6.89 (t, J=8.0 Hz, 1H), 4.95 (s, 2H).

### Example 39

### 2-(4-(6-fluoro-1H-indol-3-yl)-1H-pyrazol-1-yl)-N-methylacetamide

To a solution of 2-(4-(6-fluoro-1H-indol-3-yl)-1H-pyrazol-1-yl)acetic acid (Compound 41; 120 mg; 0.46 mmol) in THF (25 mL) was added HATU (350 mg; 0.92 mmol) and Et₃N (279 mg; 2.76 mmol) under nitrogen. The reaction mixture was stirred for 10 minutes before methylamine (0.5 mL; 1.0 mmol; 2 M in THF) was added dropwise. The reaction mixture was stirred at r.t. overnight, diluted with water (20 mL) and extracted with EtOAc (20 mL×3). The combined organic layers were concentrated, and purified by preparative TLC (EtOAc) to afford 33 mg (26%) of the title product as a white solid.

¹H NMR (400 MHz, DMSO-d₆) δ [ppm]: 11.18 (s, 1H), 8.09 (s, 1H), 7.96 (m, 1H), 7.81 (s, 1H), 7.74 (dd, J=9.0, 5.5 Hz, 1H), 7.56 (d, J=2.2 Hz, 0H), 7.17 (dd, J=10.1, 2.3 Hz, 1H), 6.92 (td, J=9.7, 2.3 Hz, 1H), 4.79 (s, 2H), 2.63 (d, J=4.6 Hz, 3H). m.p. 259.2-260.1° C.

### Compound 40: 2-(4-(6-fluoro-1H-indol-3-yl)-1H-pyrazol-1-yl)-N,N-dimethylacetamide

Following the general method as outlined in the synthesis of compound 39, starting from 2-(4-(6-fluoro-1H-indol-3-yl)-1H-pyrazol-1-yl)acetic acid (Compound 41; 120 mg; 0.46 mmol) and dimethylamine (0.5 mL; 1.0 mmol; 2 M in THF), 22 mg (17%) of the title compound was obtained as a white solid after purification by preparative TLC (EtOAc).

¹H NMR (300 MHz, DMSO-d₆) δ [ppm]: 11.18 (s, 1H), 8.01 (s, 1H), 7.73 (m, 2H), 7.56 (d, J=2.4 Hz, 1H), 7.17 (dd, J=10.0, 2.3 Hz, 1H), 6.91 (td, J=9.7, 2.3 Hz, 1H), 5.12 (s, 2H), 3.06 (s, 3H), 2.87 (s, 3H). m.p. 250.9-251.4° C.

### Compound 41: 2-(4-(6-fluoro-1H-indol-3-yl)-1H-pyrazol-1-yl)acetic acid

### Step 1: ethyl 2-(4-(6-fluoro-1-(phenylsulfonyl)-1H-indol-3-yl)-1H-pyrazol-1-yl)acetate

To a solution of 6-fluoro-1-(phenylsulfonyl)-3-(1H-pyrazol-4-yl)-1H-indole (Intermediate 5; 1.00 g; 2.93 mmol) and K₂CO₃ (1.30 g; 9.41 mmol) in DMF (40 mL) was added ethyl bromoacetate (1.50 g; 8.98 mmol). The mixture was stirred at 70° C. for 2 hours, cooled to r.t., diluted with 10% aqueous NH₄Cl (20 mL) and extracted with EtOAc (20 mL×3). The combined organic layers were washed with water (20 mL), brine (20 mL), dried over anhydrous Na₂SO₄, filtered, and concentrated to afford 1.10 g (88%) of the title compound as a yellow solid, which was used directly without further purification.

¹H NMR (300 MHz, DMSO-d₆) δ [ppm]: 8.36 (s, 1H), 8.15 (s, 1H), 8.05 (m, 3H), 7.78 (m, 3H), 7.61 (t, J=7.6 Hz, 2H), 7.24 (td, J=9.1, 2.3 Hz, 1H), 5.10 (s, 2H), 4.17 (q, J=7.1 Hz, 2H), 1.22 (t, J=7.1 Hz, 3H).

### Step 2

Following the general method as outlined in the synthesis of compound 70, starting from ethyl 2-(4-(6-fluoro-1-(phenylsulfonyl)-1H-indol-3-yl)-1H-pyrazol-1-yl)acetate (Step 1; 1.10 g; 2.57 mmol), 673 mg (100%) of the title compound was obtained as a white solid.

¹H NMR (400 MHz, DMSO-d₆) δ [ppm]: 13.07 (d, J=1.7 Hz, 1H), 11.28 (s, 1H), 8.12 (s, 1H), 7.81 (s, 1H), 7.73 (dd, J=8.7, 5.4 Hz, 1H), 7.57 (d, J=2.3 Hz, 1H), 7.18 (dd, J=9.8, 2.2 Hz, 1H), 6.92 (td, J=9.8, 2.3 Hz, 1H), 4.98 (s, 2H).

### Compound 42: methyl 2-(4-(6-fluoro-1H-indol-3-yl)-1H-pyrazol-1-yl)acetate

To a solution of 2-(4-(6-fluoro-1H-indol-3-yl)-1H-pyrazol-1-yl)acetic acid (Compound 41; 100 mg; 0.39 mmol) in MeOH (10 mL) was added concentrated sulfuric acid (0.02 mL; 0.39 mmol). The mixture was stirred at 85° C. overnight, cooled to r.t., poured into water, extracted with EtOAc (10 mL×3). The combined organic layers were washed with brine, dried over anhydrous Na₂SO₄, concentrated, and purified by preparative TLC (petroleum ether/EtOAc=1/1) to afford 33 mg (31%) of the title compound as a white solid.

¹H NMR (400 MHz, DMSO-d₆) δ [ppm]: δ 11.21 (s, 1H), 8.14 (s, 1H), 7.84 (s, 1H), 7.73 (dd, J=8.4, 5.5 Hz, 1H), 7.58 (d, J=1.9 Hz, 1H), 7.18 (dd, J=9.8, 1.7 Hz, 1H), 6.98-6.87 (m, 1H), 5.10 (s, 2H), 3.70 (s, 3H).

### Compound 43: 6-fluoro-3-(1-(piperidin-4-ylmethyl)-1H-pyrazol-4-yl)-1H-indole

Following the general method as outlined in the synthesis of compound 70, starting from 6-fluoro-1-(phenylsulfonyl)-3-(1-(piperidin-4-ylmethyl)-1H-pyrazol-4-yl)-1H-indole (Intermediate 21; 350 mg; 0.80 mmol), 37.2 mg (16%) of the title compound was obtained as a yellow solid after purification by reverse phase flash chromatography.

¹H NMR (400 MHz, DMSO-d₆) δ [ppm]: 11.16 (d, J=5.4 Hz, 1H), 8.07 (s, 1H), 7.83-7.72 (m, 2H), 7.53 (d, J=2.0 Hz, 1H), 7.16 (dd, J=10.1, 1.9 Hz, 1H), 6.95-6.85 (m, 1H), 3.98 (d, J=7.0 Hz, 2H), 3.44-3.35 (m, 1H), 2.90 (d, J=11.6 Hz, 2H), 2.40 (t, J=11.0 Hz, 2H), 1.91 (d, J=3.1 Hz, 1H), 1.49-1.39 (m, 2H), 1.13-1.04 (m, 2H). m.p. 106.3-107.5° C.

### Compound 44: 5,6-difluoro-3-(1-(piperidin-4-ylmethyl)-1H-pyrazol-4-yl)-1H-indole

Following the general method as outlined in the synthesis of compound 70, starting from 5,6-difluoro-1-(phenylsulfonyl)-3-(1-(piperidin-4-ylmethyl)-1H-pyrazol-4-yl)-1H-indole (Intermediate 46; 830 mg), 34.8 mg (19%) of the title compound was obtained as a white solid after purification by reverse phase flash chromatography.

LC-MS: m/z 317.1 [M+H⁺]⁺.

¹H NMR (400 MHz, DMSO-d₆) δ [ppm]: 11.33 (s, 1H), 8.15 (s, 1H), 7.80 (s, 1H), 7.74 (dd, J=11.6, 8.0 Hz, 1H), 7.62 (s, 1H), 7.40 (dd, J=11.2, 7.1 Hz, 1H), 4.00 (d, J=7.1 Hz, 2H), 3.17 (s, 2H), 2.99 (d, J=12.2 Hz, 2H), 1.99 (ddd, J=11.5, 9.3, 5.6 Hz, 1H), 1.49 (d, J=15.3 Hz, 2H), 1.18 (qd, J=12.5, 4.2 Hz, 2H). m.p. 167.1-168.9° C.

### Compound 45: 6-fluoro-3-(1-((1-(2,2,2-trifluoroethyl)piperidin-4-yl)methyl)-1H-pyrazol-4-yl)-1H-indole

### Step 1: 6-fluoro-1-(phenylsulfonyl)-3-(1-((1-(2,2,2-trifluoroethyl)piperidin-4-yl)methyl)-1H-pyrazol-4-yl)-1H-indole

A mixture of 6-fluoro-1-(phenylsulfonyl)-3-(1-(piperidin-4-ylmethyl)-1H-pyrazol-4-yl)-1H-indole (Intermediate 21; 277 mg; 0.63 mmol), Et₃N (0.40 mL; 2.87 mmol) and 2,2,2-trifluoroethyl trifluoromethanesulfonate (147 mg; 0.63 mmol) in toluene (40 mL) was stirred at 80° C. overnight. The reaction mixture was concentrated and purified by reverse phase flash chromatography to afford 275 mg (84%) of the title compound as a yellow oil.

LC-MS: m/z 521 [M+H]⁺.

### Step 2

Following the general method as outlined in the synthesis of compound 70, starting from 6-fluoro-1-(phenylsulfonyl)-3-(1-((1-(2,2,2-trifluoroethyl)piperidin-4-yl)methyl)-1H-pyrazol-4-yl)-1H-indole (Step 1; 275 mg; 0. 53 mmol), 135 mg (67%) of the title compound was obtained as a yellow solid after purification by preparative HPLC.

¹H NMR (400 MHz, DMSO-d₆) δ [ppm]: 11.16 (s, 1H), 8.08 (s, 1H), 7.82-7.71 (m, 1H), 7.53 (s, 1H), 7.16 (dd, J=10.1, 2.3 Hz, 1H), 7.01-6.85 (m, 1H), 4.02 (d, J=7.1 Hz, 2H), 3.12 (dt, J=20.6, 6.9 Hz, 2H), 2.99-2.84 (m, 2H), 2.42-2.22 (m, 2H), 1.84 (ddd, J=7.3, 5.4, 2.8 Hz, 1H), 1.48 (d, J=10.6 Hz, 2H), 1.26 (ddd, J=14.0, 10.7, 2.5 Hz, 2H).

### Compound 46: 6-fluoro-3-(1-((1-(2-fluoroethyl)piperidin-4-yl)methyl)-1H-pyrazol-4-yl)-1H-indole

### Step 1: 6-fluoro-3-(1-((1-(2-fluoroethyl)piperidin-4-yl)methyl)-1H-pyrazol-4-yl)-1-(phenylsulfonyl)-1H-indole

Following the general method as outlined in the synthesis of compound 81, starting from 6-fluoro-1-(phenylsulfonyl)-3-(1-(piperidin-4-ylmethyl)-1H-pyrazol-4-yl)-1H-indole (Intermediate 21; 111 mg; 0.25 mmol) and 1-bromo-2-fluoroethane (64 mg; 0.50 mmol), 122 mg (100%) of the title compound was obtained as a yellow oil, which was used directly without further purification.

LC-MS: m/z 485 [M+H]⁺.

### Step 2

Following the general method as outlined in the synthesis of compound 70, starting from 6-fluoro-3-(1-((1-(2-fluoroethyl)piperidin-4-yl)methyl)-1H-pyrazol-4-yl)-1-(phenylsulfonyl)-1H-indole (Step 1; 122 mg; 0.25 mmol), 16.5 mg (19%) of the title compound was obtained as a white solid after purification by preparative HPLC.

¹H NMR (400 MHz, MeOH-d₄) δ [ppm]: 7.90 (s, 1H), 7.79 (s, 1H), 7.77-7.69 (m, 1H), 7.40 (s, 1H), 7.11 (dd, J=9.9, 2.3 Hz, 1H), 6.95-6.83 (m, 1H), 4.66-4.45 (m, 2H), 4.07 (d, J=7.2 Hz, 2H), 2.99 (d, J=11.9 Hz, 2H), 2.78-2.58 (m, 2H), 2.08 (td, J=11.9, 2.2 Hz, 2H), 1.94 (ddd, J=11.5, 7.6, 4.0 Hz, 1H), 1.60 (d, J=12.9 Hz, 2H), 1.39 (qd, J=12.6, 3.7 Hz, 2H).

### Compound 47: 2-(4-((4-(6-fluoro-1H-indol-3-yl)-1H-pyrazol-1-yl)methyl)piperidin-1-yl)ethanol

### Step 1: 2-(4-((4-(6-fluoro-1-(phenylsulfonyl)-1H-indol-3-yl)-1H-pyrazol-1-yl)methyl)piperidin-1-yl)ethanol

Following the general method as outlined in the synthesis of compound 81, starting from 6-fluoro-1-(phenylsulfonyl)-3-(1-(piperidin-4-ylmethyl)-1H-pyrazol-4-yl)-1H-indole (Intermediate 21; 200 mg; 0.46 mmol), 200 mg (91%) of the title compound was obtained as a yellow oil, which was used directly without further purification.

LC-MS: m/z 483 [M+H]⁺.

### Step 2

Following the general method as outlined in the synthesis of compound 70, starting from 2-(4-((4-(6-fluoro-1-(phenylsulfonyl)-1H-indol-3-yl)-1H-pyrazol-1-yl)methyl)piperidin-1-yl)ethanol (Step 1; 200 mg; 0.41 mmol), 5.2 mg (4%) of the title compound was obtained as a white solid after purification by preparative HPLC.

¹H NMR (400 MHz, MeOH-d₄) δ [ppm]: 7.82 (s, 1H), 7.67 (s, 1H), 7.58 (dd, J=8.7, 5.3 Hz, 1H), 7.28 (s, 1H), 6.98 (dd, J=9.9, 2.3 Hz, 1H), 6.87-6.70 (m, 1H), 3.98 (d, J=7.1 Hz, 2H), 3.58 (t, J=6.1 Hz, 2H), 2.90 (d, J=11.9 Hz, 2H), 2.43 (t, J=6.1 Hz, 2H), 2.06-1.93 (m, 2H), 1.86 (ddd, J=11.7, 7.7, 4.1 Hz, 1H), 1.52 (d, J=13.0 Hz, 2H), 1.37-1.19 (m, 2H).

### Compound 48: 1,1,1-trifluoro-3-(4-((4-(6-fluoro-1H-indol-3-yl)-1H-pyrazol-1-yl)methyl)piperidin-1-yl)propan-2-ol

### Step 1: 1,1,1-trifluoro-3-(4-((4-(6-fluoro-1-(phenylsulfonyl)-1H-indol-3-yl)-1H-pyrazol-1-yl)methyl)piperidin-1-yl)propan-2-ol

A mixture of 6-fluoro-1-(phenylsulfonyl)-3-(1-(piperidin-4-ylmethyl)-1H-pyrazol-4-yl)-1H-indole (Intermediate 21; 111 mg; 0.25 mmol), K₂CO₃ (35 mg; 0.25 mmol) and 2-(trifluoromethyl)oxirane (142 mg; 1.27 mmol) in DMF (10 mL) was stirred overnight under nitrogen. The reaction mixture was added water (20 mL) and extracted with EtOAc (50 mL×2). The combined organic layers were dried over anhydrous Na₂SO₄, filtered, concentrated, and purified by reverse phase flash chromatograph to afford 65 mg (47%) of the title compound as a yellow solid.

LC-MS: m/z 551 [M+H]⁺.

### Step 2

Following the general method as outlined in the synthesis of compound 70, starting from 1,1,1-trifluoro-3-(4-((4-(6-fluoro-1-(phenylsulfonyl)-1H-indol-3-yl)-1H-pyrazol-1-yl)methyl)piperidin-1-yl)propan-2-ol (Step 1; 65 mg; 0.12 mmol), 16.5 mg (34%) of the title compound was obtained as a white solid after purification by reverse phase flash chromatography.

¹H NMR (400 MHz, MeOH-d₄) δ [ppm]: 7.73 (s, 1H), 7.64 (s, 1H), 7.54 (dd, J=8.7, 5.3 Hz, 1H), 7.24 (s, 1H), 6.96 (dd, J=9.9, 2.3 Hz, 1H), 6.73 (ddd, J=9.6, 8.9, 2.3 Hz, 1H), 4.05-3.93 (m, 1H), 3.90 (d, J=7.2 Hz, 2H), 2.83 (dd, J=15.1, 12.2 Hz, 2H), 2.51-2.36 (m, 2H), 2.06-1.88 (m, 2H), 1.77 (ddt, J=15.1, 7.6, 3.7 Hz, 1H), 1.43 (d, J=13.0 Hz, 2H), 1.34-1.16 (m, 2H).

### Compound 49: 2-(4-((4-(6-fluoro-1H-indol-3-yl)-1H-pyrazol-1-yl)methyl)piperidin-1-yl)acetic acid

### Step 1: ethyl 2-(4-((4-(6-fluoro-1-(phenylsulfonyl)-1H-indol-3-yl)-1H-pyrazol-1-yl)methyl)piperidin-1-yl)acetate

Following the general method as outlined in the synthesis of compound 41, starting from 6-fluoro-1-(phenylsulfonyl)-3-(1-(piperidin-4-ylmethyl)-1H-pyrazol-4-yl)-1H-indole (Intermediate 21; 185 mg; 0.42 mmol), 330 mg of the title compound was obtained as a yellow oil after purification by reverse phase flash chromatography.

LC-MS: m/z 525 [M+H]⁺.

### Step 2

Following the general method as outlined in the synthesis of compound 70, starting from ethyl 2-(4-((4-(6-fluoro-1-(phenylsulfonyl)-1H-indol-3-yl)-1H-pyrazol-1-yl)methyl)piperidin-1-yl)acetate (Step 1; 330 mg; 0.63 mmol), 190 mg (85%) of the title compound was obtained as a yellow solid after purification by reverse phase flash chromatography.

¹H NMR (400 MHz, DMSO-d₆) δ [ppm]: 11.28 (s, 1H), 8.08 (s, 1H), 7.85-7.69 (m, 2H), 7.53 (d, J=2.3 Hz, 1H), 7.17 (dd, J=10.1, 2.3 Hz, 1H), 6.90 (td, J=9.5, 2.4 Hz, 1H), 4.02 (d, J=7.0 Hz, 2H), 2.99 (d, J=11.3 Hz, 2H), 2.87 (s, 2H), 2.16 (t, J=10.8 Hz, 2H), 1.86 (ddd, J=10.9, 6.1, 3.6 Hz, 1H), 1.49 (d, J=11.5 Hz, 2H), 1.44-1.21 (m, 2H). m.p. 120.3-121.5° C.

### Compound 50: 4-(4-((4-(6-fluoro-1H-indol-3-yl)-1H-pyrazol-1-yl)methyl)piperidin-1-yl)-4-oxobutanoic acid

### Step 1: methyl 4-(4-((4-(6-fluoro-1-(phenylsulfonyl)-1H-indol-3-yl)-1H-pyrazol-1-yl)methyl)piperidin-1-yl)-4-oxobutanoate

Following the general method as outlined in the synthesis of compound 83, starting from 6-fluoro-1-(phenylsulfonyl)-3-(1-(piperidin-4-ylmethyl)-1H-pyrazol-4-yl)-1H-indole (Intermediate 21; 202 mg; 0.46 mmol) and monomethyl succinate (165 mg; 0.92 mmol), 220 mg (86%) of the title compound was obtained as a yellow oil after purification by reverse phase flash chromatography.

LC-MS: m/z 553 [M+H]⁺.

### Step 2

Following the general method as outlined in the synthesis of compound 70, starting from methyl 4-(4-((4-(6-fluoro-1-(phenylsulfonyl)-1H-indol-3-yl)-1H-pyrazol-1-yl)methyl)piperidin-1-yl)-4-oxobutanoate (Step 1; 220 mg; 0. 40 mmol), 69 mg (44%) of the title compound was obtained as a yellow solid after purification by preparative HPLC.

¹H NMR (400 MHz, MeOH-d₄) δ [ppm]: 8.01 (s, 1H), 7.86 (s, 1H), 7.76 (dd, J=8.7, 5.3 Hz, 1H), 7.46 (d, J=3.5 Hz, 1H), 7.15 (dd, J=9.9, 2.3 Hz, 1H), 7.01-6.85 (m, 1H), 4.60 (d, J=13.3 Hz, 1H), 4.14 (dd, J=19.8, 10.6 Hz, 3H), 3.23-3.06 (m, 1H), 2.78-2.62 (m, 3H), 2.55 (t, J=7.2 Hz, 2H), 2.39-2.20 (m, 1H), 1.72 (t, J=14.9 Hz, 2H), 1.46-1.15 (m, 2H). m.p. 79.2-80.1° C.

### Compound 51: 1-(4-((4-(6-fluoro-1H-indol-3-yl)-1H-pyrazol-1-yl)methyl)piperidin-1-yl)ethanone

### Step 1: 1-(4-((4-(6-fluoro-1-(phenylsulfonyl)-1H-indol-3-yl)-1H-pyrazol-1-yl)methyl)piperidin-1-yl)ethanone

Following the general method as outlined in the synthesis of compound 89, starting from 6-fluoro-1-(phenylsulfonyl)-3-(1-(piperidin-4-ylmethyl)-1H-pyrazol-4-yl)-1H-indole (Intermediate 21; 365 mg; 0.83 mmol), 400 mg (100%) of the title compound was obtained as a yellow solid, which was used directly without further purification.

LC-MS: m/z 481 [M+H]⁺.

### Step 2

Following the general method as outlined in the synthesis of compound 70, starting from 1-(4-((4-(6-fluoro-1-(phenylsulfonyl)-1H-indol-3-yl)-1H-pyrazol-1-yl)methyl)piperidin-1-yl)ethanone (Step 1; 400 mg; 0.83 mmol), 37.4 mg (13%) of the title compound was obtained as a yellow solid after purification by preparative HPLC.

¹H NMR (400 MHz, DMSO-d₆) δ [ppm]: δ 11.21 (d, J=5.7 Hz, 1H), 8.09 (s, 1H), 7.80-7.71 (m, 2H), 7.54 (d, J=2.4 Hz, 1H), 7.16 (dd, J=10.1, 2.3 Hz, 1H), 6.96-6.86 (m, 1H), 4.34 (dd, J=9.0, 6.9 Hz, 1H), 4.04 (d, J=7.1 Hz, 2H), 3.79 (d, J=13.3 Hz, 1H), 3.05-2.92 (m, 2H), 2.16-2.05 (m, 1H), 1.97 (s, 3H), 1.54 (m, 2H), 1.10 (m, 2H).

### Compound 52: 3-(1-((1-cyclopropylpiperidin-4-yl)methyl)-1H-pyrazol-4-yl)-6-fluoro-1H-indole

### Step 1: 3-(1-((1-cyclopropylpiperidin-4-yl)methyl)-1H-pyrazol-4-yl)-6-fluoro-1-(phenylsulfonyl)-1H-indole

A mixture of 6-fluoro-1-(phenylsulfonyl)-3-(1-(piperidin-4-ylmethyl)-1H-pyrazol-4-yl)-1H-indole (Intermediate 21; 277 mg; 0.63 mmol), HOAc (121 mg; 2.01 mmol), (1-ethoxycyclopropoxy)trimethylsilane (220 mg; 1.26 mmol) and NaBH₃CN (60 mg; 0.95 mmol) in DCM/MeOH/THF (10 ml/1 ml/15 ml) was stirred at 70° C. overnight. The reaction mixture was added saturated aqueous NH₄Cl (20 mL), extracted with EtOAc (50 mL×2). The combined organic layers were dried over anhydrous Na₂SO₄, filtered, concentrated, and purified by reverse phase flash chromatography to afford 137 mg (45%) of the title compound as a yellow oil.

LC-MS: m/z 479 [M+H]⁺.

### Step 2

Following the general method as outlined in the synthesis of compound 70, starting from 3-(1-((1-cyclopropylpiperidin-4-yl)methyl)-1H-pyrazol-4-yl)-6-fluoro-1-(phenylsulfonyl)-1H-indole (Step 1; 137 mg; 0.29 mmol), 17 mg (18%) of the title compound was obtained as a white solid after purification by preparative HPLC.

¹H NMR (400 MHz, MeOH-d₄) δ [ppm]: 7.80 (s, 1H), 7.67 (s, 1H), 7.58 (dd, J=8.7, 5.3 Hz, 1H), 7.28 (s, 1H), 6.98 (dd, J=9.9, 2.3 Hz, 1H), 6.77 (ddd, J=9.6, 8.9, 2.4 Hz, 1H), 3.96 (d, J=7.1 Hz, 2H), 2.94 (d, J=11.9 Hz, 2H), 2.10 (td, J=12.0, 2.4 Hz, 2H), 1.93 (s, 1H), 1.58-1.43 (m, 3H), 1.20 (dd, J=12.5, 3.3 Hz, 2H), 0.44-0.25 (m, 4H).

### Compound 53: 6-fluoro-3-(1-((1-methylpiperidin-4-yl)methyl)-1H-pyrazol-4-yl)-1H-indole

The title compound (40.7 mg; 16%) was obtained as a yellow solid after purification by reverse phase flash chromatography in step 2 of the synthesis of compound 52.

¹H NMR (400 MHz, DMSO-d₆) δ [ppm]: 11.16 (s, 1H), 8.08 (s, 1H), 7.81-7.70 (m, 2H), 7.53 (d, J=1.8 Hz, 1H), 7.16 (dd, J=10.0, 1.8 Hz, 1H), 6.91 (td, J=10.0, 2.1 Hz, 1H), 4.01 (d, J=7.1 Hz, 2H), 2.73 (d, J=11.5 Hz, 2H), 2.12 (s, 3H), 1.79 (m, 3H), 1.47 (d, J=11.7 Hz, 2H), 1.30-1.17 (m, 2H). m.p. 166.3-169.7° C.

### Compound 54: 6-fluoro-3-(1-((1-(methylsulfonyl)piperidin-4-yl)methyl)-1H-pyrazol-4-yl)-1H-indole

### Step 1: 6-fluoro-3-(1-((1-(methylsulfonyl)piperidin-4-yl)methyl)-1H-pyrazol-4-yl)-1-(phenylsulfonyl)-1H-indole

Following the general method as outlined in the synthesis of compound 25, starting from 6-fluoro-1-(phenylsulfonyl)-3-(1-(piperidin-4-ylmethyl)-1H-pyrazol-4-yl)-1H-indole (Intermediate 21; 276 mg; 0.63 mmol), 300 mg (92%) of the title compound was obtained as a yellow solid, which was used directly without further purification.

LC-MS: m/z 517 [M+H]⁺.

### Step 2

Following the general method as outlined in the synthesis of compound 70, starting from 6-fluoro-3-(1-((1-(methylsulfonyl)piperidin-4-yl)methyl)-1H-pyrazol-4-yl)-1-(phenylsulfonyl)-1H-indole (Step 1; 300 mg; 0.58 mmol), 150 mg (69%) of the title compound was obtained as a white solid after purification by preparative HPLC.

¹H NMR (400 MHz, DMSO-d₆) δ [ppm]: 11.17 (s, 1H), 8.10 (s, 1H), 7.79 (s, 1H), 7.75 (dd, J=8.6, 5.4 Hz, 1H), 7.54 (d, J=2.2 Hz, 1H), 7.16 (dd, J=10.1, 2.3 Hz, 1H), 6.99-6.83 (m, 1H), 4.08 (d, J=7.1 Hz, 2H), 3.55 (d, J=12.0 Hz, 2H), 2.83 (s, 3H), 2.76-2.61 (m, 2H), 2.07-1.91 (m, 1H), 1.64 (dd, J=8.3, 4.7 Hz, 2H), 1.28 (ddd, J=23.3, 11.8, 3.1 Hz, 2H). m.p. 82.3-83.1° C.

### Compound 55: 3-(3,5-dimethyl-1-(piperidin-4-ylmethyl)-1H-pyrazol-4-yl)-6-fluoro-1H-indole hydrochloride

### Step 1: tert-butyl 4-((4-(6-fluoro-1-(phenylsulfonyl)-1H-indol-3-yl)-3,5-dimethyl-1H-pyrazol-1-yl)methyl)piperidine-1-carboxylate

To a solution of 3-(3,5-dimethyl-1H-pyrazol-4-yl)-6-fluoro-1-(phenylsulfonyl)-1H-indole (Intermediate 50; 150 mg; 0.41 mmol) and Cs₂CO₃ (397 mg; 1.22 mmol) in DMF (5 mL) was added tert-butyl 4-((methylsulfonyloxy)methyl)piperidine-1-carboxylate (357 mg; 1.22 mmol) under nitrogen. The reaction mixture was stirred at 90° C. for 16 hours, cooled, diluted with water (30 mL) and extracted with EtOAc (50 mL×2). The combined organic layers were washed with brine (30 mL×2), dried over anhydrous Na₂SO₄, filtered, and concentrated to afford 226 mg (98%) of the title compound as a brown oil, which was used directly without further purification.

LC-MS: m/z 566.9 [M+H]⁺

### Step 2: tert-butyl 4-((4-(6-fluoro-1H-indol-3-yl)-3, 5-dimethyl-1H-pyrazol-1-yl)methyl)piperidine-1-carboxylate

Following the general method as outlined in the synthesis of compound 3, starting from 4-((4-(6-fluoro-1-(phenylsulfonyl)-1H-indol-3-yl)-3,5-dimethyl-1H-pyrazol-1-yl)methyl)piperidine-1-carboxylate (Step 1; 226 mg; 0.40 mmol), 96 mg (56%) of the title compound was obtained as a white solid after purification by preparative TLC (DCM/MeOH=40/1).

LC-MS: m/z 427.0 [M+H]⁺

### Step 3

Following the general method as outlined in synthesis of compound 2, starting from tert-butyl 4-((4-(6-fluoro-1H-indol-3-yl)-3,5-dimethyl-1H-pyrazol-1-yl)methyl)piperidine-1-carboxylate (Step 2; 96 mg; 0.23 mmol), 45 mg (55%) of the title compound was obtained as a yellow solid after purification by preparative HPLC (HCl additive).

LC-MS: m/z 327.0 [M+H]⁺

¹H NMR (300 MHz, MeOH-d₄) δ [ppm]: 7.36 (s, 1H), 7.29 (dd, J=8.7, 5.2 Hz, 1H), 7.17 (dd, J=9.7, 2.3 Hz, 1H), 6.88 (ddd, J=9.7, 8.7, 2.3 Hz, 1H), 4.35 (d, J=7.4 Hz, 2H), 3.52-3.44 (m, 2H), 3.13-2.98 (m, 2H), 2.39 (s, 3H), 2.44-2.32 (m, 1H), 2.36 (s, 3H), 1.98-1.88 (m, 2H), 1.75-1.58 (m, 2H).

### Compound 56: 6-fluoro-3-(3-methyl-1-(piperidin-4-ylmethyl)-1H-pyrazol-4-yl)-1H-indole

### Step 1: tert-butyl 4-((4-(6-fluoro-1H-indol-3-yl)-3-methyl-1H-pyrazol-1-yl)methyl)piperidine-1-carboxylate

Following the general method as outlined in synthesis of compound 3, starting from a mixture of tert-butyl 4-((4-(6-fluoro-1-(phenylsulfonyl)-1H-indol-3-yl)-3-methyl-1H-pyrazol-1-yl)methyl)piperidine-1-carboxylate and tert-butyl 4-((4-(6-fluoro-1-(phenylsulfonyl)-1H-indol-3-yl)-5-methyl-1H-pyrazol-1-yl)methyl)piperidine-1-carboxylate (Intermediate 51; 1.00 g crude; 1.41 mmol), 450 mg (78%) of a mixture of tert-butyl 4-((4-(6-fluoro-1H-indol-3-yl)-3-methyl-1H-pyrazol-1-yl)methyl)piperidine-1-carboxylate and tert-butyl 4-((4-(6-fluoro-1H-indol-3-yl)-5-methyl-1H-pyrazol-1-yl)methyl)piperidine-1-carboxylate was obtained as a colorless oil after purification by reverse phase flash chromatography.

150 mg of the title compound was obtained as a white solid after purification by preparative chiral HP

Preparative chiral HPLC: ChiralCEL OJ-H 250 mm×20 mm 5 μm; Mobile phase: Hexane/IPA/Et₂NH=80/20/0.3; Flow: 15 mL/min 254 nm ambient temperature

Analytical chiral HPLC: Chiralcel OJ-H 250 mm×4.6 mm 5 μm; Mobile phase: Hexane/IPA=70/30; Flow: 1.0 mL/min 230 nm ambient temperature; Retention time: 7.282 min.

LC-MS: 413.0 [M+H]+

¹H NMR (300 MHz, CDCl₃) δ [ppm]: 9.15 (s, 1H), 7.53 (dd, J=8.7, 5.3 Hz, 1H), 7.52 (s, 1H), 7.18 (d, J=2.3 Hz, 1H), 7.08 (dd, J=9.6, 2.2 Hz, 1H), 6.92 (ddd, J=9.7, 8.7, 2.2 Hz, 1H), 4.30-3.98 (m, 2H), 3.96 (d, J=7.2 Hz, 2H), 2.82-2.56 (m, 2H), 2.36 (s, 3H), 2.22-1.98 (m, 1H), 1.70-1.54 (m, 2H), 1.46 (s, 9H), 1.30-1.09 (m, 2H).

### Step 2

Following the general method as outlined in synthesis of compound 2, starting from tert-butyl 4-((4-(6-fluoro-1H-indol-3-yl)-3-methyl-1H-pyrazol-1-yl)methyl)piperidine-1-carboxylate (Step 1; 150 mg; 0.36 mmol), 50 mg (44%) of the title compound was obtained as a white solid after purification by preparative HPLC.

LC-MS: m/z 313.0 [M+H]⁺.

¹H NMR (300 MHz, DMSO-d₆) δ [ppm]: 11.24 (s, 1H), 7.94 (s, 1H), 7.61 (dd, J=8.7, 5.5 Hz, 1H), 7.37 (d, J=2.3 Hz, 1H), 7.17 (dd, J=10.1, 2.4 Hz, 1H), 6.90 (ddd, J=9.7, 8.7, 2.4 Hz, 1H), 3.92 (d, J=6.9 Hz, 2H), 3.02-2.82 (m, 2H), 2.48-2.32 (m, 2H), 2.26 (s, 3H), 2.02-1.80 (m, 1H), 1.53-1.38 (m, 2H), 1.17-0.99 (m, 2H).

### Compound 57: 6-fluoro-3-(5-methyl-1-(piperidin-4-ylmethyl)-1H-pyrazol-4-yl)-1H-indole

### Step 1: tert-butyl 4-((4-(6-fluoro-1H-indol-3-yl)-5-methyl-1H-pyrazol-1-yl)methyl)piperidine-1-carboxylate

150 mg of the title compound was obtained as a white solid after preparative chiral HPLC in synthesis of compound 56 step 1.

Analytical chiral HPLC: Chiralcel OJ-H 250 mm×4.6 mm 5 μm; Mobile phase: Hexane/IPA=70/30; Flow: 1.0 mL/min 230 nm ambient temperature; Retention time: 10.834 min.

LC-MS: 413.0 [M+H]⁺

¹H NMR (300 MHz, CDCl₃) δ [ppm]: 8.87 (s, 1H), 7.68 (s, 1H), 7.53 (dd, J=8.7, 5.4 Hz, 1H), 7.16-7.04 (m, 2H), 6.97-6.86 (m, 1H), 4.31-4.03 (m, 2H), 3.99 (d, J=7.3 Hz, 2H), 2.86-2.56 (m, 2H), 2.31 (s, 3H), 2.28-2.06 (m, 1H), 1.70-1.56 (m, 2H), 1.47 (s, 9H), 1.33-1.12 (m, 2H).

### Step 2

Following the general method as outlined in synthesis of compound 2, starting from tert-butyl 4-((4-(6-fluoro-1H-indol-3-yl)-5-methyl-1H-pyrazol-1-yl)methyl)piperidine-1-carboxylate (Step 1; 150 mg; 0.36 mmol), 15 mg (13%) of the title compound was obtained as a white solid after purification by preparative HPLC.

LC-MS: m/z 313.0 [M+H]⁺.

¹H NMR (300 MHz, DMSO-d₆) δ [ppm]: 11.25 (s, 1H), 7.59 (s, 1H), 7.52 (dd, J=8.7, 5.5 Hz, 1H), 7.33 (s, 1H), 7.22-7.12 (m, 1H), 6.93-6.81 (m, 1H), 3.94 (d, J=7.1 Hz, 2H), 3.02-2.80 (m, 2H), 2.48-2.32 (m, 2H), 2.30 (s, 3H), 2.03-1.79 (m, 1H), 1.56-1.32 (m, 2H), 1.26-0.98 (m, 2H).

### Compound 58: 6-fluoro-3-(1-((tetrahydro-2H-pyran-4-yl)methyl)-1H-pyrazol-4-yl)-1H-indole

### Step 1: 6-fluoro-1-(phenylsulfonyl)-3-(1-((tetrahydro-2H-pyran-4-yl)methyl)-1H-pyrazol-4-yl)-1H-indole

Following the general method as outlined in Intermediate 20, starting from 6-fluoro-1-(phenylsulfonyl)-3-(1H-pyrazol-4-yl)-1H-indole (Intermediate 5; 305 mg; 0.89 mmol) and (tetrahydro-2H-pyran-4-yl)methyl methanesulfonate (226 mg; 1.16 mmol), 398 mg of the title compound was obtained as a white solid, which was used directly without further purification.

LC-MS: m/z 440 [M+H]⁺.

### Step 2

Following the general method as outlined in the synthesis of compound 70, starting from 6-fluoro-1-(phenylsulfonyl)-3-(1-((tetrahydro-2H-pyran-4-yl)methyl)-1H-pyrazol-4-yl)-1H-indole (Step 1; 398 mg; 0.90 mmol), 102 mg (38%) of the title compound was obtained as a white solid after purification by preparative HPLC.

LC-MS: m/z 300.1 [M+H]⁺.

¹H NMR (400 MHz, DMSO-d₆) δ [ppm]: 11.16 (s, 1H), 8.09 (s, 1H), 7.78 (s, 1H), 7.75 (dd, J=8.7, 5.4 Hz, 1H), 7.54 (d, J=2.3 Hz, 1H), 7.16 (dd, J=10.0, 2.2 Hz, 1H), 6.91 (td, J=9.3, 2.3 Hz, 1H), 4.03 (d, J=7.1 Hz, 2H), 3.84 (dd, J=11.2, 2.8 Hz, 2H), 3.26 (td, J=11.6, 1.8 Hz, 2H), 2.17-2.03 (m, 1H), 1.44 (d, J=11.3 Hz, 2H), 1.27 (qd, J=12.2, 4.4 Hz, 2H). m.p. 138.9-139.6° C.

### Compound 59: 4-((4-(6-fluoro-1H-indol-3-yl)-1H-pyrazol-1-yl)methyl)tetrahydro-2H-pyran-4-ol

### Step 1: 4-((4-(6-fluoro-1-(phenylsulfonyl)-1H-indol-3-yl)-1H-pyrazol-1-yl)methyl)tetrahydro-2H-pyran-4-ol

The mixture of K₂CO₃ (221 mg; 1.6 mmol) and 1,6-dioxaspiro[2.5]octane (106 mg; 0.93 mmol) and 6-fluoro-1-(phenylsulfonyl)-3-(1H-pyrazol-4-yl)-1H-indole (Intermediate 5; 300 mg; 0.88 mmol) in DMF (2 ml) was stirred at 80° C. for 12 hours. The mixture was poured into ice-water (20 mL) and extracted with EtOAc (30 mL×3). The combined organic layers were dried over anhydrous Na₂SO₄, filtered, concentrated, and purified by a silica gel chromatography (solvent ration) to afford 300 mg (75%) of the title compound as a yellow solid.

LC-MS: m/z 456.1 [M+H]⁺.

### Step 2

Following the general method as outlined in the synthesis of compound 70, starting from 4-((4-(6-fluoro-1-(phenylsulfonyl)-1H-indol-3-yl)-1H-pyrazol-1-yl)methyl)tetrahydro-2H-pyran-4-ol (Step 1; 300 mg; 0.66 mmol), 50 mg (24%) of the title compound was obtained as a white solid after purification by preparative HPLC.

¹H NMR (400 MHz, MeOH-d₄) δ [ppm]: 7.97 (s, 1H), 7.81 (s, 1H), 7.72 (dd, J=8.7, 5.3 Hz, 1H), 7.42 (s, 1H), 7.11 (dd, J=9.9, 2.3 Hz, 1H), 6.94-6.85 (m, 1H), 4.21 (s, 2H), 3.87-3.69 (m, 4H), 1.87-1.70 (m, 2H), 1.47 (dd, J=13.9, 1.8 Hz, 2H).

¹⁹F NMR (377 MHz, MeOH-d₄) δ [ppm]: −124.51 (s, 1H). m.p. 213.5-214.5° C.

### Compound 60: 4-((4-(6-fluoro-1H-indol-3-yl)-1H-pyrazol-1-yl)methyl)tetrahydro-2H-thiopyran 1,1-dioxide

Following the general method as outlined in Intermediate 20, starting from 6-fluoro-1-(phenylsulfonyl)-3-(1H-pyrazol-4-yl)-1H-indole (Intermediate 5; 92 mg; 0.27 mmol) and (1,1-dioxidotetrahydro-2H-thiopyran-4-yl)methyl methanesulfonate (100 mg; 0.41 mmol), 30 mg (32%) of the title compound was obtained as a yellow solid after purification by preparative TLC (DCM/MeOH=20/1).

LC-MS: m/z 348.1[M+H]⁺.

¹H NMR (400 MHz, MeOH-d₄) δ [ppm]: 8.00 (s, 1H), 7.83 (s, 1H), 7.72 (dd, J=8.8, 5.3 Hz, 1H), 7.42 (s, 1H), 7.11 (dd, J=10.0, 2.3 Hz, 1H), 6.94-6.83 (m, 1H), 4.19 (d, J=7.2 Hz, 2H), 3.23-3.03 (m, 4H), 2.36-2.26 (m, 1H), 2.11-1.99 (m, 2H), 1.95-1.80 (m, 2H).

¹⁹F NMR (377 MHz, MeOH-d₄) δ [ppm]: −124.28. m.p. 193.1-194.1° C.

### Compound 61: cis-3-(4-(6-fluoro-1H-indol-3-yl)-1H-pyrazol-1-yl)cyclobutanecarboxamide

### Step 1: cis-3-(4-(6-fluoro-1-(phenylsulfonyl)-1H-indol-3-yl)-1H-pyrazol-1-yl)cyclobutanecarboxamide

A mixture of cis-3-(4-(6-fluoro-1-(phenylsulfonyl)-1H-indol-3-yl)-1H-pyrazol-1-yl)cyclobutanecarboxylic acid (Intermediate 36; 100 mg; 0.23 mmol), Et₃N (0.13 mL; 0.93 mmol), NH₄Cl (24 mg; 0.45 mmol) and HATU (173 mg; 0.45 mmol) in THF (5 mL) was stirred for 1 hour. The reaction mixture was added water (20 mL) and extracted with EtOAc (50 mL×2). The combined organic layers were dried over anhydrous Na₂SO₄, filtered, and concentrated to afford 98 mg (98%) of the title compound as a white solid, which was used directly without further purification.

LC-MS: m/z 439 [M+H]⁺.

### Step 2

Following the general method as outlined in the synthesis of compound 70, starting from cis-3-(4-(6-fluoro-1-(phenylsulfonyl)-1H-indol-3-yl)-1H-pyrazol-1-yl)cyclobutanecarboxamide (Step 1; 195 mg; 0.44 mmol), 14.9 mg (11%) of the title compound was obtained as a white solid after purification by preparative TLC (DCM/MeOH=10/1).

¹H NMR (400 MHz, DMSO-d₆) δ [ppm]: 11.18 (s, 1H), 8.13 (s, 1H), 7.82 (s, 1H), 7.77 (dd, J=8.8, 5.4 Hz, 1H), 7.56 (d, J=2.4 Hz, 1H), 7.33 (s, 1H), 7.16 (dd, J=10.1, 2.3 Hz, 1H), 6.95-6.83 (m, 2H), 4.77 (dd, J=12.5, 4.6 Hz, 1H), 2.83-2.72 (m, 1H), 2.69-2.54 (m, 4H). m.p. 225.7-226.3° C.

### Compound 62: trans-3-(4-(6-fluoro-1H-indol-3-yl)-1H-pyrazol-1-yl)cyclobutanecarboxamide

### Step 1: trans-3-(4-(6-fluoro-1-(phenylsulfonyl)-1H-indol-3-yl)-1H-pyrazol-1-yl)cyclobutanecarboxamide

Following the general method as outlined in the synthesis of compound 61, starting from trans-3-(4-(6-fluoro-1-(phenylsulfonyl)-1H-indol-3-yl)-1H-pyrazol-1-yl)cyclobutanecarboxylic acid (Intermediate 34; 63 mg; 0.14 mmol), 63 mg (100%) of the title compound was obtained as a white solid, which was used directly without further purification.

LC-MS: m/z 439 [M+H]⁺.

### Step 2

Following the general method as outlined in the synthesis of compound 70, starting from trans-3-(4-(6-fluoro-1-(phenylsulfonyl)-1H-indol-3-yl)-1H-pyrazol-1-yl)cyclobutanecarboxamide (Step 1; 63 mg; 0.14 mmol), 6.2 mg (15%) of the title compound was obtained as a white solid after purification by preparative TLC (DCM/MeOH=9/1).

¹H NMR (400 MHz, MeOH-d₄) δ [ppm]: 7.94 (d, J=5.7 Hz, 1H), 7.77 (d, J=3.9 Hz, 1H), 7.64 (dd, J=8.7, 5.2 Hz, 1H), 7.33 (s, 1H), 7.02 (dd, J=9.8, 2.2 Hz, 1H), 6.81 (ddd, J=9.6, 8.8, 2.3 Hz, 1H), 5.14-4.99 (m, 1H), 3.16 (qd, J=8.3, 3.9 Hz, 1H), 2.81 (tdd, J=9.8, 7.9, 2.3 Hz, 2H), 2.74-2.61 (m, 2H). m.p. 198.4-199.2° C.

### Compound 63: cis-3-(4-(6-fluoro-1H-indol-3-yl)-1H-pyrazol-1-yl)-N-methylcyclobutanecarboxamide

### Step 1: cis-3-(4-(6-fluoro-1-(phenylsulfonyl)-1H-indol-3-yl)-1H-pyrazol-1-yl)-N-methylcyclobutanecarboxamide

Following the general method as outlined in the synthesis of compound 61, starting from cis-3-(4-(6-fluoro-1-(phenylsulfonyl)-1H-indol-3-yl)-1H-pyrazol-1-yl)cyclobutanecarboxylic acid (Intermediate 36; 100 mg; 0.23 mmol), and MeNH₂ (0.23 mL; 0.46 mmol; 2.0 M in THF), 100 mg (96%) of the title compound was obtained as a yellow oil, which was used directly without further purification.

LC-MS: m/z 453 [M+H]⁺.

### Step 2

Following the general method as outlined in the synthesis of compound 70, starting from cis-3-(4-(6-fluoro-1-(phenylsulfonyl)-1H-indol-3-yl)-1H-pyrazol-1-yl)-N-methylcyclobutanecarboxamide (Step 1; 200 mg; 0.44 mmol), 32.6 mg (24%) of the title compound was obtained as a white solid after purification by preparative TLC (DCM/MeOH=10/1).

¹H NMR (400 MHz, DMSO-d₆) δ [ppm]: 11.18 (s, 1H), 8.12 (s, 1H), 7.89-7.70 (m, 3H), 7.56 (d, J=2.4 Hz, 1H), 7.16 (dd, J=10.0, 2.3 Hz, 1H), 6.99-6.82 (m, 1H), 4.84-4.71 (m, 1H), 2.77 (dd, J=17.7, 8.8 Hz, 1H), 2.66-2.59 (m, 8H). m.p. 173.7-174.6° C.

### Compound 64: trans-3-(4-(6-fluoro-1H-indol-3-yl)-1H-pyrazol-1-yl)-N-methylcyclobutanecarboxamide

### Step 1: trans-3-(4-(6-fluoro-1-(phenylsulfonyl)-1H-indol-3-yl)-1H-pyrazol-1-yl)-N-methylcyclobutanecarboxamide

Following the general method as outlined in the synthesis of compound 67, starting from trans-3-(4-(6-fluoro-1-(phenylsulfonyl)-1H-indol-3-yl)-1H-pyrazol-1-yl)cyclobutanecarboxylic acid (Intermediate 34; 50 mg; 0.11 mmol), 52 mg (100%) of the title compound was obtained as a yellow oil, which was used directly without further purification.

LC-MS: m/z 453 [M+H]⁺.

### Step 2

Following the general method as outlined in the synthesis of compound 70, starting from trans-3-(4-(6-fluoro-1-(phenylsulfonyl)-1H-indol-3-yl)-1H-pyrazol-1-yl)-N-methylcyclobutanecarboxamide (Step 1; 52 mg; 0.11 mmol), 20 mg (56%) of the title compound was obtained as a yellow solid after purification by preparative TLC (DCM/MeOH=9/1).

¹H NMR (400 MHz, DMSO-d₆) δ [ppm]: 11.17 (s, 1H), 8.20 (s, 1H), 7.80 (dt, J=8.8, 5.2 Hz, 3H), 7.53 (d, J=2.4 Hz, 1H), 7.16 (dd, J=10.0, 2.3 Hz, 1H), 7.00-6.82 (m, 1H), 5.12-4.99 (m, 1H), 3.03 (dd, J=9.1, 4.8 Hz, 1H), 2.77-2.66 (m, 2H), 2.63 (d, J=4.6 Hz, 3H), 2.61-2.54 (m, 2H). m.p. 177.8-178.4° C.

### Compound 65: cis-3-(4-(6-fluoro-1H-indol-3-yl)-1H-pyrazol-1-yl)cyclobutanecarboxylic acid

The title compound was obtained (10.4 mg; 8%) as a yellow solid after purification by preparative TLC (DCM/MeOH=10/1) in step 2 of the synthesis of compound 61.

¹H NMR (400 MHz, DMSO-d₆) δ [ppm]: 12.50-12.20 (m, 1H), 11.24 (d, J=11.9 Hz, 1H), 8.16 (s, 1H), 7.84 (s, 1H), 7.78 (dd, J=8.7, 5.4 Hz, 1H), 7.56 (d, J=2.4 Hz, 1H), 7.16 (dd, J=10.0, 2.3 Hz, 1H), 6.91 (ddd, J=9.7, 8.8, 2.4 Hz, 1H), 4.88-4.73 (m, 1H), 3.01-2.84 (m, 1H), 2.75-2.58 (m, 4H). m.p. 220.4-221.7° C.

### Compound 66: trans-3-(4-(6-fluoro-1H-indol-3-yl)-1H-pyrazol-1-yl)cyclobutanecarboxylic acid

The title compound was obtained (2.2 mg; 5%) as a yellow solid after purification by preparative TLC (DCM/MeOH=9/1) in step 2 of the synthesis of compound 62.

¹H NMR (400 MHz, MeOH-d₄) δ [ppm]: 7.90 (s, 1H), 7.73 (s, 1H), 7.60 (dd, J=8.8, 5.3 Hz, 1H), 7.29 (s, 1H), 6.98 (dd, J=9.9, 2.4 Hz, 1H), 6.83-6.70 (m, 1H), 5.11-4.95 (m, 1H), 3.17-3.05 (m, 1H), 2.81 (ddd, J=12.5, 10.1, 8.0 Hz, 2H), 2.75-2.62 (m, 2H).

### Compound 67: trans-4-(4-(6-fluoro-1H-indol-3-yl)-1H-pyrazol-1-yl)cyclohexane carboxamide

To a stirred solution of 4-(4-(6-fluoro-1H-indol-3-yl)-1H-pyrazol-1-yl)cyclohexanecarboxylic acid (mixture of cis and trans isomers, Intermediate 52; 400 mg; 1.22 mmol) and Et₃N (494 mg; 4.88 mmol) in anhydrous THF (15 mL) was added HATU (931 mg; 2.45 mmol) at 0° C. The mixture was stirred for 10 minutes and NH₄Cl (131 mg; 2.45 mmol) was added. The reaction mixture was stirred at room temperature for 16 hours, quenched with water (20 mL), and extracted with EtOAc (30 mL×3). The combined organic layers were washed with brine (20 mL×3), dried over anhydrous Na₂SO₄, filtered, concentrated, and purified by preparative TLC (DCM/MeOH=15/1) and preparative HPLC to afford 36 mg (9%) of the title compound as a white solid.

LC-MS: m/z 327.2 [M+H]⁺.

¹H NMR (400 MHz, DMSO-d₆) δ [ppm]: 11.17 (brs, 1H), 8.11 (s, 1H), 7.78 (dd, J=8.6, 5.6 Hz, 1H), 7.77 (s, 1H), 7.52 (d, J=2.4 Hz, 1H), 7.28 (brs, 1H), 7.16 (dd, J=10.1, 2.2 Hz, 1H), 6.90 (ddd, J=9.7, 8.6, 2.2 Hz, 1H), 6.75 (brs, 1H), 4.15 (tt, J=11.8, 4.0 Hz, 1H), 2.18 (tt, J=11.6, 3.3 Hz, 1H), 2.17-2.07 (m, 2H), 1.95-1.74 (m, 4H), 1.61-1.48 (m, 2H).

### Compound 68: cis-4-(4-(6-fluoro-1H-indol-3-yl)-1H-pyrazol-1-yl)cyclohexanol

### Step 1: 4-(4-(6-fluoro-1H-indol-3-yl)-1H-pyrazol-1-yl)cyclohexanone

To a solution of 3-(1-(1,4-dioxaspiro[4.5]decan-8-yl)-1H-pyrazol-4-yl)-6-fluoro-1H-indole (Intermediate 53; 500 mg; 1.46 mmol) in THF (20 ml) was added concentrated aqueous HCl (1.0 mL; 12 mmol; 12M). The mixture was stirred at room temperature overnight, concentrated, and purified by reverse phase flash chromatography to afford 133 mg (45%) of the title compound as a white solid.

LC-MS: m/z 298 [M+H]⁺

### Step 2

A mixture of 4-(4-(6-fluoro-1H-indol-3-yl)-1H-pyrazol-1-yl)cyclohexanone (Step 1; 133 mg; 0.45 mmol) and NaBH₄ (34 mg; 0.90 mmol) in MeOH (20 mL) was stirred at room temperature for 2 hours. The reaction mixture was concentrated and purified by reverse phase flash chromatography and preparative TLC (DCM/MeOH=20/1) to afford 15.2 mg (11%) of the title compound as a yellow solid.

LC-MS: m/z 300.1 [M+H]⁺

¹H NMR (400 MHz, MeOH-d₄) δ [ppm]: 7.98 (s, 1H), 7.78 (s, 1H), 7.71 (dd, J=8.6, 5.4 Hz, 1H), 7.41 (s, 1H), 7.10 (dd, J=9.8, 2.2 Hz, 1H), 6.89 (ddd, J=9.7, 8.6, 2.2 Hz, 1H), 4.31-4.22 (m, 1H), 4.07-4.03 (m, 1H), 2.37-2.23 (m, 2H), 2.01-1.92 (m, 4H), 1.84-1.72 (m, 2H).

### Compound 69: trans-4-(4-(6-fluoro-1H-indol-3-yl)-1H-pyrazol-1-yl)cyclohexanol

Following the general method as outlined in the synthesis of compound 70, starting from trans-4-(4-(6-fluoro-1-(phenylsulfonyl)-1H-indol-3-yl)-1H-pyrazol-1-yl)cyclohexanol (Intermediate 49; 32 mg; 0.073 mmol), 15 mg (69%) of the title compound was obtained as a white solid after purification by preparative TLC (DCM/MeOH=20/1).

¹H NMR (400 MHz, MeOH-d₄) δ [ppm]: 7.96 (s, 1H), 7.78 (s, 1H), 7.71 (dd, J=8.7, 5.3 Hz, 1H), 7.40 (s, 1H), 7.10 (dd, J=9.9, 2.3 Hz, 1H), 6.98-6.78 (m, 1H), 4.33-4.19 (m, 1H), 3.71 (ddd, J=11.0, 6.7, 4.3 Hz, 1H), 2.26-2.07 (m, 4H), 1.99 (dt, J=12.1, 6.7 Hz, 2H), 1.63-1.42 (m, 2H). m.p. 189.7-190.5° C.

### Compound 70: 6-Fluoro-3-(1H-pyrazol-4-yl)-1H-indole

To a solution of 6-fluoro-1-(phenylsulfonyl)-3-(1H-pyrazol-4-yl)-1H-indole (Intermediate 5; 183 mg; 0.54 mmol) in MeOH (20 mL) was added a solution of NaOH (142 mg; 3.55 mmol) in water (1 mL). The reaction mixture was stirred at 85° C. for 4 h, concentrated, diluted with H₂O (5 mL), and extracted with Et₂O (10 mL×3). The combined organic layers were washed with water (10 mL×2), brine (10 mL), dried over anhydrous Na₂SO₄, filtered, and concentrated. The crude product was purified by preparative HPLC. 39 mg (36%) of the title compound was obtained as a white solid.

¹H NMR (400 MHz, DMSO-d₆) δ [ppm]: 12.81 (s, 1H), 11.15 (s, 1H), 8.07 (s, 1H), 7.83-7.74 (m, 2H), 7.54-7.53 (m, 1H), 7.17-7.14 (m, 1H), 6.92-6.85 (m, 1H). m.p. 152.3-156.5° C.

### Compound 71: 5,6-difluoro-3-(1H-pyrazol-4-yl)-1H-indole

Following the general method as outlined in the synthesis of compound 70, starting from 5,6-difluoro-1-(phenylsulfonyl)-3-(1H-pyrazol-4-yl)-1H-indole hydrochloride (Intermediate 30; 150 mg; 0.39 mmol), 10 mg (12%) of the title compound was obtained as a white solid after purification by reverse phase flash chromatography.

¹H NMR (400 MHz, DMSO-d₆) δ [ppm]: 12.88 (s, 1H), 11.31 (s, 1H), 8.02 (s, 2H), 7.76 (dd, J=10.8, 8.4 Hz, 1H), 7.64 (s, 1H), 7.40 (dd, J=10.9, 7.2 Hz, 1H). m.p. 163.1-165.5° C.

### Compound 72: 3-(1H-pyrazol-4-yl)-6-(trifluoromethyl)-1H-indole

Following the general method as outlined in the synthesis of compound 70, starting from 1-(phenylsulfonyl)-3-(1H-pyrazol-4-yl)-6-(trifluoromethyl)-1H-indole (Intermediate 42; 200 mg; 0.51 mmol), 65 mg (50%) of the title compound was obtained as a white solid after purification by preparative TLC (DCM/MeOH=9/1).

¹H NMR (400 MHz, DMSO-d₆) δ [ppm]: 13.05-12.74 (m, 1H), 11.57 (s, 1H), 8.14 (s, 1H), 7.99 (d, J=8.3 Hz, 1H), 7.89 (s, 1H), 7.81 (d, J=2.4 Hz, 1H), 7.74 (s, 1H), 7.32 (dd, J=8.4, 1.4 Hz, 1H).

¹⁹F NMR (377 MHz, DMSO) δ: −58.77 (s, 1H). m.p. 185.4-186.6° C.

### Compound 73: 6-Fluoro-3-(1-methyl-H-pyrazol-4-yl)-1H-indole

Following the general method as outlined in the synthesis of compound 70, starting from 6-fluoro-3-(1-methyl-1H-pyrazol-4-yl)-1-(phenylsulfonyl)-1H-indole (Intermediate 6; 218 mg; 0.61 mmol), 65 mg (49%) of the title compound was obtained as a white solid after purification by preparative HPLC.

¹H NMR (400 MHz, DMSO-d₆) δ [ppm]: 11.19 (s, 1H), 8.07 (s, 1H), 7.76-7.73 (m, 2H), 7.54-7.53 (m, 1H), 7.18-7.15 (m, 1H), 6.93-6.88 (m, 1H), 3.87 (s, 3H). m.p. 187.7-188.4° C.

### Compound 74: 3-(1,5-dimethyl-1H-pyrazol-4-yl)-6-fluoro-1H-indole and 3-(1,3-dimethyl-1H-pyrazol-4-yl)-6-fluoro-1H-indole

Following the general method as outlined in the synthesis of compound 70, starting from 3-(1,5-dimethyl-1H-pyrazol-4-yl)-6-fluoro-1-(phenylsulfonyl)-1H-indole and 3-(1,3-dimethyl-1H-pyrazol-4-yl)-6-fluoro-1-(phenylsulfonyl)-1H-indole (Intermediate 26; 382 mg; 1.03 mmol), 17 mg (7%) of the title compound were obtained as a white solid after purification by preparative HPLC and preparative chiral HPLC (Chiralpak AD-H column, eluting with Hexane/isopropyl alcohol/diethylamine 80/20/0.3, first eluting product).

¹H NMR (400 MHz, MeOH-d₄) δ [ppm]: 7.63 (s, 1H), 7.42 (dd, J=8.7, 5.3 Hz, 1H), 7.14 (s, 1H), 6.98 (dd, J=9.9, 2.3 Hz, 1H), 6.78-6.69 (m, 1H), 3.78 (s, 3H), 2.20 (s, 3H).

### Compound 75: 3-(1,3-dimethyl-1H-pyrazol-4-yl)-6-fluoro-1H-indole

The Title Compound (26 mg; 11%) was obtained as the second-eluting product in the preparative chiral HPLC of the synthesis of compound 74.

¹H NMR (400 MHz, MeOH-d₄) δ [ppm]: 7.57 (s, 1H), 7.48 (dd, J=8.7, 5.3 Hz, 1H), 7.21 (s, 1H), 7.11 (dd, J=9.9, 2.2 Hz, 1H), 6.88-6.82 (m, 1H), 3.88 (s, 3H), 2.36 (s, 3H).

### Compound 77: 3-(1-methyl-5-(trifluoromethyl)-1H-pyrazol-4-yl)-1H-indole

### Step 1: 1,1,1-trifluoro-3-(1-(phenylsulfonyl)-1H-indol-3-yl)propan-2-one

To a mixture of 2-(1-(phenylsulfonyl)-1H-indol-3-yl)acetic acid (1.50 g; 4.76 mmol) and trifluoroacetic anhydride (3.0 mL; 22 mmol) in toluene (150 mL) at 0° C. was added pyridine (2.4 mL; 30 mmol). The reaction mixture was stirred at 60° C. overnight. The reaction mixture was quenched with ice-water (200 mL) and extracted with EtOAc (50 mL×2). The combined organic layers were washed with brine (50 mL), dried over anhydrous Na₂SO₄, filtered, concentrated, and purified by reverse phase flash chromatography and preparative HPLC to afford 0.50 g (29%) the title compound as a yellow oil.

### Step 2: 4,4,4-trifluoro-3-oxo-2-(1-(phenylsulfonyl)-1H-indol-3-yl)butanal

A mixture of 1,1,1-trifluoro-3-(1-(phenylsulfonyl)-1H-indol-3-yl)propan-2-one (Step 1; 200 mg; 0.54 mmol) and POCl₃ (0.50 mL; 5.5 mmol) in DMF (10 mL) was stirred at room temperature overnight. The reaction mixture was quenched with ice-water (50 mL) and extracted with EtOAc (50 mL×2). The combined organic layers were dried over anhydrous Na₂SO₄, filtered, and concentrated to afford 460 mg (>100%) of the title compound as a yellow oil, which was used directly without further purification.

LC-MS: m/z 394 [M−H]⁻

### Step 3: 3-(1-methyl-5-(trifluoromethyl)-1H-pyrazol-4-yl)-1-(phenylsulfonyl)-1H-indole

A solution of 4,4,4-trifluoro-3-oxo-2-(1-(phenylsulfonyl)-1H-indol-3-yl)butanal (Step 2; 230 mg crude; 0.27 mmol) and methylhydrazine (670 mg; 40% aqueous solution; 5.82 mmol) in CH₃CN (20 mL) was stirred for 1 hour. The reaction mixture was concentrated and purified by reverse phase flash chromatography to afford 54 mg (49%) of the title compound as a yellow oil.

LC-MS: m/z 406 [M+H]⁺

### Step 4

Following the general method as outlined in the synthesis of compound 3, starting from 3-(1-methyl-5-(trifluoromethyl)-1H-pyrazol-4-yl)-1-(phenylsulfonyl)-1H-indole (Step 3; 54 mg; 0.13 mmol), 20 mg (57%) of the title compound was obtained as a white solid after purification by reverse phase flash chromatography.

LC-MS: m/z 266.1 [M+H]⁺

¹H NMR (300 MHz, MeOH-d₄) δ [ppm]: 7.63 (s, 1H), 7.46 (d, J=7.9 Hz, 1H), 7.41 (d, J=8.0 Hz, 1H), 7.25 (s, 1H), 7.15 (ddd, J=8.0, 7.0, 1.0 Hz, 1H), 7.05 (ddd, J=7.9, 7.0, 1.0 Hz, 1H), 4.06 (s, 3H).

### Compound 78: 6-fluoro-3-(1-(piperidin-4-yl)-1H-pyrazol-4-yl)-1H-indole

To a solution of tert-butyl 4-(4-(6-fluoro-1H-indol-3-yl)-1H-pyrazol-1-yl)piperidine-1-carboxylate (Intermediate 16B; 230 mg; 0.60 mmol) in dioxane (3 mL) was added conc. aqueous HCl (3 mL; 36%). The reaction mixture was stirred for 0.5 hour and concentrated to dryness, neutralized with saturated aqueous NaHCO₃ (50 mL), extracted with EtOAc (50 ml×2). The combined organic layers were dried over anhydrous Na₂SO₄, filtered, concentrated. The residue was purified by preparative TLC (DCM/MeOH=10/1) to afford 70 mg (41%) of the title compound as a yellow solid.

LC-MS: m/z 285.2 [M+H]⁺.

¹H NMR (400 MHz, MeOH-d₄) δ [ppm]: 8.02 (s, 1H), 7.85 (s, 1H), 7.71 (dd, J=8.7, 5.3 Hz, 1H), 7.42 (s, 1H), 7.10 (dd, J=9.8, 2.1 Hz, 1H), 6.88 (td, J=9.4, 2.3 Hz, 1H), 4.59 (ddd, J=15.2, 10.3, 4.7 Hz, 1H), 3.58 (d, J=13.1 Hz, 2H), 3.23 (td, J=12.7, 3.9 Hz, 2H), 2.46-2.17 (m, 4H). m.p. 240.1-241.8° C.

### Compound 79: 2-(4-(4-(6-fluoro-1H-indol-3-yl)-1H-pyrazol-1-yl)piperidin-1-yl)ethanol

### Step 1: 2-(4-(4-(6-fluoro-1-(phenylsulfonyl)-1H-indol-3-yl)-1H-pyrazol-1-yl)piperidin-1-yl)ethanol

To a mixture of 6-fluoro-1-(phenylsulfonyl)-3-(1-(piperidin-4-yl)-1H-pyrazol-4-yl)-1H-indole hydrochloride (Intermediate 17; 210 mg; 0.46 mmol), K₂CO₃ (189 mg; 1.37 mmol) and KI (3 mg; 0.018 mmol) in DMF (5 mL) was added 2-bromoethanol (115 mg; 0.92 mmol) under nitrogen. The reaction mixture was stirred at 100° C. for 15 hours, concentrated, and purified by preparative TLC (DCM/MeOH=10/1) to afford 148 mg (69%) of the title compound as a white solid.

LC-MS: m/z 469.1 [M+H]⁺.

¹H NMR (400 MHz, DMSO-d₆) δ [ppm]: 8.40 (s, 1H), 8.11 (s, 1H), 8.07-7.99 (m, 3H), 7.94 (dd, J=8.8, 5.3 Hz, 1H), 7.76 (dd, J=9.9, 2.1 Hz, 1H), 7.71 (t, J=7.6 Hz, 1H), 7.61 (t, J=7.7 Hz, 2H), 7.22 (td, J=9.1, 2.2 Hz, 1H), 4.45-4.27 (m, 1H), 3.81-3.58 (m, 2H), 3.05-2.66 (m, 6H), 2.09-2.63 (m, 4H).

### Step 2

Following the general method as outlined in the synthesis of compound 70, starting from 2-(4-(4-(6-fluoro-1-(phenylsulfonyl)-1H-indol-3-yl)-1H-pyrazol-1-yl)piperidin-1-yl)ethanol (Step 1; 148 mg; 0.32 mmol), 36 mg (35%) of the title compound was obtained as a yellow solid after purification by preparative HPLC.

LC-MS: m/z 329.2 [M+H]⁺.

¹H NMR (400 MHz, DMSO-d₆) δ [ppm]: 11.17 (s, 1H), 8.12 (s, 1H), 7.83-7.71 (m, 2H), 7.53 (d, J=2.0 Hz, 1H), 7.16 (dd, J=10.1, 2.2 Hz, 1H), 6.90 (td, J=9.4, 2.2 Hz, 1H), 4.13 (dd, J=14.9, 7.7 Hz, 1H), 3.53 (t, J=6.3 Hz, 2H), 2.99 (d, J=11.4 Hz, 2H), 2.44 (t, J=6.3 Hz, 2H), 2.20-2.10 (m, 2H), 1.99-2.01 (m, 4H).

### Compound 80: 4-(4-(4-(6-fluoro-1H-indol-3-yl)-1H-pyrazol-1-yl)piperidin-1-yl)-4-oxobutanoic acid

### Step 1: methyl 4-(4-(4-(6-fluoro-1-(phenylsulfonyl)-1H-indol-3-yl)-1H-pyrazol-1-yl)piperidin-1-yl)-4-oxobutanoate

Following the general method as outlined in the synthesis of compound 83, starting from 6-fluoro-1-(phenylsulfonyl)-3-(1-(piperidin-4-yl)-1H-pyrazol-4-yl)-1H-indole hydrochloride (Intermediate 17; 400 mg; 0.86 mmol) and succinic acid monomethyl ester (249 mg; 1.88 mmol) in DMF (10 mL), 506 mg of the title compound was obtained as a yellow oil after purification by a silica gel chromatography (EA).

LC-MS: m/z 539.1 [M+H]⁺.

### Step 2

Following the general method as outlined in the synthesis of compound 70, starting from 4-(4-(4-(6-fluoro-1-(phenylsulfonyl)-1H-indol-3-yl)-1H-pyrazol-1-yl)piperidin-1-yl)-4-oxobutanoate (Step 1; 506 mg), 82.2 mg (22.8%) of the title compound was obtained as a white solid after purification by preparative HPLC.

LC-MS: m/z 385.1 [M+H]⁺.

¹H NMR (400 MHz, MeOH-d₄) δ [ppm]: 8.02 (s, 1H), 7.81 (s, 1H), 7.72 (dd, J=8.7, 5.3 Hz, 1H), 7.41 (s, 1H), 7.10 (dd, J=9.8, 2.3 Hz, 1H), 6.93-6.84 (m, 1H), 4.69 (d, J=14.1 Hz, 1H), 4.52 (td, J=11.5, 5.9 Hz, 1H), 4.21 (d, J=15.6 Hz, 1H), 2.90 (d, J=10.6 Hz, 2H), 2.76 (dd, J=15.2, 6.9 Hz, 2H), 2.63 (t, J=6.5 Hz, 2H), 2.21 (d, J=20.6 Hz, 2H), 2.15-1.96 (m, 2H). m.p. 226-228° C.

### Compound 81: 1-(4-(4-(6-fluoro-1H-indol-3-yl)-1H-pyrazol-1-yl)piperidin-1-yl)-3-methoxypropan-1-one

### Step 1: 1-(4-(4-(6-fluoro-1-(phenylsulfonyl)-1H-indol-3-yl)-1H-pyrazol-1-yl)piperidin-1-yl)-3-methoxypropan-1-one

Following the general method as outlined in the synthesis of compound 83, starting from 6-fluoro-1-(phenylsulfonyl)-3-(1-(piperidin-4-ylmethyl)-1H-pyrazol-4-yl)-1H-indole (Intermediate 21; 382 mg; 0.87 mmol) and 3-methoxypropanoic acid (180 mg; 1.73 mmol), 620 mg of the title compound was obtained as a yellow oil after purification by a silica gel chromatography (EtOAc).

LC-MS: m/z 511 [M+H]⁺.

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)-3-methoxypropan-1-one (Step 1; 440 mg), 30 mg (13%) of the title compound was obtained as a white solid after purification by preparative HPLC.

¹H NMR (400 MHz, DMSO-d₆) δ [ppm]: 11.16 (s, 1H), 8.15 (s, 1H), 7.82-7.76 (m, 2H), 7.53 (d, J=2.3 Hz, 1H), 7.16 (dd, J=10.0, 2.3 Hz, 1H), 6.90 (td, J=9.6, 2.4 Hz, 1H), 4.47 (dd, J=23.4, 12.3 Hz, 2H), 4.02 (d, J=14.4 Hz, 1H), 3.58 (t, J=6.6 Hz, 2H), 3.24 (s, 3H), 3.18 (d, J=12.4 Hz, 1H), 2.75 (t, J=12.7 Hz, 1H), 2.63 (t, J=6.6 Hz, 2H), 2.07 (t, J=13.4 Hz, 2H), 1.87 (ddd, J=46.6, 12.2, 4.7 Hz, 2H).

### Compound 82: 1-(4-(4-(6-fluoro-1H-indol-3-yl)-1H-pyrazol-1-yl)piperidin-1-yl)propan-1-one

### Step 1: 1-(4-(4-(6-fluoro-1-(phenylsulfonyl)-1H-indol-3-yl)-1H-pyrazol-1-yl)piperidin-1-yl)propan-1-one

Following the general method as outlined in the synthesis of compound 89, starting from 6-fluoro-1-(phenylsulfonyl)-3-(1-(piperidin-4-yl)-1H-pyrazol-4-yl)-1H-indole hydrochloride (Intermediate 17; 370 mg; 0.80 mmol) and propionyl chloride (179 mg; 1.93 mmol), 310 mg (80%) of the title compound was obtained as a yellow oil, which was used directly without further purification.

LC-MS: m/z 481 [M+H]⁺.

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)propan-1-one (Step 1; 310 mg; 0.65 mmol), 50 mg (23%) of the title compound was obtained as a white solid after purification by preparative HPLC.

¹H NMR (400 MHz, DMSO-d₆) δ [ppm]: 11.16 (s, 1H), 8.15 (s, 1H), 7.79 (q, J=5.6 Hz, 2H), 7.53 (d, J=2.4 Hz, 1H), 7.16 (dd, J=10.1, 2.3 Hz, 1H), 6.93-6.86 (m, 1H), 4.56-4.39 (m, 2H), 3.98 (d, J=14.1 Hz, 1H), 3.19 (t, J=12.0 Hz, 1H), 2.74 (t, J=11.5 Hz, 1H), 2.38 (q, J=7.4 Hz, 2H), 2.07 (t, J=13.6 Hz, 2H), 1.99-1.75 (m, 2H), 1.02 (t, J=7.4 Hz, 3H). m.p. 200-202° C.

### Compound 83: 2-(dimethylamino)-1-(4-(4-(6-fluoro-1H-indol-3-yl)-1H-pyrazol-1-yl)piperidin-1-yl)ethanone

### Step 1: 2-(dimethylamino)-1-(4-(4-(6-fluoro-1-(phenylsulfonyl)-1H-indol-3-yl)-1H-pyrazol-1-yl)piperidin-1-yl)ethanone

To a solution of (dimethylamino)acetic acid (165 mg; 1.60 mmol), HATU (608 mg; 1.60 mmol), and DIPEA (619 mg; 4.80 mmol) in THF (25 mL) was added 6-fluoro-1-(phenylsulfonyl)-3-(1-(piperidin-4-yl)-1H-pyrazol-4-yl)-1H-indole hydrochloride (Intermediate 17; 368 mg; 0.80 mmol) under nitrogen. The reaction mixture was stirred overnight, concentrated, dealed with saturated aqueous NaHCO₃ (50 mL), extracted with EtOAc (50 ml×2). The combined organic layers were dried over anhydrous Na₂SO₄, filtered, concentrated and purified by a silica gel chromatography (DCM/MeOH=20/1) to afford 400 mg (100%) of the title compound as a yellow oil.

LC-MS: m/z 510.1 [M+H]⁺.

### Step 2

Following the general method as outlined in the synthesis of compound 70, starting from 2-(dimethylamino)-1-(4-(4-(6-fluoro-1-(phenylsulfonyl)-1H-indol-3-yl)-1H-pyrazol-1-yl)piperidin-1-yl)ethanone (Step 1; 400 mg; 0.80 mmol), 80 mg (27%) of the title compound was obtained as a white solid after purification by preparative HPLC.

LC-MS: m/z 370.2 [M+H]⁺.

¹H NMR (400 MHz, MeOH-d₄) δ [ppm]: 7.99 (d, J=4.4 Hz, 1H), 7.81 (s, 1H), 7.71 (dd, J=8.8, 5.3 Hz, 1H), 7.40 (s, 1H), 7.15-7.05 (m, 1H), 6.96-6.79 (m, 1H), 4.69 (dd, J=15.4, 5.1 Hz, 1H), 4.51 (tt, J=11.2, 4.1 Hz, 1H), 4.25 (d, J=13.2 Hz, 1H), 3.25-3.37 (m, 1H), 2.89 (m, 1H), 2.19-2.16 (m, 2H), 2.16-1.89 (m, 2H). m.p. 193.7-196.1° C.

### Compound 84: 1-(4-(4-(6-fluoro-1H-indol-3-yl)-1H-pyrazol-1-yl)piperidin-1-yl)-2-hydroxyethanone

### Step 1: 2-(4-(4-(6-fluoro-1-(phenylsulfonyl)-1H-indol-3-yl)-1H-pyrazol-1-yl)piperidin-1-yl)-2-oxoethyl acetate

Following the general method as outlined in the synthesis of compound 89, starting from 6-fluoro-1-(phenylsulfonyl)-3-(1-(piperidin-4-yl)-1H-pyrazol-4-yl)-1H-indole hydrochloride (Intermediate 17; 368 mg; 0.80 mmol) and 2-chloro-2-oxoethyl acetate (165 mg; 1.21 mmol), 421 mg (100%) of the title compound was obtained as a yellow solid, which was used directly without further purification.

LC-MS: m/z 525.0 [M+H]⁺.

¹H NMR (400 MHz, DMSO-d₆) δ [ppm]: 8.38 (s, 1H), 8.13-7.98 (m, 4H), 7.93 (dd, J=8.8, 5.3 Hz, 1H), 7.76 (dd, J=9.8, 2.0 Hz, 1H), 7.70 (t, J=7.4 Hz, 1H), 7.60 (t, J=7.7 Hz, 2H), 7.22 (td, J=9.1, 2.1 Hz, 1H), 4.83 (s, 2H), 4.48 (ddd, J=11.6, 9.9, 4.0 Hz, 1H), 4.40 (dd, J=13.4, 1.1 Hz, 1H), 3.90-3.77 (m, 1H), 3.21 (dd, J=12.9, 12.4 Hz, 1H), 2.89-2.75 (m, 1H), 2.09 (s, 3H), 2.08-1.72 (m, 4H).

### Step 2

Following the general method as outlined in the synthesis of compound 70, starting from 2-(4-(4-(6-fluoro-1-(phenylsulfonyl)-1H-indol-3-yl)-1H-pyrazol-1-yl)piperidin-1-yl)-2-oxoethyl acetate (Step 1; 421 mg; 0.80 mmol), 26 mg (9%) of the title compound was obtained as a white solid after purification by preparative HPLC.

LC-MS: m/z 343.1 [M+H]⁺.

¹H NMR (400 MHz, DMSO-d₆) δ [ppm]: 11.18 (s, 1H), 8.15 (s, 1H), 7.78 (d, J=9.0 Hz, 2H), 7.54 (s, 1H), 7.16 (d, J=10.0 Hz, 1H), 6.90 (t, J=9.0 Hz, 1H), 4.56 (m, 1H), 4.51-4.41 (m, 2H), 4.13 (s, 2H), 3.80-3.84 (m, 1H), 3.21-3.07 (m, 1H), 2.88-2.74 (m, 1H), 2.14-1.71 (m, 5H). m.p. 99.9-102.1° C.

### Compound 85: 1-(4-(4-(6-fluoro-1H-indol-3-yl)-1H-pyrazol-1-yl)piperidin-1-yl)-2-methoxyethanone

### Step 1: 1-(4-(4-(6-fluoro-1-(phenylsulfonyl)-1H-indol-3-yl)-1H-pyrazol-1-yl)piperidin-1-yl)-2-methoxyethanone

Following the general method as outlined in the synthesis of compound 83, starting from 6-fluoro-1-(phenylsulfonyl)-3-(1-(piperidin-4-ylmethyl)-1H-pyrazol-4-yl)-1H-indole (Intermediate 21; 188 mg; 0.43 mmol) and 2-methoxyacetic acid (41 mg; 0.46 mmol), 260 mg of the title compound was obtained as a yellow oil after purification by a silica gel chromatography (EtOAc).

LC-MS: m/z 497 [M+H]⁺.

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)-2-methoxyethanone (Step 1; 260 mg), 10 mg (7%) of the title compound was obtained as a white solid after purification by preparative HPLC.

¹H NMR (400 MHz, DMSO-d₆) δ [ppm]: 11.18 (s, 1H), 8.15 (s, 1H), 7.79 (q, J=5.3 Hz, 2H), 7.54 (d, J=1.9 Hz, 1H), 7.16 (dd, J=10.1, 2.3 Hz, 1H), 6.90 (td, J=9.7, 2.3 Hz, 1H), 4.46 (t, J=11.3 Hz, 2H), 4.20-4.08 (m, 2H), 3.90 (d, J=12.9 Hz, 1H), 3.31 (s, 3H), 3.21-3.13 (m, 1H), 2.79 (t, J=12.0 Hz, 1H), 2.08 (m, 2H), 1.91 (m, 2H).

### Compound 86: 1-(4-(4-(6-fluoro-1H-indol-3-yl)-1H-pyrazol-1-yl)piperidin-1-yl)-2-methylpropan-1-one

### Step 1: 1-(4-(4-(6-fluoro-1-(phenylsulfonyl)-1H-indol-3-yl)-1H-pyrazol-1-yl)piperidin-1-yl)-2-methylpropan-1-one

Following the general method as outlined in the synthesis of compound 83, starting from 6-fluoro-1-(phenylsulfonyl)-3-(1-(piperidin-4-yl)-1H-pyrazol-4-yl)-1H-indole hydrochloride (Intermediate 17; 271 mg; 0.59 mmol) and isobutyric acid (104 mg; 1.18 mmol) in DMF (15 mL), 277 mg (90%) of the title compound was obtained as a yellow oil after purification by a silica gel chromatography (DCM/MeOH=20/1).

LC-MS: m/z 495.1 [M+H]⁺.

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)-2-methylpropan-1-one (Step 1; 277 mg; 0.56 mmol), 31 mg (16%) of the title compound was obtained as a white solid after purification by preparative HPLC.

LC-MS: m/z 355.1 [M+H]⁺.

¹H NMR (400 MHz, DMSO-d₆) δ [ppm]: 11.17 (s, 1H), 8.16 (s, 1H), 7.87-7.70 (m, 2H), 7.54 (d, J=2.4 Hz, 1H), 7.16 (dd, J=10.1, 2.3 Hz, 1H), 6.90 (td, J=9.6, 2.4 Hz, 1H), 4.60-4.35 (m, 2H), 4.14-3.92 (m, 1H), 3.22 (dd, J=13.3, 12.7 Hz, 1H), 2.93 (dt, J=13.5, 6.7 Hz, 1H), 2.69-2.76 (dd, J=20.8, 8.3 Hz, 1H), 2.08-2.15 (m, 2H), 1.98-1.67 (m, 2H), 1.03 (dd, J=9.4, 7.3 Hz, 6H). m.p. 210.2-211° C.

### Compound 87: 1-(4-(4-(6-fluoro-1H-indol-3-yl)-1H-pyrazol-1-yl)piperidin-1-yl)-2,2-dimethylpropan-1-one

### Step 1: 1-(4-(4-(6-fluoro-1-(phenylsulfonyl)-1H-indol-3-yl)-1H-pyrazol-1-yl)piperidin-1-yl)-2,2-dimethylpropan-1-one

Following the general method as outlined in the synthesis of compound 89, starting from 6-fluoro-1-(phenylsulfonyl)-3-(1-(piperidin-4-yl)-1H-pyrazol-4-yl)-1H-indole hydrochloride (Intermediate 17; 200 mg; 0.47 mmol) and pivaloyl chloride (0.12 mL; 0.98 mmol), 250 mg (>100%) of the title compound was obtained as a yellow solid, which was used directly without further purification.

LC-MS: m/z 509 [M+H]⁺.

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)-2,2-dimethylpropan-1-one (Step 1; 250 mg), 99 mg (54%) of the title compound was obtained as a white solid after purification by preparative HPLC.

LC-MS: m/z 369.1 [M+H]⁺.

¹H NMR (400 MHz, DMSO-d₆) δ [ppm]: 11.16 (s, 1H), 8.17 (s, 1H), 7.82-7.77 (m, 2H), 7.54 (d, J=2.4 Hz, 1H), 7.16 (dd, J=10.1, 2.3 Hz, 1H), 7.00-6.80 (m, 1H), 4.52-4.36 (m, 3H), 2.99 (t, J=12.7 Hz, 2H), 2.16-1.97 (m, 2H), 1.95-1.75 (m, 2H), 1.24 (s, 9H).

¹⁹F NMR (377 MHz, DMSO-d₆) δ [ppm]: −122.09 (s, 1H). m.p. 231-232° C.

### Compound 88: cyclopropyl(4-(4-(6-fluoro-1H-indol-3-yl)-1H-pyrazol-1-yl)piperidin-1-yl)methanone

### Step 1: cyclopropyl(4-(4-(6-fluoro-1-(phenylsulfonyl)-1H-indol-3-yl)-1H-pyrazol-1-yl)piperidin-1-yl)methanone

Following the general method as outlined in the synthesis of compound 83, starting from 6-fluoro-1-(phenylsulfonyl)-3-(1-(piperidin-4-yl)-1H-pyrazol-4-yl)-1H-indole hydrochloride (Intermediate 17; 300 mg; 0.65 mmol) and cyclopropanecarboxylic acid (122 mg; 1.42 mmol) in DMF (6 mL), 345 mg of the title compound was obtained as a yellow oil, which was used directly without further purification.

LC-MS: m/z 493.1 [M+H]⁺.

### Step 2

Following the general method as outlined in the synthesis of compound 70, starting from cyclopropyl(4-(4-(6-fluoro-1-(phenylsulfonyl)-1H-indol-3-yl)-1H-pyrazol-1-yl)piperidin-1-yl)methanone (Step 1; 345 mg), 118 mg (47.9%) of the title compound was obtained as a white solid after purification by preparative HPLC.

LC-MS: m/z 353.1 [M+H]⁺.

¹H NMR (400 MHz, DMSO-d₆) δ [ppm]: 11.16 (s, 1H), 8.17 (s, 1H), 7.86-7.76 (m, 2H), 7.54 (d, J=1.9 Hz, 1H), 7.17 (dd, J=10.1, 2.3 Hz, 1H), 6.95-6.82 (m, 1H), 4.54-4.43 (m, 2H), 4.39 (s, 1H), 3.32 (d, J=24.6 Hz, 1H), 2.78 (s, 1H), 2.12 (s, 1H), 2.09-1.98 (m, 2H), 1.90 (d, J=37.4 Hz, 2H), 0.73 (d, J=7.9 Hz, 4H). m.p. 190.7-191.4° C.

### Compound 89: 1-(4-(4-(6-fluoro-1H-indol-3-yl)-1H-pyrazol-1-yl)piperidin-1-yl)ethanone

### Step 1: 1-(4-(4-(6-fluoro-1-(phenylsulfonyl)-1H-indol-3-yl)-1H-pyrazol-1-yl)piperidin-1-yl)ethanone

To a solution of 6-fluoro-1-(phenylsulfonyl)-3-(1-(piperidin-4-yl)-1H-pyrazol-4-yl)-1H-indole hydrochloride (Intermediate 17; 290 mg), Et₃N (0.23 mL; 1.65 mmol) in DCM (20 mL) was added acetyl chloride (87 mg; 1.1 mmol) dropwise under nitrogen. The reaction mixture was stirred for 1 h and concentrated to afford 256 mg (100%) of the title compound as a white solid, which was used directly without further purification.

LC-MS: m/z 467.1 [M+H]⁺.

¹H NMR (400 MHz, DMSO-d₆) δ [ppm]: 8.39 (s, 1H), 8.10 (s, 1H), 8.04 (d, J=7.6 Hz, 2H), 7.99 (s, 1H), 7.94 (dd, J=8.7, 5.4 Hz, 1H), 7.76 (dd, J=9.8, 2.3 Hz, 1H), 7.71 (t, J=7.4 Hz, 1H), 7.61 (t, J=7.8 Hz, 2H), 7.22 (td, J=9.1, 2.3 Hz, 1H), 4.43-4.49 (m, 2H), 3.86-3.95 (m, 1H), 3.27-3.19 (m, 1H), 2.78-2.69 (m, 1H), 2.05 (s, 3H), 2.02-1.77 (m, 4H).

### Step 2

Following the general method as outlined in the synthesis of compound 70, starting from 1-(4-(4-(6-fluoro-1-(phenylsulfonyl)-1H-indol-3-yl)-1H-pyrazol-1-yl)piperidin-1-yl)ethanone (Step 1; 250 mg; 0.54 mmol), 80 mg (45%) of the title compound was obtained as a yellow solid after purification by preparative HPLC.

LC-MS: m/z 327.2 [M+H]⁺.

¹H NMR (400 MHz, DMSO-d₆) δ [ppm]: 11.16 (s, 1H), 8.16 (s, 1H), 7.85-7.74 (m, 2H), 7.54 (d, J=2.2 Hz, 1H), 7.16 (dd, J=10.0, 2.2 Hz, 1H), 6.90 (td, J=9.7, 2.3 Hz, 1H), 4.55-4.34 (m, 2H), 3.92-3.95 (m, 1H), 3.18-3.25 (m, 1H), 2.70-2.76 (m, 1H), 2.06 (s, 3H), 2.13-1.71 (m, 4H). m.p. 183.2-184.5° C.

### Compound 90: 4-(4-(6-fluoro-1H-indol-3-yl)-1H-pyrazol-1-yl)-N,N-dimethylpiperidine-1-carboxamide

The title compound was obtained (15 mg; 8%) as a white solid after purification by reverse phase flash chromatography in step 2 of the synthesis of compound 89.

LC-MS: m/z 356.2 [M+H]⁺.

¹H NMR (400 MHz, DMSO-d₆) δ [ppm]: 11.16 (s, 1H), 8.15 (s, 1H), 7.93-7.63 (m, 2H), 7.54 (d, J=2.3 Hz, 1H), 7.16 (dd, J=10.1, 2.3 Hz, 1H), 6.98-6.74 (m, 1H), 4.42-4.27 (m, 1H), 3.67 (d, J=13.2 Hz, 2H), 2.88 (t, J=11.1 Hz, 2H), 2.77 (s, 6H), 2.07-1.85 (m, 4H). m.p. 223.0-224.6° C.

### Compound 91: 6-fluoro-3-(1-(1-methylpiperidin-4-yl)-1H-pyrazol-4-yl)-1H-indole

### Step 1: 6-fluoro-3-(1-(1-methylpiperidin-4-yl)-1H-pyrazol-4-yl)-1-(phenylsulfonyl)-1H-indole

To a solution of 6-fluoro-1-(phenylsulfonyl)-3-(1-(piperidin-4-yl)-1H-pyrazol-4-yl)-1H-indole hydrochloride (Intermediate 17; 290 mg), Et₃N (55 mg; 0.55 mmol)cOH (1 drop), and 37% aqueous HCHO (89 mg; 1.1 mmol) in MeOH (10 mL) was added NaBH(OAc)₃ (233 mg; 1.05 mmol). The reaction mixture was stirred for 16 h and concentrated. The residue was dissolved in DCM (20 mL) and washed with saturated NaHCO₃ (20 mL). The organic layer was dried over anhydrous Na₂SO₄, filtered, and concentrated to afford 270 mg (>100%) of the title compound as a white solid.

LC-MS: m/z 439.2 [M+H]⁺.

¹H NMR (400 MHz, CDCl₃) δ [ppm]: 7.90 (d, J=7.6 Hz, 2H), 7.70-7.80 (m, 3H), 7.63-7.53 (m, 3H), 7.47 (t, J=7.7 Hz, 2H), 7.05 (td, J=8.9, 2.3 Hz, 1H), 4.25 (dd, J=9.5, 5.2 Hz, 1H), 3.09-3.13 (m, 2H), 2.43 (s, 3H), 2.39-2.21 (m, 6H).

### Step 2

Following the general method as outlined in the synthesis of compound 70, starting from 6-fluoro-3-(1-(1-methylpiperidin-4-yl)-1H-pyrazol-4-yl)-1-(phenylsulfonyl)-1H-indole (Step 1; 240 mg), 58 mg (37%) of the title compound was obtained as a white solid after purification by preparative HPLC.

LC-MS: m/z 299.2 [M+H]⁺.

¹H NMR (400 MHz, DMSO-d₆) δ [ppm]: 11.16 (s, 1H), 8.12 (s, 1H), 7.76-7.80 (m, 2H), 7.53 (d, J=2.3 Hz, 1H), 7.16 (dd, J=10.1, 2.3 Hz, 1H), 6.90 (td, J=9.6, 2.3 Hz, 1H), 4.12 (dd, J=13.3, 7.5 Hz, 1H), 2.85-2.88 (m, 2H), 2.21 (s, 3H), 2.14-1.90 (m, 6H). m.p. 222.8-223.7° C.

### Compound 92: 6-fluoro-3-(1-(1-(trifluoromethylsulfonyl)piperidin-4-yl)-1H-pyrazol-4-yl)-1H-indole

### Step: 6-fluoro-1-(phenylsulfonyl)-3-(1-(1-((trifluoromethyl)sulfonyl)piperidin-4-yl)-1H-pyrazol-4-yl)-1H-indole

To a solution of 6-fluoro-1-(phenylsulfonyl)-3-(1-(piperidin-4-yl)-1H-pyrazol-4-yl)-1H-indole hydrochloride (Intermediate 17; 217 mg; 0.47 mmol), Et₃N (0.26 mL; 1.88 mmol) in DCM (15 mL) at 0° C. was added trifluoromethanesulfonic anhydride (0.13 mL; 0.77 mmol) dropwise. The reaction mixture was stirred at room temperature for 1 hour, concentrated, and purified by preparative TLC (EtOAc) to afford 100 mg (38%) of the title compound as a white solid.

LC-MS: m/z 556.8 [M+H]⁺

¹H NMR (300 MHz, DMSO-d₆) δ [ppm]: 8.43 (s, 1H), 8.11 (s, 1H), 8.07-8.02 (m, 2H), 8.03 (s, 1H), 7.94 (dd, J=8.7, 5.4 Hz, 1H), 7.77 (dd, J=9.7, 8.7, 2.3 Hz, 1H), 7.75-7.67 (m, 1H), 7.65-7.56 (m, 2H), 7.22 (ddd, J=9.7, 8.7, 2.2 Hz, 1H), 4.60-4.47 (m, 1H), 3.99-3.87 (m, 2H), 3.54-3.39 (m, 2H), 2.29-2.16 (m, 2H), 2.14-1.96 (m, 2H).

### Step 2

Following the general method as outlined in the synthesis of compound 3, starting from 6-fluoro-1-(phenylsulfonyl)-3-(1-(1-((trifluoromethyl)sulfonyl)piperidin-4-yl)-1H-pyrazol-4-yl)-1H-indole (Step 1; 100 mg; 0.18 mmol), 60 mg (80%) of the title compound was obtained as a brown solid after purification by preparative TLC (petroleum ether/EtOAc=1/1).

LC-MS: m/z 417.0 [M+H]⁺

¹H NMR (300 MHz, DMSO-d₆) δ [ppm]: 11.20 (s, 1H), 8.21 (s, 1H), 7.83 (s, 1H), 7.81 (dd, J=8.7, 5.4 Hz, 1H), 7.56 (d, J=2.4 Hz, 1H), 7.16 (dd, J=10.1, 2.3 Hz, 1H), 6.91 (ddd, J=9.7, 8.7, 2.3 Hz, 1H), 4.53 (tt, J=11.2, 4.1 Hz, 1H), 3.99-3.87 (m, 2H), 3.54-3.38 (m, 2H), 2.29-2.16 (m, 2H), 2.15-1.97 (m, 2H).

### Compound 94: 1-(4-(4-(6-fluoro-1H-indol-3-yl)-3-methyl-1H-pyrazol-1-yl)piperidin-1-yl)ethanone

Following the general method as outlined in the synthesis of compound 3, starting from a mixture of 1-(4-(4-(6-fluoro-1-(phenylsulfonyl)-1H-indol-3-yl)-3-methyl-1H-pyrazol-1-yl)piperidin-1-yl)ethanon and 1-(4-(4-(6-fluoro-1-(phenylsulfonyl)-1H-indol-3-yl)-5-methyl-1H-pyrazol-1-yl)piperidin-1-yl)ethanone (Intermediate 54; 450 mg crude; 0.54 mmol), 73 mg (40%) of a mixture of 1-(4-(4-(6-fluoro-1H-indol-3-yl)-3-methyl-1H-pyrazol-1-yl)piperidin-1-yl)ethanone and 1-(4-(4-(6-fluoro-1H-indol-3-yl)-5-methyl-1H-pyrazol-1-yl)piperidin-1-yl)ethanone was obtained as a white solid after purification by reverse phase flash chromatography.

10.5 mg of the title compound was obtained as a white solid after purification by chiral preparative HPLC.

Preparative chiral HPLC: ChiralCel OD-H 250 mm×20 mm 5 μm; Mobile phase: CO₂/MeOH/Et₂NH=60/40/0.3; Flow: 50 mL/min 230 nm ambient temperature

Analytical chiral HPLC: Chiralcel OD-H 250 mm×4.6 mm 5 μm; Mobile phase: CO₂/MeOH/Et₂NH=60/40/0.3; Flow: 3.0 mL/min 230 nm ambient temperature

Retention time: 2.45 min.

LC-MS: m/z 341.1 [M+H]⁺

¹H NMR (300 MHz, DMSO-d₆) δ [ppm]: 11.20 (s, 1H), 8.00 (s, 1H), 7.65 (dd, J=8.7, 5.5 Hz, 1H), 7.37 (d, J=2.4 Hz, 1H), 7.17 (dd, J=10.1, 2.3 Hz, 1H), 6.88 (ddd, J=9.7, 8.7, 2.3 Hz, 1H), 4.55-4.44 (m, 1H), 4.37 (tt, J=2.4 Hz, 1H), 4.02-3.85 (m, 1H), 3.26-3.13 (m, 1H), 2.80-2.64 (m, 1H), 2.26 (s, 3H), 2.05 (s, 3H), 2.12-1.68 (m, 4H).

### Compound 95: 1-(4-(4-(6-fluoro-1H-indol-3-yl)-5-methyl-1H-pyrazol-1-yl)piperidin-1-yl)ethanone

9.7 mg of the title compound was obtained as a white solid after chiral preparative HPLC in the synthesis of compound 95.

Analytical chiral HPLC: Chiralcel OD-H 250 mm×4.6 mm 5 μm; Mobile phase: CO₂/MeOH/Et₂NH=60/40/0.3; Flow: 3.0 mL/min 230 nm ambient temperature

Retention time: 2.87 min.

LC-MS: m/z 341.1 [M+H]⁺

¹H NMR (300 MHz, DMSO-d₆) δ [ppm]: 11.21 (s, 1H), 7.59 (s, 1H), 7.51 (dd, J=8.7, 5.5 Hz, 1H), 7.31 (d, J=2.4 Hz, 1H), 7.15 (dd, J=10.1, 2.3 Hz, 1H), 6.85 (ddd, J=9.7, 8.7, 2.3 Hz, 1H), 4.56-4.40 (m, 2H), 4.02-3.85 (m, 1H), 3.32-3.16 (m, 1H), 2.82-2.62 (m, 1H), 2.34 (s, 3H), 2.04 (s, 3H), 2.08-1.72 (m, 4H).

### Compound 96: 6-fluoro-3-(1-(1-(2-methoxyethylsulfonyl)piperidin-4-yl)-1H-pyrazol-4-yl)-1H-indole

### Step 1: 6-fluoro-3-(1-(1-((2-methoxyethyl)sulfonyl)piperidin-4-yl)-1H-pyrazol-4-yl)-1-(phenylsulfonyl)-1H-indole

To a solution of 6-fluoro-1-(phenylsulfonyl)-3-(1-(piperidin-4-yl)-1H-pyrazol-4-yl)-1H-indole hydrochloride (Intermediate 17; 184 mg; 0.40 mmol) and Et₃N (121 mg; 1.20 mmol) in DCM (20 mL) at 0° C. was added 2-methoxyethanesulfonyl chloride (95 mg; 0.60 mmol) dropwise. The reaction mixture was stirred at room temperature overnight, diluted with EtOAc (80 mL), washed with water (30 mL) and brine (30 mL).

The organic layer was dried over anhydrous Na₂SO₄, filtered, concentrated, and purified by preparative TLC (DCM/MeOH=20/1) to afford 100 mg (46%) of the title compound as a yellow oil.

LC-MS: m/z 546.8 [M+H]⁺

### Step 2

Following the general method as outlined in the synthesis of compound 3, starting from 6-fluoro-3-(1-(1-((2-methoxyethyl) sulfonyl)piperidin-4-yl)-1H-pyrazol-4-yl)-1-(phenylsulfonyl)-1H-indole (Step 1; 100 mg; 0.18 mmol), 50 mg (67%) of the title compound was obtained as a white solid after purification by preparative TLC (DCM/MeOH=20/1).

LC-MS: m/z 407.1 [M+H]⁺

¹H NMR (300 MHz, DMSO-d₆) δ [ppm]: 11.17 (s, 1H), 8.18 (s, 1H), 7.81 (s, 1H), 7.80 (dd, J=8.7, 5.4 Hz, 1H), 7.54 (d, J=2.4 Hz, 1H), 7.16 (dd, J=10.1, 2.4 Hz, 1H), 6.90 (ddd, J=9.7, 8.8, 2.4 Hz, 1H), 4.35 (tt, J=11.0, 4.2 Hz, 1H), 3.75-3.65 (m, 2H), 3.68 (t, J=6.0 Hz, 2H), 3.37 (t, J=6.0 Hz, 2H), 3.29 (s, 3H), 3.08-2.96 (m, 2H), 2.20-1.94 (m, 4H).

### Compound 97: 3-(1-(1-(cyclopropylsulfonyl)piperidin-4-yl)-1H-pyrazol-4-yl)-6-fluoro-1H-indole

### Step 1: 3-(1-(1-(cyclopropylsulfonyl)piperidin-4-yl)-1H-pyrazol-4-yl)-6-fluoro-1-(phenylsulfonyl)-1H-indole

To a solution of 6-fluoro-1-(phenylsulfonyl)-3-(1-(piperidin-4-yl)-1H-pyrazol-4-yl)-1H-indole (Intermediate 17; 200 mg; 0.47 mmol) and Et₃N (142 mg; 1.40 mmol) in DCM (10 mL) at 0° C. was added cyclopropanesulfonyl chloride (100 mg; 0.71 mmol) dropwise. The reaction mixture was stirred at room temperature for 30 minutes, concentrated, and purified by reverse phase flash chromatography to afford 136 mg (55%) of the title compound as a white solid.

¹H NMR (300 MHz, DMSO-d₆) δ [ppm]: 8.40 (s, 1H), 8.10 (s, 1H), 8.08-8.01 (m, 2H), 8.01 (s, 1H), 7.94 (dd, J=8.6, 5.2 Hz, 1H), 7.76 (dd, J=9.9, 2.3 Hz, 1H), 7.79-7.67 (m, 1H), 7.65-7.56 (m, 2H), 7.22 (ddd, J=9.7, 8.6, 2.3 Hz, 1H), 4.45-4.34 (m, 1H), 3.79-3.66 (m, 2H), 3.13-3.00 (m, 2H), 2.70-2.58 (m, 1H), 2.22-1.95 (m, 4H), 1.07-0.90 (m, 4H).

### Step 2

Following the general method as outlined in the synthesis of compound 3, starting from 3-(1-(1-(cyclopropylsulfonyl)piperidin-4-yl)-1H-pyrazol-4-yl)-6-fluoro-1-(phenylsulfonyl)-1H-indole (Step 1; 136 mg; 0.26 mmol), 60 mg (60%) of the title compound was obtained as a yellow solid after purification by preparative HPLC.

LC-MS: m/z 389.1 [M+H]⁺

¹H NMR (300 MHz, DMSO-d₆) δ [ppm]: 11.17 (s, 1H), 8.17 (d, J=0.6 Hz, 1H), 8.82 (d, J=0.6 Hz, 1H), 7.80 (dd, J=8.7, 5.4 Hz, 1H), 7.55 (d, J=2.4 Hz, 1H), 7.16 (dd, J=10.1, 2.3 Hz, 1H), 6.90 (ddd, J=9.7, 8.7, 2.3 Hz, 1H), 4.38 (tt, J=11.1, 4.3 Hz, 1H), 3.82-3.67 (m, 2H), 3.14-2.98 (m, 2H), 2.64 (tt, J=7.7, 5.1 Hz, 1H), 2.24-1.96 (m, 4H), 1.10-0.88 (m, 4H).

### Compound 98: 3-(1-(1-(ethylsulfonyl)piperidin-4-yl)-1H-pyrazol-4-yl)-6-fluoro-1H-indole

### Step 1: 3-(1-(1-(ethylsulfonyl)piperidin-4-yl)-1H-pyrazol-4-yl)-6-fluoro-1-(phenylsulfonyl)-1H-indole

To a solution of 6-fluoro-1-(phenylsulfonyl)-3-(1-(piperidin-4-yl)-1H-pyrazol-4-yl)-1H-indole (Intermediate 17; 200 mg; 0.47 mmol), Et₃N (142 mg; 1.40 mmol) in DCM (20 mL) at 0° C. was added ethanesulfonyl chloride (91 mg; 0.71 mmol) dropwise. The reaction mixture was stirred at room temperature overnight, diluted with EtOAc (80 mL), washed with water (30 mL) and brine (30 mL). The organic layer was dried over anhydrous Na₂SO₄, filtered, concentrated, and purified by preparative TLC (DCM/MeOH=20/1) to afford 125 mg (51%) of the title compound as a yellow oil.

LC-MS: m/z 516.8 [M+H]⁺

### Step 2

Following the general method as outlined in the synthesis of compound 3, starting from 3-(1-(1-(ethylsulfonyl)piperidin-4-yl)-1H-pyrazol-4-yl)-6-fluoro-1-(phenylsulfonyl)-1H-indole (Step 1; 125 mg; 0.24 mmol), 67 mg (74%) of the title compound was obtained as a white solid after purification by preparative TLC (DCM/MeOH=20/1).

LC-MS: m/z 377.1 [M+H]⁺

¹H NMR (300 MHz, DMSO-d₆) δ [ppm]: 11.22 (s, 1H), 8.18 (s, 1H), 7.81 (s, 1H), 7.80 (dd, J=8.7, 5.4 Hz, 1H), 7.54 (d, J=2.1 Hz, 1H), 7.16 (dd, J=10.2, 2.1 Hz, 1H), 6.90 (ddd, J=9.7, 8.7, 2.1 Hz, 1H), 4.44-4.31 (m, 1H), 3.78-3.68 (m, 2H), 3.19-2.98 (m, 4H), 2.20-1.93 (m, 4H), 1.24 (t, J=7.3 Hz, 3H).

### Compound 99: 6-fluoro-3-(1-(1-(isopropylsulfonyl)piperidin-4-yl)-1H-pyrazol-4-yl)-1H-indole

### Step 1: 6-fluoro-3-(1-(1-(isopropylsulfonyl)piperidin-4-yl)-1H-pyrazol-4-yl)-1-(phenylsulfonyl)-1H-indole

To a solution of 6-fluoro-1-(phenylsulfonyl)-3-(1-(piperidin-4-yl)-1H-pyrazol-4-yl)-1H-indole (Intermediate 17; 200 mg; 0.47 mmol), Et₃N (0.19 mL; 1.41 mmol) in DCM (10 mL) at 0° C. was added propane-2-sulfonyl chloride (101 mg; 0.71 mmol) dropwise.

The reaction mixture was stirred for 1 hour and concentrated to afford 60 mg (24%) of the title compound as a yellow solid, which was used directly without further purification.

LC-MS: m/z 531.1 [M+H]⁺

### Step 2

Following the general method as outlined in the synthesis of compound 3, starting from 6-fluoro-3-(1-(1-(isopropylsulfonyl)piperidin-4-yl)-1H-pyrazol-4-yl)-1-(phenylsulfonyl)-1H-indole (Step 1; 60 mg; 0.11 mmol), 30 mg (68%) of the title compound was obtained as a white solid after purification by preparative TLC (petroleum ether/EtOAc=1/1).

LC-MS: m/z 391.1 [M+H]⁺.

¹H NMR (300 MHz, DMSO-d₆) δ [ppm]: 11.17 (s, 1H), 8.16 (s, 1H), 7.79 (s, 1H), 7.79 (dd, J=8.6, 5.5 Hz, 1H), 7.53 (d, J=2.4 Hz, 1H), 7.14 (dd, J=10.1, 2.4 Hz, 1H), 6.88 (ddd, J=9.7, 8.6, 2.4 Hz, 1H), 4.38 (tt, J=11.2, 4.0 Hz, 1H), 3.80-3.71 (m, 2H), 3.36 (sept, J=6.8 Hz, 1H), 3.16-3.05 (m, 2H), 2.15-2.05 (m, 2H), 2.04-1.92 (m, 2H), 1.23 (d, J=6.8 Hz, 6H).

### Compound 102: 1-(4-(4-(6-fluoro-1H-indol-3-yl)-1H-pyrazol-1-yl)piperidin-1-yl)prop-2-en-1-one

### Step 1: 1-(4-(4-(6-fluoro-1-(phenylsulfonyl)-1H-indol-3-yl)-1H-pyrazol-1-yl)piperidin-1-yl)-3-(methylsulfonyl)propan-1-one

A mixture of 3-(methylsulfonyl) propionic acid (110 mg; 0.72 mmol), HATU (547 mg; 1.44 mmol) and DIPEA (279 mg; 2.16 mmol) in DMF (3 mL) was stirred at room temperature for 10 minutes before 6-fluoro-1-(phenylsulfonyl)-3-(1-(piperidin-4-yl)-1H-pyrazol-4-yl)-1H-indole (Intermediate 17; 365 mg; 0.86 mmol) was added. The reaction mixture was stirred at room temperature for 2 hours, concentrated, and purified by reverse phase flash chromatography to afford 160 mg (40%) of the title compound as a white solid.

LC-MS: m/z 559.2 [M+H]⁺

### Step 2

To a mixture of 1-(4-(4-(6-fluoro-1-(phenylsulfonyl)-1H-indol-3-yl)-1H-pyrazol-1-yl)piperidin-1-yl)-3-(methylsulfonyl)propan-1-one (Step 1; 160 mg; 0.29 mmol) in MeOH (10 mL) was added a solution of NaOH (150 mg; 3.8 mmol) in water (1.0 mL).

The reaction mixture was stirred at 80° C. overnight. The reaction mixture was concentrated, redissolved with EtOAc (30 mL), and washed with water (15 mL) and brine (15 mL). The organic layer was dried over anhydrous Na₂SO₄, filtered, concentrated, and purified by preparative TLC (DCM/MeOH=20/1) and preparative TLC (EtOAc) to afford 8 mg (8%) of the title compound as a white solid.

LC-MS: m/z 339.1 [M+H]⁺

¹H NMR (400 MHz, MeOH-d₄) δ [ppm]: 8.02 (s, 1H), 7.81 (s, 1H), 7.72 (dd, J=8.6, 5.3 Hz, 1H), 7.41 (s, 1H), 7.10 (dd, J=9.8, 2.3 Hz, 1H), 6.89 (ddd, J=9.7, 8.6, 2.3 Hz, 1H), 6.86 (dd, J=16.8, 10.7 Hz, 1H), 6.25 (dd, J=16.8, 1.9 Hz, 1H), 5.80 (dd, J=10.7, 1.9 Hz, 1H), 4.80-4.70 (m, 1H), 4.60-4.50 (m, 1H), 4.38-4.27 (m, 1H), 3.43-3.33 (m, 1H), 3.05-2.92 (m, 1H), 2.30-2.21 (m, 2H), 2.15-2.00 (m, 2H).

### Compound 103: 1-(4-(4-(6-fluoro-1H-indol-3-yl)-1H-pyrazol-1-yl)piperidin-1-yl)-4-(methylsulfonyl)butan-1-one

### Step 1: 1-(4-(4-(6-fluoro-1-(phenylsulfonyl)-H-indol-3-yl)-H-pyrazol-1-yl)piperidin-1-yl)-4-(methylsulfonyl)butan-1-one

A mixture of 4-(methylsulfonyl)butanoic acid (187 mg; 1.13 mmol), HATU (430 mg; 1.13 mmol) and DIPEA (0.30 mL; 1.81 mmol) in THF (20 mL) was stirred at room temperature for 0.5 hour before 6-fluoro-1-(phenylsulfonyl)-3-(1-(piperidin-4-yl)-1H-pyrazol-4-yl)-1H-indole hydrochloride (Intermediate 17; 260 mg; 0.56 mmol) was added. The reaction mixture was stirred at room temperature for 1 hour and diluted with EtOAc (50 mL) and water (50 mL). The aqueous layer was extracted with EtOAc (50 mL×2). The combined organic layers were washed with brine (10 mL), dried over anhydrous Na₂SO₄, filtered, and concentrated to afford 323 mg (100%) of the title compound as a white solid, which was used directly without further purification.

LC-MS: m/z 573.1 [M+H]⁺

### Step 2

Following the general method as outlined in the synthesis of compound 3, starting from 1-(4-(4-(6-fluoro-1-(phenylsulfonyl)-1H-indol-3-yl)-1H-pyrazol-1-yl)piperidin-1-yl)-4-(methylsulfonyl)butan-1-one (Step 1; 320 mg; 0.56 mmol), 43 mg (18%) of the title compound was obtained as a white solid after purification by silica gel chromatography (DCM/MeOH=50/1-20/1).

LC-MS: m/z 433.1 [M+H]⁺

¹H NMR (300 MHz, DMSO-d₆) δ [ppm]: 11.16 (s, 1H), 8.15 (s, 1H), 7.79 (s, 1H), 7.79 (dd, J=8.7, 5.4 Hz, 1H), 7.54 (d, J=2.3 Hz, 1H), 7.16 (dd, J=10.1, 2.3 Hz, 1H), 6.90 (ddd, J=9.7, 8.7, 2.3 Hz, 1H), 4.56-4.39 (m, 2H), 4.02-3.91 (m, 1H), 3.27-3.15 (m, 1H), 3.15 (t, J=7.5 Hz, 2H), 2.98 (s, 3H), 2.83-2.70 (m, 1H), 2.55 (t, J=7.2 Hz, 2H), 2.15-2.02 (m, 2H), 2.01-1.75 (m, 4H).

### Compound 104: 1-(4-(4-(6-fluoro-1H-indol-3-yl)-1H-pyrazol-1-yl)piperidin-1-yl)-3-hydroxypropan-1-one

### Step 1: 1-(4-(4-(6-fluoro-1-(phenylsulfonyl)-1H-indol-3-yl)-1H-pyrazol-1-yl)piperidin-1-yl)-3-hydroxypropan-1-one

A mixture of 3-hydroxypropionic acid (90 mg; 1.0 mmol), HATU (380 mg; 1.0 mmol) and DIPEA (193 mg; 1.5 mmol) in THF (20 mL) was stirred at room temperature for 10 minutes before 6-fluoro-1-(phenylsulfonyl)-3-(1-(piperidin-4-yl)-1H-pyrazol-4-yl)-1H-indole (Intermediate 17; 200 mg; 0.47 mmol) was added. The reaction mixture was stirred at room temperature overnight, diluted with EtOAc (50 mL), washed with saturated aqueous NaHCO₃ (20 mL), brine (20 mL), dried over anhydrous Na₂SO₄, filtered, concentrated, and purified by preparative TLC (EtOAc/MeOH=20/1) to afford 131 mg (56%) of the title compound as a white solid.

LC-MS: m/z 497.1 [M+H]⁺

### Step 2

Following the general method as outlined in the synthesis of compound 3, starting from 1-(4-(4-(6-fluoro-1-(phenylsulfonyl)-1H-indol-3-yl)-1H-pyrazol-1-yl)piperidin-1-yl)-3-hydroxypropan-1-one (Step 1; 131 mg; 0.26 mmol), 33 mg (35%) of the title compound was obtained as a yellow solid after purification by preparative TLC (EtOAc).

LC-MS: m/z 357.2 [M+H]⁺

¹H NMR (300 MHz, DMSO-d₆) δ [ppm]: 11.17 (s, 1H), 8.15 (s, 1H), 7.85-7.74 (m, 2H), 7.54 (d, J=2.2 Hz, 1H), 7.16 (dd, J=10.0, 2.3 Hz, 1H), 6.96-6.84 (m, 1H), 4.60-4.38 (m, 3H), 4.10-3.95 (m, 1H), 3.66 (dt, J=12.0, 6.3 Hz, 2H), 3.27-3.13 (m, 1H), 2.82-2.68 (m, 1H), 2.54 (t, J=6.3 Hz, 2H), 2.15-1.99 (m, 2H), 1.98-1.70 (m, 2H).

### Compound 105: 1-(4-(4-(6-fluoro-1H-indol-3-yl)-1H-pyrazol-1-yl)piperidin-1-yl)-3-(methylsulfonyl)propan-1-one

To a solution of 3-(methylsulfonyl)propanoic acid (92 mg; 0.60 mmol), HATU (380 mg; 1.00 mmol) in DMF (2 mL) was added DIPEA (194 mg; 1.50 mmol). The reaction mixture was stirred at room temperature for 10 minutes before 6-fluoro-3-(1-(piperidin-4-yl)-1H-pyrazol-4-yl)-1H-indole (Compound 78; 143 mg; 0.50 mmol) was added. The reaction mixture was stirred at room temperature for 2 hours, concentrated, and purified by preparative HPLC to afford 80 mg (38%) of the title compound as a white solid.

LC-MS: m/z 419.1 [M+H]⁺

¹H NMR (400 MHz, DMSO-d₆) δ [ppm]: 11.16 (s, 1H), 8.15 (s, 1H), 7.79 (s, 1H), 7.79 (dd, J=8.7, 5.4 Hz, 1H), 7.54 (d, J=2.3 Hz, 1H), 7.16 (dd, J=10.1, 2.3 Hz, 1H), 6.90 (ddd, J=9.7, 8.7, 2.3 Hz, 1H), 4.52-4.43 (m, 2H), 4.07-3.99 (m, 1H), 3.35 (t, J=7.5 Hz, 2H), 3.29-3.19 (m, 1H), 3.01 (s, 3H), 2.87 (t, J=7.6 Hz, 2H), 2.90-2.77 (m, 1H), 2.16-1.78 (m, 4H).

### Compound 106: 6-fluoro-3-(1-(1-(methylsulfonyl)piperidin-4-yl)-1H-pyrazol-4-yl)-1H-indole

### Step 1: 6-fluoro-3-(1-(1-(methylsulfonyl)piperidin-4-yl)-1H-pyrazol-4-yl)-1-(phenylsulfonyl)-1H-indole

To a solution of 6-fluoro-1-(phenylsulfonyl)-3-(1-(piperidin-4-yl)-1H-pyrazol-4-yl)-1H-indole (Intermediate 17; 200 mg; 0.47 mmol), Et₃N (142 mg; 1.40 mmol) in DCM (15 mL) at 0° C. was added methanesulfonyl chloride (108 mg; 0.94 mmol) dropwise. The reaction mixture was stirred at room temperature for 1 hour and concentrated to afford 230 mg (97%) of the title compound as a yellow solid, which was used directly without further purification.

LC-MS: m/z 503.1 [M+H]⁺

### Step 2

Following the general method as outlined in the synthesis of compound 3, starting from 6-fluoro-3-(1-(1-(methylsulfonyl)piperidin-4-yl)-1H-pyrazol-4-yl)-1-(phenylsulfonyl)-1H-indole (Step 1; 200 mg crude; 0.40 mmol), 90 mg (62%) of the title compound was obtained as a white solid after purification by preparative TLC (EtOAc).

LC-MS: m/z 363.1 [M+H]⁺

¹H NMR (400 MHz, DMSO-d₆) δ [ppm]: 11.17 (s, 1H), 8.18 (s, 1H), 7.81 (s, 1H), 7.81 (dd, J=8.7, 5.5 Hz, 1H), 7.54 (d, J=2.3 Hz, 1H), 7.16 (dd, J=10.1, 2.2 Hz, 1H), 6.90 (ddd, J=9.7, 8.7, 2.2 Hz, 1H), 4.35 (tt, J=11.2, 4.2 Hz, 1H), 3.73-3.65 (m, 2H), 3.01-2.94 (m, 2H), 2.93 (s, 3H), 2.21-2.13 (m, 2H), 2.13-1.99 (m, 2H).

### Compound 107: 6-fluoro-3-(1-(pyridazin-3-yl)-1H-pyrazol-4-yl)-1H-indole

### Step 1: 6-fluoro-1-(phenylsulfonyl)-3-(1-(pyridazin-3-yl)-1H-pyrazol-4-yl)-1H-indole

A mixture of 3-(1-(6-chloropyridazin-3-yl)-1H-pyrazol-4-yl)-6-fluoro-1-(phenylsulfonyl)-1H-indole (Intermediate 44; 180 mg; 0.40 mmol), Pd/C (10 mg) and Et₃N (40 mg; 0.40 mmol) was stirred for 0.5 hour at r.t. under a hydrogen balloon. The reaction mixture was filtered, concentrated, and purified by a silica gel chromatography (petroleum ether/EtOAc=5/1 to 3/1) to afford 100 mg (60%) of the title compound as a white solid.

¹H NMR (400 MHz, DMSO-d₆) δ [ppm]: 9.37 (s, 1H), 9.24 (d, 1H, J=4.4 Hz), 8.55 (s, 1H), 8.42 (s, 1H), 8.27 (d, 1H, J=8.8 Hz), 8.11-8.05 (m, 3H), 7.95-7.92 (m, 1H), 7.80 (d, 1H, J=10.0 Hz), 7.74-7.70 (m, 1H), 7.65-7.61 (m, 1H), 7.25 (t, 1H, J=5.6 Hz).

### Step 2

Following the general method as outlined in the synthesis of compound 70, starting from 6-fluoro-1-(phenylsulfonyl)-3-(1-(pyridazin-3-yl)-1H-pyrazol-4-yl)-1H-indole (Step 1; 83 mg; 0.20 mmol), 50 mg (90%) of the title compound was obtained as a white solid after purification by a silica gel chromatography (petroleum ether/EtOAc=4/1 to 2/1).

¹H NMR (400 MHz, DMSO-d₆) δ [ppm]: 11.3 (br, 1H), 9.20 (d, 1H, J=4.8 Hz), 9.12 (s, 1H), 8.40 (s, 1H), 8.26 (d, 1H, J=8.8 Hz), 7.93-7.89 (m, 2H), 7.85 (s, 1H), 7.22 (d, 1H, J=10.0 Hz), 6.96 (t, 1H, J=9.6 Hz).

### Compound 108: 3-(1-(6-chloropyridazin-3-yl)-1H-pyrazol-4-yl)-6-fluoro-1H-indole

A mixture of 3-(1-(6-chloropyridazin-3-yl)-1H-pyrazol-4-yl)-6-fluoro-1-(phenylsulfonyl)-1H-indole (Intermediate 44; 226 mg; 0.50 mmol) in THF (20 mL) was added NaOH (80 mg; 2.0 mmol). The reaction mixture was stirred at 75° C. for 16 hours, concentrated, and purified by preparative HPLC to afford 45 mg (28%) of the title compound as a yellow solid.

¹H NMR (400 MHz, DMSO-d₆) δ [ppm]: 12.3 (br, 1H), 9.08 (s, 1H), 8.44 (s, 1H), 8.31 (d, 1H, J=8.8 Hz), 8.08 (d, 1H, J=9.2 Hz), 7.94-7.90 (m, 1H), 7.87 (s, 1H), 7.22 (d, 1H, J=10.4 Hz), 7.00-6.84 (m, 1H).

### Compound 109: 6-(4-(6-fluoro-1H-indol-3-yl)-1H-pyrazol-1-yl)pyridazin-3-amine

### Step 1: N-(2,4-dimethoxybenzyl)-6-(4-(6-fluoro-1-(phenylsulfonyl)-1H-indol-3-yl)-1H-pyrazol-1-yl)pyridazin-3-amine

A mixture of 3-(1-(6-chloropyridazin-3-yl)-1H-pyrazol-4-yl)-6-fluoro-1-(phenylsulfonyl)-1H-indole (Intermediate 44; 136 mg; 0.30 mmol) and 2,4-dimethoxybenzylamine (100 mg; 0.60 mmol) in NMP (2 mL) was stirred at 200° C. for 2 hours under a microwave reactor. The reaction mixture was diluted with EtOAc (30 mL), washed water (10 mL), brine (10 mL), dried over anhydrous Na₂SO₄, filtered, concentrated, and purified by a silica gel column chromatography (petroleum ether/EtOAc=4/1 to 2/1) to afford 120 mg (68%) of the title compound as a white solid.

¹H NMR (300 MHz, DMSO-d₆) δ [ppm]: 9.09 (s, 1H), 8.35 (d, 1H, J=13.2 Hz), 8.09 (d, 2H, J=6.9 Hz), 8.02-7.97 (m, 1H), 7.88 (d, 1H, J=9.3 Hz), 7.81 (d, 1H, J=9.9 Hz), 7.70 (d, 1H, J=5.7 Hz), 7.64-7.59 (m, 2H), 7.34-7.30 (m, 1H), 7.24 (d, 1H, J=11.7 Hz), 7.12 (d, 1H, J=9.3 Hz), 4.48 (s, 2H), 3.82 (s, 3H), 3.74 (s, 3H).

### Step 2: N-(2,4-dimethoxybenzyl)-6-(4-(6-fluoro-1H-indol-3-yl)-1H-pyrazol-1-yl)pyridazin-3-amine

Following the general method as outlined in the synthesis of compound 70, starting from N-(2,4-dimethoxybenzyl)-6-(4-(6-fluoro-1-(phenylsulfonyl)-1H-indol-3-yl)-1H-pyrazol-1-yl)pyridazin-3-amine (145 mg; 0.25 mmol), 80 mg (73%) of the title compound was obtained as a white solid after purification by a silica gel column chromatography (petroleum ether/EtOAc=4/1 to 2/1).

¹H NMR (400 MHz, DMSO-d₆) δ [ppm]: 11.34 (br, 1H), 8.85 (s, 1H), 8.21 (s, 1H), 8.23 (d, 1H, J=9.2 Hz), 7.89-7.84 (m, 2H), 7.76 (s, 1H), 7.28-7.25 (m, 2H), 7.22-7.19 (m, 2H), 7.11 (d, 1H, J=10.0 Hz), 6.99-6.94 (m, 1H), 6.58 (s, 1H), 6.50-6.47 (m, 1H), 4.48 (s, 2H), 3.83 (s, 3H), 3.73 (s, 3H).

### Step 3

A mixture of N-(2,4-dimethoxybenzyl)-6-(4-(6-fluoro-1H-indol-3-yl)-1H-pyrazol-1-yl)pyridazin-3-amine (80 mg; 0.18 mmol) and anisole (2.0 mL) in TFA (2.0 mL) was stirred at 120° C. for 2 hours in a microwave reactor. The reaction mixture was concentrated, and purified by a silica gel column chromatography (PE/EtOAc=4/1 to 2/1) to afford 30 mg (57%) of the title compound as a yellow solid.

¹H NMR (400 MHz, DMSO-d₆) δ [ppm]: 11.34 (br, 1H), 8.85 (s, 1H), 8.21 (s, 1H), 8.23 (d, 1H, 11.39 (br, 1H), 8.79 (s, 1H), 8.32 (s, 1H), 8.23 (d, 1H, J=9.2 Hz), 7.88-7.84 (m, 1H), 7.81 (s, 1H), 7.39 (d, 1H, J=10.0 Hz), 7.21 (d, 1H, J=10.0 Hz), 6.99-6.94 (m, 1H).

### Compound 110: 6-fluoro-3-(1-(pyridazin-4-yl)-1H-pyrazol-4-yl)-1H-indole

### Step 1: 3-(1-(3, 6-dichloropyridazin-4-yl)-1H-pyrazol-4-yl)-6-fluoro-1-(phenylsulfonyl)-1H-indole

To a solution of 6-fluoro-1-(phenylsulfonyl)-3-(1H-pyrazol-4-yl)-1H-indole (Intermediate 5; 200 mg; 0.59 mmol) in MeCN (10 mL) was added 3,4,6-trichloropyridazine (97 mg; 0.529 mmol) and K₂CO₃ (146 mg; 1.06 mmol) under nitrogen. The mixture was stirred at 85° C. for 24 hours, diluted with EtOAc (100 mL), washed with water (50 mL×3), brine (50 mL), dried over anhydrous Na₂SO₄, filtered, concentrated, and purified by reversed phase flash chromatography to afford 100 mg (39%) of the title compound a the yellow solid.

LC-MS: m/z 488 [M+H]⁺.

¹H NMR (400 MHz, CDCl₃) δ [ppm]: 8.77 (s, 1H), 8.15 (s, 1H), 8.12 (s, 1H), 7.96 (s, 1H), 7.94 (s, 1H), 7.80 (dd, J=10.0, 2.4 Hz, 1H), 7.76 (s, 1H), 7.63-7.56 (m, 2H), 7.54-7.48 (m, 2H), 7.12 (td, J=8.8, 2.4 Hz, 1H).

### Step 2: 6-fluoro-1-(phenylsulfonyl)-3-(1-(pyridazin-4-yl)-1H-pyrazol-4-yl)-1H-indole

A mixture of 3-(1-(3,6-dichloropyridazin-4-yl)-1H-pyrazol-4-yl)-6-fluoro-1-(phenylsulfonyl)-1H-indole (Step 1; 80 mg; 0.16 mmol) and Pd/C (30 mg; 10%) in MeOH (15 mL) was stirred for 1 hour under a hydrogen balloon. The reaction mixture was filtered and concentrated to afford 50 mg (64%) of the title compound as a yellow solid, which was used directly without further purification.

LC-MS: m/z 420 [M+H]⁺.

### Step 3

Following the general method as outlined in the synthesis of compound 70, starting from 6-fluoro-1-(phenylsulfonyl)-3-(1-(pyridazin-4-yl)-1H-pyrazol-4-yl)-1H-indole (Step 2; 50 mg; 0.12 mmol), 13 mg (39%) of the title compound was obtained as a white solid after purification by reverse phase flash chromatography.

¹H NMR (400 MHz, DMSO-d₆) δ [ppm]: 11.51 (s, 1H), 9.95 (d, J=2.8 Hz, 1H), 9.28 (d, J=5.2 Hz, 1H), 9.16 (s, 1H), 8.40 (s, 1H), 8.20-8.17 (m, 1H), 8.07-8.03 (m, 1H), 7.83 (d, J=2.4 Hz, 1H), 7.23 (dd, J=10, 2.4 Hz, 1H), 6.99 (dt, J=8.8, 2.4 Hz 1H).

### Compound 111: 6-fluoro-3-(1-(pyridin-2-yl)-1H-pyrazol-4-yl)-1H-indole

Following the general method as outlined in the synthesis of compound 70, starting from 6-fluoro-1-(phenylsulfonyl)-3-(1-(pyridin-2-yl)-1H-pyrazol-4-yl)-1H-indole (Intermediate 15; 230 mg; 0.55 mmol), 17.2 mg (11%) of the title compound was obtained as a yellow solid after purification by preparative HPLC.

¹H NMR (400 MHz, MeOH-d₄) δ [ppm]: 8.75 (s, 1H), 8.36 (d, J=4.9 Hz, 1H), 7.99 (s, 1H), 7.85 (m, 2H), 7.69 (dd, J=8.7, 5.2 Hz, 1H), 7.44 (s, 1H), 7.20 (dd, J=8.5, 3.2 Hz, 1H), 7.02 (dd, J=9.8, 2.3 Hz, 1H), 6.82 (td, J=9.3, 2.3 Hz, 1H). m.p. 171.3-172.5° C.

### Compound 112: 6-fluoro-3-(1-(pyridin-3-yl)-1H-pyrazol-4-yl)-1H-indole

Following the general method as outlined in the synthesis of compound 70, starting from 6-fluoro-1-(phenylsulfonyl)-3-(1-(pyridin-3-yl)-1H-pyrazol-4-yl)-1H-indole (Intermediate 12; 362 mg; 0.87 mmol), 63 mg (26%) of the title compound was obtained as a yellow solid after purification by preparative HPLC.

¹H NMR (400 MHz, DMSO-d₆) δ [ppm]: 11.38 (s, 1H), 9.25 (d, J=2.2 Hz, 1H), 8.98 (s, 1H), 8.53 (d, J=4.2 Hz, 1H), 8.36 (d, J=8.0 Hz, 1H), 8.25 (s, 1H), 8.03 (dd, J=8.6, 5.5 Hz, 1H), 7.77 (d, J=1.7 Hz, 1H), 7.57 (dd, J=8.2, 4.7 Hz, 1H), 7.23 (dd, J=10.0, 2.0 Hz, 1H), 6.98 (m, 1H). m.p. 203.0-204.6° C.

### Compound 113: 6-fluoro-3-(1-(pyridin-4-yl)-1H-pyrazol-4-yl)-1H-indole

Following the general method as outlined in the synthesis of compound 70, starting from 6-fluoro-1-(phenylsulfonyl)-3-(1-(pyridin-4-yl)-1H-pyrazol-4-yl)-1H-indole (Intermediate 10; 363 mg; 0.87 mmol), 146 mg (60%) of the title compound was obtained as a yellow solid after purification by preparative HPLC.

¹H NMR (400 MHz, DMSO-d₆) δ [ppm]: 11.40 (d, 1H, J=1.6 Hz), 9.04 (s, 1H), 8.66 (d, 2H, J=4.7 Hz), 8.30 (s, 1H), 8.02 (m, 3H), 7.80 (s, 1H), 7.23 (d, 1H, J=9.5 Hz), 6.99 (t, 1H, J=8.8 Hz). m.p.>280° C.

### Compound 114: trans-4-(4-(6-fluoro-1H-indol-3-yl)-1H-pyrazol-1-yl)-N,N-dimethylcyclohexanecarboxamide

Following the general method as outlined in the synthesis of compound 67, starting from 4-(4-(6-fluoro-1H-indol-3-yl)-1H-pyrazol-1-yl)cyclohexanecarboxylic acid (as a mixture of cis and trans isomers) (Intermediate 52; 180 mg; 0.55 mmol) and dimethylamine (0.50 mL; 1.00 mmol; 2 M in THF), 40 mg (21%) of the title compound was obtained as a yellow solid after purification by preparative TLC (DCM/MeOH=20/1) and preparative HPLC.

LC-MS: m/z 355.1 [M+H]⁺

¹H NMR (300 MHz, MeOH-d₄) δ [ppm]: 7.95 (s, 1H), 7.76 (s, 1H), 7.68 (dd, J=8.8, 5.3 Hz, 1H), 7.37 (s, 1H), 7.07 (dd, J=9.9, 2.4 Hz, 1H), 6.86 (ddd, J=9.7, 8.8, 2.4 Hz, 1H), 4.24 (tt, J=11.7, 4.0 Hz, 1H), 3.15 (s, 3H), 2.94 (s, 3H), 2.81 (tt, J=11.8, 3.3 Hz, 1H), 2.27-2.16 (m, 2H), 2.05-1.89 (m, 4H), 1.80-1.62 (m, 2H).

### Compound 115: cis-4-(4-(6-fluoro-1H-indol-3-yl)-1H-pyrazol-1-yl)cyclohexanecarboxamide

16 mg (4%) of the title compound was obtained as a white solid after purification by preparative TLC (DCM/MeOH=15/1) and preparative HPLC in the synthesis of compound 67.

LC-MS: m/z 325.1 [M−H]⁻

¹H NMR (400 MHz, MeOH-d₄) δ [ppm]: 7.94 (s, 1H), 7.76 (s, 1H), 7.68 (dd, J=8.4, 5.5 Hz, 1H), 7.37 (s, 1H), 7.11-7.05 (m, 1H), 6.90-6.82 (m, 1H), 4.33-4.24 (m, 1H), 2.40-2.25 (m, 1H), 2.40-2.25 (m, 2H), 2.15-1.94 (m, 4H), 1.84-1.70 (m, 2H).

### Compound 116: cis-4-(4-(6-fluoro-1H-indol-3-yl)-1H-pyrazol-1-yl)-N-methylcyclohexanecarboxamide

25 mg (13%) of the title compound was obtained as a white solid after purification by preparative TLC (DCM/MeOH=20/1) and preparative HPLC in the synthesis of compound 118.

LC-MS: m/z 341.1 [M+H]⁺

¹H NMR (300 MHz, MeOH-d₄) δ [ppm]: 7.97 (s, 1H), 7.76 (s, 1H), 7.69 (dd, J=8.8, 5.3 Hz, 1H), 7.39 (s, 1H), 7.08 (dd, J=9.9, 2.4 Hz, 1H), 6.86 (ddd, J=9.7, 8.8, 2.4 Hz, 1H), 4.31 (tt, J=11.7, 4.0 Hz, 1H), 2.73 (s, 3H), 2.55-2.45 (m, 1H), 2.41-2.26 (m, 2H), 2.11-1.93 (m, 4H), 1.82-1.68 (m, 2H).

### Compound 117: cis-4-(4-(6-fluoro-1H-indol-3-yl)-1H-pyrazol-1-yl)-N,N-dimethylcyclohexanecarboxamide

15 mg (8%) of the title compound was obtained as a white solid after purification by preparative TLC (DCM/MeOH=20/1) and preparative HPLC in the synthesis of compound 114.

LC-MS: m/z 355.1 [M+H]⁺

¹H NMR (300 MHz, MeOH-d₄) δ [ppm]: 7.98 (s, 1H), 7.76 (s, 1H), 7.69 (dd, J=8.8, 5.3 Hz, 1H), 7.38 (s, 1H), 7.09 (dd, J=10.0, 2.4 Hz, 1H), 6.86 (ddd, J=9.8, 8.8, 2.4 Hz, 1H), 4.31 (tt, J=7.4, 3.8 Hz, 1H), 3.08 (s, 3H), 2.97-2.87 (m, 1H), 2.90 (s, 3H), 2.54-2.39 (m, 2H), 2.06-1.82 (m, 4H), 1.77-1.64 (m, 2H).

### Compound 118: trans-4-(4-(6-fluoro-1H-indol-3-yl)-1H-pyrazol-1-yl)-N-methylcyclohexanecarboxamide

Following the general method as outlined in the synthesis of compound 67, starting from 4-(4-(6-fluoro-1H-indol-3-yl)-1H-pyrazol-1-yl)cyclohexanecarboxylic acid (as a mixture of cis and trans isomers) (Intermediate 52; 180 mg; 0.55 mmol) and methylamine (0.50 mL; 1.00 mmol; 2 M in THF), 48 mg (25%) of the title compound was obtained as a yellow solid after purification by preparative TLC (DCM/MeOH=20/1) and preparative HPLC.

LC-MS: m/z 341.1 [M+H]⁺

¹H NMR (300 MHz, DMSO-d₆) δ [ppm]: 11.15 (brs, 1H), 8.11 (s, 1H), 7.81-7.70 (m, 2H), 7.76 (s, 1H), 7.52 (d, J=2.4 Hz, 1H), 7.16 (dd, J=10.1, 2.4 Hz, 1H), 6.90 (ddd, J=9.7, 8.8, 2.4 Hz, 1H), 4.16 (tt, J=11.6, 3.8 Hz, 1H), 2.58 (d, J=4.6 Hz, 3H), 2.18 (tt, J=11.9, 3.3 Hz, 1H), 2.16-2.05 (m, 2H), 1.95-1.72 (m, 4H), 1.67-1.47 (m, 2H).

### Compound 119: 4-(4-(6-fluoro-1H-indol-3-yl)-1H-pyrazol-1-yl)piperidine-1-carboxamide

### Step 1: 4-(4-(6-fluoro-1-(phenylsulfonyl)-1H-indol-3-yl)-1H-pyrazol-1-yl)piperidine-1-carboxamide

To the mixture of 6-fluoro-1-(phenylsulfonyl)-3-(1-(piperidin-4-yl)-1H-pyrazol-4-yl)-1H-indole hydrochloride (Intermediate 17; 200 mg; 0.43 mmol) in DCM (10 mL) was added Et₃N (214 mg; 2.11 mmol) and trimethylsilyl isocyanate (92 mg; 0.80 mmol). After 3 hours, another portion of trimethylsilyl isocyanate (50 mg; 0.43 mmol) was added and the reaction mixture was stirred at room temperature overnight. The reaction mixture was concentrated to afford 453 mg (>100%) of the title compound as a white solid, which was used directly without further purification.

LC-MS: m/z 468.1 [M+H]⁺

### Step 2

Following the general method as outlined in the synthesis of compound 3, starting from 4-(4-(6-fluoro-1-(phenylsulfonyl)-1H-indol-3-yl)-1H-pyrazol-1-yl)piperidine-1-carboxamide (Step 1; 450 mg crude; 0.43 mmol), 37 mg (26%) of the title compound was obtained as a white solid after purification by preparative HPLC.

LC-MS: m/z 328.1 [M+H]⁺

¹H NMR (400 MHz, DMSO-d₆) δ [ppm]: 11.17 (s, 1H), 8.15 (s, 1H), 7.80 (dd, J=8.6, 5.5 Hz, 1H), 7.79 (s, 1H), 7.54 (d, J=2.0 Hz, 1H), 7.16 (dd, J=10.0, 2.4 Hz, 1H), 6.89 (ddd, J=9.8, 8.6, 2.4 Hz, 1H), 6.02 (brs, 2H), 4.36 (tt, J=11.3, 4.0 Hz, 1H), 4.12-4.02 (m, 2H), 2.91-2.79 (m, 2H), 2.05-1.96 (m, 2H), 1.92-1.78 (m, 2H).

### Compound 120: methyl 4-(4-(6-fluoro-1H-indol-3-yl)-1H-pyrazol-1-yl)piperidine-1-carboxylate

To a solution of 6-fluoro-3-(1-(piperidin-4-yl)-1H-pyrazol-4-yl)-1H-indole hydrochloride (compound 78; 300 mg; 0.94 mmol) and Et₃N (237 mg; 2.34 mmol) in DCM (10 mL) was added methyl chloroformate (115 mg; 1.22 mmol) at −10° C. dropwise. The reaction mixture was stirred at 5-10° C. for 3 hours and quenched with aqueous NaOH (30 mL). The organic layer was separated and the aqueous layer was extracted with DCM (50 mL×2) and EtOAc (50 mL×2). The combined organic layers were washed with brine, dried over anhydrous Na₂SO₄, filtered, concentrated and purified by preparative HPLC to afford 100 mg (31%) of the title compound as a white solid.

LC-MS: m/z 343.1 [M+H]⁺

¹H NMR (400 MHz, DMSO-d₆) δ [ppm]: 11.18 (s, 1H), 8.17 (s, 1H), 7.80 (dd, J=8.6, 5.5 Hz, 1H), 7.79 (s, 1H), 7.54 (d, J=2.0 Hz, 1H), 7.16 (dd, J=10.0, 2.3 Hz, 1H), 6.90 (ddd, J=9.8, 8.6, 2.3 Hz, 1H), 4.41 (tt, J=11.5, 3.9 Hz, 1H), 4.21-3.99 (m, 2H), 3.63 (s, 3H), 3.16-2.89 (m, 2H), 2.15-2.00 (m, 2H), 1.97-1.83 (m, 2H).

### Compound 121: 2-amino-1-(4-(4-(6-fluoro-1H-indol-3-yl)-1H-pyrazol-1-yl)piperidin-1-yl)ethanone

### Step 1: tert-butyl (2-(4-(4-(6-fluoro-1-(phenylsulfonyl)-1H-indol-3-yl)-1H-pyrazol-1-yl)piperidin-1-yl)-2-oxoethyl)carbamate

To a solution of 2-((tert-butoxycarbonyl)amino)acetic acid (229 mg; 1.31 mmol) and DIPEA (449 mg; 3.47 mmol) in DMF (4 mL) was added HATU (498 mg; 1.31 mmol). The reaction mixture was stirred at room temperature for 10 minutes before a pre-mixed solution of 6-fluoro-1-(phenylsulfonyl)-3-(1-(piperidin-4-yl)-1H-pyrazol-4-yl)-1H-indole hydrochloride (Intermediate 17; 400 mg; 0.87 mmol) and DIPEA (113 mg; 0.87 mmol) in DMF (1 mL) was added dropwise. The reaction mixture was stirred at room temperature for 30 minutes and added dropwise to H₂O (50 mL) with vigorous stirring. The resulting precipitate was collected by vacuum filtration to afford 484 mg (96%) of the title compound as a white solid, which was used directly without further purification.

LC-MS: m/z 525.8 [M+H−tBu]⁺

### Step 2: tert-butyl (2-(4-(4-(6-fluoro-1H-indol-3-yl)-1H-pyrazol-1-yl)piperidin-1-yl)-2-oxoethyl)carbamate

Following the general method as outlined in the synthesis of compound 3, starting from tert-butyl (2-(4-(4-(6-fluoro-1-(phenylsulfonyl)-1H-indol-3-yl)-1H-pyrazol-1-yl)piperidin-1-yl)-2-oxoethyl)carbamate (Step 1; 480 mg; 0.83 mmol), 230 mg (63%) of the title compound was obtained as a white solid, which was used directly without further purification.

LC-MS: m/z 442.1 [M+H]⁺

### Step 3

Following the general method as outlined in Step 2 of Intermediate 14, starting from tert-butyl (2-(4-(4-(6-fluoro-1H-indol-3-yl)-1H-pyrazol-1-yl)piperidin-1-yl)-2-oxoethyl)carbamate (Step 2; 230 mg; 0.52 mmol), 100 mg (56%) of the title compound was obtained as a white solid after purification by preparative HPLC.

LC-MS: m/z 342.1 [M+H]⁺

¹H NMR (400 MHz, DMSO-d₆) δ [ppm]: 11.18 (s, 1H), 8.16 (s, 1H), 7.82-7.76 (m, 1H), 7.79 (s, 1H), 7.54 (d, J=2.2 Hz, 1H), 7.16 (dd, J=10.1, 2.3 Hz, 1H), 6.90 (ddd, J=9.9, 8.6, 2.3 Hz, 1H), 4.56-4.41 (m, 2H), 3.97-3.80 (m, 1H), 3.40 (s, 2H), 3.21-3.08 (m, 1H), 2.86-2.73 (m, 1H), 2.22 (brs, 2H), 2.12-2.03 (m, 2H), 2.01-1.75 (m, 2H).

### Compound 122: (S)-2-amino-1-(4-(4-(6-fluoro-1H-indol-3-yl)-1H-pyrazol-1-yl)piperidin-1-yl)-3-methylbutan-1-one

### Step 1: (S)-tert-butyl (1-(4-(4-(6-fluoro-1H-indol-3-yl)-1H-pyrazol-1-yl)piperidin-1-yl)-3-methyl-1-oxobutan-2-yl)carbamate

To a solution of Boc-L-Valine (254 mg; 1.17 mmol) and DIPEA (402 mg; 3.11 mmol) in DMF (4 mL) was added HATU (445 mg; 1.17 mmol). The reaction mixture was stirred at room temperature for 10 minutes before 6-fluoro-3-(1-(piperidin-4-yl)-1H-pyrazol-4-yl)-1H-indole (compound 78; 222 mg; 0.78 mmol) in DMF (1 mL) was added dropwise. The reaction mixture was stirred at room temperature for 30 minutes and added dropwise to water (50 mL) with vigorous stirring. The resulting precipitate was collected by vacuum filtration to afford 453 mg (>100%) of the title compound as a white solid, which was used directly without further purification.

LC-MS: m/z 484.0 [M+H]⁺

### Step 2

Following the general method as outlined in Step 2 of Intermediate 14, starting from (S)-tert-butyl (1-(4-(4-(6-fluoro-1H-indol-3-yl)-1H-pyrazol-1-yl)piperidin-1-yl)-3-methyl-1-oxobutan-2-yl)carbamate (Step 1; 450 mg crude; 0.78 mmol), 80 mg (27%) of the title compound was obtained as a white solid after purification by preparative HPLC.

LC-MS: m/z 384.2 [M+H]⁺

¹H NMR (400 MHz, DMSO-d₆) δ [ppm]: 11.18 (s, 1H), 8.15 (s, 1H), 7.82-7.76 (m, 1H), 7.80 (s, 1H), 7.54 (s, 1H), 7.16 (dd, J=10.1, 2.3 Hz, 1H), 6.95-6.87 (m, 1H), 4.62-4.41 (m, 2H), 4.12-4.01 (m, 1H), 3.57-3.46 (m, 1H), 3.26-3.14 (m, 1H), 2.85-2.72 (m, 1H), 2.20-1.65 (m, 7H), 0.95-0.88 (m, 3H, possible rotamer), 0.86-0.76 (m, 3H, possible rotamer).

[α]²⁰_{D}=−7.0 (c=0.002, MeOH)

### Compound 123: 3-amino-1-(4-(4-(6-fluoro-1H-indol-3-yl)-1H-pyrazol-1-yl)piperidin-1-yl)propan-1-one

### Step 1: tert-butyl (3-(4-(4-(6-fluoro-1H-indol-3-yl)-1H-pyrazol-1-yl)piperidin-1-yl)-3-oxopropyl)carbamate

Following the general method as outlined in Step 1 of the synthesis of compound 122, starting from 3-((tert-butoxycarbonyl)amino)propanoic acid (179 mg; 0.95 mmol) and 6-fluoro-3-(1-(piperidin-4-yl)-1H-pyrazol-4-yl)-1H-indole (compound 78; 177 mg; 0.62 mmol), 180 mg (63%) of the title compound was obtained as a white solid, which was used directly without further purification.

LC-MS: m/z 456.0 [M+H]⁺

### Step 2

Following the general method as outlined in Step 2 of Intermediate 14, starting from tert-butyl (3-(4-(4-(6-fluoro-1H-indol-3-yl)-1H-pyrazol-1-yl)piperidin-1-yl)-3-oxopropyl)carbamate (Step 1; 180 mg; 0.40 mmol), 60 mg (43%) of the title compound was obtained as a white solid after purification by preparative HPLC.

LC-MS: m/z 356.2 [M+H]⁺

¹H NMR (400 MHz, DMSO-d₆) δ [ppm]: 11.18 (s, 1H), 8.16 (s, 1H), 7.83-7.76 (m, 1H), 7.79 (s, 1H), 7.54 (s, 1H), 7.16 (dd, J=10.1, 2.2 Hz, 1H), 6.90 (ddd, J=9.7, 8.8, 2.2 Hz, 1H), 4.56-4.40 (m, 2H), 4.04-3.93 (m, 1H), 3.25-3.13 (m, 2H), 2.82-2.69 (m, 2H), 2.56-2.43 (m, 2H), 2.13-2.01 (m, 2H), 2.01-1.74 (m, 2H).

### Compound 124: (R)-2-amino-1-(4-(4-(6-fluoro-1H-indol-3-yl)-1H-pyrazol-1-yl)piperidin-1-yl)-3-methylbutan-1-one

### Step 1: (R)-tert-butyl (1-(4-(4-(6-fluoro-1H-indol-3-yl)-1H-pyrazol-1-yl)piperidin-1-yl)-3-methyl-1-oxobutan-2-yl)carbamate

Following the general method as outlined in Step 1 of the synthesis of compound 122, starting from Boc-D-Valine (254 mg; 1.17 mmol) and 6-fluoro-3-(1-(piperidin-4-yl)-1H-pyrazol-4-yl)-1H-indole (compound 78; 222 mg; 0.78 mmol), 248 mg (66%) of the title compound was obtained as a white solid, which was used directly without further purification.

LC-MS: m/z 484.0 [M+H]⁺

### Step 2

Following the general method as outlined in Step 2 of Intermediate 14, starting from (R)-tert-butyl (1-(4-(4-(6-fluoro-1H-indol-3-yl)-1H-pyrazol-1-yl)piperidin-1-yl)-3-methyl-1-oxobutan-2-yl)carbamate (Step 1; 248 mg; 0.51 mmol), 172 mg (87%) of the title compound was obtained as a white solid after purification by preparative HPLC.

LC-MS: m/z 384.2 [M+H]⁺

¹H NMR (300 MHz, DMSO-d₆) δ [ppm]: 11.18 (s, 1H), 8.15 (s, 1H), 7.84-7.75 (m, 1H), 7.80 (s, 1H), 7.54 (s, 1H), 7.16 (dd, J=10.1, 2.3 Hz, 1H), 6.96-6.85 (m, 1H), 4.62-4.40 (m, 2H), 4.13-4.00 (m, 1H), 3.57-3.45 (m, 1H), 3.27-3.13 (m, 1H), 2.86-2.69 (m, 1H), 2.20-1.60 (m, 7H), 0.95-0.87 (m, 3H, possible rotamer), 0.86-0.75 (m, 3H, possible rotamer).

### Compound 125: 4-(4-(6-fluoro-1H-indol-3-yl)-1H-pyrazol-1-yl)-N-methylpiperidine-1-carboxamide

A mixture of methylamine (0.70 mL; 1.40 mmol; 2 M in THF) and CDI (227 mg; 1.40 mmol) in THF (10 mL) was stirred at room temperature for 1.5 hours and added to a solution of 6-fluoro-3-(1-(piperidin-4-yl)-1H-pyrazol-4-yl)-1H-indole hydrochloride (compound 78; 300 mg; 0.94 mmol) and Et₃N (157 mg; 1.55 mmol) in THF (20 mL) dropwise. The reaction mixture was stirred at room temperature overnight, concentrated, and purified by preparative TLC (EtOAc/MeOH=8/1) to afford 60 mg (19%) of the title compound as a white solid.

LC-MS: m/z 342.2 [M+H]⁺

¹H NMR (400 MHz, DMSO-d₆) δ [ppm]: 11.17 (s, 1H), 8.15 (s, 1H), 7.80 (dd, J=8.6, 5.5 Hz, 1H), 7.79 (s, 1H), 7.54 (d, J=2.3 Hz, 1H), 7.16 (dd, J=10.0, 2.3 Hz, 1H), 6.89 (ddd, J=9.8, 8.6, 2.3 Hz, 1H), 6.51 (q, J=4.2 Hz, 1H) 4.38 (tt, J=11.4, 4.0 Hz, 1H), 4.13-4.02 (m, 2H), 2.91-2.79 (m, 2H), 2.59 (d, J=4.2 Hz, 3H), 2.05-1.95 (m, 2H), 1.91-1.77 (m, 2H).

### Compound 126: (S)-2-amino-1-(4-(4-(6-fluoro-1H-indol-3-yl)-1H-pyrazol-1-yl)piperidin-1-yl)propan-1-one

### Step 1: (S)-tert-butyl (1-(4-(4-(6-fluoro-1H-indol-3-yl)-1H-pyrazol-1-yl)piperidin-1-yl)-1-oxopropan-2-yl)carbamate

Following the general method as outlined in Step 1 of the synthesis of compound 122, starting from Boc-L-Alanine (221 mg; 1.17 mmol) and 6-fluoro-3-(1-(piperidin-4-yl)-1H-pyrazol-4-yl)-1H-indole (compound 78; 222 mg; 0.78 mmol), 200 mg (56%) of the title compound was obtained as a yellow solid, which was used directly without further purification.

LC-MS: m/z 455.9 [M+H]⁺

### Step 2

Following the general method as outlined in Step 2 of Intermediate 14, starting from (S)-tert-butyl (1-(4-(4-(6-fluoro-1H-indol-3-yl)-1H-pyrazol-1-yl)piperidin-1-yl)-1-oxopropan-2-yl)carbamate (Step 1; 200 mg; 0.44 mmol), 120 mg (77%) of the title compound was obtained as a white solid after purification by preparative HPLC.

LC-MS: m/z 356.1 [M+H]⁺

¹H NMR (400 MHz, DMSO-d₆) δ [ppm]: 11.18 (s, 1H), 8.16 (s, 1H), 7.85-7.75 (m, 1H), 7.80 (s, 1H), 7.54 (s, 1H), 7.21-7.12 (m, 1H), 6.96-6.86 (m, 1H), 4.58-4.40 (m, 2H), 4.13-3.98 (m, 1H), 3.87-3.74 (m, 1H), 3.27-3.11 (m, 1H), 2.86-2.69 (m, 1H), 2.25-1.73 (m, 6H), 1.18-1.02 (m, 3H, possible rotamer)

### II. Biology Examples

### II.1. Assay for TDO2 Enzymatic Activity Determination

The compounds of the present invention inhibit the enzymatic activity of human TDO2.

To measure the TDO2 activity, the reaction mixture contained (final concentrations) potassium phosphate buffer (50 mM, pH 7.5), ascorbic acid (0.25 M), methylene blue (0.125 μM), catalase (40 units/mL, from bovine liver, Sigma), and human recombinant TDO2 enzyme (prepared as described in Dolusic et al. J. Med. Chem.; 2011, 54, 5320-5334; 0.9 μg) without or with the compounds of the present invention at the indicated concentrations (total volume 112.5 μL). The reaction was initiated by the addition of 37.5 μL of L-Trp (final concentration 1 mM) at room temperature. The reaction was conducted at room temperature during one hour and stopped by the addition of 30 μL of 30% (w/v) trichloroacetic acid.

To convert N-formylkynurenine into kynurenine, the reaction mixture was incubated at 65° C. for 30 min. Then 150 μL of the reaction mixture was mixed with 120 μL of 2.5% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism software (GraphPad Software, Inc.).

The biological activity of representative Compounds is summarized in the following table (*: 10 μM<IC₅₀<100 μM; **: 1 μM<IC₅₀<10 μM; ***: IC₅₀<1 μM):

| Compound number | IC₅₀ |
|---|---|
| 2 | *** |
| 3 | ** |
| 4 | ** |
| 5 | ** |
| 6 | *** |
| 7 | *** |
| 8 | ** |
| 9 | ** |
| 13 | ** |
| 14 | ** |
| 15 | ** |
| 16 | ** |
| 17 | * |
| 18 | ** |
| 19 | ** |
| 20 | ** |
| 21 | ** |
| 22 | ** |
| 23 | ** |
| 24 | *** |
| 25 | ** |
| 26 | *** |
| 27 | ** |
| 28 | * |
| 29 | ** |
| 30 | ** |
| 31 | ** |
| 32 | ** |
| 33 | * |
| 35 | ** |
| 36 | ** |
| 37 | ** |
| 38 | ** |
| 39 | ** |
| 40 | ** |
| 41 | * |
| 42 | ** |
| 43 | *** |
| 44 | ** |
| 45 | ** |
| 46 | *** |
| 47 | *** |
| 48 | *** |
| 49 | ** |
| 50 | * |
| 51 | ** |
| 52 | *** |
| 53 | *** |
| 54 | ** |
| 55 | * |
| 56 | *** |
| 57 | ** |
| 58 | ** |
| 59 | ** |
| 60 | ** |
| 61 | ** |
| 62 | ** |
| 63 | ** |
| 64 | ** |
| 65 | * |
| 66 | * |
| 68 | ** |
| 69 | ** |
| 70 | *** |
| 71 | *** |
| 72 | * |
| 73 | *** |
| 74 | ** |
| 75 | ** |
| 77 | * |
| 78 | ** |
| 79 | ** |
| 80 | * |
| 81 | ** |
| 82 | ** |
| 83 | ** |
| 84 | ** |
| 85 | ** |
| 86 | ** |
| 87 | ** |
| 88 | ** |
| 89 | ** |
| 90 | ** |
| 91 | ** |
| 92 | ** |
| 94 | * |
| 95 | ** |
| 96 | ** |
| 97 | ** |
| 98 | ** |
| 99 | ** |
| 102 | ** |
| 103 | * |
| 104 | ** |
| 105 | ** |
| 106 | ** |
| 107 | ** |
| 108 | * |
| 109 | ** |
| 110 | ** |
| 111 | *** |
| 112 | *** |
| 113 | *** |

### II.2. Cellular Assays for TDO2 Activity Determination

II.2.a hTDO2-Overexpressing P815 Cells

The compounds of the present invention inhibit the activity of human TDO2 in cells.

The assay was performed in 96-well flat bottom plates seeded with murine mastocytoma P815 cells overexpressing hTDO2 (prepared as described in Pilotte et al., PNAS, 2012, 109(7), 2497-2502), at a concentration of 5×10⁴ cells/well in a final volume of 200 μL. To determine TDO or IDO activity, the cells were incubated overnight at 37° C. at 5% CO₂ in IMDM (Invitrogen) supplemented with 2% FBS and 2% penicillin/streptomycin in the presence of the compounds of the present invention, at different concentrations.

The plates were then centrifuged 5 min at 1000 rpm, and 100 μL of the supernatant were collected in a conical plate, 30 uL of TCA 30% were added and a further centrifugated at 3000×g for 10 minutes. 100 μL of the supernatant were collected in a flat bottomed plate and 100 μL of 2% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism software (GraphPad Software, Inc.).

The biological activity of representative Compounds is summarized in the following table (*: 10 μM<IC₅₀<100 μM; **: 1 μM<IC₅₀<10 μM; ***: IC₅₀<1 μM):

| Compound number | IC₅₀ |
|---|---|
| 2 | *** |
| 6 | *** |
| 7 | *** |
| 19 | *** |
| 20 | ** |
| 22 | ** |
| 24 | ** |
| 25 | *** |
| 26 | *** |
| 27 | *** |
| 29 | *** |
| 31 | *** |
| 35 | *** |
| 36 | *** |
| 37 | *** |
| 38 | ** |
| 39 | ** |
| 40 | ** |
| 43 | *** |
| 44 | *** |
| 46 | *** |
| 47 | ** |
| 48 | *** |
| 51 | *** |
| 52 | *** |
| 53 | *** |
| 54 | ** |
| 58 | ** |
| 59 | ** |
| 60 | ** |
| 61 | *** |
| 62 | ** |
| 67 | ** |
| 68 | *** |
| 69 | ** |
| 70 | *** |
| 71 | *** |
| 73 | *** |
| 74 | *** |
| 75 | *** |
| 77 | ** |
| 78 | *** |
| 85 | ** |
| 89 | *** |
| 91 | *** |
| 103 | ** |
| 104 | *** |
| 105 | *** |
| 106 | *** |
| 107 | *** |
| 109 | *** |
| 110 | ** |
| 111 | *** |
| 112 | *** |
| 113 | *** |

II.2.b A172 Cells

The compounds of the present invention inhibit the activity of human TDO2 in cells that constitutively express TDO2, such as A172 cells.

The assay was performed in 96-well flat bottom plates seeded with murine mastocytoma A172 cells, naturally expressing hTDO2 (prepared as described in Tilman et al., Mol Cancer, 2007, 17(6), 80), at a concentration of 1.25×10⁴ cells/well in a final volume of 200 μL. To determine TDO, the cells were incubated overnight at 37° C. at 5% CO₂ in IMDM (Invitrogen) supplemented with 2% FBS and 2% penicillin/streptomycin in the presence of the compounds of the present invention, at different concentrations.

The plates were then centrifuged 5 min at 1000 rpm, and 100 μL of the supernatant were collected in a conical plate, 30 uL of TCA 30% were added and a further centrifugated at 3000×g for 10 minutes. 100 μL of the supernatant were collected in a flat bottomed plate and 100 μL of 2% (w/v) 4-(dimethylamino)-benzaldehyde in acetic acid and incubated for 5 min at room temperature. Kynurenine concentrations were determined by measuring the absorbance at 480 nm. A standard curve was made with pure kynurenine. The TDO activity was measured as described above using ten serial concentrations of the compounds of the present invention. Data were fitted using the Prism software (GraphPad Software, Inc.).

Compounds 43, 85 and 106 had an IC50<1 μM in this assay.

### II.3. Pharmacodynamic Assay for TDO2 In Vivo Activity Determination: Increase of Blood Tryptophan Levels in Mice

The compounds of the present invention increase the amount of Tryptophan in mouse blood.

Briefly, mice were treated with either a suspension of one of the compounds of the present invention in 0.5% HPMC K4M/0.25% Tween 20 at different doses (30, 60 and 100 mg/kg), or with a vehicle control (0.5% HPMC K4M/0.25% Tween 20), by the oral route by gavage (dosing volume 5 mL/kg, 10 mice per group). After two hours, blood was harvested, plasma was prepared and the amount of Tryptophan present was determined by LC-MS-MS (HPLC column Unison UK-Phenyl, 75×4.6, 3 μm, flow rate 0.8 mL/min, 8 minutes gradient from 95% water+0.1% formic acid/5% Acetonitrile+0.1% formic acid to 5% water+0.1% formic acid/95% Acetonitrile+0.1% formic acid, retention time 2.4 min; API4000 MS-MS system from AB Sciex, ESI+ mode, parent ion 205.1, daughter ion 146.1).

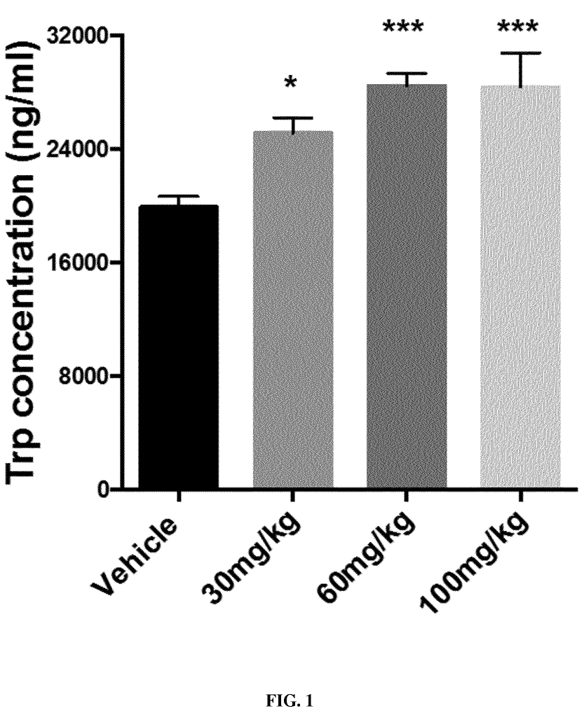

Compound 89 increased circulating Tryptophan by 26% at 30 mg/kg (p<0.05), by 43% at 30 mg/kg (p<0.001) and by 43% at 100 mg/kg (p<0.001) as evidenced in the table below and in FIG. 1.

| | Vehicle | 30 mg/kg | 60 mg/kg | 100 mg/kg |
|---|---|---|---|---|
| Tryptophan | 19892 ± | 25126 ± | 28452 ± | 28374 ± |
| concentration | 759 | 1062 | 871 | 2385 |
| in plasma | ng/mL | ng/mL | ng/mL | ng/mL |
| (average ± | | | | |
| standard error | | | | |
| of the mean) | | | | |

1. A method for treating a patient with bladder cancer associated with tryptophan 2,3-doxygenase (TDO2), comprising delivering to the patient a compound of Formula I: 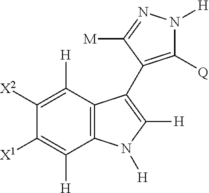
or a pharmaceutically acceptable enantiomer, salt or solvate thereof wherein:
X¹ and X² represent each independently H, halogen or haloalkyl;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, or alkylheteroaryl;
A represents:
a hydrogen atom;
aryl, optionally substituted with halogen, hydroxyl, nitro, amido, carboxy, amino, cyano, haloalkoxy, haloalkyl, alkyl;
heteroaryl, optionally substituted with halogen, hydroxyl, nitro, amido, carboxy, amino, cyano, haloalkoxy, haloalkyl, or alkyl;
C1-C10 alkyl, linear or branched, optionally substituted with up to three substituents selected from the group comprising halogen, hydroxyl, COOR¹, CONR¹R², NR¹COR², NR¹R², SO₂R¹, SO₂NR¹R², NR¹SO₂R², SOR¹, wherein R¹ and R² represent each independently a hydrogen atom or a group, optionally substituted, selected from C1-C6 alkyl, aryl, heteroaryl, amino;
heterocyclyl; optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me;
C1-C3 alkyl-heterocyclyl; wherein both the C1-C3 alkyl and the heterocyclyl are optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², or SOR¹, wherein R¹ and R² represent each independently a hydrogen atom or a group, optionally substituted, selected from C1-C6 alkyl, cycloalkyl, alkene, aryl, heteroaryl and amino, optionally substituted by one or more groups selected from halogen, hydroxyl, alkoxy, COOH, amino, or SO₂Me;
cycloalkyl, optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, or SO₂Me;
C1-C3 alkyl-cycloalkyl, optionally substituted with up to three substituents selected from the group comprising alkyl, the alkyl group being optionally substituted by one or more groups selected from halogen, hydroxyl or COOH; cycloalkyl, halogen, hydroxyl, oxo, alkoxy, COOR¹, COR¹, CONR¹R², NR¹COR², NR¹R², SO₂R¹, SO₂NR¹R², NR¹SO₂R², SOR¹, wherein R¹ and R² represent each independently a group selected from C1-C6 alkyl, cycloalkyl, alkene, aryl, heteroaryl and amino, optionally substituted by one or more groups selected from halogen, hydroxyl, alkoxy, COOH, amino, SO₂Me
under the condition that the compound of Formula I is not:
2-(4-(1H-indol-3-yl)-3,5-dimethyl-1H-pyrazol-1-yl)ethanamine
3-(1-(tert-butyl)-1H-pyrazol-4-yl)-1H-indole
3-(1-ethyl-1H-pyrazol-4-yl)-1H-indole
3-(1-methyl-1H-pyrazol-4-yl)-1H-indole
3-(1-(4-fluorophenyl)-1H-pyrazol-4-yl)-1H-indole
3-(1-(4-chlorophenyl)-1H-pyrazol-4-yl)-1H-indole
3-(1-(4-bromophenyl)-1H-pyrazol-4-yl)-1H-indole
3-(1-(4-methoxyphenyl)-1H-pyrazol-4-yl)-1H-indole
3-(1-(p-tolyl)-1H-pyrazol-4-yl)-1H-indole
3-(1-phenyl-1H-pyrazol-4-yl)-1H-indole
3-(1H-pyrazol-4-yl)-1H-indole
4-(1H-indol-3-yl)-1H-pyrazol-3-ol.

2. The method according to claim 1, the compound having Formula Ia: 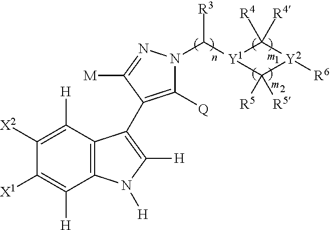
or a pharmaceutically acceptable enantiomer, salt or solvates thereof, wherein:
X¹ and X² represent each independently H, halogen or haloalkyl;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl;
n represents an integer equal to 0, 1, 2 or 3;
m₁ represent each independently an integer equal to 1 or 2;
Y¹ and Y² represent each independently CR⁷, N, O, SO₂, wherein R⁷ represents H or hydroxyl;
R³ represents H, alkyl;
R⁴, R^{4′}, R⁵ and R^{5′} represent each independently H, hydroxyl, alkyl, alkoxy, haloalkyl or R⁴ and R^{4′} form together an oxo moiety or R⁵ and R^{5′} form together an oxo moiety;
R⁶ is absent or represents H, alkyl, the alkyl group being optionally substituted by one or more groups selected from halogen, hydroxyl or COOH; cycloalkyl, halogen, hydroxyl, oxo, COR¹, COOR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me.

3. The method according to claim 1, the compound having Formula Ia-1: 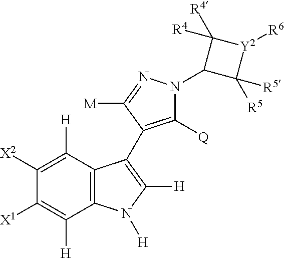
or a pharmaceutically acceptable enantiomer, salt or solvate thereof, wherein:
X¹ and X² represent each independently H or F;
M and Q represent each independently H, C1-C6 alkyl optionally substituted by one or more halogen;
Y² represents N or CH;
R⁴, R^{4′}, R⁵ and R^{5′} represent each independently H, hydroxyl, alkyl, alkoxy, haloalkyl or R⁴ and R^{4′} form together an oxo moiety or R⁵ and R^{5′} form together an oxo moiety;
R⁶ represents H, alkyl, the alkyl group being optionally substituted by one or more groups selected from halogen, hydroxyl or COOH; cycloalkyl, halogen, hydroxyl, oxo, COOR¹, COR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me; preferably R⁶ represents H, COOH, COMe, CONH₂, CONHMe.

4. The method according to claim 1, the compound having Formula Ia-2: 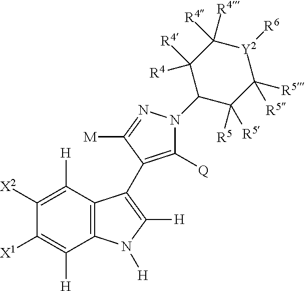
or a pharmaceutically acceptable enantiomer, salt or solvate thereof, wherein:
X¹ and X² represent each independently H or F;
M and Q represent each independently H, C1-C6 alkyl optionally substituted one or more alogen;
Y² represents N or CH; preferably Y is N;
R⁴, R^{4′}, R^{4″}, R^{4′″}, R⁵, R^{5′}, R^{5″} and R^{5′″} represent each independently H, hydroxyl, alkyl, alkoxy, haloalkyl or R⁴ and R^{4′} form together an oxo moiety or R^{4″} and R^{4′″} form together an oxo moiety or R⁵ and R^{5′} form together an oxo moiety or R^{5″} and R^{5′″} form together an oxo moiety, preferably R⁴, R^{4′}, R^{4″}, R^{4′″}, R⁵, R^{5′}, R^{5″} and R^{5′″} represent H or oxo;
R⁶ represents
—H;
alkyl, the alkyl group being optionally substituted by one or more groups selected from halogen, hydroxyl or COOH;
cycloalkyl;
halogen;
hydroxyl;
oxo;
COR¹, SO₂R¹, wherein R¹ represents a group selected from C1-C6 alkyl, or cycloalkylalkene, amino; wherein R¹ groups are optionally substituted by one or more groups selected from halogen; hydroxyl; alkoxy; COOH; amino; or SO₂Me;
COOR¹, wherein R¹ represents a group selected from C1-C6 alkyl cycloalkyl; alkene; or amino; wherein R¹ groups are optionally substituted by one or more groups selected from halogen; hydroxyl; alkoxy; COOH; amino; SO₂Me.

5. The method according to claim 1, the compound having Formula Ia-2′: 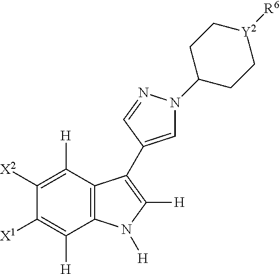
and pharmaceutically acceptable enantiomers, salts and solvates thereof, wherein:
X¹ and X² represent each independently H or F;
Y² represents N or CH; preferably Y is N;
R⁶ represents
—H;
alkyl, the alkyl group being optionally substituted by one or more groups selected from halogen, hydroxyl or COOH;
cycloalkyl;
halogen;
hydroxyl;
oxo;
COR¹, SO₂R¹, wherein R¹ represents a group selected from C1-C6 alkyl; cycloalkyl; or amino; wherein R¹ groups are optionally substituted by one or more groups selected from halogen, alkoxy, or amino.

6. The method according to claim 1, the compound having Formula Ia-3: 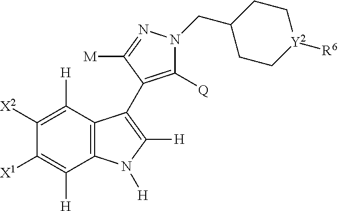
or a pharmaceutically acceptable enantiomer, salt and solvate thereof, wherein:
X¹ and X² represent each independently H or F;
M and Q represent each independently H, C1-C6 alkyl optionally substituted one or more halogen;
Y² represents N or CH;
R⁶ represents
—H;
alkyl, the alkyl group being optionally substituted by one or more groups selected from halogen, hydroxyl or COOH;
cycloalkyl;
COR¹, SO₂R¹, wherein R¹ represents a group selected from C1-C6 alkyl; cycloalkyl; alkene; amino; wherein R¹ groups are optionally substituted by one or more groups selected from halogen; hydroxyl; alkoxy; COOH; amino; SO₂Me.

7. The method according to claim 1, the compound having Formula Ib: 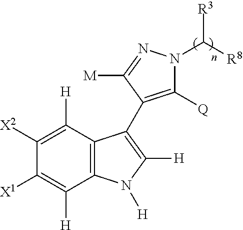
or a pharmaceutically acceptable enantiomer, salt or solvate thereof, wherein:
X¹ and X² represent each independently H, halogen or haloalkyl;
M and Q represent each independently H, halogen, hydroxyl, C1-C6 alkyl optionally substituted by one or more substituents selected from the group comprising halogen, hydroxyl, CONR¹R², NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl;
n represents an integer equal to 1, 2 or 3, preferably 1 or 2;
R³ represents H, alkyl;
R⁸ represents H, alkyl, the alkyl group being optionally substituted by one or more groups selected from halogen, hydroxyl, COOH, CONH₂; cycloalkyl, halogen, hydroxyl, oxo, COR¹, COOR¹, CONR¹R², NR¹COR², NR¹R², SO₂R¹, SO₂NR¹R², NR¹SO₂R², SOR¹, wherein R¹ and R² represent each independently a hydrogen atom or a group selected from C1-C6 alkyl, cycloalkyl, alkene, aryl, heteroaryl and amino, optionally substituted by one or more groups selected from halogen, hydroxyl, alkoxy, COOH, amino, SO₂Me.

8. The method according to claim 1, the compound having Formula Ic: 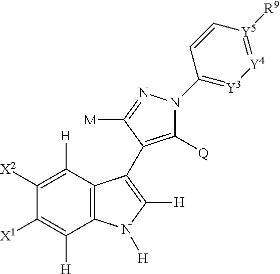
or pharmaceutically acceptable enantiomer, salt or solvate thereof, wherein:
X¹ and X² represent each independently H, halogen or haloalkyl;
M and Q represent each independently H, halogen, hydroxyl, C1-C6 alkyl optionally substituted one or more substituents selected from the group comprising halogen, hydroxyl, CONR¹R¹, NR¹COR² wherein R¹ and R² represent each independently a group, optionally substituted, selected from C1-C6 alkyl, aryl, arylalkyl, alkylaryl, heteroaryl, heteroarylalkyl, alkylheteroaryl;
Y³, Y⁴, Y⁵ represent each independently N or CH;
R⁹ is absent or represents H; halogen; amino.

9. The method according to claim 1, wherein the compound is selected from the group consisting of:
3-(4-(6-fluoro-1H-indol-3-yl)-1H-pyrazol-1-yl)azetidin-2-one,
3-(1-(azetidin-3-yl)-1H-pyrazol-4-yl)-6-fluoro-1H-indole,
1-(3-(4-(6-fluoro-1H-indol-3-yl)-1H-pyrazol-1-yl)azetidin-1-yl)ethanone,
3-(4-(6-fluoro-1H-indol-3-yl)-1H-pyrazol-1-yl)azetidine-1-carboxamide,
3-(4-(6-fluoro-1H-indol-3-yl)-1H-pyrazol-1-yl)-N-methylazetidine-1-carboxamide,
3-(1-(azetidin-3-ylmethyl)-1H-pyrazol-4-yl)-6-fluoro-1H-indole,
3-(4-(6-fluoro-1H-indol-3-yl)-1H-pyrazol-1-yl)propanamide,
3-(4-(5,6-difluoro-1H-indol-3-yl)-1H-pyrazol-1-yl)propanamide,
3-(4-(6-fluoro-1H-indol-3-yl)-3,5-dimethyl-1H-pyrazol-1-yl)propanamide,
3-(4-(6-fluoro-1H-indol-3-yl)-3-methyl-1H-pyrazol-1-yl)propanamide,
3-(4-(6-fluoro-1H-indol-3-yl)-5-methyl-1H-pyrazol-1-yl)propanamide,
3-(4-(1H-indol-3-yl)-5-(trifluoromethyl)-1H-pyrazol-1-yl)propanamide,
N-(2-(dimethylamino)ethyl)-3-(4-(6-fluoro-1H-indol-3-yl)-1H-pyrazol-1-yl)propanamide,
3-(4-(6-fluoro-1H-indol-3-yl)-1H-pyrazol-1-yl)-N-methylpropanamide,
3-(4-(6-fluoro-1H-indol-3-yl)-1H-pyrazol-1-yl)-N,N-dimethylpropanamide,
3-(4-(6-fluoro-1H-indol-3-yl)-1H-pyrazol-1-yl)propanoic acid,
3-(4-(5,6-difluoro-1H-indol-3-yl)-1H-pyrazol-1-yl)propanoic acid,
1-(2-(4-(6-fluoro-1H-indol-3-yl)-1H-pyrazol-1-yl)ethyl)imidazolidin-2-one,
6-fluoro-3-(1-(2-(4-methylpiperazin-1-yl)ethyl)-1H-pyrazol-4-yl)-1H-indole,
4-(2-(4-(6-fluoro-1H-indol-3-yl)-1H-pyrazol-1-yl)ethyl)morpholine,
N-(2-(4-(6-fluoro-1H-indol-3-yl)-1H-pyrazol-1-yl)ethyl)acetamide,
1-(2-(4-(6-fluoro-1H-indol-3-yl)-1H-pyrazol-1-yl)ethyl)urea,
1-(2-(4-(6-fluoro-1H-indol-3-yl)-1H-pyrazol-1-yl)ethyl)-3-methylurea,
2-(4-(6-fluoro-1H-indol-3-yl)-1H-pyrazol-1-yl)-N-methylethanamine,
N-(2-(4-(6-fluoro-1H-indol-3-yl)-1H-pyrazol-1-yl)ethyl)methanesulfonamide,
2-(4-(6-fluoro-1H-indol-3-yl)-1H-pyrazol-1-yl)ethanol,
6-fluoro-3-(1-(2-(piperazin-1-yl)ethyl)-1H-pyrazol-4-yl)-1H-indole,
1-(4-(2-(4-(6-fluoro-1H-indol-3-yl)-1H-pyrazol-1-yl)ethyl)piperazin-1-yl)ethanone,
6-fluoro-3-(1-(2-(pyrrolidin-1-yl)ethyl)-1H-pyrazol-4-yl)-1H-indole,
1-(2-(4-(6-fluoro-1H-indol-3-yl)-1H-pyrazol-1-yl)ethyl)pyrrolidin-2-one,
6-fluoro-3-(1-(2-(methylsulfonyl)ethyl)-1H-pyrazol-4-yl)-1H-indole,
5,6-difluoro-3-(1-(2-(methylsulfonyl)ethyl)-1H-pyrazol-4-yl)-1H-indole,
3-(3,5-dimethyl-1-(2-(methylsulfonyl)ethyl)-1H-pyrazol-4-yl)-6-fluoro-1H-indole,
3-(1-(2-(methylsulfonyl)ethyl)-5-(trifluoromethyl)-1H-pyrazol-4-yl)-1H-indole,
(−)-3-(4-(6-fluoro-1H-indol-3-yl)-1H-pyrazol-1-yl)-2-methylpropanamide,
(+)-3-(4-(6-fluoro-1H-indol-3-yl)-1H-pyrazol-1-yl)-2-methylpropanamide,
3-(4-(6-fluoro-1H-indol-3-yl)-1H-pyrazol-1-yl)-2-hydroxypropanamide,
2-(4-(6-fluoro-1H-indol-3-yl)-1H-pyrazol-1-yl)acetamide,
2-(4-(6-fluoro-1H-indol-3-yl)-1H-pyrazol-1-yl)-N-methylacetamide,
2-(4-(6-fluoro-1H-indol-3-yl)-1H-pyrazol-1-yl)-N,N-dimethylacetamide,
2-(4-(6-fluoro-1H-indol-3-yl)-1H-pyrazol-1-yl)acetic acid,
methyl 2-(4-(6-fluoro-1H-indol-3-yl)-1H-pyrazol-1-yl)acetate,
6-fluoro-3-(1-(piperidin-4-ylmethyl)-1H-pyrazol-4-yl)-1H-indole,
5,6-difluoro-3-(1-(piperidin-4-ylmethyl)-1H-pyrazol-4-yl)-1H-indole,
6-fluoro-3-(1-((1-(2,2,2-trifluoroethyl)piperidin-4-yl)methyl)-1H-pyrazol-4-yl)-1H-indole,
6-fluoro-3-(1-((1-(2-fluoroethyl)piperidin-4-yl)methyl)-1H-pyrazol-4-yl)-1H-indole,
2-(4-((4-(6-fluoro-1H-indol-3-yl)-1H-pyrazol-1-yl)methyl)piperidin-1-yl)ethanol,
1,1,1-trifluoro-3-(4-((4-(6-fluoro-1H-indol-3-yl)-1H-pyrazol-1-yl)methyl)piperidin-1-yl)propan-2-ol,
2-(4-((4-(6-fluoro-1H-indol-3-yl)-1H-pyrazol-1-yl)methyl)piperidin-1-yl)acetic acid,
4-(4-((4-(6-fluoro-1H-indol-3-yl)-1H-pyrazol-1-yl)methyl)piperidin-1-yl)-4-oxobutanoic acid,
1-(4-((4-(6-fluoro-1H-indol-3-yl)-1H-pyrazol-1-yl)methyl)piperidin-1-yl)ethanone,
3-(1-((1-cyclopropylpiperidin-4-yl)methyl)-1H-pyrazol-4-yl)-6-fluoro-1H-indole,
6-fluoro-3-(1-((1-methylpiperidin-4-yl)methyl)-1H-pyrazol-4-yl)-1H-indole,
6-fluoro-3-(1-((1-(methylsulfonyl)piperidin-4-yl)methyl)-1H-pyrazol-4-yl)-1H-indole,
3-(3,5-dimethyl-1-(piperidin-4-ylmethyl)-1H-pyrazol-4-yl)-6-fluoro-1H-indole,
6-fluoro-3-(3-methyl-1-(piperidin-4-ylmethyl)-1H-pyrazol-4-yl)-1H-indole,
6-fluoro-3-(5-methyl-1-(piperidin-4-ylmethyl)-1H-pyrazol-4-yl)-1H-indole,
6-fluoro-3-(1-((tetrahydro-2H-pyran-4-yl)methyl)-1H-pyrazol-4-yl)-1H-indole,
4-((4-(6-fluoro-1H-indol-3-yl)-1H-pyrazol-1-yl)methyl)tetrahydro-2H-pyran-4-ol,
4-((4-(6-fluoro-1H-indol-3-yl)-1H-pyrazol-1-yl)methyl)tetrahydro-2H-thiopyran 1,1-dioxide,
(1S,3 S)-3-(4-(6-fluoro-1H-indol-3-yl)-1H-pyrazol-1-yl)cyclobutanecarboxamide,
(1R,3R)-3-(4-(6-fluoro-1H-indol-3-yl)-1H-pyrazol-1-yl)cyclobutanecarboxamide,
(1S,3 S)-3-(4-(6-fluoro-1H-indol-3-yl)-1H-pyrazol-1-yl)-N-methylcyclobutanecarboxamide,
(1R,3R)-3-(4-(6-fluoro-1H-indol-3-yl)-1H-pyrazol-1-yl)-N-methylcyclobutanecarboxamide,
(1 S,3 S)-3-(4-(6-fluoro-1H-indol-3-yl)-1H-pyrazol-1-yl)cyclobutanecarboxylic acid,
(1R,3R)-3-(4-(6-fluoro-1H-indol-3-yl)-1H-pyrazol-1-yl)cyclobutanecarboxylic acid,
(1R,4R)-4-(4-(6-fluoro-1H-indol-3-yl)-1H-pyrazol-1-yl)cyclohexanecarboxamide,
(1R,4R)-4-(4-(6-fluoro-1H-indol-3-yl)-1H-pyrazol-1-yl)cyclohexanol,
(1R,4R)-4-(4-(6-fluoro-1H-indol-3-yl)-1H-pyrazol-1-yl)cyclohexanol,
6-fluoro-3-(1H-pyrazol-4-yl)-1H-indole,
5,6-difluoro-3-(1H-pyrazol-4-yl)-1H-indole,
3-(1H-pyrazol-4-yl)-6-(trifluoromethyl)-1H-indole,
6-fluoro-3-(1-methyl-1H-pyrazol-4-yl)-1H-indole,
3-(1,5-dimethyl-1H-pyrazol-4-yl)-6-fluoro-1H-indole,
3-(1,3-dimethyl-1H-pyrazol-4-yl)-6-fluoro-1H-indole,
6-fluoro-3-(1,3,5-trimethyl-1H-pyrazol-4-yl)-1H-indole,
3-(1-methyl-5-(trifluoromethyl)-1H-pyrazol-4-yl)-1H-indole,
6-fluoro-3-(1-(piperidin-4-yl)-1H-pyrazol-4-yl)-1H-indole,
2-(4-(4-(6-fluoro-1H-indol-3-yl)-1H-pyrazol-1-yl)piperidin-1-yl)ethanol,
4-(4-(4-(6-fluoro-1H-indol-3-yl)-1H-pyrazol-1-yl)piperidin-1-yl)-4-oxobutanoic acid,
1-(4-(4-(6-fluoro-1H-indol-3-yl)-1H-pyrazol-1-yl)piperidin-1-yl)-3-methoxypropan-1-one,
1-(4-(4-(6-fluoro-1H-indol-3-yl)-1H-pyrazol-1-yl)piperidin-1-yl)propan-1-one,
2-(dimethylamino)-1-(4-(4-(6-fluoro-1H-indol-3-yl)-1H-pyrazol-1-yl)piperidin-1-yl)ethanone,
1-(4-(4-(6-fluoro-1H-indol-3-yl)-1H-pyrazol-1-yl)piperidin-1-yl)-2-hydroxyethanone,
1-(4-(4-(6-fluoro-1H-indol-3-yl)-1H-pyrazol-1-yl)piperidin-1-yl)-2-methoxyethanone,
1-(4-(4-(6-fluoro-1H-indol-3-yl)-1H-pyrazol-1-yl)piperidin-1-yl)-2-methylpropan-1-one,
1-(4-(4-(6-fluoro-1H-indol-3-yl)-1H-pyrazol-1-yl)piperidin-1-yl)-2,2-dimethylpropan-1-one,
cyclopropyl(4-(4-(6-fluoro-1H-indol-3-yl)-1H-pyrazol-1-yl)piperidin-1-yl)methanone,
1-(4-(4-(6-fluoro-1H-indol-3-yl)-1H-pyrazol-1-yl)piperidin-1-yl)ethanone,
4-(4-(6-fluoro-1H-indol-3-yl)-1H-pyrazol-1-yl)-N,N-dimethylpiperidine-1-carboxamide,
6-fluoro-3-(1-(1-methylpiperidin-4-yl)-1H-pyrazol-4-yl)-1H-indole,
6-fluoro-3-(1-(1-((trifluoromethyl)sulfonyl)piperidin-4-yl)-1H-pyrazol-4-yl)-1H-indole,
1-(4-(4-(1H-indol-3-yl)-5-(trifluoromethyl)-1H-pyrazol-1-yl)piperidin-1-yl)ethanone,
1-(4-(4-(6-fluoro-1H-indol-3-yl)-3-methyl-1H-pyrazol-1-yl)piperidin-1-yl)ethanone,
1-(4-(4-(6-fluoro-1H-indol-3-yl)-5-methyl-1H-pyrazol-1-yl)piperidin-1-yl)ethanone,
6-fluoro-3-(1-(1-((2-methoxyethyl)sulfonyl)piperidin-4-yl)-1H-pyrazol-4-yl)-1H-indole,
3-(1-(1-(cyclopropyl sulfonyl)piperidin-4-yl)-1H-pyrazol-4-yl)-6-fluoro-1H-indole,
3-(1-(1-(ethylsulfonyl)piperidin-4-yl)-1H-pyrazol-4-yl)-6-fluoro-1H-indole,
6-fluoro-3-(1-(1-(isopropylsulfonyl)piperidin-4-yl)-1H-pyrazol-4-yl)-1H-indole,
4-(4-(6-fluoro-1H-indol-3-yl)-1H-pyrazol-1-yl)piperidin-2-one,
4-(4-(6-fluoro-1H-indol-3-yl)-1H-pyrazol-1-yl)-1-methylpiperidin-2-one,
1-(4-(4-(6-fluoro-1H-indol-3-yl)-1H-pyrazol-1-yl)piperidin-1-yl)prop-2-en-1-one,
1-(4-(4-(6-fluoro-1H-indol-3-yl)-1H-pyrazol-1-yl)piperidin-1-yl)-4-(methylsulfonyl)butan-1-one,
1-(4-(4-(6-fluoro-1H-indol-3-yl)-1H-pyrazol-1-yl)piperidin-1-yl)-3-hydroxypropan-1-one,
1-(4-(4-(6-fluoro-1H-indol-3-yl)-1H-pyrazol-1-yl)piperidin-1-yl)-3-(methylsulfonyl)propan-1-one,
6-fluoro-3-(1-(1-(methyl sulfonyl)piperidin-4-yl)-1H-pyrazol-4-yl)-1H-indole
6-fluoro-3-(1-(pyridazin-3-yl)-1H-pyrazol-4-yl)-1H-indole,
3-(1-(6-chloropyridazin-3-yl)-1H-pyrazol-4-yl)-6-fluoro-1H-indole,
6-(4-(6-fluoro-1H-indol-3-yl)-1H-pyrazol-1-yl)pyridazin-3-amine,
6-fluoro-3-(1-(pyridazin-4-yl)-1H-pyrazol-4-yl)-1H-indole,
6-fluoro-3-(1-(pyridin-2-yl)-1H-pyrazol-4-yl)-1H-indole,
6-fluoro-3-(1-(pyridin-3-yl)-1H-pyrazol-4-yl)-1H-indole,
6-fluoro-3-(1-(pyridin-4-yl)-1H-pyrazol-4-yl)-1H-indole,
(1R,4R)-4-(4-(6-fluoro-1H-indol-3-yl)-1H-pyrazol-1-yl)-N,N-dimethylcyclohexanecarboxamide,
(1 S,4 S)-4-(4-(6-fluoro-1H-indol-3-yl)-1H-pyrazol-1-yl)cyclohexanecarboxamide,
(1 S,4 S)-4-(4-(6-fluoro-1H-indol-3-yl)-1H-pyrazol-1-yl)-N-methylcyclohexanecarboxamide,
(1 S,4 S)-4-(4-(6-fluoro-1H-indol-3-yl)-1H-pyrazol-1-yl)-N,N-dimethylcyclohexanecarboxamide,
(1R,4R)-4-(4-(6-fluoro-1H-indol-3-yl)-1H-pyrazol-1-yl)-N-methylcyclohexanecarboxamide,
4-(4-(6-fluoro-1H-indol-3-yl)-1H-pyrazol-1-yl)piperidine-1-carboxamide,
methyl 4-(4-(6-fluoro-1H-indol-3-yl)-1H-pyrazol-1-yl)piperidine-1-carboxylate,
2-amino-1-(4-(4-(6-fluoro-1H-indol-3-yl)-1H-pyrazol-1-yl)piperidin-1-yl)ethanone,
(S)-2-amino-1-(4-(4-(6-fluoro-1H-indol-3-yl)-1H-pyrazol-1-yl)piperidin-1-yl)-3-methylbutan-1-one,
3-amino-1-(4-(4-(6-fluoro-1H-indol-3-yl)-1H-pyrazol-1-yl)piperidin-1-yl)propan-1-one,
(R)-2-amino-1-(4-(4-(6-fluoro-1H-indol-3-yl)-1H-pyrazol-1-yl)piperidin-1-yl)-3-methylbutan-1-one,
4-(4-(6-fluoro-1H-indol-3-yl)-1H-pyrazol-1-yl)-N-methylpiperidine-1-carboxamide, or
(S)-2-amino-1-(4-(4-(6-fluoro-1H-indol-3-yl)-1H-pyrazol-1-yl)piperidin-1-yl)propan-1-one,
or a pharmaceutically acceptable enantiomer, salt or solvate thereof.

10-14. (canceled)

15. The method according to claim 1, wherein X¹ and X² represent each independently H, F or CF₃.

16. The method according to claim 15, wherein X¹ and X² represent each independently H or F.

17. The method according to claim 1, wherein M and Q represent each independently H, methyl or CF₃.

18. The method according to claim 17, wherein M and Q represent each independently H or methyl.

19. The method according to claim 1, wherein A represents a heteroaryl which is selected from a substituted or unsubstituted pyridyl or pyridazine.

20. The method according to claim 19, wherein A represents substituted or unsubstituted pyridyl.

21. The method according to claim 1, wherein A represents a heterocyclyl selected from azetidine, piperidine, morpholine, piperazine, tetrahydrofurane, tetrahydropyrane, tetrahydro-thiopyran-dioxide, dioxane, imidazolidinone, pyrrolidine, or pyrrolidinone.

22. The method according to claim 1, wherein A represents C1-C3 alkyl-heterocyclyl selected from azetidine, piperidine, morpholine, piperazine, tetrahydrofurane, tetrahydropyrane, tetrahydro-thiopyran-dioxide, dioxane, imidazolidinone, pyrrolidine, or pyrrolidinone.

23. The method according to claim 1, wherein A represents a cycloalkyl selected from cyclobutane or cyclohexyl,

24. The method according to claim 3, wherein R⁴, R^{4′}, R⁵ and R^{5′} represent H or oxo.

25. The method according to claim 4, wherein R⁶ represents methyl or —CH₂—CH₂—OH.

26. The method according to claim 4, wherein R¹ represents Me, Et, iPr, or tBu.

27. The method according to claim 4, wherein R⁶ represents cycloalkyl which is cyclopropane.

28. The method according to claim 4, wherein R⁶ represents the alkene which is ethylene.

29. The method according to claim 4, wherein R⁶ represents the amino and is NMe₂.

30. A 4-(indol-3-yl)-pyrazole compound selected from the group consisting of:
(1R,4R)-4-(4-(6-fluoro-1H-indol-3-yl)-1H-pyrazol-1-yl)-N,N-dimethylcyclohexanecarboxamide,
(1 S,4 S)-4-(4-(6-fluoro-1H-indol-3-yl)-1H-pyrazol-1-yl)cyclohexanecarboxamide,
(1 S,4 S)-4-(4-(6-fluoro-1H-indol-3-yl)-1H-pyrazol-1-yl)-N-methylcyclohexanecarboxamide,
(1S,4 S)-4-(4-(6-fluoro-1H-indol-3-yl)-1H-pyrazol-1-yl)-N,N-dimethylcyclohexanecarboxamide,
(1R,4R)-4-(4-(6-fluoro-1H-indol-3-yl)-1H-pyrazol-1-yl)-N-methylcyclohexanecarboxamide,
4-(4-(6-fluoro-1H-indol-3-yl)-1H-pyrazol-1-yl)piperidine-1-carboxamide,
methyl 4-(4-(6-fluoro-1H-indol-3-yl)-1H-pyrazol-1-yl)piperidine-1-carboxylate,
2-amino-1-(4-(4-(6-fluoro-1H-indol-3-yl)-1H-pyrazol-1-yl)piperidin-1-yl)ethanone,
(S)-2-amino-1-(4-(4-(6-fluoro-1H-indol-3-yl)-1H-pyrazol-1-yl)piperidin-1-yl)-3-methylbutan-1-one,
3-amino-1-(4-(4-(6-fluoro-1H-indol-3-yl)-1H-pyrazol-1-yl)piperidin-1-yl)propan-1-one,
(R)-2-amino-1-(4-(4-(6-fluoro-1H-indol-3-yl)-1H-pyrazol-1-yl)piperidin-1-yl)-3-methylbutan-1-one,
4-(4-(6-fluoro-1H-indol-3-yl)-1H-pyrazol-1-yl)-N-methylpiperidine-1-carboxamide,
(S)-2-amino-1-(4-(4-(6-fluoro-1H-indol-3-yl)-1H-pyrazol-1-yl)piperidin-1-yl)propan-1-one,
or a pharmaceutically acceptable enantiomer, salt or solvate thereof

31. A pharmaceutical composition compound according to claim 30, or a pharmaceutically acceptable enantiomer, salt or solvate thereof, and at least one pharmaceutically acceptable carrier, diluent, excipient and/or adjuvant.

32. A method of inhibiting tryptophan-2,3-dioxygenase (TDO2), comprising contacting TDO2 with the compound of claim 30 or a pharmaceutically acceptable enantiomer or salt thereof.
